SEQUENCE LISTING (120 sequences)

SEQ ID NO: 1 - protein, 9 aa, Artificial Sequence
  Synthetic
GGSGGSGGS

SEQ ID NO: 2 - protein, 30 aa, Artificial Sequence
  Synthetic
GCCTGTCTTTGCCTGTCTTTGCCTGTCTTT

SEQ ID NO: 3 - protein, 8 aa, Artificial Sequence
  Synthetic
GTGTCCCC

SEQ ID NO: 4 - protein, 5 aa, Artificial Sequence
  Synthetic
APRPG

SEQ ID NO: 5 - protein, 8 aa, Artificial Sequence
  Synthetic
GPLGIAGQ

SEQ ID NO: 6 - protein, 5 aa, Artificial Sequence
  Synthetic
RGBYC

SEQ ID NO: 7 - protein, 6 aa, Artificial Sequence
  Synthetic
KCCYSL

SEQ ID NO: 8 - protein, 7 aa, Artificial Sequence
  Synthetic
YEVGHRC

SEQ ID NO: 9 - DNA, 31 bp, Artificial Sequence
  Synthetic
ccccttcagtctggaaaggaggacactgagg

SEQ ID NO: 10 - DNA, 31 bp, Artificial Sequence
  Synthetic
ccccttcagtatggaaaggaggacactgagg

SEQ ID NO: 11 - DNA, 31 bp, Artificial Sequence
  Synthetic
atcagtccagccccttcagtctggaaaggag

SEQ ID NO: 12 - DNA, 31 bp, Artificial Sequence
  Synthetic
atcagtccagccccttcagtatggaaaggag

SEQ ID NO: 13 - DNA, 36 bp, Artificial Sequence
  Synthetic
aatcagtccagccccttcagtctggaaaggaggaca

SEQ ID NO: 14 - DNA, 37 bp, Artificial Sequence
  Synthetic
aatcagtccagccccttcaagtctggaaaggaggaca

SEQ ID NO: 15 - DNA, 37 bp, Artificial Sequence
  Synthetic
aatcagtccagccccttcacgtctggaaaggaggaca

SEQ ID NO: 16 - DNA, 34 bp, Artificial Sequence
  Synthetic
caatcagtccagccccttcagtctggaaaggagg

SEQ ID NO: 17 - DNA, 37 bp, Artificial Sequence
  Synthetic
caatcagtccagccccttcacgggtctggaaaggagg

SEQ ID NO: 18 - DNA, 35 bp, Artificial Sequence
  Synthetic
caatcagtccagccccttcaagtctggaaaggagg

SEQ ID NO: 19 - DNA, 30 bp, Artificial Sequence
  Synthetic
cagtccagccccttcagtctggaaaggagg

SEQ ID NO: 20 - DNA, 31 bp, Artificial Sequence
  Synthetic
tcagtccagccccttcagtctggaaaggagg

SEQ ID NO: 21 - DNA, 32 bp, Artificial Sequence
  Synthetic
tcagtccagccccttcaagtctggaaaggagg

SEQ ID NO: 22 - DNA, 30 bp, Artificial Sequence
  Synthetic
tcagtccagccccttcagtctggaaaggag

SEQ ID NO: 23 - DNA, 31 bp, Artificial Sequence
  Synthetic
tcagtccagccccttcaagtctggaaaggag

SEQ ID NO: 24 - DNA, 31 bp, Artificial Sequence
  Synthetic
tcagtccagcccctttcagtctggaaaggag

SEQ ID NO: 25 - DNA, 31 bp, Artificial Sequence
  Synthetic
tcagtccagccccttcaggtctggaaaggag

SEQ ID NO: 26 - DNA, 31 bp, Artificial Sequence
  Synthetic
tcagtccagccccttcatgtctggaaaggag

SEQ ID NO: 27 - protein, 15 aa, Artificial Sequence
  Synthetic
GGGGGGGGGGGGGGG

SEQ ID NO: 28 - protein, 15 aa, Artificial Sequence
  Synthetic
GGGGSGGGGSGGGGS

SEQ ID NO: 29 - protein, 7 aa, Simian virus 40
PKKKRKV

SEQ ID NO: 30 - protein, 11 aa, Simian virus 40
PKKKRKVEASS

SEQ ID NO: 31 - protein, 16 aa, Artificial Sequence
  Synthetic
KRPAATKKAGQAKKKK

SEQ ID NO: 32 - protein, 9 aa, Homo sapiens
PAAKRVKLD

SEQ ID NO: 33 - protein, 11 aa, Homo sapiens
RQRRNELKRSP

SEQ ID NO: 34 - protein, 38 aa, Homo sapiens
NQSSNFGPMKGGNFGGRSSGPYGGGGQYFAKPRNQGGY

SEQ ID NO: 35 - protein, 42 aa, Homo sapiens
RMRIZFKNKGKDTAELRRRRVEVSVELRKAKKDEQILKRRNV

SEQ ID NO: 36 - protein, 8 aa, Homo sapiens
VSRKRPRP

SEQ ID NO: 37 - protein, 8 aa, Homo sapiens
PPKKARED

SEQ ID NO: 38 - protein, 8 aa, Homo sapiens
PQPKKKPL

SEQ ID NO: 39 - protein, 12 aa, Mus sp.
SALIKKKKKMAP

SEQ ID NO: 40 - protein, 5 aa, Influenza virus
DRLRR

SEQ ID NO: 41 - protein, 7 aa, Influenza virus
PKQKKRK

SEQ ID NO: 42 - protein, 10 aa, Hepatitis D virus
RKLKKKIKKL

SEQ ID NO: 43 - protein, 10 aa, Mus sp.
REKKKFLKRR

SEQ ID NO: 44 - protein, 20 aa, Homo sapiens
KRKGDEVDGVDEVAKKKSKK

SEQ ID NO: 45 - protein, 17 aa, Homo sapiens
RKCLQAGMNLEARKTKK

SEQ ID NO: 46 - protein, 401 aa, Ktedonobacter racemifer
MSTDATLIRTTPSHAEADATDTLVATPLMPPRRVISPWPGPGEGQSLMRIPVVDIRGMALMPCTPAKARHLLKSGNARPKRNKLGLFYVQLSYEQEPDNQSLVAGVDPGSKFEGLSVVGTKDTVLNLMVEAPDHVKGAVQTRRTMRRARRQRKWRRPKRFHNRLNRMQRIPPSTRSRWEAKARIVAHLRTILPFTDVVVEDVQAVTRKGKGGTWNGSFSPVQVGKEHLYRLLRAMGLTLHLREGWQTKELREQHGLKKTKSKSKQSFESHAVDSWVLAASISGAEHPTCTRLWYMVPAILHRRQLHRLQASKGGVRKPYGGTRSLGVKRGTLVEHKKYGRCTVGGVDRKRNTISLHEYRTNTRLTQAAKVETCRVLTWLSWRSWLLRGKRTSSKGKGSHSS

SEQ ID NO: 47 - protein, 432 aa, Oscillatoriales cyanobacterium
MQPAKQQNWVFQINGDKQPLDMINPGRCRELQNRGKLASFRRFPYVVIQQQTIENPQTKEYILKIDPGSQWTGFAIQCGNDILFRAELNHRGEAIKFDLVKRAWFRRGRRSRNLRYRKKRLNRAKPEGWLAPSIRHRVLTVETWIKRFMRYCPIAWIEIEQVRFDTQKLANPEIDGVEYQQGELQGYEVREYLLQKWGRKCAYCGTENVPLEVEHIQSKSKGGSSRIGNLTLACHVCNVKKGNLDVRDFLAKSPDILNQVLENSTKPLKDAAAVNSTRYAIVKMAKSICENVKCSSGARTKMNRVRQGLEKTHSLDAACVGESGASIRVLTDRPLLITCKGHGSRQSIRVNASGFPAVKNAKTVFTHIAAGDVVRFTIGKDRKKAQAGTYTARVKTPTPKGFEVLIDGARISLSTMSNVVFVHRSDGYGYEL

SEQ ID NO: 48 - protein, 461 aa, Acidhalobacter prosperus
MAVFVIDKHKRPLMPCSEKRARLLLERGRAVVHRQVPFVIRLKDRTVQHSAVQPLRVALDPGSRATGMALVREKNTVDTGTGEVYRERIALNLFELVHRGHRIREQLDQRRNFRRRRRGANLRYRAPRFDNRRRPPGWLAPSLQHRVDTTMAWVRRLCRWAPASAIGIETVRFDTQRLQNPEISGVEYQQGALAGCEVREYLLEKWGRKCAYCGAENVPLEIEHIVPKSRGGSDRVSNLALACRACNQAKGNRDVRAFLADQPERLARILAQAKAPLKDAAAVNATRWALYRALVDTGLPVEAGTGGRTKWNRTRLGLPKTHALDALCVGQVDQVRHWRVPVLGIRCAGRGSYRRTRLTRHGFPRGYLTRNKSAFGFQTGDLIRAVVTKGKKAGTYLGRIAIRASGSFNIQTPMGVVQGIHHRFCTLLQRADGYGYFVQPKPTEAALSSPRLKAGVSSAGN

SEQ ID NO: 49 - protein, 439 aa, Tolypothrix campylonemoides
MTTNVVFVIDTNQKPLQPCSAAVARKLLLRGKAAMFRRYPAVIILKKEVDSVGKPKIELRIDPGSKYTGFALVDSKDNADFIIWGTELEHRGAAICKELTKRSAIRRSRRNRKTRYRKKRFERRKPEGWLAPSLQHRVDTTLTWVKRICKFVPIMSISVEQVKFDLQKLENSDIQGIEYQQGTLAGYTLREALLEHWGRKCAYCDVENVFLEIEHIYPKSKGGSDKFSNLTLACHKCNINKGNKSIDEFLLSDHKRLEQIKLHQKKTLKDAAAVNATRKKLVTTLQEKTFLNVLVSDGASTKMTRLSSSLAKRHWIDAGCVNTTLIVILKTLQPLQVKCNGHGNKQFVTMDAYGFPRKSYEPKKVRKDWKAGDIIRVTKKDGTMLMGRVKKAAKKLVYIPFGGKEASFSSENAKAIHRSDGYRYSFAAIDSELLQKMAT

SEQ ID NO: 50 - protein, 419 aa, Halobacteroides halobius
MPNKYAFVLDSKGKLLDPTKSKKAWYLIRKGKASLVEEYPLIIKLKREVPKDQVNSDKLILGIDDGTKKVGFALVQKCQTKNKVLFKAVMEQRQDVSKKMEERRGYRRYRRSHKRYRPARFDNRSSSKRKGRIPPSILQKKQAILRVVNKLKKYIRIDKIVLEDVSIDIRKLTEGRELYNWEYQESNRLDENLRKATLYRDDCTCQLCGTTETMLHAHHIMPRRDGGADSIYNLITLCKACHKDKVDNNEYQYKDQFLAIIDSKELSDLKSASHVMQGKTWLRDKLSKIAQLEITSGGNTANKRIDYEIEKSHSNDAICTTGLLPVDNIDDIKEYYIKPLRKKSKAKIKELKCFRQRDLVKYTKRNGETYTGYITSLRIKNNKYNSKVCNFSTLKGKIFRGYGFRNLTLLNRPKGLMIV

SEQ ID NO: 51 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 52 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 53 - protein, 1365 aa, Streptococcus pyogenes
KYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 54 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 55 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 56 - protein, 5 aa, Artificial Sequence
  Synthetic
VITYF

SEQ ID NO: 57 - protein, 5 aa, Artificial Sequence
  Synthetic
VVVVV

SEQ ID NO: 58 - protein, 7 aa, Artificial Sequence
  Synthetic
KPWWPRR

SEQ ID NO: 59 - protein, 9 aa, Artificial Sequence
  Synthetic
NIVNVSLVK

SEQ ID NO: 60 - protein, 5 aa, Artificial Sequence
  Synthetic
LPFFD

SEQ ID NO: 61 - protein, 4 aa, Artificial Sequence
  Synthetic
KLVF

SEQ ID NO: 62 - protein, 7 aa, Artificial Sequence
  Synthetic
LVEALYL

SEQ ID NO: 63 - protein, 6 aa, Artificial Sequence
  Synthetic
RGFFYT

SEQ ID NO: 64 - DNA, 18 bp, Artificial Sequence
  Synthetic
aggaaccctgggggccca

SEQ ID NO: 65 - DNA, 18 bp, Artificial Sequence
  Synthetic
cgtggcctcccgagggtg

SEQ ID NO: 66 - DNA, 24 bp, Artificial Sequence
  Synthetic
acactgagacttctagaagactgg

SEQ ID NO: 67 - DNA, 22 bp, Artificial Sequence
  Synthetic
gcttaatcttccgataacgtga

SEQ ID NO: 68 - DNA, 27 bp, Artificial Sequence
  Synthetic
gctatgacactattggaagactggtgc

SEQ ID NO: 69 - DNA, 18 bp, Artificial Sequence
  Synthetic
ccggacctccccgtgaag

SEQ ID NO: 70 - DNA, 21 bp, Artificial Sequence
  Synthetic
gagctggaccatatgaccacc

SEQ ID NO: 71 - DNA, 21 bp, Artificial Sequence
  Synthetic
tcactgtcctgggtcggactt

SEQ ID NO: 72 - DNA, 22 bp, Artificial Sequence
  Synthetic
gctagcatcctttcacgtttgg

SEQ ID NO: 73 - DNA, 22 bp, Artificial Sequence
  Synthetic
gtaaccggatagacttggtgtc

SEQ ID NO: 74 - DNA, 22 bp, Artificial Sequence
  Synthetic
atgaaacagcgaaggaagcctc

SEQ ID NO: 75 - DNA, 20 bp, Artificial Sequence
  Synthetic
tcggtgacgtaggtcgtgta

SEQ ID NO: 76 - DNA, 20 bp, Artificial Sequence
  Synthetic
gccgtggctctattagatga

SEQ ID NO: 77 - DNA, 20 bp, Artificial Sequence
  Synthetic
gggaactcaggaaagcactc

SEQ ID NO: 78 - DNA, 22 bp, Artificial Sequence
  Synthetic
gttatcacattcctccaggacg

SEQ ID NO: 79 - DNA, 18 bp, Artificial Sequence
  Synthetic
gtcgtaccagtggcggta

SEQ ID NO: 80 - DNA, 18 bp, Artificial Sequence
  Synthetic
gctgctgcgggtggttcc

SEQ ID NO: 81 - DNA, 18 bp, Artificial Sequence
  Synthetic
agtaggacgtcacgacgc

SEQ ID NO: 82 - DNA, 20 bp, Artificial Sequence
  Synthetic
gatagaaccctctgaagact

SEQ ID NO: 83 - DNA, 20 bp, Artificial Sequence
  Synthetic
tgagtctactacttgaactg

SEQ ID NO: 84 - DNA, 18 bp, Artificial Sequence
  Synthetic
gctgctgcgggtggttcc

SEQ ID NO: 85 - DNA, 18 bp, Artificial Sequence
  Synthetic
agtaggacgtcacgacgc

SEQ ID NO: 86 - DNA, 20 bp, Artificial Sequence
  Synthetic
gcagcccctatcccaaactt

SEQ ID NO: 87 - DNA, 20 bp, Artificial Sequence
  Synthetic
gcactgttcgtacgactagc

SEQ ID NO: 88 - DNA, 21 bp, Artificial Sequence
  Synthetic
gcttctggtgcatctggcaac

SEQ ID NO: 89 - DNA, 24 bp, Artificial Sequence
  Synthetic
tgggacaaacaggctcgatactaa

SEQ ID NO: 90 - DNA, 18 bp, Artificial Sequence
  Synthetic
ccagcgaaagcctccaag

SEQ ID NO: 91 - DNA, 22 bp, Artificial Sequence
  Synthetic
ctctgcggcgttttacttaaac

SEQ ID NO: 92 - DNA, 25 bp, Artificial Sequence
  Synthetic
tgtgcctgttgtaatcacctggtct

SEQ ID NO: 93 - DNA, 24 bp, Artificial Sequence
  Synthetic
gtcggtggtaagaagatggtgtac

SEQ ID NO: 94 - DNA, 24 bp, Artificial Sequence
  Synthetic
tgtttacagtgccacaaccgaatt

SEQ ID NO: 95 - DNA, 22 bp, Artificial Sequence
  Synthetic
gaggtctagtcgtactcgtaga

SEQ ID NO: 96 - DNA, 20 bp, Artificial Sequence
  Synthetic
aggtgtcccgtaggtactga

SEQ ID NO: 97 - DNA, 20 bp, Artificial Sequence
  Synthetic
gggtggagcttctagaacaa

SEQ ID NO: 98 - DNA, 20 bp, Artificial Sequence
  Synthetic
gcgagtaggcgcggaaggcg

SEQ ID NO: 99 - DNA, 20 bp, Artificial Sequence
  Synthetic
ggcccgtgggcggcagctcg

SEQ ID NO: 100 - DNA, 20 bp, Artificial Sequence
  Synthetic
agcgtgcttcgcgagcagcg

SEQ ID NO: 101 - DNA, 20 bp, Artificial Sequence
  Synthetic
cgctgctcgcgaagcacgct

SEQ ID NO: 102 - DNA, 20 bp, Artificial Sequence
  Synthetic
gggcgcggcatgcactgctt

SEQ ID NO: 103 - DNA, 20 bp, Artificial Sequence
  Synthetic
agcaatttaggttctcaaga

SEQ ID NO: 104 - DNA, 20 bp, Artificial Sequence
  Synthetic
tgcagatgctgatgcatatg

SEQ ID NO: 105 - DNA, 20 bp, Artificial Sequence
  Synthetic
tctgtatccggttatgcacc

SEQ ID NO: 106 - DNA, 20 bp, Artificial Sequence
  Synthetic
gctttcacggaggttcgacg

SEQ ID NO: 107 - DNA, 20 bp, Artificial Sequence
  Synthetic
atgttgcagttcggctcgat

SEQ ID NO: 108 - DNA, 20 bp, Artificial Sequence
  Synthetic
agaggggcttcactcccctg

SEQ ID NO: 109 - DNA, 295 bp, Artificial Sequence
  Synthetic
gctaatagcgactgctctgaatgaatatgttgaatgtatgcttctgttgtcatttacaggaacaggcgggttttaagaaccaaaagacgccaaccacgagggtcccaggatccagggctccctccccaggccaccatgtatccctatgacgtgcccgattatgccgctgctgcgggtggttcctccaactgcccgccccctccccctccccctcctcccaacaacaacaacaacaacaacaccccaaagagcccaggcgtgcctgacgccgaagatgatgatgaacgcagacacg

SEQ ID NO: 110 - DNA, 20 bp, Artificial Sequence
  Synthetic
tctttttcaaagcggctggc

SEQ ID NO: 111 - DNA, 19 bp, Artificial Sequence
  Synthetic
tcgaggttggtggtgttca

SEQ ID NO: 112 - DNA, 20 bp, Artificial Sequence
  Synthetic
ggtgagtgagtgtgtgcgtg

SEQ ID NO: 113 - DNA, 20 bp, Artificial Sequence
  Synthetic
gcagcgttacctctatcgta

SEQ ID NO: 114 - DNA, 32 bp, Artificial Sequence
  Synthetic
  misc_feature (29)..(29): n is a, c, g, or t
accagtcttcagagcacactcctttccanacc

SEQ ID NO: 115 - protein, 11 aa, Artificial Sequence
  Synthetic
TSLQSTLLSIP

SEQ ID NO: 116 - DNA, 57 bp, Artificial Sequence
  Synthetic
  misc_feature (9)..(9): n is a, c, g, or t
ccacgacgngatagaaccctctgaagactcatttgagacgatgatggagcttaagaa

SEQ ID NO: 117 - protein, 19 aa, Artificial Sequence
  Synthetic
  misc_feature (3)..(3): Xaa can be any naturally occurring amino acid
DAXICPSCDSFCTMMCLKH

SEQ ID NO: 118 - DNA, 109 bp, Artificial Sequence
  Synthetic
accagtcttcagagcacactcctttccacaccatgtatccctatgacgtgccccattatgccatagaaccctctgaagactcatttgagacgatgatggagcttaagaa

SEQ ID NO: 119 - RNA, 80 nt, Artificial Sequence
  Synthetic
ucuuuacaaagaagcugauacaaacacaggccaugcugauuucuuacagagggagagaagaggaagagaagaagaaagag

SEQ ID NO: 120 - DNA, 19 bp, Artificial Sequence
  Synthetic
  misc_feature (10)..(10): n is a, c, g, or t
cctgtctttnaacactccc

(19) United States
(12) Patent Application Publication
(10) Pub. No.: US 2024/0084330 A1
(43) Pub. Date: Mar. 14, 2024

(54) COMPOSITIONS AND METHODS FOR DELIVERING CARGO TO A TARGET CELL

(71) Applicants: THE BROAD INSTITUTE, INC., Cambridge, MA (US); MASSACHUSETTS INSTITUTE OF TECHNOLOGY, Cambridge, MA (US)

(72) Inventors: Feng Zhang, Cambridge, MA (US); Michael Segel, Cambridge, MA (US); Blake Lash, Cambridge, MA (US)

(21) Appl. No.: 18/272,158

(22) PCT Filed: Jan. 28, 2022
(86) PCT No.: PCT/US2022/014428

§ 371 Date: Jul. 13, 2023

Related U.S. Application Data
(60) Provisional application No. 63/142,852, filed on Jan. 28, 2021.
(60) Provisional application No. 63/191,067, filed on May 20, 2021.
(60) Provisional application No. 63/234,591, filed on Aug. 18, 2021.
(60) Provisional application No. 63/296,444, filed on Jan. 4, 2022.

(51) Int. Cl. C12N 15/88; C07K 14/005; C07K 14/47; C12N 9/22; C12N 15/11; C12N 15/86

(52) U.S. Cl. CPC C12N 15/88; C07K 14/005; C07K 14/47; C12N 9/22; C12N 15/11; C12N 15/86; C12N 2310/20; C12N 2740/16023; C12N 2740/16043; C12N 2740/16045; C12N 2760/20222

(57) ABSTRACT

Provided herein are compositions, systems, and methods for delivering cargo to a target cell. The compositions, systems, and methods comprise one or more polynucleotides encoding one or more LTR retroelement polypeptides for forming a delivery vesicle and one or more capture moieties for packaging a cargo within the delivery vesicle. The one or more LTR retroelement polypeptides for forming a delivery vesicle may comprise two or more of an LTR retroelement gag protein, a retroelement envelope protein, a an LTR retroelement reverse transcriptase, or a combination thereof. The LTR retroelement polypeptide alone, the LTR retroelement envelope protein alone, or both the LTR retroelement-derived polypeptide and LTR retroelement envelope protein may be endogenous.

### CROSS-REFERENCE TO RELATED APPLICATIONS

This application claims the benefit of U.S. Provisional Application No. 63/142,852, filed Jan. 28, 2021; U.S. Provisional Application No. 63/191,067, filed May 20, 2021; U.S. Provisional Application No. 63/234,591, filed Aug. 18, 2021; and U.S. Provisional Application No. 63/296,444, filed Jan. 4, 2022. The entire contents of the above-identified applications are hereby fully incorporated herein by reference.

### STATEMENT REGARDING FEDERALLY SPONSORED RESEARCH

This invention was made with government support under Grant No. HL141201 and HG009761 awarded by the National Institutes of Health. The government has certain rights in the invention.

### REFERENCE TO AN ELECTRONIC SEQUENCE LISTING

The contents of the electronic sequence listing (“BROD-5335WP_ST25.txt,” size is 77,703 bytes and it was created on Jan. 28, 2022) is herein incorporated by reference in its entirety.

### TECHNICAL FIELD

The subject matter disclosed herein is generally directed to engineered delivery agents, compositions, systems and uses thereof.

### BACKGROUND

Delivery systems are important aspects to efficacy of a treatment. Delivery of therapeutics to the inside of a cell presents many challenges, including but not limited to, limiting off-target effects, delivery efficiency, degradation, and the like. Viruses and virus-like particles have been used to deliver various cargos (e.g., gene therapy agents) to target cells. However, currently used vesicles and particles may be large in size and difficult to generate in a consistent manner. As such, there exists a need for simpler and improved delivery systems.

### SUMMARY

Described in certain example embodiments herein are engineered delivery vesicle generation systems comprising (a) a polynucleotide encoding an endogenous long-terminal repeat (LTR) retroelement polypeptide comprising a capsid domain, a nucleocapsid domain, a protease domain, and a reverse transcriptase domain; (b) one or more heterologous cargo polynucleotides; and (c) one or more packaging elements operatively coupled to the one or more heterologous cargo polynucleotides.

In certain example embodiments, the engineered delivery vesicle generation system further comprises (d) a polynucleotide encoding a fusogenic polypeptide.

In certain example embodiments, the endogenous LTR retroelement polypeptide is an endogenous Gag polypeptide, optionally a Sushi family polypeptide or orthologue thereof. In certain example embodiments, the Gag polypeptide is a PEG10 polypeptide or orthologue thereof, an RTL1 polypeptide or orthologue thereof, an RTL3 polypeptide or orthologue thereof, an RTL5 polypeptide or orthologue thereof, an RTL6 polypeptide or orthologue thereof, or any combination thereof.

In certain example embodiments, the polynucleotide encoding the endogenous LTR retroelement polypeptide comprises one or more modifications that enhance binding specificity and/or packaging of the cargo polynucleotide and/or reduce endogenous LTR retroelement polypeptide binding to an endogenous LTR retroelement polypeptide mRNA. In certain example embodiments, the one or more modifications are in the polynucleotide encoding the endogenous LTR retroelement polypeptide at the boundary between the nucleocapsid domain encoding region and protease domain encoding region.

In certain example embodiments, the one or more packaging elements are capable of complexing with one or more domains of the endogenous LTR retroelement polypeptide.

In certain example embodiments, the one or more packaging elements comprise one or more 5′ untranslated regions (UTRs) or portion thereof, one or more 3′ UTRs or portion thereof, or both, and wherein one or more the 5′ UTR or portion thereof, the 3′ UTRs or portion thereof or both are capable of complexing with one or more domains of the endogenous LTR retroelement polypeptide, and optionally wherein at least one of the one or more 3′ UTRs or portion thereof comprises about 500 bp of a proximal end of a 3′UTR of an mRNA encoding an endogenous LTR retroelement polypeptide.

In certain example embodiments, one or more of the one or more 5′ UTRs or portion thereof are derived from a UTR of an mRNA encoding an endogenous LTR polypeptide, wherein one or more of the one or more 3′ UTRs are derived from a UTR of an mRNA encoding an endogenous LTR polypeptide, or both.

In certain example embodiments, one or more of the one or more packaging elements comprises at least a 3′ UTR or portion thereof derived from a UTR of an mRNA encoding an endogenous LTR retroelement polypeptide.

In certain example embodiments, the 3′UTR or portion thereof comprises about 500 bp of a proximal end of a 3′UTR of an mRNA encoding an endogenous LTR retroelement polypeptide.

In certain example embodiments, the mRNA encoding an endogenous LTR retroelement polypeptide is an mRNA encoding an endogenous Gag polypeptide, optionally a Sushi family protein. In certain example embodiments, the mRNA encoding an endogenous LTR retroelement polypeptide is an mRNA encoding a PEG10 polypeptide or orthologue thereof, an RTL1 polypeptide or orthologue thereof, an RTL3 polypeptide or orthologue thereof, an RTL5 polypeptide or orthologue thereof, an RTL6 polypeptide or orthologue thereof.

In certain example embodiments, the packaging element is a polynucleotide comprising a polynucleotide motif having a sequence of UNNUU, wherein each N is independently selected from A, T, C, G, or U.

In certain example embodiments, the fusogenic polypeptide is specific for a target cell type to which the cargo polynucleotide is targeted for delivery. In certain example embodiments, the fusogenic polypeptide is a tetraspanin (TSPAN), a G envelope protein, an epsilon-sarcoglycan (SGCE), a syncitin, or a combination thereof. In certain example embodiments, the TSPAN is CD81, CD9, CD63 or a combination thereof. In certain example embodiments, the G envelope protein is a vesicular stomatitis virus G envelope protein (VSV-G).

In certain example embodiments, (a), (b), (c), and (d) are encoded on one or more vectors comprising one or more regulatory elements, and wherein (a), (b), (c) and/or (d) are optionally operatively coupled to the one or more regulatory elements. In certain example embodiments, (a), (b), and (c) are encoded on the same vector.

In certain example embodiments, at least one or more heterologous cargo polynucleotides are RNA, DNA, or hybrid RNA/DNA.

In certain example embodiments, the at least one or more heterologous cargo polynucleotides comprise one or more modifications capable of modifying the functionality, packaging ability, stability, localization, or any combination thereof, of the at least one or more heterologous cargo polynucleotides.

In certain example embodiments, at least one of the one or more heterologous cargo polynucleotides encodes an RNA guided nuclease system or component thereof, optionally an RNA guided nuclease. In certain example embodiments, the RNA guided nuclease system is a Cas-based system or an IscB system and wherein the optional RNA guided nuclease is a Cas polypeptide or an IscB polypeptide. In certain example embodiments, at least one of the one or more heterologous cargo polynucleotides comprises a guide polynucleotide and/or a polynucleotide encoding a guide polynucleotide, optionally wherein the guide polynucleotide and/or the polynucleotide encoding a guide polynucleotide are operatively coupled to one or more of the one or more packaging elements. In certain example embodiments, at least one of the one or more heterologous cargo polynucleotides that encodes an RNA guided nuclease further comprises a guide polynucleotide or a polynucleotide encoding a guide polynucleotide. In certain example embodiments, the guide polynucleotide or the polynucleotide encoding a guide polynucleotide is operatively coupled to the same packaging elements as one or more at least one heterologous cargo polynucleotides that encodes an RNA guided nuclease.

In certain example embodiments, the polynucleotide encoding an endogenous long-terminal repeat (LTR) retroelement polypeptide, optionally the capsid domain, comprises a targeting moiety and wherein the polynucleotide is configured such that the targeting moiety is present on an external capsid surface when expressed and formed into a capsid.

Described in certain example embodiments herein are engineered delivery vesicles comprising (a) a polynucleotide encoding an endogenous LTR retroelement polypeptide comprising a capsid domain, a nucleocapsid domain, a protease domain, and a reverse transcriptase domain; (b) one or more heterologous cargo polynucleotides; (c) one or more packaging elements, wherein the one or more packaging elements are operatively coupled to at least one of the one or more heterologous cargo polynucleotides; and (d) a fusogenic polypeptide.

In certain example embodiments, the endogenous LTR retroelement polypeptide is an endogenous Gag polypeptide, optionally a Sushi family polypeptide or orthologue thereof. In certain example embodiments, the Sushi family polypeptide is a PEG10 polypeptide or orthologue thereof, an RTL1 polypeptide or orthologue thereof, an RTL3 polypeptide or orthologue thereof, an RTL5 polypeptide or orthologue thereof, an RTL6 polypeptide or orthologue thereof, or any combination thereof.

In certain example embodiments, the endogenous LTR retroelement polypeptide comprises one or more modifications that enhance the binding specificity and/or packaging of a heterologous cargo polynucleotide and/or reduce the endogenous LTR retroelement polypeptide binding to an endogenous LTR retroelement polypeptide mRNA.

In certain example embodiments, the one or more modifications are at or near the boundary of the nucleocapsid domain and the protease domain of the endogenous LTR retroelement polypeptide. In certain example embodiments, the one or more packaging elements are capable of complexing with one or more domains of the endogenous LTR retroelement polypeptide. In certain example embodiments, the one or more packaging elements comprise one or more 5′ untranslated regions (UTRs) or portion thereof, one or more 3′ UTRs or portion thereof, or both, and wherein the one or more 5′ UTRs or portion thereof, the one or more 3′ UTRs or portion thereof, or both are capable of complexing with one or more domains of the endogenous LTR retroelement polypeptide, and optionally wherein at least one of the one or more 3′ UTRs or portion thereof comprises about 500 bp of a proximal end of a 3′UTR of an mRNA encoding an endogenous LTR retroelement polypeptide. In certain example embodiments, one or more of the one or more 5′ UTRs or portion thereof are derived from a UTR of an mRNA encoding an endogenous LTR polypeptide, wherein one or more of the one or more 3′ UTRs are derived from a UTR of an mRNA encoding an endogenous LTR polypeptide, or both. In certain example embodiments, one or more of the one or more packaging elements comprises at least a 3′ UTR or portion thereof derived from a UTR of an mRNA encoding an endogenous LTR retroelement polypeptide. In certain example embodiments, the 3′UTR or portion thereof comprises about 500 bp of a proximal end of a 3′UTR of an mRNA encoding an endogenous LTR retroelement polypeptide.

In certain example embodiments, the mRNA encoding an endogenous LTR retroelement polypeptide is an mRNA encoding an endogenous Gag polypeptide, optionally a Sushi family protein. In certain example embodiments, the mRNA encoding an endogenous LTR retroelement polypeptide is an mRNA encoding a PEG10 polypeptide or orthologue thereof, an RTL1 polypeptide or orthologue thereof, an RTL3 polypeptide or orthologue thereof, an RTL5 polypeptide or orthologue thereof, an RTL6 polypeptide or orthologue thereof.

In certain example embodiments, the packaging element is a polynucleotide comprising a polynucleotide motif having a sequence of UNNUU, wherein each N is independently selected from A, T, C, G, or U.

In certain example embodiments, the fusogenic polypeptide is specific for a target cell type to which the cargo polynucleotide is targeted for delivery. In certain example embodiments, the fusogenic polypeptide is a tetraspanin (TSPAN), a G envelope protein, an epsilon-sarcoglycan (SGCE), a syncitin, or a combination thereof. In certain example embodiments, the TSPAN is CD81, CD9, CD63 or a combination thereof. In certain example embodiments, the G envelope protein is a vesicular stomatitis virus G envelope protein (VSV-G).

In certain example embodiments, the at least one or more heterologous cargo polynucleotides are RNA, DNA, or hybrid RNA/DNA.

In certain example embodiments, the at least one or more heterologous cargo polynucleotides comprise one or more modifications capable of modifying the functionality, packaging ability, stability, localization, or any combination thereof, of the at least one or more heterologous cargo polynucleotides.

In certain example embodiments, at least one of the one or more heterologous cargo polynucleotides encodes an RNA guided nuclease system or component thereof, optionally an RNA guided nuclease. In certain example embodiments, the RNA guided nuclease system is a Cas-based system or an IscB system and wherein the optional RNA guided nuclease is a Cas polypeptide or an IscB polypeptide.

In certain example embodiments, at least one of the one or more heterologous cargo polynucleotides comprises a guide polynucleotide and/or a polynucleotide encoding a guide polynucleotide, optionally wherein the guide polynucleotide and/or the polynucleotide encoding a guide polynucleotide are operatively coupled to one or more of the one or more packaging elements. In certain example embodiments, the at least one of the one or more heterologous cargo polynucleotides that encodes an RNA guided nuclease further comprises a guide polynucleotide or a polynucleotide encoding a guide polynucleotide. In certain example embodiments, the guide polynucleotide or the polynucleotide encoding a guide polynucleotide is operatively coupled to the same packaging elements as one or more at least one heterologous cargo polynucleotides that encodes an RNA guided nuclease.

Described in certain example embodiments herein are methods of generating engineered delivery vesicles loaded with one or more cargo polynucleotides, comprising delivering to and/or incubating a delivery vesicle generation system as described herein in one or more bioreactors; and isolating generated engineered delivery vesicles from the one or more bioreactors. In certain example embodiments, the one or more bioreactors are one or more cells, optionally one or more eukaryotic cells or prokaryotic cells. In certain example embodiments, the cells are cultured in suspension during incubation. In certain example embodiments, the method further comprises purifying isolated engineered delivery vesicles. In certain example embodiments, the method further comprises concentrating the isolated and/or purified engineered delivery vesicles, optionally 1-5000×.

Described in certain example embodiments herein are engineered delivery vesicles, wherein the engineered delivery vesicles are generated by a system of any one of the engineered delivery vesicle generation systems and/or methods described herein.

Described in certain example embodiments herein are a cell or cells that each comprise an engineered delivery vesicle generation system described herein and/or one or more engineered delivery vesicles described herein.

Described in certain example embodiments herein are co-culture systems comprising two or more cell types, wherein at least one cell type of the two or more cell types, all cell types of the two or more cell types, or a sub-combination of cell-types of the two or more cell types comprise an engineered delivery system described herein.

Described in certain example embodiments herein are methods of cellular delivery of a cargo comprising delivering, to a donor cell type, an engineered delivery vesicle generation system described herein, wherein expression of the engineered delivery vesicle generation system in the donor cell types results in generation of the engineered delivery vesicles and thereby delivery of the engineered delivery vesicles to one or more recipient cell types. In certain example embodiments, expression of the engineered delivery vesicle generation system and generation of the engineered delivery vesicles, delivery of the engineered delivery vesicles to the one or more recipient cell types, or any combination thereof of, each independently occurs in vitro, ex vivo, or in vivo.

Described in certain example embodiments herein are methods delivering one or more engineered delivery vesicles described herein to a cell.

Described in certain example embodiments herein are formulations comprising (a) an engineered delivery vesicle generation system described herein; (b) one or more engineered delivery vesicles described herein; (c) a cell or cell population described herein; (d) a co-culture system described herein; (e) or any combination thereof. In certain example embodiments, the formulation comprises a pharmaceutically acceptable carrier.

Described in certain example embodiments herein are methods comprising delivering, to a subject, (a) an engineered delivery vesicle generation system described herein; (b) one or more engineered delivery vesicles described herein; (c) a cell or cell population described herein; (d) a co-culture system described herein; (e) a formulation described herein, or (f) any combination thereof.

Described in certain example embodiments herein are formulations comprising an engineered delivery vesicle generation system described herein; and a buffer optimized for RNA binding and/or encapsidation. In certain example embodiments, the buffer comprises an optimized concentration of a salt, optionally NaCl, and an optimized concentration of ZnSO₄. In certain example embodiments, the optimized concentration of NaCl ranges from 0 mM to 1 M. In certain example embodiments, the optimized concentration of ZnSO₄ ranges from 0 μM to 1 mM. In certain example embodiments, the optimized concentration of NaCl is about 1 M and the optimized concentration of ZnSO₄ is about 0.5 mM. In certain example embodiments, the optimized concentration of NaCl is about 0 M and the optimized concentration of ZnSO₄ ranges from about 0.05 mM to about 0.5 mM. In certain example embodiments, the optimized concentration of ZnSO₄ is about 0.05 mM or about 0.5 mM. In certain example embodiments, the formulation further comprises a pharmaceutically acceptable carrier.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Shows expression of various env proteins in HEK293T cells, with increased expression shown for Envw1, Envk1, and Envfrd.

FIG. 2—Shows expression of various endogenous retroviral glycoproteins from particles that are pseudotyped with lentiviral proteins.

FIG. 3—Shows expression of a Pnma3-RFP fusion construct (illustrated at the top) compared to a lentivirus-RFP reporter in mouse neuronal cells. Micrographs show organotypic culture slices from the prefrontal cortex.

FIG. 4—Shows maps of various endogenous gag proteins tested for their ability to form capsids, secrete proteins, and transfer materials to a new cell.

FIG. 5—Representative images of transmission electron micrographs showing the ability of various endogenous gag protein candidates to form capsids.

FIG. 6—Shows ability of various endogenous gag proteins to be secreted from cells.

FIGS. 7A-7B—Shows gag constructs containing Cas9/gRNA complexes in the absence (FIG. 7A) and presence (FIG. 7B) of membrane fusion protein VSV-G.

FIG. 8—A schematic illustrating the experimental outline.

FIGS. 9A-9B—Alignment of sequences showing the number of mutations introduced with CRISPR complexes transferred in vesicles comprising RTL1 (FIG. 9B) versus control vesicles (FIG. 9A).

FIG. 10—A graph showing the of number indels induced by editing complexes in vesicles comprising various gag-homology proteins.

FIGS. 11A-11C—Illustrate the ability of (FIG. 11A) PNMA4, (FIG. 11B) PEG10, and (FIG. 11C) RTL1 to transfer Cas9/gRNA complexes to a new cell.

FIG. 12—Alignment of sequences of knock-in mice that expressed an HA-tag on endogenous RTL-1.

FIG. 13—Representative nitrocellulose gel showing HA-tagged PEG10 and RTL1.

FIGS. 14A-14D—Representative immunofluorescence images illustrating the ability of various gag-homology proteins (FIGS. 14B-14D) to form vesicles in the presence of VSV-G compared to control particles (FIG. 14A).

FIGS. 15A-15B—Graphs showing copy numbers of vesicles produced in the presence of various gag-homology proteins.

FIG. 16—A graph showing fold change in viral infectivity when various gag-homology proteins are overexpressed.

FIG. 17—A schematic showing various putative endogenous signaling systems on a scale of decreasing immunogenicity.

FIG. 18—A schematic showing the requirements for an enveloped VLP.

FIG. 19—Representative electron micrographs showing the ability of various gag-homology proteins to spontaneously form vesicles from cells.

FIG. 20—Representative electron micrographs showing the ability of various gag-homology proteins to spontaneously form vesicles from cells.

FIG. 21—Representative immunoprecipitation assays showing various gag-homology proteins secreted from cells.

FIG. 22—A schematic showing an assay used for determining whether GAGs are taken up by cells.

FIGS. 23A-23D—(FIGS. 23A-23B) show the ability of various gag constructs to be taken up by cells and introduce indels into target sequences; (FIG. 23A) SEQ ID NO:9-18; (FIG. 23B) SEQ ID NO:19-26; (FIGS. 23C-23D) graphs showing the ability of vesicles to be taken up into HEK293FT cells in the (FIG. 23C) absence and (FIG. 23D) presence of VSV-G.

FIG. 24—Representative immunoprecipitation assay showing the ability of various constructs to be taken up by cells in the absence (left) and presence (right) of VSV-G.

FIG. 25—A schematic showing the two overlapping reading frames of PEG10.

FIG. 26—A representative immunoprecipitation gel showing bands for both translated ORF1 and ORF1/2 of PEG10.

FIG. 27—Representative immunoprecipitation reactions from whole cell lysates of cells transfected with various PEG10 constructs.

FIG. 28—Representative immunoprecipitation reactions from whole cell lysates and VLP fractions of cells transfected with various PEG10 constructs.

FIG. 29—Representative immunoprecipitation assay analyzing the ability of VSV-G and SGCE to boost PEG10 secretion and uptake into target cells.

FIG. 30—Representative immunoprecipitation gels showing the ability of sucrose cushions of various concentrations to boost the delivery efficiency of PEG10.

FIG. 31—A graph showing percent INDEL generation by use of various constructs.

FIG. 32—Representative Western blots and immunofluorescent stains slowing the location of PEG10 in both the serum and cortex neurons in the brain.

FIG. 33—A graph showing that knockout mice lacking PEG10 show early embryonic lethality, indicating the importance of this gene in embryonic development.

FIG. 34—RNA-seq gene ontology analysis of primary mouse neurons revealed three groups of differentially expressed genes: 1) genes involved in chromatin remodeling; 2) genes involved in the trans-Golgi network and exocytosis, and 3) SNAREs and other genes coding for endosomal proteins.

FIG. 35—Representative fluorescent micrographs showing expression of GFP/PEG10 reporter constructs.

FIG. 36—A schematic showing a DNA methyltransferase identification mechanism (DamID) to map binding sites of DNA- and chromatin-binding proteins. DamID identifies binding sites by expressing the proposed DNA-binding protein as a fusion protein with DNA methyltransferase.

FIG. 37—A schematic of DamID mapping.

FIG. 38—PEG10-DAMID fusion constructs were analyzed for their ability to bind DNA and RNA by cross-referencing DamID mapping data with ATAC-seq data.

FIG. 39—Shows results of a mass-spectrometry analysis of enriched proteins in VLP fractions from N2A cells.

FIG. 40—A schematic for how PEG10 mediates secretion from cells.

FIG. 41—A schematic showing constructs that form RNA-containing gag vesicles.

FIG. 42—A graph showing the ability of various gag-homology proteins to produce RNA-containing vesicles in the absence of VSV-G.

FIG. 43—A graph showing the ability of various gag-homology proteins to produce RNA-containing vesicles in the presence of VSV-G.

FIG. 44—A schematic showing protocol for genome-wide screen for native proteins that cross the blood-brain barrier.

FIG. 45—A schematic of a modification of the protocol shown in FIG. 44 by transfecting passaged cells in step 1 with a 2^{nd} generation packaging vector to reactivate the provirus.

FIG. 46—Shows the frequency with which guide RNAs end up internalized in target cells.

FIG. 47—Shows a nuclear sort of CNS sub-populations 14 days post tail-vein.

FIG. 48—Representative fluorescence micrographs showing the ability of different exemplary fusogens (Arghap32 and Clmp) to further efficiency of internalization.

FIG. 49—A schematic showing protocol for transfection of constructs and evaluating ability of generating INDELs. Fusion of Cas9 to PEG10 and overexpression in cells allows for generation of INDELs in target cells.

FIG. 50—An analysis of various gag-homology proteins for their ability to act as native fusogens.

FIG. 51—Representative fluorescence micrographs showing the ability of different fusogens (Arghap32 and CXADR) to further efficiency of internalization.

FIG. 52—A graph showing results of analysis of various gags carrying Cas9 for their ability to be secreted from cells.

FIG. 53—A graph showing analysis of select gags from FIG. 52 for their ability to be secreted from cells in the presence of VSV-G.

FIG. 54—Shows graphs showing percent INDEL generation from gags from FIG. 53 (left) when compared to HIV (right).

FIG. 55—Shows results from an analysis of the ability of various gag-IRES-Cas9 constructs to generate INDELs in the presence of various fusogens.

FIG. 56—A schematic of PEG10 and Western blot showing cleavage pattern of overexpressed N- and C-terminal tagged mouse PEG10 in HEK293FT cells.

FIGS. 57A-57F—(FIG. 57A) Western blot of PEG10 cleavage pattern and graph showing peptide abundance of full PEG10; (FIG. 57B) Western blot of PEG10 cleavage pattern and graph showing peptide abundance of the first reading frame of PEG10; (FIG. 57C) Western blot of PEG10 cleavage pattern and graph showing peptide abundance of NC cleavage products; (FIG. 57D) Western blot of PEG10 cleavage pattern and graph showing peptide abundance after cleavage at the protease domain of the second reading frame of PEG10; (FIG. 57E) Western blot of PEG10 cleavage pattern and graph showing peptide abundance after cleavage at the RT domain of the second reading frame of PEG10; (FIG. 57F) Western blot of PEG10 cleavage pattern and graph showing peptide abundance after C-terminal cleavage of the second reading frame of PEG10.

FIGS. 58A-58B—Show a representative Western blot and schematic of protease cleavage sites of PEG10 and the resulting protein fragments (FIG. 58A) with and (FIG. 58B) a putative cleave prior to the Gag domain.

FIG. 59—A schematic of the PEG10 ORF1/2 gene and Western blots showing cleavage patterns of proteins isolated from VLP fraction and whole cell lysate.

FIG. 60—A schematic of the PEG10 protein showing that a CCHC deletion in the NC domain renders it unable to bind a specific sequence (SEQ ID NO: 2) bound by a known myelin expression factor (MYEF).

FIG. 61—An exemplary protocol for binding experiments to determine whether PEG10 binds DNA and graph confirming that PEG10 binds DNA.

FIG. 62—A schematic showing estimation of location of ORF1 cleavage site and experiment done to confirm the location.

FIG. 63—A schematic showing location of ORF1 cleavage site and assessment of payload secretion.

FIG. 64—Representative fluorescent micrographs showing expression of GFP fusion constructs of various ORFs.

FIG. 65—A schematic of hypotheses for the putative functions of various domains when they interact with DNA.

FIG. 66—A schematic of PEG10 with mutations in various domains to determine its function.

FIG. 67—A schematic showing that if PEG10 is nuclear and can bind DNA, (like MYEF), then if follows that PEG10 regulates transcription.

FIG. 68—A schematic showing that mutations in the nucleocapsid domain led to a reduced ability to bind the MYEF motif (SEQ ID NO: 3).

FIG. 69—Shows results from a ootprinting assay to determine function of individual motifs in the PEG10 protein.

FIG. 70—A representative western blot showing quantification of PEG10 in the blood of transgenic mice.

FIGS. 71A-71D—The diversity of genes encoding retrovirus Gag-derived proteins with capsid forming potential in mammalian genomes. (FIG. 71A) Domain architectures of mammalian Gag homologs compared to that of typical retrovirus proteins. Selecting Capsid (CA) containing Gag homologs that are conserved across mammals and are broadly expressed in adult tissue. The listed genes were focused on. Each group of Gag homologs contains a distinct combination of predicted CA, Nucleocapsid (NC), Protease (PR), and Reverse Transcriptase (RT) domains. Proteins were produced in E. coli, purified using affinity chromatography, and further purified with size exclusion chromatography. (FIG. 71B) Proportion of the total bacterially-produced protein that forms oligomers (>600 kD) versus a monomer, as determined by size exclusion chromatography. (FIG. 71C) Representative negative stain transmission electron micrographs of the purified CA-containing proteins confirms that many of the murine orthologues of the CA-domain containing proteins have the capacity to oligomerize and form capsids. Scale bar represents 100 nm. (FIG. 71D) Representative electron micrographs using cryogenic electron microscopy from a selected subset of the identified CA-domain containing proteins further confirm that these proteins form capsids. Scale bar represents 50 nm.

FIGS. 72A-72H—PEG10 protein and mRNA are actively secreted by cells in vitro. (FIG. 72A) Outlining of the method for detecting extracellular forms of CA-domain containing homologs. (FIG. 72B) Western blots showing that PEG10 is the most abundant protein in the cell-free fraction. CD81 was used as loading control for the ultracentrifuged cell-free fraction. Whole cell and VLP fraction blots for the endoplasmic reticulum marker CALNEXIN ensure equal loading of whole cell protein and the purity of cell-free VLP fraction. (FIG. 72C) Quantification of the protein levels in the western blot in (FIG. 72B). (FIG. 72D) Outline of the strategy used to identify nucleic acids that are secreted within CA-domain containing proteins. (FIG. 72E) Log₂ fold change in expression of each of the transcriptionally activated genes compared to cells with control non-targeting gRNAs, as determined by whole cell mRNA sequencing of n=3 biological replicates. (FIG. 72F) Of the 4 genes transcriptionally activated, Peg10 was the only significant mRNA enriched for in the VLP fraction. (FIG. 72G) Alignment of sequencing reads showing high read map coverage of Peg10 mRNA in the VLP fraction. (FIG. 72H) N2a cells were transfected with DNA over-expression vectors with deletions of the predicted nucleocapsid (ANC) and reverse transcriptase (ART) domains of Peg10. Peg10 qPCR of n=3 replicates results show that CCHC containing zinc finger domain is essential for the secretion of Peg10 mRNA in the VLP fraction.

FIGS. 73A-73I—PEG10 is an efficiently processed polyprotein that binds to target mRNAs and modifies their stability. (FIG. 73A) The four domains of PEG10 are translated into two isoforms which are proteolytically processed into fragments. (FIG. 73B) Representative sequencing alignment histogram of the PEG10 locus generated from eCLIP data of n=3 HA-PEG10 and n=3 untagged animals. (FIG. 73C) Log₂ Fold change and significance of bound RNAs from eCLIP data comparing HA-GFP to WT HA-PEG10, we identified at least 900 mRNAs that are bound by PEG10, including Peg10 itself. (FIG. 73D) Ddit4 mRNA is one of the most highly bound mRNAs by PEG10, and this binding is dependent on both the NC and RT domains of PEG10. (FIG. 73E) To understand the effect of depleting PEG10 on target mRNA bioavailability, we depleted PEG10 in the postnatal developing brain. (FIG. 73F) Sorting of neuronal nuclei shows high levels of Peg10 indels from n=3 animals but not animals transduced with a non-targeting gRNA (NT gRNA). (FIG. 73G) mRNA sequencing from neuronal nuclei harvested from the cortex of n=3 animals identifies ˜500 genes differentially expressed (q-value ≤0.10) in animals in which Peg10 has been depleted. (FIG. 73H) Venn diagram showing that, of the significant downregulated genes in Peg10 depleted neurons, many are significantly bound by PEG10 in the brain, demonstrated in eCLIP data. (FIG. 73I) mRNA sequencing of PEG10 VLPs generated by transient transfection in N2a cells confirms that only Peg10 is packaged inside the VLPs.

FIGS. 74A-74F—Flanking cargo with PEG10 5′ and 3′ UTRs leads to functional transfer of cargoRNA into a target cell. (FIG. 74A) Schematic representing the grafting of Peg10 5′ and 3′ UTRs onto cargo. (FIG. 74B) VSV-G pseudotyped Peg10 particles are capable of transferring a cargoRNA encoding a Cre recombinase into a loxP-GFP N2a reporter cell line. Efficient transfer is dependent on the presence of a fusogen (VSV-G) and PEG10. Data quantified by flow cytometry 72 hours after VLP addition on N2a loxP-GFP reporter cells, n=3 replicates. (FIG. 74C) Representative images depicting loxP-GFP recombination in N2a reporter cells 72 hours after addition of PEG10-Cre VLPs. Scale bar represents 100 um. (FIG. 74D) Tiling of the Peg10 3′ UTR reveals the 3′ packaging signal can be shortened to the first 500 bp of sequence and retain functional transfer capability. UTRs are required for transfer. Data quantified by flow cytometry 72 hours after VLP addition, n=3 replicates. (FIG. 74E) Functional transfer of Cre cargoRNA using WT Peg10 or Peg10 with 500 bp bins codon swapped to prevent decoy self-binding. Data quantified by flow cytometry 72 hours after addition to loxP-GFP N2a reporter cells, n=3 replicates. For all panels *p<0.05, **p<0.01, ***p<0.001, ****p<0.0001, One-Way ANOVA. (FIG. 74F) Functional transfer of Peg10v.4 and a further optimized cargoRNA enables a highly efficient mRNA transfer system as compared to an integrating Cre lentivirus. Data quantified by flow cytometry 72 hours after addition to loxP-GFP N2a reporter cells, n=3 replicates. For all panels *p<0.05, **p<0.01, ****p<0.0001, One-Way ANOVA.

FIG. 75—Human tissue wide mRNA expression of CA-containing genes. The shown heatplot from the Broad Institute GTEx portal (gtexportal.org) showing widespread expression of many of the mined CA-containing genes in human tissues. The final list of CA-containing genes were filtered based on conservation between human and mice and detectable gene expression in adult human tissues. Peg10 in particular shows strong expression in the brain and adrenal gland.

FIGS. 76A-76C—Bacterially produced mouse orthologues of CA-containing proteins form oligomers/capsids by size exclusion and EM. (FIG. 76A) Representative chromatogram from size exclusion of mouse PEG10 on a Superdex 200 Increase 10/300 GL. The first annotated peak represents high molecular weight oligomers in the void fraction of the column, while the second represents monomers. The fraction oligomerized was calculated by taking fractions of the area under the curve. (FIG. 76B) Widefield negative stain transmission electron micrographs of bacterially produced CA-containing proteins confirm PFAM predictions for capsid forming proteins. Scale bar represents 200 nm. (FIG. 76C) Additional electron micrographs from cryogenic transmission electron microscopy confirm with high resolution the findings from the negative stain images. Scale bar represents 100 nm.

FIGS. 77A-77C—Many endogenous CA-containing proteins are secreted into the supernatant and found in mouse tissues, but only PEG10 secretes its own mRNA into the VLP fraction. (FIG. 77A) Whole cell lysate from HEK293FT cells in which each of the HA-tagged CA-containing proteins are overexpressed with transient plasmid transfection (as with FIG. 2B). (FIG. 77B) Western blots forPEG10, RTL1, and MOAP1 from cell-free serum and blood of n=6 adult female C57BL/6 mice. While all proteins are abundantly expressed in the brain, only PEG10 is abundant in blood serum. (FIG. 77C) Example sequencing alignment histogram for Arc following transcriptional activation with CRISPRa. While Arc is efficiently transcriptionally activated, no significant mRNA reads are detectable in the ultracentrifuged VLP fraction.

FIGS. 78A-78G—PEG10 is cleaved by its own protease into its constitutive domains, which associate with a number of proteins inside the cell. (FIG. 78A) Western blot for HA and actin of N-term and C-term HA tagged PEG10 confirms PEG10 to be a highly processed polypeptide. By generating the protease mutant D491A, we confirm that the protease is responsible for this processing. When we provide 3× molar excess (+++) of untagged PEG10 in trans, we do not observe processing—demonstrating that the PEG10 protease domain self-processes. (FIG. 78B) The denoted bands from the western blot in (FIG. 78A) correspond to the annotated cleavage sites in this diagram which are approximately between the various domains of PEG10 including the CA, NC, PR, and RT. (FIG. 78C) Peptide landmarks from co-immunoprecipitation mass spectrometry of HA-tagged PEG10 confirm the approximate cleavage sites detailed in (FIG. 78B). (FIG. 78D) Western blot from Co-IP of N and C term labeled PEG10. (FIGS. 78E-78F) Log₂ fold change and significance of proteins immunoprecipitating with N-term and C-term HA tagged PEG10 transfected into N2a cells. The N-term CA domain associates with endoplasmic reticulum proteins while the C-term nucleic acid binding domains associate with RNA splicing and stability proteins. (FIG. 78G) Gene ontology enrichment (GO) indicates that PEG10 is likely secreted via the rough endoplasmic reticulum and is involved in RNA binding and processing.

FIGS. 79A-79G—PEG10 binds a number of mRNAs dependent on its nucleocapsid and reverse transcriptase domains. (FIG. 79A) Schematic for generating HA tagged PEG10 in embryonic mice to study PEG10 interactions in its native context in vivo. (FIG. 79B) Public gene expression data of Peg10 in the mouse frontal cortex shows expression in many cell types (18). (FIG. 79C) eCLIP results from UV cross linked immunoprecipitated HA tagged PEG10 in P30 frontal cortex shows PEG10 binds a wide range of transcripts in the postnatal mouse brain, including Shank1 and App. Fold enrichment is a comparison between n=3 HA-tagged and n=3 wild-type P27-P35 Bl6 mice. (FIG. 79D) Sequencing alignment histogram of Peg10 shows preferentially binding of PEG10 to the 3′UTR of a wide range of mRNAs. (FIG. 79E) Schematic outlining the strategy for determining in vitro the domains responsible for PEG10 nucleic acid binding by systematically deleting each of the predicted nucleic acid binding domains. (FIG. 79F) Western blot against HA from each of the immunoprecipitated PEG10 mutants that were excised for eCLIP. (FIG. 79G) Sequencing alignment histogram at the Peg10 locus from eCLIP of PEG10 bound mRNA. Binding of mRNA is dependent mostly on the zinc finger, and secondarily on the reverse transcriptase.

FIGS. 80A-80C—Many genes are downregulated upon knockout of PEG10 in neonatal mouse brains. (FIG. 80A) Representative image of GFP+ sorted neuronal nuclei from the cortex of P25 Cas9 mice injected with PHP.eB carrying KASH-GFP under the hSyn1 promoter and guides against Peg10. (FIG. 80B) Sequencing alignment histogram from mRNA sequencing of sorted GFP+ nuclei shows near complete loss of Peg10 expression in P25 mouse neurons. (FIG. 80C) Gene ontology analysis of genes downregulated upon knockout of PEG10 in the cortex of neonatal mice. The pathways involved bolster the in vitro CO-IP mass spectrometry results that PEG10 is involved in mRNA processing.

FIGS. 81A-81B—PEG10 particles are secreted in exosomes and carry RNA cargo, not protein. (FIG. 81A) Western blot against HA for PEG10 VLPs produced with co-transfection of mouse CD63, CD81, or both and immunoprecipitated for CD63. Unbound and bound fraction shown. Co-expression of both CD63 and CD81 boosts PEG10 found in the exosome fraction (co-immunoprecipitated with CD63). (FIG. 81B) Western blot of PEG10 VLPs produced in HEK293FT cells with wildtype PEG10 and various domain mutants. Blots for HA show expression (right) and secretion of protein (left) but no Cre protein is present in the VLP fraction. CD81 is used as a loading control in the VLP fraction and calnexin as a loading control and marker of cell contamination.

FIGS. 82A-82B—HsPEG10 is also secreted and capable of mRNA transfer. (FIG. 82A) Western blot of the whole cell lysate and VLP fraction from HEK293FT cells transfected with HsPeg10 shows secretion and similar processing compared to MmPEG10. (FIG. 82B) HsPEG10 VLPs can mediate transfer of cargoRNA carrying Cre into loxP-GFP N2a reporter cells. ***p<0.001, n=2 per condition.

FIG. 83—Representative flow cytometry gating scheme for functional transfer experiments. Cells were first gated on FSC and SSC to remove debris. Following this singlets were gated on SSC, dead cells were removed by gating on the Zombie NIR live/dead stain. GFP+ cells were gated based on untreated controls.

FIG. 84—Peg10 is highly expressed in the human developing thymus compared to other endogenous CA-containing proteins. Dot plot showing expression of endogenous CA-containing genes across several epithelial cell types in the human developing thymus. Dot size represents relative expression level. Peg10 is the most highly expressed endogenous CA-containing gene in many thymus epithelial cell types. Data was generated from the Human Cell Atlas Developmental web portal using data derived from (36). Plot was generated from the human fetal thymus epithelium dataset using the interactive_heatmap_dotplot tool developed by Dorin-Mirel Popescu.

FIG. 85—Effect of Overexpression of TSPANs on P10 secretion. Experimental outline and results from overexpressing TSPANs in HEK/N2A cells to evaluate the effect of overexpression of TSPANs on P10 secretion.

FIG. 86—Experimental outline and results determining if PEG10 is an exosome. C-terminal HA tagged PEG10 virus like particles (VLPs) were produced in N2As with the noted proteins and ability to produce exosomes was examined.

FIG. 87—Further results determining if PEG10 is an exosome. As in FIG. 86, FIG. 87 presents further results from the experiment described in relation to FIG. 86 designed to determine if PEG10 is an exosome.

FIG. 88—A schematic of Production/Purification Strategy and Results from Production of Gag mutants. As shown in the schematic, gag mutants were expressed as fusion proteins with an MBP cleavable tag. The protein gels demonstrate that PEG10 Gag mutants produced well from the production and purification strategy.

FIG. 89—Chromatograms demonstrating effect of phosphate on oligomerization of wild type (WT) PEG10. As demonstrated by the decrease in presence of capsid/oligomers in the presence of phosphate buffer can indicate that phosphate may be detrimental to oligomerization of PEG10.

FIG. 90—A chromatogram of the Gag Mutant 1 that demonstrates a complete capsid loss and an inability to oligomerize. This chromatogram can demonstrate the mutated region in Gag1 mutant plays a role oligomerization of the polypeptide.

FIG. 91—A Coomassie stained protein gel demonstrating proteins in the void fraction from chromatography. As demonstrated by this gel the void fraction and monomers are the same protein.

FIG. 92—A chromatograms of other gag mutants produced. FIG. 92 can demonstrate that other gag mutants do not show as impaired of capsid assembly as compared to gag mutant 1.

FIG. 93—A photomicrographic image demonstrating that PEG10 protein produced from E. coli forms capsids. Capsids produced had an average diameter of about 20-30 nm.

FIG. 94—Experimental strategy and Protein gel demonstrating effect of co-transfection with a membrane fusion protein on secretion. As demonstrated by FIG. 94, PEG10 is still secreted when co-transfected with SynA.

FIG. 95—A fluorescent microscopy image demonstrating that SynA induces substantial membrane fusion in HEK cells. SynA was co-transfected with CMV-GFP in HEK 293FT cells which demonstrated that SynA can induce substantial membrane fusion.

FIG. 96—Shows construct maps for evaluating a trans packaging strategy with PEG10 untranslated regions (UTRs).

FIG. 97—Shows results that can at least demonstrate that the addition of UTRs to a cargo can allows for packaging and delivery of the cargo. As per the strategy shown in FIG. 96, Cas9, when flanked with PEG10 UTRs, demonstrated higher activity (as measured by % indels generated) as compared to when UTRs were not flanking the Cas9.

FIGS. 98-100—Show an experimental strategy and results that can demonstrate the effect of PEG10 VLPs on the transcriptional state of target cells.

FIG. 101—Shows a venn diagram demonstrating DE gene overlap in response to the introduction of PEG10 VLPs to cells as shown in relation to FIGS. 98-100. PEG10 was upregulated in all conditions. HIST and RT mutations were observed to reduce or eliminate the this observed effect. The NC mutant and WT PEG10 were observed to have overlapping DE genes.

FIG. 102—Shows construct maps for evaluating trans packaging with PEG10 UTR flanked cargos and results demonstrating transfer of functional mRNA from the vesicles carrying the packaged cargo. Cre was flanked with PEG10 UTRs and constructs were co-transfected in HEK cells with VSV-G. Vesicles were purified as shown in the purification schematic and added to N2A Cre reporter cells. 3 days later results were obtained using flow cytometry. The results in shown in the graphs and images demonstrate that the UTR flanked Cre VLPs transferred functional mRNA.

FIG. 103—Adult tissue expression of CA-containing proteins. FIG. 103 shows a Heatplot from the Broad Institute GTEx portal (gtexportal.org) of tissue-specific expression of CA-containing genes in human tissues.

FIGS. 104A-104G—Identification of mammalian retroelement derived Gag homologs that form capsids and are secreted. (FIG. 104A) Domain architectures of selected Capsid (CA)-containing mammalian Gag homologs compared to that of typical retrovirus and LTR retrotransposons. Each group of Gag homologs contains a distinct combination of predicted CA, Nucleocapsid (NC), Protease (PR), and Reverse Transcriptase (RT) domains. LTR, long terminal repeat; MA, matrix; IN, integrase. (FIG. 104B) Fraction of the total bacterially-produced protein that forms oligomers (>600 kD), as determined by size exclusion chromatography. (FIG. 104C) Representative negative stain transmission electron micrographs (TEM) of the Mus musculus (Mm) orthologues of the CA-domain containing proteins. Scale bar, 100 nm. (FIG. 104D) Representative electron micrographs using cryogenic electron microscopy (cryoTEM) of a selected subset of the identified CA-domain containing proteins. Scale bar, 50 nm. (FIG. 104E) Method for detecting extracellular forms of CA-domain containing homologs. (FIG. 104F) Representative blots of CA-domain containing proteins in the cell-free fraction. CD81 was used as loading control for the ultracentrifuged cell-free fraction. Whole cell (W.C.) and VLP fraction blots for the endoplasmic reticulum marker CALNEXIN (CNX) ensure equal loading of whole cell protein and the purity of cell-free VLP fraction. (FIG. 104G) Quantification of extracellular CA-domain containing proteins (as in F) based on n=3 replicates.

FIGS. 105A-105C—Bacterially produced mouse orthologues of CA-containing proteins form oligomers/capsids as assayed by size exclusion and EM. (FIG. 105A) Representative chromatogram from size exclusion of MmPEG10 on a Superdex 200 Increase 10/300 GL. The first annotated peak represents high molecular weight oligomers in the void fraction of the column, while the second represents monomers. The fraction oligomerized was calculated by taking fractions of the area under the curve. (FIG. 105B) Widefield negative stain transmission electron micrographs (TEM) of bacterially produced CA-containing proteins. Arrows indicate capsids. Scale bar, 200 nm. (FIG. 105C) Additional electron micrographs from cryogenic transmission electron microscopy (cryo TEM) of bacterially produced CA-containing proteins. Arrows indicate capsids. Scale bar, 100 nm.

FIGS. 106A-106B—Shows that some CA-containing proteins are secreted into the supernatant and detectable in mouse tissues. (FIG. 106A) Whole cell lysate and VLP fraction from HEK293FT cells in which each of the N-terminal HA-tagged CA-containing mouse proteins are overexpressed with transient plasmid transfection in n=3 replicates (quantified in FIG. 104G and representative Western blot with n=1 replicates displayed in FIG. 104F). (FIG. 106B) Western blots for endogenous PEG10, RTL1, and MOAP1 from cell-free serum and blood of n=6 adult female C57BL/6 mice.

FIGS. 107A-107I—MmPEG10 protein and mRNA is secreted in vesicles by cells in vitro. (FIG. 107A) Method for identifying nucleic acids that are secreted in the VLP fraction upon gene activation of CA-domain containing proteins. (FIG. 107B) Differential RNA abundance and significance in the VLP fraction from N2a cells after CRISPR activation of endogenous MmPeg10. (FIG. 107C) Alignment of sequencing reads showing sequencing coverage of the MmPeg10 mRNA from (FIG. 107B). (FIG. 107D) Differential RNA abundance and significance in the VLP fraction from N2a cells after heterologous transfection of MmPeg10. n=3 replicates. (FIG. 107E) The four domains of MmPEG10 are translated into two isoforms. These are self-processed by the PEG10 protease into separate domains, of which the NC and RT bind RNA. (FIG. 107F) Fold enrichment of MmPeg10 mRNA compared to GFP in the VLP fraction from N2a cells transfected with wildtype MmPeg10 or deletions of the predicted nucleocapsid (ANC) and reverse transcriptase (ART) domains. (FIG. 107G) Log₂ fold change and significance of bound RNAs from eCLIP data comparing HA-GFP to WT MmPEG10-HA. (FIG. 107H) Representative sequencing alignment histogram of the MmDdit4 locus generated from eCLIP of N2a cells transfected with wildtype or mutant MmPeg10. (FIG. 107I) Representative sequencing alignment histogram of the MmPeg10 locus generated from eCLIP data of n=3 HA-PEG10 and n=3 untagged animals.

FIGS. 108A-108G—MmPEG10 is cleaved by its own protease into its constitutive domains, which associate with a number of proteins inside the cell. (FIG. 108A) Log 2 fold change in expression of each of the transcriptionally activated genes compared to cells with control non-targeting gRNAs, as determined by whole cell mRNA sequencing of n=3 biological replicates. (FIG. 108B) The denoted bands from the western blot in (FIG. 108C) correspond to the annotated cleavage sites in this diagram which are approximately between the various domains of MmPEG10 including the CA, NC, PR, and RT. (FIG. 108C) Western blot for HA and actin of N-term and C-term HA tagged MmPEG10 with and without a protease mutation at D491A and with 3× molar excess (+++) of untagged MmPEG10 in trans. (FIG. 108D) Peptide landmarks from immunoprecipitation mass spectrometry of HA-tagged MmPEG10 corresponding to the approximate cleavage sites detailed in (FIG. 108C). (FIG. 108E) Log₂ fold change and significance of proteins that co-immunoprecipitated with N-term HA tagged MmPEG10 transfected into N2a cells. Log 2 fold change represents the mean fold change across n=3 replicates. (FIG. 108F) Log 2 fold change and significance of proteins that co-immunoprecipitated with C-term HA tagged MmPEG10 transfected into N2a cells. Log 2 fold change represents the mean fold change across n=3 replicates. (FIG. 108G) Top 10 gene ontology terms (GO) from significant enriched for proteins from the MmPEG10-HA CO-IP mass-spec results.

FIGS. 109A-109G—MmPEG10 binding of mRNA is dependent on its nucleocapsid and reverse transcriptase domains. (FIG. 109A) Schematic outlining the strategy for determining the domains responsible for MmPEG10 nucleic acid binding in vitro by deleting each of the predicted nucleic acid binding domains. (FIG. 109B) Western blot against HA from each of the immunoprecipitated MmPEG10 mutants that were excised for eCLIP. (FIG. 109C) Sequencing alignment histogram at the MmPeg10 locus from eCLIP of each of the MmPEG10 domain mutants. (FIG. 109D) Schematic for generating HA tagged MmPEG10 in embryonic mice to study MmPEG10 interactions in its native context in vivo. (FIG. 109E) Public gene expression data of MmPeg10 in the mouse frontal cortex (22). (FIG. 109F) Plot of eCLIP results from UV cross linked immunoprecipitated HA tagged MmPEG10 in P30 frontal cortex. Fold enrichment is a comparison between n=3 HA-tagged and n=3 wild-type P27-P35 C57BL/6 mice. (FIG. 109G) Sequencing alignment histograms of MmPeg10 bound mRNAs.

FIGS. 110A-110F—Many genes are downregulated upon knockout of MmPeg10 in neonatal mouse brains. (FIG. 110A) Schematic outlining approach for knocking down MmPEG10 in the postnatal developing brain of spCas9 mice using sgRNAs packaged by PHP.eb. (FIG. 110B) Representative image of GFP+ sorted neuronal nuclei from the cortex of P25 spCas9 mice injected with PHP.eB carrying KASH-GFP under the hSyn1 promoter and sgRNAs against MmPeg10. (FIG. 110C) Indel rates at MmPeg10 genomic locus from GFP+ sorted neuronal nuclei from n=3 animals injected with AAV encoding MmPeg10 targeting sgRNAs and n=e animals injected with AAVs encoding a non-targeting (NT) sgRNA. (FIG. 110D) Volcano plot of mRNA sequencing results from neuronal nuclei harvested from the frontal cortex of animals transduced with AAVs encoding MmPeg10 targeting sgRNAs. Log 2 fold change represents the mean fold expression between two cohorts of animals, each with of n=3 injected mice. (FIG. 110E) Gene ontology analysis of genes downregulated upon knock-down of MmPEG10 in the cortex of neonatal mice. (FIG. 110F) Venn diagram showing that, of the significant downregulated genes in MmPeg10 knock-down neurons, 49 are significantly bound by MmPEG10 in the brain, demonstrated in eCLIP data. P-value represents significance of gene overlap, hypergeometric test.

FIGS. 111A-111H—Flanking mRNA with MmPeg10 5′ and 3′ UTRs enables functional intercellular transfer of mRNA into a target cell. (FIG. 111A) Schematic showing reprogramming MmPEG10 for functional delivery of a cargo RNA flanked with the MmPeg10 5′ and 3′ UTRs (hereafter, “cargo(RNA)”). (FIG. 111B) Representative TEM micrographs of VLP fraction immunogold labeled for MmPEG10. Text labels indicate transfection of cells with MmPeg10 or mock (negative). Arrowheads indicate gold labeling. Scale bar, 50 nm. (FIG. 111C) Representative images of loxP-GFP N2a cells treated with VSVg pseudotyped MmPEG10 VLPs produced by transfecting Mm.cargo(Cre) or Cre mRNA, and a lentivirus encoding Cre. Scale bar, 100 um. (FIG. 111D) Functional transfer of RNA into loxP-GFP N2a cells mediated by VSVg pseudotyped MmPEG10 VLPs. Data quantified by flow cytometry 72 hours after VLP addition, n=3 replicates. (FIG. 111E) Functional transfer of RNA into loxP-GFP N2a cells mediated by VSVg pseudotyped VLPs produced with MmPeg10 or mCherry and Mm.cargo(Cre) constructs encoding tiles of the MmPeg10 3′UTR. Data quantified by flow cytometry 72 hours after VLP addition, n=3 replicates. (FIG. 111F) Functional transfer of RNA into loxP-GFP N2a cells mediated by VSVg pseudotyped VLPs produced with HsPEG1010 or mCherry and Hs.cargo(Cre) constructs encoding tiles of the HsPeg10 3′UTR. Data quantified by flow cytometry 72 hours after VLP addition, n=3 replicates. (FIG. 111G) Functional transfer of RNA into loxP-GFP N2a cells mediated by VSVg pseudotyped VLPs produced with rMmPeg10 and Mm.cargo(Cre) or Cre mRNA. Data quantified by flow cytometry 72 hours after VLP addition, n=3 replicates. (FIG. 111H) Functional transfer of RNA into loxP-GFP N2a cells mediated by VSVg pseudotyped VLPs produced with rHsPeg10 and Hs.cargo(Cre) or Cre mRNA. Data quantified by flow cytometry 72 hours after VLP addition, n=3 replicates. For all panels *p<0.05, **p<0.01, ***p<0.001, ****p<0.0001, One-Way ANOVA.

FIG. 112—Representative flow cytometry gating scheme for functional transfer experiments. Cells were first gated on FSC and SSC to remove debris. Following this, singlets were gated on SSC, dead cells were removed by gating on the Zombie NIR live/dead stain. GFP+ cells were gated based on untreated controls.

FIGS. 113A-113C—MmPEG10 particles carry RNA cargo, not protein. (FIG. 113A) Western blot for HA and exosome marker TSG101 of MmPEG10 VLPs pelleted through a 20% sucrose cushion and then further purified using a 8-30% iodixanol step gradient for immunogold labeling and electron microscopy. (FIG. 113B) TEM micrographs of the VLP fraction derived from HEK293FT cells transfected with or without MmPeg10, both conditions included Mm.cargo(Cre) and VSVg. Scale bar represents 50 nm. (FIG. 113C) Western blot of MmPEG10 VLPs produced in HEK293FT cells with wildtype MmPEG10 and various domain mutants. Blots for HA show expression (right) and vesicular secretion of protein (left) but no Cre protein is present in the VLP fraction. CD81 is used as a loading control in the VLP fraction and calnexin as a loading control and marker of cell contamination.

FIGS. 114A-114B—HsPEG10 is also secreted and capable of mRNA transfer. (FIG. 114A) Western blot of the whole cell lysate and VLP fraction from HEK293FT cells transfected with HsPEG10. CD81 and calnexin shown as loading controls and markers of cell contamination, respectively. (FIG. 114B) Mammalian conservation map of the entire MmPeg10 locus generated using UCSC genome browser.

FIGS. 115A-115C—MmPeg10 and HsPEG10 recoding boosts packaging and functional transfer of cargo(RNA). (FIG. 115A) Functional transfer of Mm or Hs.cargo(Cre) using WT MmPeg10, HsPEG10 or Peg10/PEG10 with 500 bp bins of recoded (rc) sequences. Data quantified by flow cytometry 72 hours after addition to loxP-GFP N2a reporter cells, n=3 replicates per condition. (FIG. 115B) Log 2 fold change (AACT) of Mm.cargo(Cre) over ‘self’ Peg10/PEG10 in the VLP fraction of cells transfected with wildtype or recoded MmPeg10 and HsPeg10. Means represent average fold change (AACT) from n=2 replicates. For all panels *p<0.05, **p<0.01, ***p<0.001, ****p<0.0001, One-Way ANOVA. (FIG. 115C) Western blot of the VLP fraction of codon swaps of MmPeg10.

FIGS. 116A-116E—Molecular and functional titration of SEND demonstrates it has a reduced titer compared to lentivirus. (FIG. 116A) Representative images 72 hours following VLP delivery of Mm.cargo(H2B-mCherry) using SEND. Scale bar represents 200 μm. (FIG. 116B) Quantification of mean fluorescence intensity across entire tile imaged wells from n=3 replicates 72 hours following lentiviral delivery of H2B-mCherry, rMmPEG10 mediated delivery of Mm.cargo(H2B-mCherry), or rMmPEG10ΔNC mediated delivery of Mm.cargo(H2B-mCherry). (FIG. 116C) Digital droplet RT-PCR of equivalent volumes of MNAse treated VLP fractions showing RNA copy number of Cre mRNA in SEND VLPs versus lentivirus. (FIG. 116D) Titration of lentivirus and SEND delivering Cre on a volume per volume basis in loxP-GFP N2a cells. ***p<0.001, n.s. p>0.05. (FIG. 116E) Determination of the functional titer of SEND after overnight freezing at −80° C. or overnight storage at 4° C. * p<0.05. For (A) and (B) data quantified by flow cytometry 72 hours after addition of SEND(rMmPeg10, VSVg, Mm.cargo(Cre)) or lentivirus. Two-Way ANOVA with Tukey correction, n=3 per condition per timepoint.

FIGS. 117A-117C—Endogenous fusogens are co-expressed in Peg10/PEG10 expressing cells. (FIG. 117A) ENCODE tissue wide mRNA sequencing data of MmPeg10 and MmSyna across multiple mouse tissues. (FIG. 117B) Sequencing alignment histogram of MmSynA RNA from re-analyzed MmPEG10 eCLIP in trophoblast stem cells in vitro (13). (FIG. 117C) Single cell sequencing scatter plots of human placental cells of HsPEG10 co-expression with the endogenous fusogens t and HsENVFRD-1 (7).

FIGS. 118A-118H—SEND is a modular system capable of delivering gene editing tools into human and mouse cells. (FIG. 118A) Representative images demonstrating functional transfer of Mm.cargo(Cre) or Cre mRNA in rMmPEG10 VLPs pseudotyped with VSVg (V), MmSYNA (A), or MmSYNB (B) in Ai9 (loxP-tdTomato) tail tip fibroblasts. Scale bar, 200 μm. (FIG. 118B) Percent of tdTomato positive cells out of the total number of H2A stained nuclei from high content imaging of n=3 replicates of (FIG. 118A). (FIG. 118C) Schematic representing the retooling of SEND for genome engineering. (FIG. 118D) Indels at the MmKras locus in MmKras1-sgRNA-N2a cells treated with SEND (VSVg pseudotyped rMmPEG10 VLPs) containing either SpCas9 mRNA, Mm.UTR(SpCas9), or Mm.cargo(SpCas9) and a lentivirus encoding SpCas9. Indels quantified by NGS 72 hours after VLP or lentivirus addition, n=3 replicates. (FIG. 118E) Indels at the mouse MmKras locus in a constitutively expressing SpCas9 N2a cell line either transfected with a plasmid carrying the MmKras sgRNA or treated with SEND (rMmPEG10, VSVg, MmKras sgRNA). Indels quantified by NGS after 72 hours, n=3 replicates. (FIG. 118F) Indels at the MmKras locus in N2a cells treated with SEND (VSVg pseudotyped rMmPeg10 SEND VLPs) containing either SpCas9 mRNA or Mm.cargo(SpCas9) and sgRNA. Indels quantified by NGS 72 hours after VLP addition, n=3 replicates. (FIG. 118G) Indels at the HsVEGFA locus in HEK293FT cells treated with SEND (VSVg pseudotyped rHsPEG10 VLPs) containing either SpCas9 mRNA or Hs.cargo(SpCas9) and an unmodified sgRNA. Indels determined by NGS 72 hours after VLP addition, n=3 replicates. (FIG. 118H) SEND is a modular delivery platform combining an endogenous Gag homolog, cargo mRNA, and fusogen, which can be tailored for specific contexts.

FIGS. 119A-119B-MmPeg10 VLPs carrying MmPeg10 mRNA and pseudotyped with MmSynA induce transcriptional changes in primary mouse cortical neurons. (FIG. 119A) Volcano plot of mRNA transcriptome changes compared to naive cells after MmSYNA pseudotyped SEND VLPs containing cargoPeg10 were transferred onto primary mouse cortical neurons for 72 hours. Data shows log 2 fold change and −log₁₀(FDR) of n=3 replicates. Red dots indicate FDR≤0.1. (FIG. 119B) GO pathway enrichment in neurons treated with cargoPeg10 VLPs compared to naïve cells.

FIGS. 120A-120F—Mm.cargo(Peg10) induces significant transcriptional changes in recipient cells. (FIG. 120A) Log₂ fold change and −log₁₀(FDR) of differentially expressed genes between naïve N2a cells and those treated with SEND(rMmPeg10, VSVg, Mm.cargo(Peg10)), red indicates genes with FDR<0.05 (Benjamini-Hochberg correction) n=3 per condition. (FIG. 120B) Log₂ fold change and −log₁₀(FDR) of differentially expressed genes between naïve N2a cells and those treated with SEND(rMmPeg10, VSVg, Mm.cargo(Cre)), red indicates genes with FDR<0.05 (Benjamini-Hochberg correction) n=3 per condition. (FIG. 120C) Venn diagram showing number of genes differentially expressed in N2a cells treated with SEND(rMmPeg10, VSVg, Mm.cargo(Peg10)) or SEND(rMmPeg10, VSVg, Mm.cargo(Cre)) shows 20 overlapping genes between the two conditions. This overlap is significant with p<2.28e-32, hypergeometric test. (FIG. 120D) Fold enrichment of top 10 gene ontology pathways (FDR<0.05) of genes enriched upon Mm.cargo(Peg10) VLP treatment (FIG. 120A). (FIG. 120E) Fold enrichment of top 10 gene ontology pathways (FDR<0.05) of genes depleted upon Mm.cargo(Peg10) VLP treatment (FIG. 120A). (FIG. 120F) Fold enrichment of top 10 gene ontology pathways (FDR<0.05) of genes enriched upon Mm.cargo(Cre) VLP treatment (FIG. 120B).

FIG. 121—HsPEG10 is highly expressed in the human developing thymus compared to other endogenous CA-containing proteins. Dot plot showing expression of endogenous CA-containing genes across several epithelial cell types in the human developing thymus. Dot size represents relative expression level. HsPeg10 is the most highly expressed endogenous CA-containing gene in many thymic epithelial cell types. Data was generated from the Human Cell Atlas Developmental web portal using data derived from (33). Plot was generated from the human fetal thymus epithelium dataset using the interactive_heatmap_dotplot tool developed by Dorin-Mirel Popescu.

FIGS. 122A-122B—Evolutionary provenance of PEG10. (FIG. 122A) Phylogenetic tree of the CA-domain of PEG10 and its homologs encoded by LTR retrotransposons and retroviruses. PEG10 homologs were identified using BLASTP against the non-redundant protein sequence database at the NCBI Sequences were selected to cover the diversity of eukaryotic lineages. Multiple alignment was made using Muscle PMID: 15034147 and trimmed manually to 265 positions (See Supplementary Data). The tree was constructed using FastTree 2 with default parameters (WAG evolutionary model, gamma-distributed site rates) PMID: 20224823. The sequences are denoted by their species of origin and their identifiers in the NCBI protein database. The numbers at forks indicate bootstrap support (percentage points) that was calculated by FastTree. (FIG. 122B) Multiple sequence alignment used to generate FIG. 122A.

FIG. 123—Diversity across several Sushi Family members. Sushi Family members, including RTL1, contain a gag homolog and other LTR retroelement polypeptides. Several include a protease domain.

FIG. 124—Sushi Family members containing a protease are processed. HA-tagged Sushi Family members were expressed in N2A cells and western blotting was performed to characterize processing of the expressed Sushi Family members.

FIG. 125—Some Sushi Family members are secreted into the VLP fraction. HA-tagged Sushi Family members were co-transfected in HEK293FT cells with a VSV-G expression construct for VSV-G pseudotyping. The VLP containing fraction was obtained and western blotting was performed against the HA tag to determine if the tagged Sushis were secreted into the VLP fraction.

FIG. 126—Sushi Family members are likely secreted in membrane vesicle bodies (MVBs) as capsids. HA-tagged Sushi Family members were transfected into HEK293FT cells, were disassociated after 72 hours and were cryosectioned and stained against the HA tag.

FIG. 127—Western blots show secretion of C-terminally HA tagged mouse sushi family member orthologues in HEK293FT cells.

FIG. 128—Western blot shows mouse sushi family members MmRTL1, MmRTL2, MmRTL3, MmRTL4, MmRTL5, MmRTL6, MmRTL7, MmRTL8a, MmRTL8b, MmRTL8c, MmRTL9 and MmRT10 are expressed.

FIG. 129—An exemplary approach to identifying protein-RNA binding motifs, e.g., packaging signal.

FIG. 130—A graph showing packaging signal(s) (a protein-RNA binding motif) across the Mm.Peg10 sequence.

FIG. 131—A representative TBE gel of in vitro transcribed hits.

FIG. 132—Weblogo motif enrichment of in vitro transcribed hits for identification of protein-RNA binding motifs.

FIG. 133—Measurement of exemplary generated in vitro protein capsids with increasing NaCl concentration from 10 mM to 1 M.

FIG. 134—Peg10 RF1 in vitro capsid assembly with varying NaCl concentration.

FIG. 135—Representative images of fractions 1, 7, and 13 exploring generation of exemplary protein capsids in vitro.

FIG. 136—A representative electrophoretic mobility shift assay (EMSA) of tiled mutations along motif bound by PEG10; italic indicates inversion of base.

FIG. 137—Shows exploration of buffer conditions for promotion of RNA binding and encapsidation.

FIG. 138—Representative image of animals injected intravenously with an example embodiment PEG10 VLP with Cre cargo.

FIG. 139—Representative cryo-electron microscopy (cryo-EM) images that show that endogenous capsid-containing proteins (e.g., PEG10) can be reprogrammed to mediate functional transfer of mRNA.

FIG. 140—Shows production of engineered delivery vesicles in producer cells grown in suspension.

FIG. 141—Shows an exemplary strategy for producing engineered delivery vesicles in producer cells grown in suspension.

FIG. 142—Shows an exemplary strategy for producing engineered delivery vesicles in producer cells grown in suspension.

FIG. 143—Shows results of optimization of purification of PEG10 engineered delivery vesicles (“P”) and control lentiviral particles (“L”) produced in cells grown in suspension and a comparison of production from producer cells grown in suspension and plated.

FIG. 144—Shows a production strategy for engineered delivery vesicles that includes nuclease removal and a graph demonstrating titer results from demonstrating delivery from engineered PEG10 delivery vesicles (SEND) (“P”) and control lentiviral particles (“L”) via the production strategy.

FIG. 145—Shows a production strategy for engineered delivery vesicles and a comparison of delivery of a cargo to Ai9 mouse tail tip fibroblasts by engineered delivery vehicles produced by producer cells grown in suspension and producer cells grown on plates.

FIG. 146—Shows graphs demonstrating that engineered PEG10 delivery vesicles produced in producer cells grown in suspension are more effective at cargo delivery than PEG10 delivery vesicles produced in producer cells grown on plates.

FIG. 147—Production of PEG10 engineered delivery vesicles produced suspension for in vivo delivery.

FIG. 148—A strategy to evaluate stimulation of the innate immune response by various delivery particles (including the engineered PEG10 delivery particles (“Peg10 VLPs”).

FIG. 149—A graph showing the effect of delivery via engineered PEG10 delivery particles on genes involved in the innate immune response.

FIG. 150—Shows genes downregulated in engineered PEG10 delivery vesicle mediated delivery vs mRNA lipoplex delivery.

FIG. 151—A graph showing that the IFNa response is marginally upregulated in response to mRNA liposome delivery as compared to engineered PEG10 delivery vesicle mediated delivery (SEND).

FIG. 152—A graph showing that the IFNβ/λ response is upregulated when delivering mRNA in liposomes compared to delivery with engineered PEG10 delivery vesicles (SEND).

FIG. 153—A graph showing that delivery via engineered PEG10 delivery vesicles (SEND) circumvents activation of IFITs.

FIG. 154—A graph showing that IRF1, 7, and 9 are upregulated by liposomal delivery but not by engineered PEG10 delivery vesicle (SEND) delivery.

FIG. 155—A graph showing inflammatory cytokines that are upregulated by mRNA liposome delivery as compared to engineered PEG10 delivery vesicle mediated delivery.

FIG. 156—A graph showing the effect of mRNA liposome delivery as compared to engineered PEG10 delivery vesicle mediated delivery on genes associated with the anti-viral response.

FIG. 157—Diversity across several Sushi Family members. Sushi Family members, including RTL1, contain a gag homolog and other LTR retroelement polypeptides. Several include a protease domain and/or capsid domain.

FIG. 158—A graph showing packaging signal(s) (a protein-RNA binding motif) across the Mm.Rtl1 sequence.

FIG. 159—A graph showing packaging signal(s) (a protein-RNA binding motif) across the Mm.Rtl4 sequence.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The terms “high,” “higher,” “increased,” “elevated,” or “elevation” refer to increases above basal levels, e.g., as compared to a control. The terms “low,” “lower,” “reduced,” or “reduction refer to decreases below basal levels, e.g., as compared to a control.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control patient (can be a stored sample or previous sample measurement) with a known outcome; normal tissue, fluid, or cells isolated from a subject, such as a normal patient or the patient having a condition of interest.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

In one aspect, example embodiments disclosed herein are directed to engineered delivery vesicle generation systems. These systems comprise delivery vesicle generation systems derived from retroelements endogenous to a mammalian genome, which may include retroviruses and retrotransposons. As used herein, the term “endogenous” when used in connection with retroelement polypeptide refers to a retroelement polypeptide that has become incorporated into a host genome and is capable of being expressed by the host genome. These systems comprise retroelement proteins (also referred to interchangeably herein as a retroelement polypeptide) capable of forming a vesicle and packaging various cargo molecules within the formed vesicle. As used herein, a “vesicle” refers to a particle having an outer shell comprised of retroelement polypeptides that further define an inner cavity or space which may then used to hold one or more cargo molecules as further defined herein. In some embodiments, the retroelement protein is an endogenous long-terminal repeat (LTR) retroelement protein that is capable of forming a vesicle and packaging various cargo molecules within the formed vesicle. As used herein, the term “LTR retroelement” encompasses elements from retroviruses and/or LTR retrotransposons and polypeptides derived therefrom. In certain example embodiments, the endogenous LTR retroelement polypeptide is derived from a mouse genome. In certain other example embodiments, the endogenous LTR retroelement polypeptide is derived from a human genome. While not being bound by a particular scientific theory, because these endogenous retroelement polypeptides are endogenously expressed in mammalian genomes, the resulting delivery vesicles are expected to be less immunogenic than a particle derived directly from a virus, like an adeno-associated virus (AAV).

As disclosed herein, Applicants have further identified elements, referred to herein as “packaging elements,” responsible for helping facilitate packaging of cargo molecules into the delivery particles. Accordingly, in some embodiments, the delivery vesicle generating systems disclosed herein can be programmed to select specific cargo molecules through manipulation of the packaging elements. For example, elements that specifically interact with one or more domains on the retroelement polypeptide can be engineered onto a desired cargo molecule such that when the retroelement polypeptide is expressed in the presence of the cargo molecule, for example in a cell or other bioreactor, the desired cargo molecule is specifically incorporated into the delivery vesicle thus increasing the number of vesicles generated that contain the desired cargo molecule. Also disclosed herein are modifications to the retroelement polypeptide that decrease non-specific packaging of non-cargo molecules. Tailoring of the system will allow for both cell-specific and cell-non-specific delivery methods.

In certain example embodiments, the engineered delivery vesicle generating systems further comprise a fusogenic polypeptide, which may facilitate entry of the delivery vesicle to a target cell type. In certain example embodiments, the fusogenic protein confers a trophism on the delivery vesicle for a specific cell type. In some embodiments, the fusogenic protein is an endogenous fusogenic protein. As used in this context herein “endogenous” refers to a fusogenic protein or encoding sequence that has become incorporated into a host genome and is capable of being expressed by the host genome. In other example embodiments, trophism may be determined by further engineering of the delivery vesicle to display a targeting moiety specific to a particular cell type on the outer surface of the delivery vesicle.

In another aspect, embodiments disclosed herein are directed to a method of generating engineered delivery vesicles loaded with cargo molecules. For example, polynucleotides, such as a vector, encoding the retroelement polypeptide, such as an endogenous retroelement polypeptide, may be delivered to cell or bioreactor along with a cargo molecule, leading to generation of delivery vesicles loaded with the desired cargo molecules. The delivery vesicles may then be isolated from the cell or bioreactor.

In another aspect, embodiments disclosed herein are directed to cargo-loaded delivery vesicles derived from the delivery vesicle generating systems disclosed herein. Such vesicles may then be used to deliver the cargo to a desired cell type or cell population in vitro, ex vivo, or in vivo. Accordingly, in another aspect, embodiments disclosed herein are directed to methods of delivering cargo molecules to specific cell types or cell populations via the delivery vesicles.

In another aspect, embodiments disclosed herein are directed to co-culture system comprising two or more cell types. One or more cell types in the co-culture system may be modified to express one or more delivery vesicle generating systems disclosed herein. The programmable nature of the delivery vesicles, both to the type of cargo packaged and cell-type delivered to, may be used to set up synthetic connections are made between the various cell types wherein one cell may produce and package a given cargo molecule and deliver said cargo molecule to one or more other cell types in the co-culture system.

### Engineered Delivery Vesicle Generation Systems

Generally, embodiments disclosed herein relate to engineered delivery vesicle generation systems and delivery vehicles produced therefrom. The engineered delivery vesicle generation systems can include cargo molecules, such as cargo polynucleotides, that can be packaged within the delivery vesicles. In this way, the systems and compositions described herein can be used to package and/or deliver a cargo molecule to a subject, such as a cell.

As described in greater detail elsewhere herein, the engineered delivery vesicle generation systems can include one or more retroelements endogenous to a mammalian genome that are capable of recognizing and/or interacting with one or more packaging elements contained in the system. As described in several exemplary embodiments herein the packaging element(s) can be operatively coupled to one or more cargo molecules to facilitate packaging of a cargo molecule into a delivery vesicle. The one or more retroelements endogenous to a mammalian genome included in the system can be capable of binding and/or packaging self-encoding mRNA. The one or more retroelements elements endogenous to a mammalian genome included in the system can also be capable generating vesicles that can be exported from a cell. As described in several example embodiments, such retroelements are Gag homologs. In one specific example, the Gag homolog is PEG10. PEG10 is an exemplary LTR retrotransposon-derived polypeptide. In another specific example, the Gag homolog is a Sushi Class protein.

As provided in further detail below, a variety of cargo molecules, including cargo polynucleotides, may be packaged within the delivery vesicles disclosed herein. As previously mentioned, and described in greater detail below, the cargo molecule may be modified with one or more packaging elements that complex or bind to the LTR retroelement polypeptide and facilitate packaging of the cargo molecule into the delivery vesicle. While the term “cargo molecule” is referred to in the singular, it is contemplated that multiple copies of a cargo molecule, depending on type and other readily recognizable size constraints of the delivery vesicle, may be packaged within a single delivery vesicle.

In some exemplary embodiments, an engineered delivery vesicle generation system is composed of (a) a polynucleotide encoding an endogenous retroelement polypeptide comprising a capsid domain, a nucleocapsid domain, a protease domain, and a reverse transcriptase domain; (b) one or more heterologous cargo polynucleotides; and (c) one or more packaging elements operatively coupled to the one or more heterologous cargo polynucleotides. The engineered delivery vesicle generation system can further include (d) a polynucleotide encoding a fusogenic polypeptide. Exemplary endogenous Gag polypeptides, heterologous cargo polynucleotides, and packaging elements are described in greater detail elsewhere herein.

In some embodiments, the system or component(s) thereof are modified to increase and/or enhance packaging of a cargo, and/or reduce endogenous retroelement polypeptide binding to endogenous retroelement polypeptide RNA. Exemplary modifications include, but are not limited to, changes to the polynucleotide sequence in the polynucleotide encoding the endogenous Gag polypeptide at the boundary between the nucleocapsid domain encoding region and the protease domain encoding region. In some embodiments, about 1, 2, 3, 4, 5, 6, 7, 8, 9, 10, 15, 20, 25, 30, 35, or 40 or more nucleotide modifications are made. Exemplary modifications are described elsewhere herein.

In some embodiments, the packaging elements are untranslated regions or portions thereof of a polynucleotide encoding (e.g., an mRNA) of an endogenous retroelement polypeptide. In some embodiments, the UTR(s) or portions thereof are from the same endogenous Gag polypeptide that is included in the system. Exemplary packaging elements are further discussed and described elsewhere herein.

Similarly, exemplary engineered delivery vesicles include (a) polynucleotide encoding an endogenous retroelement polypeptide (and/or an endogenous Gag polypeptide) comprising a capsid domain, a nucleocapsid domain, a protease domain, and a reverse transcriptase domain; (b) one or more heterologous cargo polynucleotides; (c) one or more packaging elements, wherein the one or more packaging elements are operatively coupled to at least one of the one or more heterologous cargo polynucleotides; and (d) a fusogenic polypeptide.

### Endogenous LTR Retroelement Polypeptides

The endogenous LTR retroelement polypeptides used in the generation of the delivery vesicles are derived from endogenous genomic sequences of a host genome that have resulted from stable incorporation of various LTR retroelement-derived coding sequences that are actively expressed from the host genome. More specifically, the endogenous LTR retroelement polypeptides are capable of and do form vesicles upon expression. As further detailed herein, some of these endogenous LTR retroelement polypeptides are also able to capture and package their own mRNA into such vesicles. Other endogenous LTR retroelement polypeptides can be engineered to include a domain or other element capable of capturing a cargo molecule. As detailed further below, in both instances, elements responsible for binding and/or complexing with the endogenous LTR retroelement-polypeptide(s) may be incorporated onto various cargo molecules to facilitate their packaging into delivery vesicles.

In general, the system may include endogenous LTR retroelement polypeptides may be encoded on one or more polynucleotides and/or vectors. Vector(s) having the encoding polynucleotides can be delivered, e.g., to a cell where they can be expressed to, for example, generate engineered delivery vesicles and/or package cargo(s) into engineered delivery vesicles described herein.

In some embodiments, the endogenous LTR retroelement polypeptide encompasses a capsid domain and a nucleocapsid domain. In certain other example embodiments, the endogenous LTR retroelement polypeptide encompasses a capsid domain, a nucleocapsid domain, a protease domain, and a reverse transcriptase domain.

The endogenous LTR retroelement polypeptide can be modified such that its ability to bind self-encoding mRNA is reduced or eliminated and/or its ability to package a cargo polynucleotide operatively coupled to the packaging element(s) is increased. Such modification can include, but is not limited to, a modification to one or more domains of the endogenous LTR retroelement polypeptides such that it has reduced binding and/or packaging ability of its own mRNA. As demonstrated in the Working Examples here (e.g., Examples 12-13) in the context of PEG10, nucleotides in the PEG10 encoding polynucleotide were modified near the near the boundary of the nucleocapsid domain and the protease domain of the PEG10 polypeptide such that binding of PEG10 mRNA was reduced, which resulted in an increase in packaging efficiency of a cargo polynucleotide. Such approaches and methods of confirming the effect of a given modification can be used to identify other suitable modifications in PEG10 as well as suitable modifications in other endogenous LTR retroelement polypeptides.

In some embodiments, the LTR retroelement is PEG10 or ortholog thereof. In some LTR retroelement is any one of the following PEG10 orthologs: Mirabilis mosaic virus (GenBank Accession No. NP_659396.1); Cauliflower mosaic virus (GenBank Accession No. NP_056727); Carnation etched ring virus (GenBank Accession No. ADY76948.1); Banana streak OL virus (GenBank Accession No. AFH88829.1); Banana streak GF virus (GenBank Accession No. AHM92951.1); Dioscorea bacilliform virus (GenBank Accession No. ABI47986.1); Dracena mottle virus (GenBank Accession No. YP_610965.1; Taro bacilliform virus (GenBank Accession No. NP_758808.1); Copia polyprotein Drosophila willistoni (GenBank Accession No. AAF06364.1); Equine infections anemia virus (GenBank Accession No. AGC82153.1); Jaagsiekte sheep retrovirus (GenBank Accession No. AF009966.1); human immunodeficiency virus (GenBank Accession No. AAN77283.1); Python molurus endogenous retrovirus (GenBank Accession No. AAN77283.1); Bovine leukemia virus (GenBank Accession No.NP_777381.1); Mous mammary I virus (GenBank Accession No. NP_955569.1); Smittium culicis (GenBank Accession No. OMJ19218.1); Labeo rohita (GenBank Accession No.RXN25002.1); Dicentrarchus labrax (GenBank Accession No. CBN80957.1); Dicentrachus labrax (GenBank Accession No. CBN81178.1); Pimephales promelas (GenBank Accession No. KAG1931208.1); Collicthys lucidus (GenBank Accession No. TKS65685.1); Zancudomyces culisetae (GenBank Accession No.OMH78677.1); Plasmodiophora brassicae (GenBank Accession No. CE094710.1); Ceratodon purpureus (GenBank Accession No. KAG0578666.1); Ceratodon purpureus (GenBank Accession No. KAG0614891.1); Xenopus laevis (GenBank Accession No. XP_031796629.1); Xenopus laevis (GenBank Accession No. OCT57199.1); Ophinophagus Hannah (GenBank Accession No. ETE58569.1); Sarcophilus harrisii (GenBank Accession No. XP_031796629.1; PEG10 Mus musculus (GenBank Accession No. NP_570947.2); PEG10 Homo sapiens (GenBank Accession No. NP_001165909.1); or Choloepus didactylus (GenBank Accession No. XP_037692625.1).

### Gag Homologs

In certain example embodiments, the endogenous LTR retroelement polypeptide is an endogenous Gag polypeptide or Gag homolog. In native retroviruses, Gag (after group-specific antigen) is the core structural protein the forms the capsid within the infectious retrovirus virion. The capsid protects the viral genome and enables efficient transfer of virus genomes between host cells. Applicants mined mammalian genomes to identify endogenous gag homologs. In certain example embodiments, the gag homolog) encompasses a capsid domain and a nucleocapsid domain. In certain other example embodiments, the gag homolog encompasses a capsid domain, a nucleocapsid domain, a protease domain, and a reverse transcriptase domain.

In one example embodiment, the Gag homolog is selected from Arc, ASPRV1, a Sushi-Class (or Sushi Family) protein, a SCAN protein, or a PNMA protein. In another example embodiment, the Gag-homology protein is a PNMA protein. In one example embodiment, the PNMA protein is selected from the group consisting of: ZCC18, ZCH12, PNM8B, PNM6A, PNMA6E_i2, PMA6F, PMAGE, PNMA1, PNMA2, PNM8A, PNMA3, PNMA4, PNMA5, PNMA6, PNMA7, PNMA1, MOAP1, ZCCHC12 or CCD8. In another example embodiment, the Gag homolog is an Arc protein. In one example embodiment, the Arc protein is hARC or dARC1. In another example embodiment, the Gag homolog is ASPRV1. In another example embodiment, the Gag homolog is a SCAN protein. In one example embodiment, the SCAN protein is PGBD1. In some embodiments, the Gag homolog is a Sushi-Class protein. In some embodiments, the Sushi-Class protein has a protease domain. In another example embodiment, the Gag homolog is selected from the group consisting of; PEG10, RTL1, RTL2, RTL3, and RTL10. In one example embodiment, the Gag homolog is PEG10. In another example embodiment, the PEG10 is PEG10_i6 or PEG10_i2. In some embodiments, the Gag homolog is RTL1, RTL3, RTL5, or RTL6. In some embodiments, the Gag homolog is RTL1, RTL3, or RTL6. In some embodiments, the Gag homolog is RTL1. In some embodiments the Gag homolog is RTL1, RTL2, RTL3, RTL4, RTL5, RTL6, RTL7, RTL8 (including RTL8a, RTL 8b, and RTL8c), RTL9, or RTL10. In some embodiments, the Gag homolog is RTL1 (PEG 11). In some embodiments, the Gag homolog is RTL2 (PEG10). In some embodiments, the Gag homolog is RTL3. In some embodiments, the Gag homolog is RTL4. In some embodiments, the Gag homolog is RTL5. In some embodiments, the Gag homolog is RTL6. In some embodiments, the Gag homolog is RTL7. In some embodiments, the Gag homolog is RTL8. In some embodiments, the Gag homolog is RTL9. In some embodiments, the Gag homolog is RTL10. In some embodiments, the Gag homolog is as in any one or more of Tables 4, 5, 6, 9, 10, 12, and/or 13 in the Working Examples elsewhere herein.

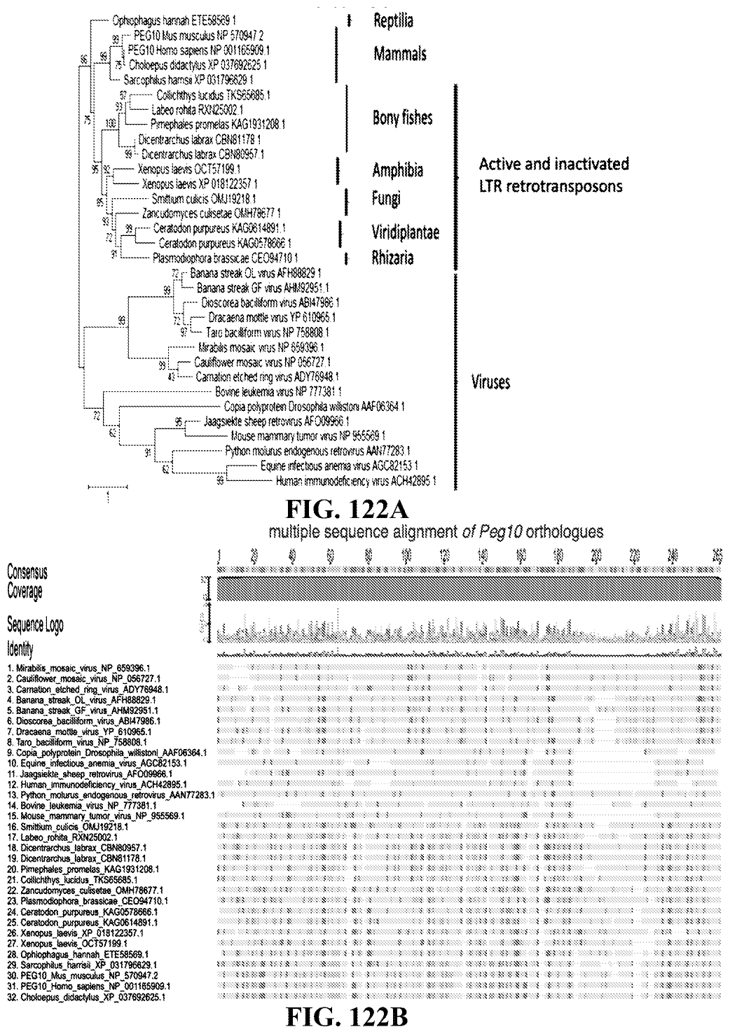

In some embodiments, the gag homolog is a PEG10 ortholog. In some embodiments the gag homolog is any one of the PEG10 orthologs noted in e.g., FIG. 122B. In some embodiments the gag homolog is any one of the following PEG10 orthologs: Mirabilis mosaic virus (GenBank Accession No. NP_659396.1); Cauliflower mosaic virus (GenBank Accession No. NP_056727); Carnation etched ring virus (GenBank Accession No. ADY76948.1); Banana streak OL virus (GenBank Accession No. AFH88829.1); Banana streak GF virus (GenBank Accession No. AHM92951.1); Dioscorea bacilliform virus (GenBank Accession No. ABI47986.1); Dracena mottle virus (GenBank Accession No. YP_610965.1; Taro bacilliform virus (GenBank Accession No. NP_758808.1); Copia polyprotein Drosophila willistoni (GenBank Accession No. AAF06364.1); Equine infections anemia virus (GenBank Accession No. AGC82153.1); Jaagsiekte sheep retrovirus (GenBank Accession No. AF009966.1); human immunodeficiency virus (GenBank Accession No. AAN77283.1); Python molurus endogenous retrovirus (GenBank Accession No. AAN77283.1); Bovine leukemia virus (GenBank Accession No.NP_777381.1); Mous mamItumor virus (GenBank Accession No. NP_955569.1); Smittium culicis (GenBank Accession No. OMJ19218.1); Labeo rohita (GenBank Accession No.RXN25002.1); Dicentrarchus labrax (GenBank Accession No. CBN80957.1); Dicentrachus labrax (GenBank Accession No. CBN81178.1); Pimephales promelas (GenBank Accession No. KAG1931208.1); Collicthys lucidus (GenBank Accession No. TKS65685.1); Zancudomyces culisetae (GenBank Accession No. OMH78677.1); Plasmodiophora brassicae (GenBank Accession No. CE094710.1); Ceratodon purpureus (GenBank Accession No. KAG0578666.1); Ceratodon purpureus (GenBank Accession No. KAG0614891.1); Xenopus laevis (GenBank Accession No. XP_031796629.1); Xenopus laevis (GenBank Accession No. OCT57199.1); Ophinophagus Hannah (GenBank Accession No. ETE58569.1); Sarcophilus harrisii (GenBank Accession No. XP_031796629.1; PEG10 Mus musculus (GenBank Accession No. NP_570947.2); PEG10 Homo sapiens (GenBank Accession No. NP_001165909.1); or Choloepus didactylus (GenBank Accession No. XP_037692625.1).

Methods and techniques to identify gag homologs suitable for use in the present invention and/or corresponding packaging elements are described in the Working Examples and elsewhere herein, such as those set forth with respect to PEG10 and RTL1, RTL3, RTL4, RTL5, RTL6, RTL7, RTL8, RTL9, and RTL10.

The gag homolog can be modified such that its ability to bind self-encoding mRNA is reduced or eliminated and/or its ability to package a cargo polynucleotide operatively coupled to the packaging element(s) is increased. Such modification can include a modification to one or more domains of the gag homolog such that it has reduced binding and/or packaging ability of its own mRNA. As demonstrated in the Working Examples here (e.g., Examples 12-13) in the context of PEG10, nucleotides in the PEG10 encoding polynucleotide were modified near the near the boundary of the nucleocapsid domain and the protease domain of the PEG10 polypeptide such that binding of PEG10 mRNA was reduced, which resulted in an increase in packaging efficiency of a cargo polynucleotide. Such approaches and methods of confirming the effect of a given modification can be used to identify other suitable modifications in PEG10 as well as suitable modifications in other gag homologs.

### Cargo Binding Domains

In some embodiments, the LTR retroelement polypeptide (including, but not limited to, a Gag homolog) or functional domain thereof may comprise both the export compartment domain and the nucleic acid-binding domain. In some embodiments, the nucleic-acid binding domain is a native nucleic acid-binding domain. In specific embodiments, the nucleic acid binding-domain may be modified relative to the native nucleic acid-binding domain of the LTR retroelement polypeptides. In specific embodiments, the nucleic acid-binding domain may be a non-native nucleic acid-binding domain relative to the LTR retroelement polypeptide (e.g., a Gag-homology protein). In some embodiments, LTR retroelement polypeptide or one or more associated proteins comprise a cargo-binding (or nucleic acid-binding) domain. In some embodiments, the cargo-binding domain is a hairpin loop-binding element. In some embodiments, the hairpin loop-binding element is an MS2 aptamer.

In some embodiments, the gag-homology protein may contain a DNA-binding motif or domain. In some embodiments, the cargo binding domain is a native DNA binding domain. In some embodiments, the DNA binding domain is an engineered or non-native binding domain. As a specific example, and as discussed in e.g., Example 4 and others, PEG10 comprises a DNA binding motif that allows for packaging of DNA of specific sequences.

In some embodiments, the cargo binding domain is an RNA binding domain. In some embodiments, the cargo binding domain is a native RNA binding domain. In some embodiments, the RNA binding domain is an engineered or non-native binding domain. Different LTR retroelement polypeptides (such as Gag proteins) evolved diverse RNA-binding domains for mediating specific encapsidation of their RNA genomes. The RNA-binding sequence specificity of the human or other organisms LTR retroelement polypeptides (such as Gag homology proteins) can be tested through protein pull-down and sequencing of associated RNA and/or through sequencing of the extracellular vesicle fraction from HEK293 cells that over-express each protein. The nucleic-acid-binding domains can be swapped between proteins, or additional RNA-binding domains with known specificity can be fused to test the extent to which binding specificity can be reprogrammed. Accordingly, the LTR retroelement polypeptide (e.g., a Gag-homology protein) or functional domain thereof can comprise both the export compartment domain and nucleic acid-binding domain. It will be appreciated that a nucleic-acid binding domain when it binds a cargo can also be referred to as a cargo-binding domain.

In an embodiment, the invention provides for introduction of an RNA sequence into a transcript recruitment sequence that forms a loop secondary structure and binds to an adapter protein. In an aspect the invention provides a herein-discussed composition, wherein the insertion of distinct RNA sequence(s) that bind to one or more adaptor proteins is an aptamer sequence. In an aspect the invention provides a herein-discussed composition, wherein the aptamer sequence is two or more aptamer sequences specific to the same adaptor protein. In an aspect the invention provides a herein-discussed composition, wherein the aptamer sequence is two or more aptamer sequences specific to a different adaptor protein. In an aspect the invention provides a herein-discussed composition, wherein the adaptor comprises MS2, PP7, Qβ, F2, GA, fr, JP501, M12, R17, BZ13, JP34, JP500, KU1, M11, MX1, TW18, VK, SP, FL, ID2, NL95, TW19, AP205, ϕCb5, ϕCb8r, ϕCb12r, ϕCb23r, 7s, PRR1. In an aspect the invention provides a herein-discussed composition, wherein the cell is a eukaryotic cell. In an aspect the invention provides a herein-discussed composition, wherein the eukaryotic cell is a mammalian cell, optionally a mouse cell. In an aspect the invention provides a herein-discussed composition, wherein the mammalian cell is a human cell. Aspects of the invention encompass embodiments relating to MS2 adaptor proteins described in Konermann et al. “Genome-scale transcriptional activation by an engineered CRISPR-Cas9 complex” Nature. 2014 Dec. 10. doi: 10.1038/naturel4136, the contents of which are herein incorporated by reference in its entirety.

In some embodiments, the adaptor protein domain is an RNA-binding protein domain. The RNA-binding protein domain recognises corresponding distinct RNA sequences, which may be aptamers. For example, the MS2 RNA-binding protein recognises and binds specifically to the MS2 aptamer (or vice versa).

Similarly, an MS2 variant adaptor domain may also be used, such as the N55 mutant, especially the N55K mutant. This is the N55K mutant of the MS2 bacteriophage coat protein (shown to have higher binding affinity than wild type MS2 in Lim, F., M. Spingola, and D. S. “Peabody.” Altering the RNA binding specificity of a translational “repressor.” Journal of Biological Chemistry 269.12 (1994): 9006-9010).

In some embodiments, the envelope protein may comprise a cargo-binding domain. In some embodiments, the cargo-binding domain is a hairpin loop-binding element. In some embodiments, the hairpin loop-binding element is an MS2 aptamer.

In some embodiments, the cargo binding domain is an RNA/DNA binding domain. In some embodiments, the cargo binding domain is a native RNA/DNA binding domain. In some embodiments, the RNA/DNA binding domain is an engineered or non-native binding domain.

In some embodiments, the LTR retroelement-polypeptide (such as a gag homology protein) and an LTR retroelement envelope protein (e.g., a retroviral envelope protein) are both endogenous. In some embodiments, the LTR retroelement polypeptide (such as a gag homology protein) is endogenous and the LTR retroelement envelope protein (e.g., a retroviral envelope protein) is of viral origin. In some embodiments, the LTR retroelement envelope protein (e.g., a retroviral envelope protein) is endogenous and the LTR retroelement polypeptide (such as a gag homology protein) is of viral origin.

In some embodiments, the LTR retroelement envelope protein is from a Gammaretrovirus or a Deltaretrovirus. In some embodiments, the envelope protein is selected from envH1, envH2, envH3, envK1, envK2_1, envK2_2, envK3, envK4, envK5, envK6, envT, envW, envW1, envfrd, envR(b), envR, envF(c)2, or envF(c)1.

In some embodiments, the vesicles comprise one or more capture moieties, e.g., for packaging a cargo and/or recruiting specific cargo(s) into the vesicle.

The term “nucleic acid capture moiety” or simply “capture moiety”, as used herein, refers to a moiety which binds selectively to a target molecule. Optionally, the moiety can be immobilized on an insoluble support, as in a microarray or to microparticles, such as beads. When used as a primer, a probe of the invention would likely not be anchored to a solid support. A capture moiety can “capture” a target molecule by hybridizing to the target and thereby immobilizing the target. In cases wherein the moiety itself is immobilized, the target too becomes immobilized. Such binding to a solid support may be through a linking moiety, which is bound to either the capture moiety or to the solid support.

The capture moiety may comprise one or more genes endogenous to the polynucleotide or plasmid, for example genes capable of recruiting the plasmid into the vesicle. The capture moiety may comprise exogenous genes or may comprise molecules capable of recruiting or capturing cargo molecules for the vesicles. In some examples, the capture moieties may interact with the cargo. The capture moieties may be nucleic acid-binding molecules, e.g., DNA, RNA, DNA-binding proteins, RNA-binding proteins, or a combination thereof. In some embodiments, the capture moieties may be protein-binding molecules, e.g., DNA, RNA, antibodies, nanobodies, antigens, receptors, ligands, fragments thereof, or a combination thereof. The capture moieties can be fused to endogenous genes or exogenous genes.

In some embodiments, the one or more capture moieties comprise DNA-binding moieties, RNA-binding moieties, protein-binding moieties, or a combination thereof.

In certain embodiments, the capture moiety may be labelled, as with, e.g., a fluorescent moiety, a radioisotope (e.g., ³²P), an antibody, an antigen, a lectin, an enzyme (e.g., alkaline phosphatase or horseradish peroxidase, which can be used in calorimetric methods), chemiluminescence, bioluminescence or other labels well known in the art. In certain embodiments, binding of a target strand to a capture moiety can be detected by chromatographic or electrophoretic methods. In embodiments in which the capture moiety does not contain a detectable label, the target nucleic acid sequence may be so labelled, or, alternatively, labelled secondary probes may be employed. A “secondary probe” includes a nucleic acid sequence which is complementary to either a region of the target nucleic acid sequence or to a region of the capture moiety. Region G of a probe (which will most often not be complementary to the target), might be useful in capturing a secondary labelled nucleic acid probe.

In some embodiments, the capture moiety is a nucleic acid hairpin. “The terms “nucleic acid” “hairpin”, “hairpin capture moiety”, or simply “hairpin”, as used herein, refer to a unimolecular nucleic acid-containing structure which comprises at least two mutually complementary nucleic acid regions such that at least one intramolecular duplex can form. Hairpins are described in, for example, Cantor and “Schimmel, “Biophysical “Chemistry”, Part III, p. 1183 (1980). In certain embodiments, the mutually complementary nucleic acid regions are connected through a nucleic acid strand; in these embodiments, the hairpin comprises a single strand of nucleic acid. A region of the capture moiety which connects regions of mutual complementarity is referred to herein as a “loop” or “linker”. In some embodiments, a loop comprises a strand of nucleic acid or modified nucleic acid. In some embodiments, the linker is not a hydrogen bond. In other embodiments, the loop comprises a linker region which is not nucleic-acid-based; however, capture moieties in which the loop region is not a nucleic acid sequence are referred to herein as hairpins. Examples of non-nucleic-acid linkers suitable for use in the loop region are known in the art and include, for example, alkyl chains (see, e.g., Doktycz et al. (1993) Biopolymers 33:1765). While it will be understood that a loop can be a single-stranded region of a hairpin, for the purposes of the discussion below, a “single-stranded region” of a hairpin refers to a non-loop region of a hairpin. In embodiments in which the loop is a nucleic acid strand, the loop preferably comprises 2-20 nucleotides, more preferably 3-8 nucleotides. The size or configuration of the loop or linker is selected to allow the regions of mutual complementarity to form an intramolecular duplex. In preferred embodiments, hairpins useful in the present invention will form at least one intramolecular duplex having at least 2 base-pairs, more preferably at least 4 base-pairs, and still more preferably at least 8 base-pairs. The number of base-pairs in the duplex region, and the base composition thereof can be chosen to assure any desired relative stability of duplex formation. For example, to prevent hybridization of non-target nucleic acids with the intramolecular duplex-forming regions of the hairpin, the number of base-pairs in the intramolecular duplex region will generally be greater than about 4 base-pairs. The intramolecular duplex will generally not have more than about 40 base-pairs. In preferred embodiments, the intramolecular duplex is less than 30 base-pairs, more preferably less than 20 base-pairs in length.

A hairpin may be capable of forming more than one loop. For example, a hairpin capable of forming two intramolecular duplexes and two loops is referred to herein as a “double hairpin”. In preferred embodiments, a hairpin will have at least one single-stranded region which is substantially complementary to a target nucleic acid “sequence. “Substantially complementary” means capable of hybridizing to a target nucleic acid sequence under the conditions employed. In preferred embodiments, a “substantially complementary” single-stranded region is exactly complementary to a target nucleic acid sequence. In preferred embodiments, hairpins useful in the present invention have a target-complementary single-stranded region having at least 5 bases, more preferably at least 8 bases. In preferred embodiments, the hairpin has a target-complementary single-stranded region having fewer than 30 bases, more preferably fewer than 25 bases. The target-complementary region will be selected to ensure that target strands form stable duplexes with the capture moiety. In embodiments in which the capture moiety is used to detect target strands from a large number of non-target sequences (e.g., when screening genomic DNA), the target-complementary region should be sufficiently long to prevent binding of non-target sequences. A target-specific single-stranded region may be at either the 3′ or the 5′ end of the capture moiety strand, or it may be situated between two intramolecular duplex regions (for example, between two duplexes in a double hairpin).

### Modifications to Increase to Enhance Cargo Loading Specificity or Efficiency

In some embodiments, the endogenous LTR retroelement polypeptide comprises one or more modifications to enhance binding specificity and/or packaging of the cargo molecule. In some embodiments, the one or more packaging elements binds to one or more domains of the endogenous LTR retroelement polypeptide.

In some embodiments, an engineered polynucleotide encoding one or more LTR retroelement polypeptides, such as a retroviral gag protein, is genetically recoded such that binding of a cargo, delivery of a cargo, or both are increased as compared to non-recoded control. In some embodiments, the engineered polynucleotide is genetically recoded such that one or more codons are swapped to activate, inactivate, or modify the function or activity of one or more domains of a polypeptide product produced from the genetically recoded engineered polynucleotide. In some embodiments, the LTR retroelement polynucleotide that is genetically recoded is a gag homolog encoding polynucleotide. In some embodiments, the LTR retroelement polynucleotide that is genetically recoded is a PEG10 encoding polynucleotide. In some embodiments, one or more codons present on the boundary of a nucleocapsid and a protease domain of an LTR retroelement polypeptide (e.g., a retroviral gag protein or gag homolog), including but not limited to PEG10, or other LTR retroelement, are swapped. In some embodiments, one or more codons present in a region of the LTR retroelement polynucleotide that is/are genetically recoded are codons present in a region of the LTR retroelement polynucleotide that is capable of self-binding (i.e., binding of RNA encoding the retroviral gag protein to the LTR retroelement polypeptide encoded by said RNA). In some embodiments and without being bound by theory, such recoding may result in a decrease of self-binding of the retroviral gag polypeptide to its encoding RNA and thus reduce competitive binding of non-cargo molecules and increase packaging of cargo molecules.

### Targeting Moiety

In some embodiments, the system includes a targeting moiety (or polynucleotide encoding said targeting moiety) configured for presentation on the engineered delivery vesicle surface to direct cell-specific binding of the delivery vesicle to a target cell type. In some embodiments, the targeting moiety is a capsid protein or other protein or molecule that confers a tropism to the delivery vesicle. Exemplary targeting moieties are described in greater detail elsewhere herein.

In some embodiments, the engineered delivery system may further comprise a targeting moiety (or polynucleotide encoding said targeting moiety). that is capable of specifically binding to a target cell. To efficiently target a delivery vesicle to cells, such as cancer cells, it is useful that the targeting moiety have an affinity for a cell surface receptor and to link the targeting moiety in sufficient quantities to have optimum affinity for the cell surface receptors; and determining these aspects are within the ambit of the skilled artisan. In the field of active targeting, there are a number of cell i.e., tumor-, specific targeting ligands. Targeting moieties can be, without limitation, an aptamer, antibody, protein, peptide, small molecule, carbohydrate, or a combination thereof.

Also, as to active targeting, with regard to targeting cell surface receptors such as cancer cell surface receptors, targeting ligands on liposomes can provide attachment of liposomes to cells, e.g., vascular cells, via a non-internalizing epitope; and this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, ‘such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid bilayers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which may enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirus or AAV Tf is a monomeric serum glycoprotein of approximately 80 KDa involved in the transport of iron throughout the body. Tf binds to the TfR and translocates into cells via receptor-mediated endocytosis. The expression of TfR can be higher in certain cells, such as tumor cells (as compared with normal cells) and is associated with the increased iron demand in rapidly proliferating cancer cells Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, cancer cells the mouth such as oral tumor cells.

Also, as to active targeting, a lipid entity of the invention can be multifunctional, i.e., employ more than one targeting moiety such as CPP, along with Tf; a bifunctional system; e.g., a combination of Tf and poly-L-arginine which can provide transport across the endothelium of the blood-brain barrier. EGFR is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid entity of the invention. HER-2 is often overexpressed in patients with breast cancer, and is also associated with lung, bladder, prostate, brain and stomach cancers. HER-2, encoded by the ERBB2 gene. The invention comprehends a HER-2-targeting lipid entity of the invention, e.g., an anti-HER-2-antibody (or binding fragment thereof)-lipid entity of the invention, a HER-2-targeting-PEGylated lipid entity of the invention (e.g., having an anti-HER-2-antibody or binding fragment thereof), a HER-2-targeting-maleimide-PEG polymer-lipid entity of the invention (e.g., having an anti-HER-2-antibody or binding fragment thereof). Upon cellular association, the receptor-antibody complex can be internalized by formation of an endosome for delivery to the cytoplasm. With respect to receptor-mediated targeting, the skilled artisan takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bilayer). The microenvironment of a cell mass such as a tumor microenvironment can be targeted; for instance, it may be advantageous to target cell mass vasculature, such as the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) e.g., via or with a PEG terminus), tumor-homing peptide APRPG (SEQ ID NO: 4) such as APRPG-PEG-modified. VCAM, the vascular endothelium plays a key role in the pathogenesis of inflammation, thrombosis and atherosclerosis. CAMs are involved in inflammatory disorders, including cancer, and are a logical target; E- and P-selectins, VCAM-1 and ICAMs can be used to target a lipid entity of the invention, e.g., with PEGylation Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix. An antibody or fragment thereof such as a Fab′ fragment can be used in the practice of the invention such as for an antihuman MT1-MMP monoclonal antibody linked to a lipid entity of the invention, e.g., via a spacer such as a PEG spacer. αβ-integrins or integrins are a group of transmembrane glycoprotein receptors that mediate attachment between a cell and its surrounding tissues or extracellular matrix. Integrins contain two distinct chains (heterodimers) called α- and ρ-subunits. The tumor tissue-specific expression of integrin receptors can be utilized for targeted delivery in the invention, e.g., whereby the targeting moiety can be an RGD peptide such as a cyclic RGD. Aptamers are ssDNA or RNA oligonucleotides that impart high affinity and specific recognition of the target molecules by electrostatic interactions, hydrogen bonding and hydrophobic interactions as opposed to Watson-Crick base-pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer). The targeting moiety can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the particular environment or microenvironment such as an endocytic vacuole or the acidotic tumor mass. pH-sensitive copolymers can also be incorporated in embodiments of the invention and can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)). Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at a site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature-triggered system can employ lysolipid temperature-sensitive liposomes. The invention also comprehends redox-triggered delivery: The difference in redox potential between normal and inflamed or tumor tissues, and between the intra- and extra-cellular environments has been exploited for delivery, e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction-sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization, leading to release of payload. Calcein release from reduction-sensitive lipid entity of the invention containing a disulfide conjugate can be more useful than a reduction-insensitive embodiment. Enzymes can also be used as a trigger to release payload. Enzymes, including MMPs (e.g., MMP2), phospholipase A2, alkaline phosphatase, transglutaminase or phosphatidylinositol-specific phospholipase C, have been found to be overexpressed in certain tissues, e.g., tumor tissues. In the presence of these enzymes, an engineered enzyme-sensitive lipid entity of the invention can be disrupted and release the payload. An MMP2-cleavable octapeptide (Gly-Pro-Leu-Gly-Ile-Ala-Gly-Gln) (SEQ ID NO: 5) can be incorporated into a linker, and can have antibody targeting, e.g., antibody 2C5. The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefore can be a benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe3O4 or 7-Fe2O3, e.g., those that are less than 10 nm in size. Targeted delivery can then be by exposure to a magnetic field.

Also, as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides destabilize the endosomal membrane after the conformational transition/activation at a lowered pH. Amines are protonated at an acidic pH and cause endosomal swelling and rupture by a buffer effect. Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA, cholesteryl-GALA and PEG-GALA may show a highly efficient endosomal release; a pore-forming protein listeriolysin O may provide an endosomal escape mechanism; and, histidine-rich peptides have the ability to fuse with the endosomal membrane, resulting in pore formation, and can buffer the proton pump, causing membrane lysis. In some embodiments, the engineered delivery vesicles can contain a lipid layer, such as a lipid outer layer or contain lipids in their outer surface.

Also, as to active targeting, cell-penetrating peptides (CPPs) facilitate uptake of macromolecules through cellular membranes and, thus, enhance the delivery of CPP-modified molecules inside the cell. CPPs can be split into two classes: amphipathic helical peptides, such as transportan and MAP, where lysine residues are major contributors to the positive charge; and Arg-rich peptides, such as TATp, Antennapedia or penetratin. TATp is a transcription-activating factor with 86 amino acids that contains a highly basic (two Lys and six Arg among nine residues) protein transduction domain, which brings about nuclear localization and RNA-binding. Other CPPs that have been used for the modification of liposomes include the following: the minimal protein transduction domain of Antennapedia, a Drosophilia homeoprotein, called penetratin, which is a 16-mer peptide (residues 43-58) present in the third helix of the homeodomain; a 27-amino acid-long chimeric CPP, containing the peptide sequence from the amino terminus of the neuropeptide galanin bound via the Lys residue, mastoparan, a wasp venom peptide; VP22, a major structural component of HSV-1 facilitating intracellular transport and transportan (18-mer) amphipathic model peptide that translocates plasma membranes of mast cells and endothelial cells by both energy-dependent and -independent mechanisms. The invention comprehends a lipid entity of the invention modified with CPP(s), for intracellular delivery that may proceed via energy dependent 54 icropinocytosis followed by endosomal escape. The invention further comprehends organelle-specific targeting. A lipid entity of the invention surface-functionalized with the triphenylphosphonium (TPP) moiety or a lipid entity of the invention with a lipophilic cation, rhodamine 123 can be effective in delivery of cargo to mitochondria. DOPE/sphingomyelin/stearyl-octa-arginine can deliver cargos to the mitochondrial interior via membrane fusion. A lipid entity of the invention surface-modified with a lysosomotropic ligand, octadecyl rhodamine B can deliver cargo to lysosomes. Ceramides are useful in inducing lysosomal membrane permeabilization; the invention comprehends intracellular delivery of a lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhance accumulation in a desired site and/or promote organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

An embodiment of the invention includes the delivery system comprising an actively targeting lipid particle or nanoparticle or liposome or lipid bilayer delivery system; or comprising a lipid particle or nanoparticle or liposome or lipid bilayer comprising a targeting moiety whereby there is active targeting or wherein the targeting moiety is an actively targeting moiety. A targeting moiety can be one or more targeting moieties, and a targeting moiety can be for any desired type of targeting such as, e.g., to target a cell such as any herein-mentioned; or to target an organelle such as any herein-mentioned; or for targeting a response such as to a physical condition such as heat, energy, ultrasound, light, pH, chemical such as enzymatic, or magnetic stimuli; or to target to achieve a particular outcome such as delivery of payload to a particular location, such as by cell penetration.

It should be understood that as to each possible targeting or active targeting moiety herein-discussed, there is an aspect of the invention wherein the delivery system comprises such a targeting or active targeting moiety. Likewise, Table 1 provides exemplary targeting moieties that can be used in the practice of the invention, and, as to each an aspect of the invention provides a delivery system that comprises such a targeting moiety.

TABLE 1

| Targeting Moiety | Target Molecule | Target Cell or Tissue |
|---|---|---|
| folate | folate receptor | cancer cells |
| transferrin | transferrin | cancer cells |
| | receptor | |
| Antibody CC52 | rat CC531 | rat colon adeno- |
| | | carcinoma CC531 |
| anti-HER2 antibody | HER2 | HER2-overexpressing |
| | | tumors |
| anti-GD2 | GD2 | neuroblastoma, |
| | | melanoma |
| anti-EGFR | EGFR | tumor cells over- |
| | | expressing EGFR |
| pH-dependent fusogenic | | ovarian carcinoma |
| peptide diINF-7 | | |
| anti-VEGFR | VEGF Receptor | tumor vasculature |
| anti-CD19 | CD19 | leukemia, lymphoma |
| | (B cell marker) | |
| cell-penetrating peptide | | blood-brain barrier |
| cyclic arginine-glycine- | avβ3 | glioblastoma cells, |
| aspartic acid-tyrosine- | | human umbilical vein |
| cysteine peptide | | endothelial cells, tumor |
| (c(RGDyC)-LP) | | angiogenesis |
| (SEQ ID NO: 6) | | |
| ASSHN peptide | | endothelial progenitor |
| | | cells; anti-cancer |
| PR_b peptide | α5β1 integrin | cancer cells |
| AG86 peptide | α6β4 integrin | cancer cells |
| KCCYSL (P6.1 peptide) | HER-2 receptor | cancer cells |
| (SEQ ID NO: 7) | | |
| affinity peptide LN | Aminopeptidase N | APN-positive tumor |
| (YEVGHRC) | (APN/CD13) | |
| (SEQ ID NO: 8) | | |
| synthetic somatostatin | Somatostatin | breast cancer |
| analogue | receptor 2 | |
| | (SSTR2) | |
| anti-CD20 monoclonal | B-lymphocytes | B cell lymphoma |
| antibody | | |

Thus, in an embodiment of the delivery system, the targeting moiety comprises a receptor ligand, such as, for example, hyaluronic acid for CD44 receptor, galactose for hepatocytes, or antibody or fragment thereof such as a binding antibody fragment against a desired surface receptor, and as to each of a targeting moiety comprising a receptor ligand, or an antibody or fragment thereof such as a binding fragment thereof, such as against a desired surface receptor, there is an aspect of the invention wherein the delivery system comprises a targeting moiety comprising a receptor ligand, or an antibody or fragment thereof such as a binding fragment thereof, such as against a desired surface receptor, or hyaluronic acid for CD44 receptor, galactose for hepatocytes (see, e.g., Surace et al, “Lipoplexes targeting the CD44 hyaluronic acid receptor for efficient transfection of breast cancer cells,” J. Mol Pharm 6(4):1062-73; doi: 10.1021/mp800215d (2009); Sonoke et al, “Galactose-modified cationic liposomes as a liver-targeting delivery system for small interfering RNA,” Biol Pharm Bull. 34(8):1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2): 189-204 (2008); Gao J et al, “Antibody-targeted immunoliposomes for cancer treatment,” Mini. Rev. Med. Chem. 13(14): 2026-2035 (2013); Molavi et al, “Anti-CD30 antibody conjugated liposomal doxorubicin with significantly improved therapeutic efficacy against anaplastic large cell lymphoma,” Biomaterials 34(34):8718-25 (2013), each of which and the documents cited therein are hereby incorporated herein by reference).

Moreover, in view of the teachings herein the skilled artisan can readily select and apply a desired targeting moiety in the practice of the invention as to a lipid entity of the invention. The invention comprehends an embodiment wherein the delivery system comprises a lipid entity having a targeting moiety.

In some embodiments, the target cell may be a mammalian cell. In some embodiments, the mammalian cell may be a cancer cell, as described further below.

In some embodiments, the mammalian cell may be infected with a pathogen. In some embodiments, the pathogen may be a virus, as described further below.

In some embodiments, the targeting moiety comprises or is a membrane fusion protein (e.g., a fusogen). Fusogens are also described elsewhere herein. In some embodiments, the membrane fusion protein is the G envelope protein of vesicular stomatitis virus (VSV-G). In some embodiments the membrane fusion protein is a membrane fusion protein described in greater detail elsewhere herein. In some embodiments, the membrane fusion protein is a viral glycoprotein. Non-limiting exemplary viral glycoproteins include Influenza virus glycoproteins (e.g., hemagglutinin, neuraminidase), SARS-CoV glycoproteins (e.g., spike (S) glycoprotein, hepatitis C virus glycoproteins (e.g. E1 and E2), HIV-1 glycoproteins (e.g., gp120, gp160, gp41, HIV-2 (e.g., env-encoded glycoprotein) Ebola virus glycoproteins (e.g., Spike protein Gp1-Gp2), Dengue virus glycoproteins (e.g., E (dimer)), Chikungynya virus glycoproteins (e.g., E1 and E2), vesicular stomatitis virus glycoprotein (VSV-G), Lassa virus (e.g. gp1); HTLV-1 (e.g. gp21), measles virus glycoproteins (e.g., haemagglutinin and fusion (F) protein), rabies virus glycoprotein (e.g., RGP or RVG), Nipah virus and Hendra virus Glycoproteins (e.g., NiV-G and HeV-G, collectively referred to as HNV-G proteins), Marburg virus glycoprotein (e.g., MARV-G or MARV-GP), respiratory syncytial Virus glycoprotein (e.g., RSV-G), rhabdovirus glycoprotein G, foamy virus envelope glycoproteins (including but not limited to bovine foamy virus glycoprotein, equine foamy virus glycoprotein, feline foamy virus glycoprotein, Eastern chimpanzee and foamy virus glycoprotein), Aujeszky's disease virus (e.g., gB, gC, gD, gE, gG, gH, gI, gK, gL, gM, and gM), human endogenous retrovirus type W (HERV-W) envelope glycoprotein (Env), Simian retrovirus envelope glycoprotein (Env), Feline leukemia virus surface glycoproteins (FeLV-SU), equine infectious anemia glycoprotein (e.g. gp90), murine leukemia virus (MuLV) envelope surface (SU) glycoproteins, a gammaretrovirus glycoprotein, a delta retrovirus glycoprotein, a lentivirus glycoprotein, a herpesvirus glycoprotein (G) (e.g., gB), a group 1 alphabaculovirus glycoprotein (e.g., gp64), Epstein-Barr virus glycoprotein (G), baculovirus glycoprotein (e.g., gp64), and combinations thereof.

Membrane fusion is a universal and important biological phenomenon that occurs when two separate lipid membranes merge into a single continuous bilayer. Fusion reactions share common features but are catalyzed by diverse proteins. These proteins mediate the initial recognition of the membranes that are destined for fusion and pull the membranes close together to destabilize the lipid/water interface and to initiate mixing of the lipids. A single fusion protein may do everything, or assemblies of protein complexes may be required for intracellular fusion reactions to guarantee rigorous regulation in space and time. Cellular fusion machines are adapted to fit the needs of different reactions but operate by similar principles in order to achieve merging of the bilayers.

Membrane fusion can range from cell fusion and organelle dynamics to vesicle trafficking and viral infection. Without exception, all of these fusion events are driven by membrane fusion proteins, also known as fusogens. The common fusion process mediated by fusion proteins consists of a series of steps that includes the approach of two opposing lipid membranes, breaking the lipid bilayers, and finally merging the two lipid bilayers into one. Much of our understanding of membrane fusion comes from studies of vesicle fusion, which is driven by a special kind of protein called SNARE. The SNARE proteins on vesicles (v-SNARE) and those on target membranes (t-SNARE) provide not only recognition specificity but also the energy needed for vesicle fusion.

In some embodiments, the system includes a tetraspanin (TSPAN) transmembrane protein or TSPAN encoding polynucleotide. In some embodiments, the TSPAN is CD81, CD9, CD63, or any combination thereof.

In some embodiments, the system includes a transmembrane protein selected from Syncytin A (SynA), Syncytin B, Syncytin 1, Syncytin 2, or a combination thereof.

Viral fusion is another important fusion event. Enveloped viruses that are encapsulated by membranes derived from host cells release genomes after the fusion between viral envelope and host cellular membrane. Viral fusion proteins dominate the uncoating stage. According to their structural characteristics, viral fusion proteins are classified into three types: I, II and III. Despite longstanding knowledge of viral fusion proteins, the underlying fusion mechanism remains mysterious. One such previously identified type III viral fusion protein is vesicular stomatitis virus G protein (VSV-G). Previous studies have revealed that VSV-G-triggered membrane fusion in acidic environments relies on reversible conformational changes, which return to their original state under neutral conditions. VSV-G and the fusion proteins of related rhabdoviruses (e.g., rabies virus) is the sole surface-expressed protein on the bullet-shaped virions. It mediates both attachment and low-pH-induced fusion.

The engineered delivery system can include one or more moieties that can confer a cell-specific tropism to the engineered delivery vesicles produced therefrom and described herein. The cell specific tropism can be based upon tropism of virus particles that infect one or more specific particular cell types. In some embodiments, the tropism cell-specific tropism can be conferred by inclusion of one or more ligands for a viral cell receptor on a cell. Suitable ligands that can be capable of conferring a cell specific tropism are discussed in Schneider-Schaulies. 2000. J. Gen. Virol. 81:1413-1429. Techniques employed to alter AAV, lentiviral, or other viral tropism can be used to modify the tropism of the engineered delivery systems and delivery vesicles produced therefrom described herein. The approach described in Gleyzer at al. 2016. Microsc. Microanal. 22 (Suppl 3) 1098. to alter lentiviral tropism can be modified and applied to modify the tropism of the engineered delivery systems and delivery vesicles produced therefrom described herein. In some embodiments, a tropism switching gene cassette can be incorporated into the engineered delivery system described herein. Such host range variation systems can be found bacterium that have a Mu and/or P1 genetic system.

Cytokines can also be used to alter cellular, tissue, and/or organ tropism of the engineered delivery systems and delivery vesicles produced therefrom described herein. Exemplary cytokines and other approaches that can provide a cell, tissue, or organ specific tropism that can be used in or with the engineered delivery systems and delivery vesicles produced therefrom described herein are discussed in McFadden et al., 2009. Nat. Rev. Immunol. 9(9): 645-655.

In some embodiments, the targeting moiety is a viral capsid protein or a portion thereof, that confers a tropism to the delivery particle. In some embodiments, the targeting moiety is an AAV capsid protein or portion thereof. In some embodiments, the targeting moiety is such that the delivery vesicle has the cell-specificity or tropism of an AAV 1, AAV2, AAV3, AAV4, AAV5, AAV6, AAV7, AAV8, AAV9 serotype, or a combination thereof.

In some embodiments, the targeting moiety is an amino acid motif that can optionally be integrated with or operably coupled to another polypeptide, such as a capsid polypeptide or other polypeptide of the engineered delivery vesicles described herein. In some embodiments, the amino acid motif confers tissue and/or cell specificity to the composition to which it is coupled to or integrated with. In some embodiments, the amino acid motif contains an “RGD” motif (see e.g., Weinmann et al. Nature Com. (2020) 11:5432 | https://doi.org/10.1038/s41467-020-19230-w and International Patent Application Publication WO 2019207132). In some embodiments, when the targeting moiety is an amino acid containing an RGD motif, the targeting moiety is capable of targeting muscle cells.

### Isolation Tags

In some embodiments, a delivery system described herein further includes an isolation tag (or polynucleotide encoding the same) that is configured for presentation on the delivery vesicle surface to enable isolation of the delivery vesicle. Accordingly, the polynucleotide may further encode a protein affinity tag. Location of the affinity tag on the expressed protein will be dictated by the need to ensure the affinity tag is added to the retroviral polypeptide such that it is presented on the outer surface of the delivery vesicle once formed.

One or more of the polypeptides of the engineered delivery vesicles described herein can be operably linked, fused to, or otherwise modified to include (such inserted between two amino acids between the N- and C-terminus of the polypeptide) a selectable marker, affinity, or other protein tag. It will be appreciated that the polynucleotide encoding such selectable markers or tags can be incorporated into a polynucleotide encoding one or more components of the engineered delivery system described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure. Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; fluorescence tags, such as GFP and mCherry; protein tags that may allow specific enzymatic modification (such as biotinylation by biotin ligase) or chemical modification (such as reaction with FlAsH-EDT2 for fluorescence imaging). Selectable markers and tags can be operably linked to one or more components of the engineered delivery system described herein via suitable linker, such as a glycine or glycine serine linkers as short as GS or GG up to (GGGGG)₃ (SEQ ID NO: 27) or (GGGGS)₃ (SEQ ID NO: 28). Other suitable linkers are generally known in the art and/or described elsewhere herein.

Examples of additional selectable markers and/or isolation tags include, but are not limited to, DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); the generation of new primer sites for PCR (e.g., the juxtaposition of two DNA sequences not previously juxtaposed), the inclusion of DNA sequences not acted upon or acted upon by a restriction endonuclease or other DNA modifying enzyme, chemical, etc.; epitope tags (e.g. GFP, FLAG- and His-tags), and, the inclusion of a DNA sequences required for a specific modification (e.g., methylation) that allows its identification. Other suitable markers will be appreciated by those of skill in the art. Further it will be appreciated that such markers and tags can be provided in an engineered delivery vesicle generation system in the form of an encoding polynucleotide. In other words, the engineered delivery vesicle generation system can include one or more isolation tag and/or selectable marker encoding polynucleotide that can be operably coupled with, integrated with, or otherwise associated with one or more of the other components of the system.

Such markers and tags can be used for identification, isolation, and/or purification of the engineered delivery vesicles and/or encoding polynucleotides. In some embodiments, the engineered delivery system polynucleotide(s) include one or more tags such that when expressed and incorporated into a delivery vesicle the tag or marker is expressed on the outside of the delivery vesicle.

### Cargo Molecules

The delivery vesicle generation system may further include a cargo molecule that is delivered with the polynucleotide encoding the LTR retroelement polypeptide for packaging. In certain example embodiments, the delivery vesicle generation systems may only consist of the LTR retroelement polypeptide with the cargo to be provided by a cell into which the delivery vesicle generation system is delivered. A wide range of cargo molecules, limited by the size parameters of the delivery vesicles, may be packaged into the delivery vesicles including, polynucleotides, polypeptides, polysaccharides, ribonucleoprotein (RNP) complexes, and small molecules. An expanded list of example cargo molecules is provided below. In some embodiments, where the cargo to be packaged, the cargo is a polynucleotide. The polynucleotide may be delivered on a vector. The cargo polynucleotide may be delivered on the same vector as the LTR retroelement polypeptide or on a separate vector.

### Packaging Elements

In certain example embodiments, the cargo molecule may be modified with one or more packaging elements. As used in this and similar contexts herein a “packaging element” is polynucleotide element capable of complexing with one or more domains of the LTR retroelement polypeptide to facilitate packaging of the cargo molecule into the delivery vesicle.

In some embodiments, the one or more packing elements are optionally linked to a cargo(s) via one or more linkers. In some embodiments, one or more of the one or more linkers is cleavable by an enzyme (e.g., a protease) or is sensitive to a specific environmental condition (e.g., pH) such that when in the presence of the cleaving enzyme in a target/recipient cell or specific environmental condition (like acidic pH at the brush border membrane of the intestine or within a lysosome), the linker is cleaved and the cargo(s) is/are released. In some embodiments, a producer cell (a cell used to generate the delivery vesicles and/or package cargo(s)) can be deficient in an enzyme capable of cleaving a linker present between the cargo and packaging element and/or does not contain an environment to which linkers present are sensitive to such that a cargo is not prematurely released or packaging of the cargo(s) is impeded or inhibited. In some embodiments, the producer cells can be engineered such that they do not contain a linker cleaving enzyme or specific environment to which the linker is sensitive. It will be appreciated that the cleaving enzyme can be endogenous to a target/recipient cell or a target cell can be engineered or modified to contain a cleaving enzyme.

In certain example embodiments, the LTR retroelement polypeptide is capable of packaging its own mRNA through binding to a 5′ UTR, 3′UTR or both. Thus, in certain example embodiments, the one or more packaging elements comprise a 5′ UTR, 3′ UTR, or both or a functional portion thereof derived from the mRNA encoding the LTR retroelement polypeptide. In certain example embodiments, the 5′ UTR and/or 3′ UTR can be shorted to a minimal segment needed to facilitate packaging into the delivery vesicles.

In certain example embodiments, the mRNA encoding the LTR retroelement polypeptide from which the packaging element is derived is mRNA encoding a Sushi class protein such as PEG10, RTL1, RTL3, RTL4, RTL5, RTL6, RTL 7, RTL8, RTL 9, or RTL10. In some embodiments, the mRNA encoding an endogenous LTR retroelement polypeptide is an mRNA encoding a PEG10 polypeptide or orthologue thereof, an RTL1 polypeptide or orthologue thereof, an RTL3 polypeptide or orthologue thereof, an RTL5 polypeptide or orthologue thereof, an RTL6 polypeptide or orthologue thereof.

Methods for selecting a minimal UTR segment are provided in further detail below in the Example PEG10 embodiment and in the Working Examples herein. In some embodiments, the minimal packaging element is about 500 bp of the proximal region of the 3′UTR of an LTR retroelement polypeptide mRNA. In some embodiments, the minimal packaging element is about 500 bp of the proximal region of the 3′UTR of a gag homolog mRNA. In some embodiments, the minimal packaging element is about 500 bp of the proximal region of the 3′UTR of a PEG10 mRNA. In some embodiments, the minimal packaging element is about 500, about 450 bp, about 400 bp, about 350 bp, about 300 bp, about 250 bp, about 200 bp or less of the proximal region of the 3′UTR of a gag homolog mRNA.

In certain example embodiments, the packaging element is a polynucleotide comprising a polynucleotide motif having a sequence of UNNUU, wherein each N is independently selected from A, T, C, G, or U.

In some embodiments, a native packaging element binds a native or engineered domain of an LTR retroelement polypeptide. In certain other example embodiments, a packaging element may be engineered to bind with a domain on the LTR retroelement polypeptide. The domain may be a natural domain of the LTR retroelement polypeptide or a cargo binding domain engineered into the LTR retroelement polypeptide. For example, if the cargo binding domain of the LTR retroelement polypeptide is a MS2 variant adaptor domain, the packaging element may be the MS2 hairpin recognized by the MS2 variant adapter domain.

### Fusogenic Polypeptides

In certain example embodiments, the delivery vesicle generation systems may further comprise a fusogenic polypeptide. The fusogenic polypeptide may be encoded by a polynucleotide and expressed along with the LTR retroelement polypeptide. The fusogenic likewise may be encoded on a vector under the control of one or more regulatory elements. The fusogenic polypeptide may be encoded on the same or a separate vector. In some embodiments, the fusogenic polypeptide is an endogenous fusogenic polypeptide. In some embodiments, the fusogenic polypeptide is non-endogenous (i.e., is exogenous).

In some embodiments, the engineered system and/or vesicle includes one or more fusogenic polypeptide or membrane fusion molecules. The membrane fusion molecule (also known by the term of art as a fusogen or fusogenic lipid or protein) can be viral or non-viral. In some embodiments, a system, vesicle and/or particle of the present invention can include one or more membrane fusion molecules. In some embodiments, the fusogen(s) are proteins, In some embodiments, the fusogen(s) are lipids. In some embodiments, the system, vesicle, or particle of the present invention include both fusogenic proteins and fusogenic lipids. Exemplary membrane fusion proteins include, but are not limited to, SNARE proteins (e.g., v-SNARE (vesicle SNARE proteins), t-SNARE (target SNARE proteins), VAMPs (vesicle associated membrane proteins)(e.g., VAMP1, VAMP2, VAMP3, VAMP4, VAMP5, VAMP7, VAMP8), tetraspanins (TSPANs) (e.g., CD81, CD9, and CD63), syncytins (e.g., Syncytin A (SynA), Syncytin B, Syncytin 1, Syncytin 2), epsilon-sarcoglycan (SGCE), a viral fusion protein (e.g., viral glycoproteins and envelope proteins (also described in greater detail elsewhere herein), an flavivirus fusion protein (e.g. E), an alphavirus fusion protein (e.g., E1), a bunyavirus fusion protein, paramyxovirus fusion (F) protein), a Class IV viral fusion protein (also known as fusion-associated small transmembrane (FAST) proteins (e.g., a reovirus fusion protein), a Class II viral fusion protein (e.g. an envelope protein from Flaviviridae (E) (e.g., West Nile Virus or Dengue virus E protein), a Class I viral fusion protein (e.g., Orthomyxoviridae or Paramyxoviride hemagluttinin, Retroviridae family glycoprotein 41), EVR3 envelope protein, Hendra virus (F) protein)), a gag-homology protein (e.g., Arghap32, Clmp, and CXDAR, and others described in greater detail herein (including but not limited to those genes/gene products therefrom listed in e.g., Tables 7, 8, and 10 in the Working Examples herein), cell penetrating peptides (described in greater detail elsewhere herein, pH-dependent fusogenic peptide diINF-7, and combinations thereof. Exemplary membrane fusion lipids include, but are not limited to, lipid GALA, cholesteryl-GALA, PEG-GALA, DOPE, 1,2-dioleoyl-3-trimethylammonium-propane (DOTAP), PE, DAG, lyso phospholipids, phosphatidic acid, L-a-dioleoyl phosphatidyl choline (DOPC). Other exemplary fusogens are also described elsewhere herein and can be included in the engineered system and/or vesicles of the present invention. Other fusogenic polypeptides are described elsewhere herein, such as in the “targeting moieties” section.

In some embodiments, the fusogenic polypeptide is specific for a target cell type to which the cargo polynucleotide is targeted for delivery.

In some embodiments, the fusogenic polypeptide is a tetraspaninn (TSPAN), a G envelope protein, a SGCE, a syncitin, or a combination thereof. In some embodiments, the TSPAN is CD81, CD9, CD63 or a combination thereof. In some embodiments, the G envelope protein is a vesicular stomatitis virus G envelope protein (VSV-G).

In some embodiments, the fusogens included improve production of the engineered delivery vesicles. For example, fusogens that reduce or eliminate fusion of producer cells or that result in a higher titer of particles produced can be used.

In some embodiments, the fusogens included reduce the immunogenicity of the engineered delivery vesicles.

In some embodiments, the fusogenic polypeptide is one or more from Table 7 and/or Table 8.

Fusogens suitable for use as fusogenic polypeptides in the present invention can be identified as Applicant did as demonstrated in the Working Examples herein (see e.g., at least Tables 7 and 8 and Examples 12 and 13). Generally, tissue-wide sequencing/expression databases, many of which are publicly available, can be searched to determine what tissues that the (e.g., endogenous) LTR retroelement polypeptide included in the system is highly expressed in. Once those tissues have been identified, the tissue-wide sequencing/expression database can be searched to see what fusogenic polypeptides are expressed in those same tissues. Suitable fusogenic polypeptides are thus those that are expressed in the same tissues and/or cell types that have high or significant expression of the (e.g., endogenous) LTR retroelement polypeptide included in the system. Fusogens identified using such a co-expression screening method can be experientially verified in cell lines that are capable of transduction with the identified fusogens, as is also demonstrated and as discussed in e.g., Working Examples 12-13 herein. Delivery vesicles pseduotyped with a candidate fusogen can be incubated with cells known to be transduced by the candidate fusogen. The delivery vesicles can be loaded with a reporter cargo that can be measured to determine transduction efficiency of the cell, which can allow for confirmation of suitable fusogens for the present invention.

Briefly and as discussed in e.g., Working Examples 12-13 herein, it was observed that PEG10 was highly expressed in placenta and placenta also expresses SYNA and SYNB. The literature identified SYNA and SYNB as effective to pseudotype lentivirus for efficient transgene delivery and a reanalysis of PEG10 CLIP data in mouse trophoblast stem cells showed a direct interaction between PEG10 and the mouse syncytin transcript, thus confirming that co-expression data can reveal suitable fusogens. This was further supported by single-cell sequence databases of human synctiotrophoblasts that showed analogous endogenous fusogens to the mouse SYNA and SYNB are expressed in the same cell types as human PEG10. Candidate fusogens were experimentally verified by incubating delivery vesicles pseudotyped with the candidate fusogens and carrying a reporter (in this case a Cre recombinase that will modify a fluorescent reporter gene expressed in the cell) with cells capable of being transduced by the candidate fusogen and transduction was then determined by measuring the fluorescence of the reporter.

### Vectors

Also provided herein are vectors that can contain one or more polynucleotides that encode one or more of the engineered delivery vesicle generation system polypeptides. In some embodiments, the vectors can be used for expression and production of engineered delivery vesicles. In some embodiments, the vector(s) comprising the engineered delivery vesicle generation system polynucleotides described herein can be delivered to a cell, such as donor cell, which can be then included in a co-culture system or be delivered to a subject, such as in cell therapy. In aspects, the vector can contain one or more polynucleotides encoding one or more elements of an engineered delivery vesicle generation system described herein. The vectors can be useful in producing bacterial, fungal, yeast, plant cells, animal cells, and transgenic animals that can express one or more components of the engineered delivery vesicle generation system described herein and/or generate delivery vesicles and/or packaging cargo within the delivery vesicles. Within the scope of this disclosure are vectors containing one or more of the polynucleotide sequences described herein. One or more of the polynucleotides that are part of the engineered delivery vesicle generation system described herein can be included in a vector or vector system. The vectors and/or vector systems can be used, for example, to express one or more of the polynucleotides in a cell, such as a producer cell, to produce engineered delivery vesicles described elsewhere herein. Other uses for the vectors and vector system are also within the scope of this disclosure.

A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Engineered delivery vesicle generation system encoding polynucleotide(s) can be codon optimized for expression in a specific cell-type and/or subject type. An example of a codon optimized sequence is, in this instance, a sequence optimized for expression in a eukaryote, e.g., humans (i.e., being optimized for expression in a human or human cell), or for another eukaryote, animal or mammal as herein discussed is within the ambit of the skilled artisan. It will be appreciated that other examples are possible and codon optimization for a host species other than human, or for codon optimization for specific organs is known. In some embodiments, an enzyme coding sequence encoding one or more elements of the engineered delivery vesicle generation system described herein is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton BR, J Mol Evol. 1998 April; 46(4):449-59.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Vectors for and that result in expression in a eukaryotic cell can be referred to herein as “eukaryotic expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” and “operatively-linked are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells.

With regards to recombination and cloning methods, mention is made of U.S. Pat. Pub 2004/0171156, the contents of which are herein incorporated by reference in their entirety.

Advantageous vectors include lentiviruses and adeno-associated viruses, and types of such vectors can also be selected for targeting particular types of cells.

In particular embodiments, use is made of bicistronic vectors for one or more elements of the engineered delivery vesicle generation system described herein. In some embodiments, expression of elements of the engineered delivery vesicle generation system described herein can be driven by the CBh promoter. Where the element of the engineered delivery vesicle generation system is an RNA, its expression can be driven by a Pol III promoter, such as a U6 promoter. In some embodiments, the two are combined.

Vectors can be designed for expression of one or more elements of the engineered delivery vesicle generation system described herein (e.g., nucleic acid transcripts, proteins, or enzymes) in prokaryotic or eukaryotic cells. For example, one or more elements of the engineered delivery vesicle generation system described herein can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89). In some embodiments, a vector is a yeast expression vector. Examples of vectors for expression in yeast Saccharomyces cerivisae include pYepSecI (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170: 31-39).

As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors may contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other aspects can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more elements of engineered delivery vesicle generation system so as to drive expression of the one or more elements of the engineered delivery vesicle generation system described herein.

In some embodiments, one or more vectors driving expression of one or more elements of an engineered delivery vesicle generation system described herein are introduced into a host cell such that expression of the elements of the engineered delivery vesicle generation system described herein direct formation of the engineered delivery vesicle described herein (including but not limited to an engineered delivery vesicle), which is described in greater detail elsewhere herein). For example, different elements of the engineered delivery system described herein could each be operably linked to separate regulatory elements on separate vectors. RNA(s) of different elements of the engineered delivery vesicle generation system described herein can be delivered to an animal or mammal or cell thereof to produce an animal or mammal or cell thereof that constitutively or inducibly or conditionally expresses different elements of the engineered delivery vesicle generation system described herein that incorporates one or more engineered delivery vesicle generation system described herein or contains one or more cells that incorporates and/or expresses one or more elements of the engineered delivery vesicle generation system described herein. Alternatively, two or more of the elements expressed from the same or different regulatory elements, may be combined in a single vector, with one or more additional vectors providing any components of the system not included in the first vector. Engineered delivery vesicle generation system polynucleotides that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter or other regulatory element drives expression of a transcript encoding one or more engineered delivery vesicle generation system proteins, embedded within one or more intron sequences (e.g., each in a different intron, two or more in at least one intron, or all in a single intron). In some embodiments, the engineered delivery system polynucleotides can be operably linked to and expressed from the same promoter. In some embodiments, no two encoding polynucleotides of engineered delivery vesicle generation system elements are operably linked to the same regulatory element. In some embodiments, two or more encoding polynucleotides of engineered delivery vesicle generation system elements are operably linked to different regulatory elements. In some embodiments, two or more encoding polynucleotides of engineered delivery vesicle generation system elements are operably linked to the same regulatory element(s). In some embodiments, a polynucleotide encoding an endogenous gag homology polypeptide is operably linked to a different regulatory element as a polynucleotide encoding a cargo polynucleotide and/or one or more packaging elements. In some embodiments, a polynucleotide encoding an endogenous gag homology polypeptide is operably linked to the same regulatory element as a polynucleotide encoding a cargo polynucleotide and/or one or more packaging elements.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

In some aspects, a vector capable of expressing a engineered delivery system polynucleotide in a cell can be composed of or contain a minimal promoter operably linked to a polynucleotide sequence encoding the an engineered delivery system polypeptide described herein and a second minimal promoter operably linked to a polynucleotide sequence encoding at least one guide RNA, wherein the length of the vector sequence comprising the minimal promoters and polynucleotide sequences is less than 4.4 Kb. In an embodiment, the vector can be a viral vector. In aspects, the viral vector is an is an adeno-associated virus (AAV) or an adenovirus vector.

The vectors can include one or more regulatory elements, which can optionally operably be coupled to a polynucleotide that encodes one or more elements of the engineered delivery vesicle generation system described herein. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g., nuclear localization signals). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6, 7SK, and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). Specific configurations of the gRNAs, reporter gene and pol II and pol III promoters in the context of the present invention are described in greater detail elsewhere herein.

In some embodiments, the regulatory sequence can be a regulatory sequence described in U.S. Pat. No. 7,776,321, U.S. Pat. Pub. No. 2011/0027239, and International Patent Publication No. WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

The regulatory elements, such as promoters, can be optimized in embodiments of an engineered delivery vesicle generation system described herein. In some embodiments, the promoters selected to drive expression of each component of the system are chosen to reduce or eliminate promoter competition. In some embodiments, at least two of the engineered delivery vesicle generation system components are expressed from different promoters. In some embodiments, each of the engineered delivery vesicle generation system components are expressed from different promoters. By way of example, the LTR retroelement polypeptide (e.g., a Sushi family protein such as PEG10 or an RTL polypeptide) can be expressed from am SFFV promoter, the cargo polynucleotide (e.g., a cargo RNA) can be expressed from CMV promoter, and the fusogen (e.g., VSVG) can be expressed from an EF1alpha promoter. Without being bound by theory promoter selection to reduce competition can improve production of the engineered delivery vesicles from an engineered delivery vesicle generation system. Expression of the individual components can be tuned so as to improve production of the engineered delivery vesicles from an engineered delivery vesicle system described herein, such as specific promoter selection, inclusion of enhancer regulatory elements, and other approaches. When in a bacterial producer cell system choosing optimal vectors to control copy number can be used. Codon optimization (described elsewhere herein) can also be employed to tune expression and, e.g., improve, production of the engineered delivery vesicles from an engineered delivery vesicle system described herein. The expression of each component of the engineered delivery vesicle system can be tuned (up or down) relative to each other to optimize production of the engineered delivery vesicles from an engineered delivery vesicle system described herein.

In some embodiments, the codon of the LTR retroelement polypeptides are optimized to increase trans packaging, e.g., of a cargo, and reduce or eliminate cis (e.g., self RNA) packaging. In some embodiments, the PEG10 is codon optimized such that it's RNA has a reduced number of binding motifs such that self-packaging of its own RNA is reduced. This approach can be paired with the inclusion of the PEG10 bind motif in a cargo. As discussed elsewhere herein, an exemplary PEG10 binding motif is UNNUU.

### Exemplary PEG10 System

The following describes an example PEG10-based system. Similar systems, such as those including other Gag homologs or other (e.g., endogenous) LTR retroelement polypeptides, may be designed using the general guidance provided in this section. As disclosed herein, PEG10 systems can package their own mRNA and VLPs containing the packaged PEG10 mRNA are exported from cells. As discovered by Applicants, packaging of PEG10 mRNA is facilitated by a 5′ and 3′UTR derived from the PEG10 mRNA. Thus, alternate systems using other (e.g., endogenous) LTR retroelement polypeptides that also package their own mRNA via UTRs may be modified as further described in this section. Likewise, if the (e.g., endogenous) LTR retroelement polypeptide does not package its own mRNA, then the (e.g., endogenous) LTR retroelement polypeptide may be modified to contain one or more cargo-binding domains described above and/or elsewhere herein, which can be paired with a corresponding packaging element(s) added to the cargo molecule similar to how the PEG10 UTR packaging elements are described in this section and in the Working Examples section below.

Described in several embodiments herein are engineered delivery system comprising (a) a polynucleotide encoding an endogenous PEG10 polypeptide; and (b) a one or more a cargo polynucleotides and (c) one or more packaging elements. The packaging elements can be operatively coupled to the one or more packaging elements. In this context, “operatively coupled” refers to the association, physical location/proximity, temporal overlap, and the like, between the cargo polynucleotide(s) and the packaging element(s) such that packaging by the (e.g., endogenous) LTR retroelement polypeptide (e.g., PEG10 in this specific example) occurs. In the context of PEG10 in this example, operatively coupled refers to the physical location of the packaging elements relative to the cargo polynucleotide to be packaged. As described and demonstrated in the Working Examples herein, that “operatively coupled” in some embodiments refers to the placement of the packaging elements such that they flank the cargo polynucleotide at the 5′ and/or 3′ end of the cargo polynucleotide. It will be appreciated that other positions may also work, and the Working Examples herein demonstrate exemplary assays to determine packaging efficiency by a construct which can be adapted to identify cargos and packaging elements that are operatively coupled and those that are not. In some embodiments, the cargo polynucleotide is on the same polynucleotide as the packaging elements.

In some embodiments the system further includes (d) a polynucleotide encoding a fusogenic polypeptide. Fusogenic polypeptides (also referred to in the art as fusogens) are polypeptides that promote and/or mediate fusion between two membranes. As used in the context of the present invention, fusogenic polypeptides are polypeptides that promote and/or mediate the fusion of a delivery vesicle to another membrane, such as a cell membrane. In some embodiments, the fusogenic polypeptides can mediate delivery vesicle—target membrane fusion or interaction in a specific (such as cell-specific or membrane-specific) manner. Such specificity can be facilitated by a protein-protein interaction (such as ligand-receptor) or other interaction between the fusogenic polypeptide and one or more other molecules present in or on a target membrane.

In some embodiments, the PEG10 polypeptide comprises a capsid domain, a nucleocapsid domain, a protease domain, and a reverse transcriptase domain. In some embodiments the polynucleotide encoding the endogenous PEG10 polypeptide comprises one or more modifications to enhance binding specificity and/or packaging of the cargo polynucleotide. In some embodiments, the one or more modifications are made in the polynucleotide encoding the endogenous PEG polypeptide at the boundary between the nucleocapsid and protease domain.

In some embodiments, the one or more packaging elements are a 5′ and/or 3′ UTRs, or portions thereof sufficient to enable complexing with one or more domains of the PEG10 polypeptide, derived from a mRNA encoding the PEG10 polypeptide. In some embodiments, the one or more packaging elements comprising a 5′ UTR of and a portion of the 3′ UTR is derived from the mRNA encoding the PEG10 polypeptide. In some embodiments, the portion of the 3′ UTR includes 500 bp of a proximal end of the 3′ UTR.

In some embodiments, features (a), (b), (c) and/or (d) are encoded on a vector comprising one or more regulatory elements. In some embodiments, one or more or all of the features (a), (b), (c), and/or (d) are operatively coupled to the regulatory elements. In this context, “operatively coupled” is used as it is described elsewhere herein in relation to polynucleotide expression and vectors. In some embodiments features (a), (b), (c) and/or (d), when present are each controlled by a different regulatory element. In some embodiments, (a), (b), (c), and (d) are controlled by the same regulatory element. In some embodiments (a), (b), and (c) are controlled by the same regulatory element. In some embodiments (a), (b), and (d) are controlled by the same regulatory element. In some embodiments (a), (c), and (d) are controlled by the same regulatory element. In some embodiments (b), (c), and (d) are controlled by the same regulatory element. In some embodiments (a) and (b) are controlled by the same regulatory element. In some embodiments, (a) and (c) are controlled by the same regulatory element. In some embodiments, (b) and (c) are controlled by the same regulatory element. In some embodiments (a) and (d) are controlled by the same regulatory element. In some embodiments (b) and (d) are controlled by the same regulatory element. In some embodiments (c) and (d) are controlled by the same regulatory element.

In some embodiments, features (a) (b), and (c) are encoded on the same vector. In some embodiments where features (a), (b), and (c) are encoded on the same vector, they are each encoded by different regulatory element(s). In some embodiments where features (a), (b), and (c) are encoded on the same vector, at least two (e.g. (a) and (b), (b) and (c), or (a) and (c)) are controlled by the same regulatory element(s). In some embodiments where features (a), (b), and (c) are encoded on the same vector, at least one feature is controlled by a different regulatory element(s) than at least one other feature. In some embodiments where features (a), (b), and (c) are encoded on the same vector, they are each encoded by the same regulatory element(s). Vectors and suitable regulatory elements, such as those for driving expression or regulating expression of the polynucleotides, are described in greater detail elsewhere below.

In yet other example embodiments, the mammalian host is a human.

In some embodiments, the one or more packaging elements are a 5′ and/or 3′ UTRs, or a portion thereof sufficient to enable complexing with one or more domains of the (e.g., endogenous) LTR retroelement polypeptide, derived from a mRNA encoding the (e.g., endogenous) LTR retroelement polypeptide. In some embodiments, the 5′ and 3′ UTRs are derived from a mRNA encoding a Sushi family protein, such as PEG10, RTL1, RTL3, RTL4, RTL5, RTL6, RTL7, RTL8, RTL 9, or RTL 10.

In some embodiments, a 5′ UTR present in an engineered delivery system described herein is about 3 to about 5,000 nucleotides in length. In some embodiments, a 5′ UTR present in an engineered delivery system described herein is or ranges from about 3 or/to 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399,400, 401, 402, 403,404, 405, 406, 407,408, 409, 410, 411, 412,413, 414, 415, 416,417, 418, 419, 420,421, 422, 423, 424,425, 426, 427, 428,429, 430, 431, 432, 433, 434, 435, 436, 437,438, 439, 440, 441,442, 443, 444, 445,446, 447, 448, 449, 450,451, 452, 453, 454,455, 456, 457, 458, 459, 460,461, 462, 463, 464,465, 466, 467, 468, 469, 470, 471, 472, 473, 474, 475, 476, 477, 478, 479, 480, 481, 482, 483, 484, 485, 486, 487, 488,489, 490, 491, 492,493, 494, 495, 496,497, 498, 499, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725, 2750, 2775, 2800, 2825, 2850, 2875, 2900, 2925, 2950, 2975, 3000, 3025, 3050, 3075, 3100, 3125, 3150, 3175, 3200, 3225, 3250, 3275, 3300, 3325, 3350, 3375, 3400, 3425, 3450, 3475, 3500, 3525, 3550, 3575, 3600, 3625, 3650, 3675, 3700, 3725, 3750, 3775, 3800, 3825, 3850, 3875, 3900, 3925, 3950, 3975, 4000, 4025, 4050, 4075, 4100, 4125, 4150, 4175, 4200, 4225, 4250, 4275, 4300, 4325, 4350, 4375, 4400, 4425, 4450, 4475, 4500, 4525, 4550, 4575, 4600, 4625, 4650, 4675, 4700, 4725, 4750, 4775, 4800, 4825, 4850, 4875, 4900, 4925, 4950, 4975, or 5000, or any range or numerical value therein.

In some embodiments, a 3′ UTR present in an engineered delivery system described herein is about 3 to about 8,000 nucleotides in length. In some embodiments, a 3′ UTR present in an engineered delivery system described herein is or ranges from about 3 or/to 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399,400, 401, 402, 403,404, 405, 406, 407,408, 409, 410, 411, 412,413, 414, 415, 416,417, 418, 419, 420,421, 422, 423, 424,425, 426, 427, 428, 429, 430, 431,432, 433, 434, 435, 436, 437,438, 439, 440, 441,442, 443, 444, 445,446, 447, 448, 449, 450,451, 452, 453, 454,455, 456, 457, 458, 459, 460,461, 462, 463, 464,465, 466, 467, 468, 469, 470, 471, 472, 473, 474, 475, 476, 477, 478, 479, 480, 481, 482, 483, 484, 485, 486, 487, 488,489, 490, 491, 492,493, 494, 495, 496,497, 498, 499, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725, 2750, 2775, 2800, 2825, 2850, 2875, 2900, 2925, 2950, 2975, 3000, 3025, 3050, 3075, 3100, 3125, 3150, 3175, 3200, 3225, 3250, 3275, 3300, 3325, 3350, 3375, 3400, 3425, 3450, 3475, 3500, 3525, 3550, 3575, 3600, 3625, 3650, 3675, 3700, 3725, 3750, 3775, 3800, 3825, 3850, 3875, 3900, 3925, 3950, 3975, 4000, 4025, 4050, 4075, 4100, 4125, 4150, 4175, 4200, 4225, 4250, 4275, 4300, 4325, 4350, 4375, 4400, 4425, 4450, 4475, 4500, 4525, 4550, 4575, 4600, 4625, 4650, 4675, 4700, 4725, 4750, 4775, 4800, 4825, 4850, 4875, 4900, 4925, 4950, 4975, 5000, 5025, 5050, 5075, 5100, 5125, 5150, 5175, 5200, 5225, 5250, 5275, 5300, 5325, 5350, 5375, 5400, 5425, 5450, 5475, 5500, 5525, 5550, 5575, 5600, 5625, 5650, 5675, 5700, 5725, 5750, 5775, 5800, 5825, 5850, 5875, 5900, 5925, 5950, 5975, 6000, 6025, 6050, 6075, 6100, 6125, 6150, 6175, 6200, 6225, 6250, 6275, 6300, 6325, 6350, 6375, 6400, 6425, 6450, 6475, 6500, 6525, 6550, 6575, 6600, 6625, 6650, 6675, 6700, 6725, 6750, 6775, 6800, 6825, 6850, 6875, 6900, 6925, 6950, 6975, 7000, 7025, 7050, 7075, 7100, 7125, 7150, 7175, 7200, 7225, 7250, 7275, 7300, 7325, 7350, 7375, 7400, 7425, 7450, 7475, 7500, 7525, 7550, 7575, 7600, 7625, 7650, 7675, 7700, 7725, 7750, 7775, 7800, 7825, 7850, 7875, 7900, 7925, 7950, 7975, 8000, 8025, 8050, 8075, 8100, 8125, 8150, 8175, 8200, 8225, 8250, 8275, 8300, 8325, 8350, 8375, 8400, 8425, 8450, 8475, 8500, 8525, 8550, 8575, 8600, 8625, 8650, 8675, 8700, 8725, 8750, 8775, 8800, 8825, 8850, 8875, 8900, 8925, 8950, 8975, 9000, or any range or numerical value therein.

In some embodiments, the 5′ and/or 3′ UTRs are from the same gene as the (e.g., endogenous) LTR retroelement polypeptide used in the delivery vesicle system (e.g., PEG 10, RTL1, etc.). In some embodiments, the 5/and/or the 3′ UTR is/are from a gene encoding an ortholog of the gene encoding the (e.g., endogenous) LTR retroelement polypeptide used in the delivery vesicle system (e.g., PEG 10, RTL1, etc.). By way of a non-limiting example, a 5′ or 3′ UTR can be from a mouse gene while the gene encoding the (e.g., endogenous) LTR retroelement polypeptide included in the delivery vesicle system is a human ortholog of the mouse gene. In some embodiments, the 5′ and/or 3′ UTRs are from an (e.g., endogenous) LTR retroelement polypeptide gene that is different from the gene encoding the (e.g., endogenous) LTR retroelement polypeptide used in the delivery vesicle system to form the delivery vesicle.

In some embodiments, the fusogenic polypeptide is a tetraspanin (TSPAN), a G envelope protein, a SGCE, a syncitin, or combination thereof. In some embodiments, the TSPAN is CD81, C9, CD63, or a combination thereof. In some embodiments, the G envelope protein is vesicular stomatitis virus G envelope protein (VSV-G).

Genes encoding viral polypeptides capable of self-assembly into defective, non-self-propagating viral particles can be obtained from the genomic DNA of a DNA virus or the genomic cDNA of an RNA virus or from available subgenomic clones containing the genes. These genes will include those encoding viral capsid proteins (i.e., proteins that comprise the viral protein shell) and, in the case of enveloped viruses, such as retroviruses, the genes encoding viral envelope glycoproteins. Additional viral genes may also be required for capsid protein maturation and particle self-assembly. These may encode viral proteases responsible for processing of capsid protein or envelope glycoproteins. As an example, the genomic structure of picornaviruses has been well characterized, and the patterns of protein synthesis leading to virion assembly are clear. Rueckert, R. in Virology (1985), B. N. Fields et al. (eds.) Raven Press, New York, pp 705-738. In picornaviruses, the viral capsid proteins are encoded by an RNA genome containing a single long reading frame, and are synthesized as part of a polyprotein which is processed to yield the mature capsid proteins by a combination of cellular and viral proteases. Thus, the picornavirus genes required for capsid self-assembly include both the capsid structural genes and the viral proteases required for their maturation. Another virus class from which genes encoding self-assembling capsid proteins can be isolated is the lentiviruses, of which HIV is an example. Like the picornaviral capsid proteins, the HIV gag protein is synthesized as a precursor polypeptide that is subsequently processed, by a viral protease, into the mature capsid polypeptides. However, the gag precursor polypeptide can self-assemble into virus-like particles in the absence of protein processing. Gheysen et al., Cell 59:103 (1989); Delchambre et al., The EMBO J. 8:2653-2660 (1989). Unlike picornavirus capsids, HIV capsids are surrounded by a loose membranous envelope that contains the viral glycoproteins. These are encoded by the viral env gene.

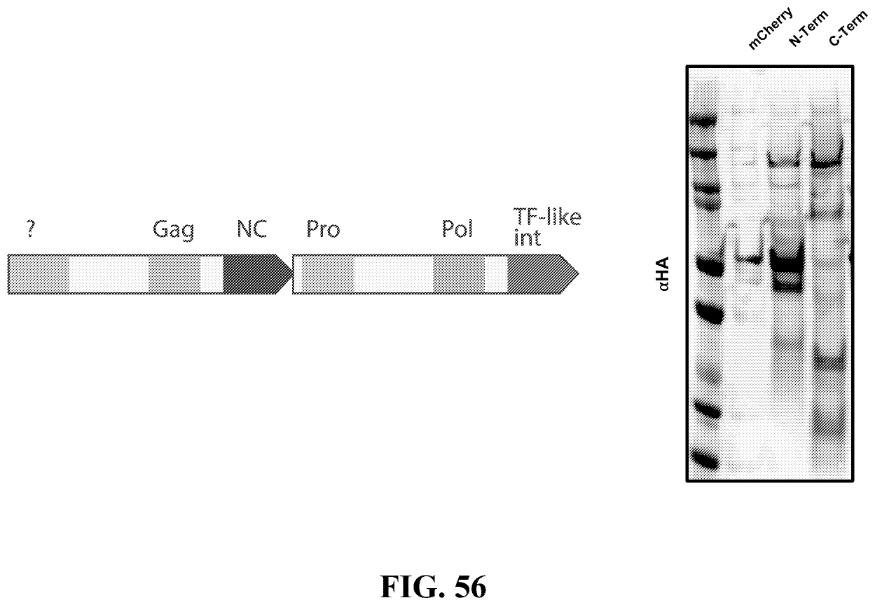

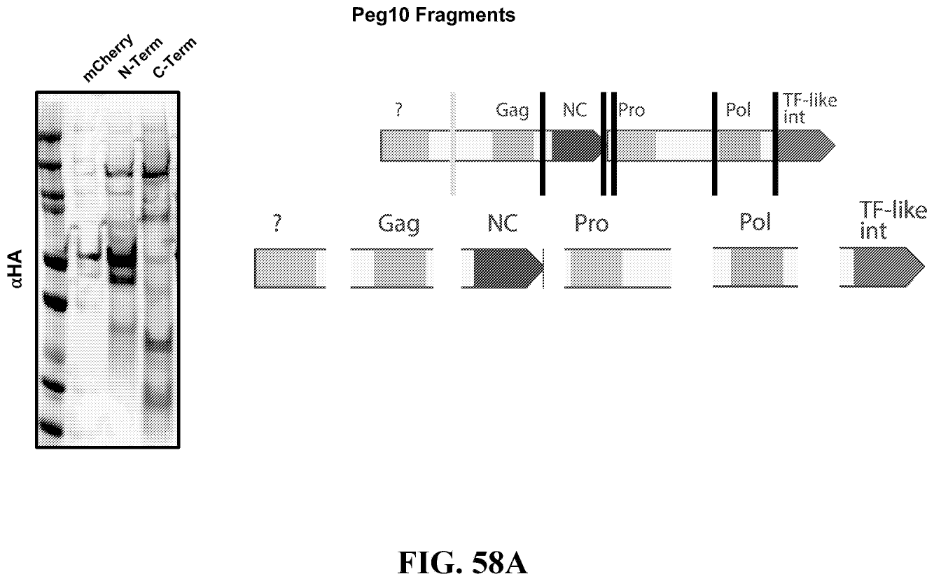

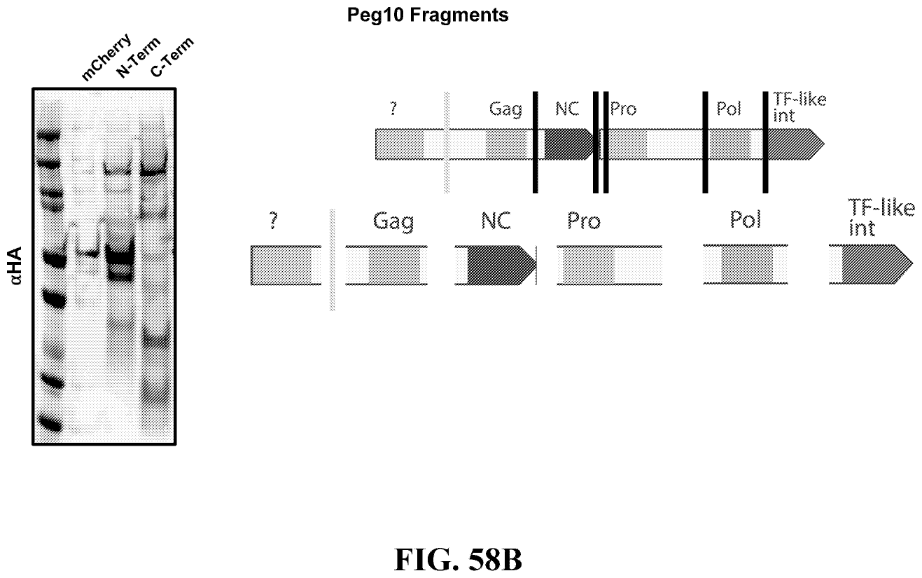

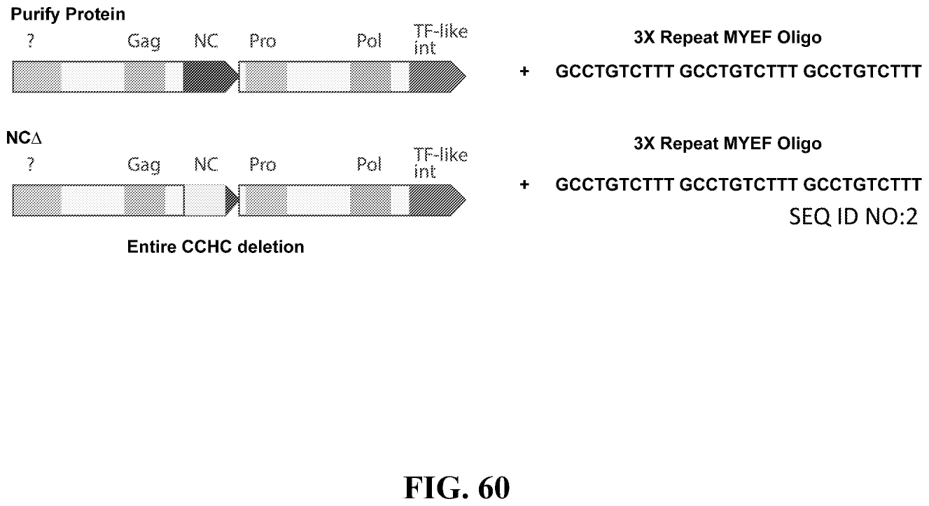

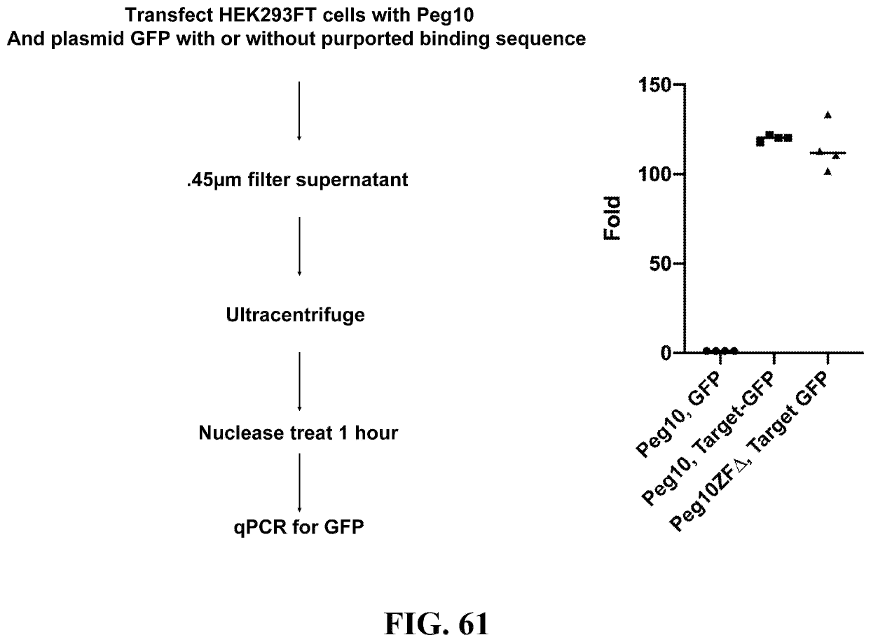

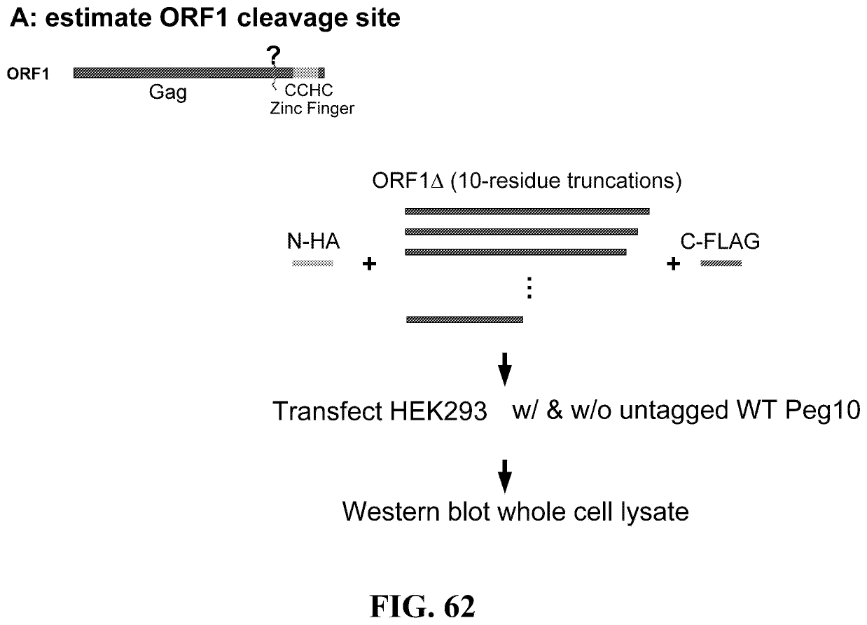

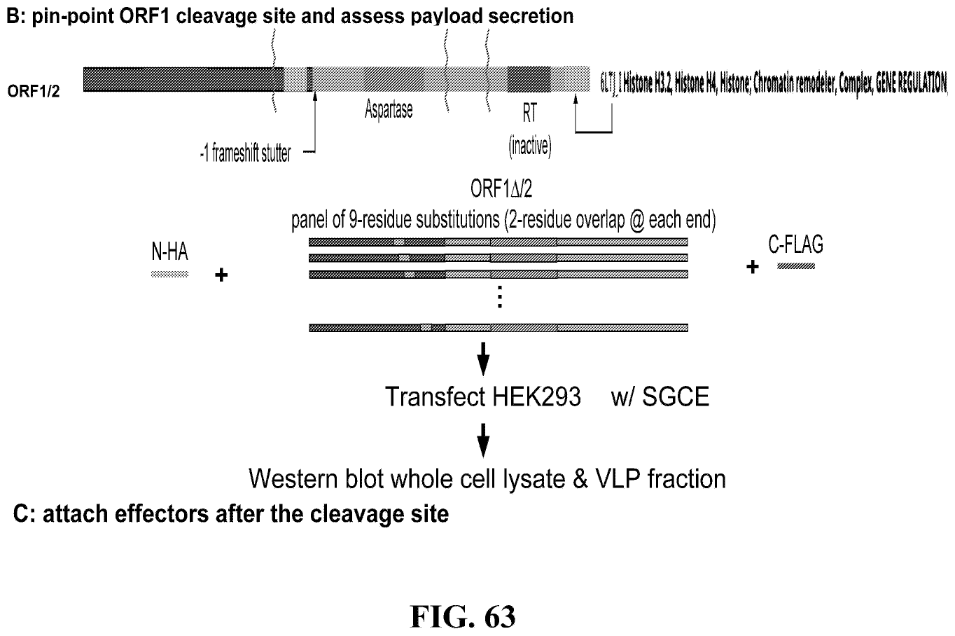

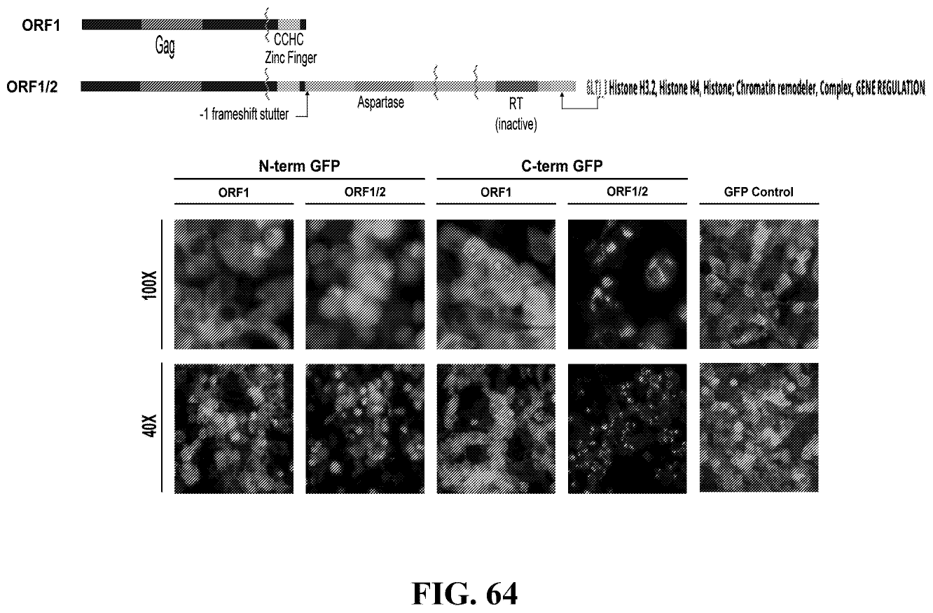

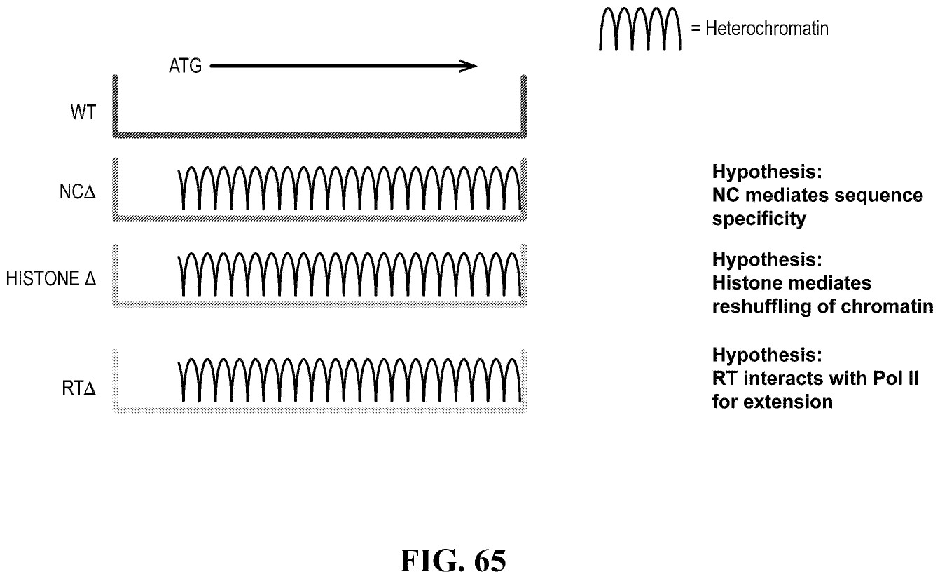

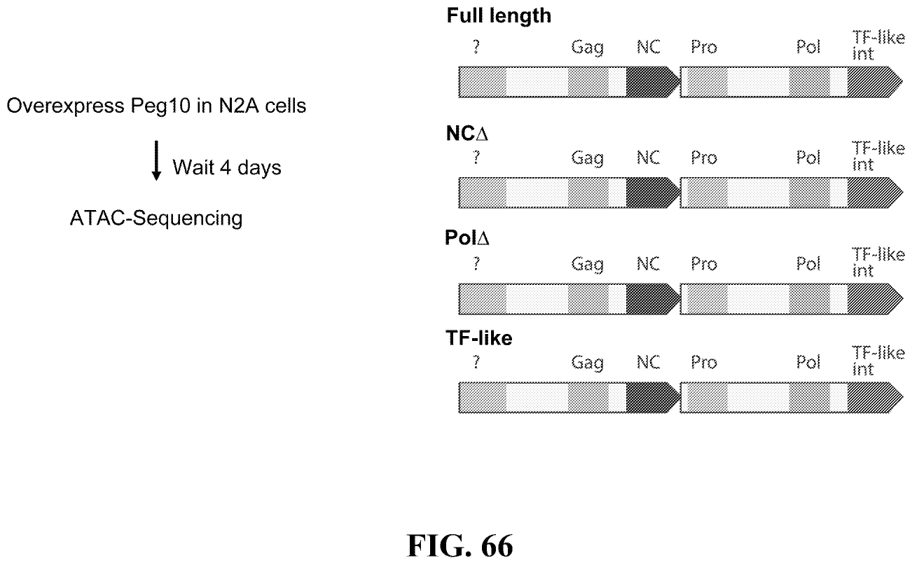

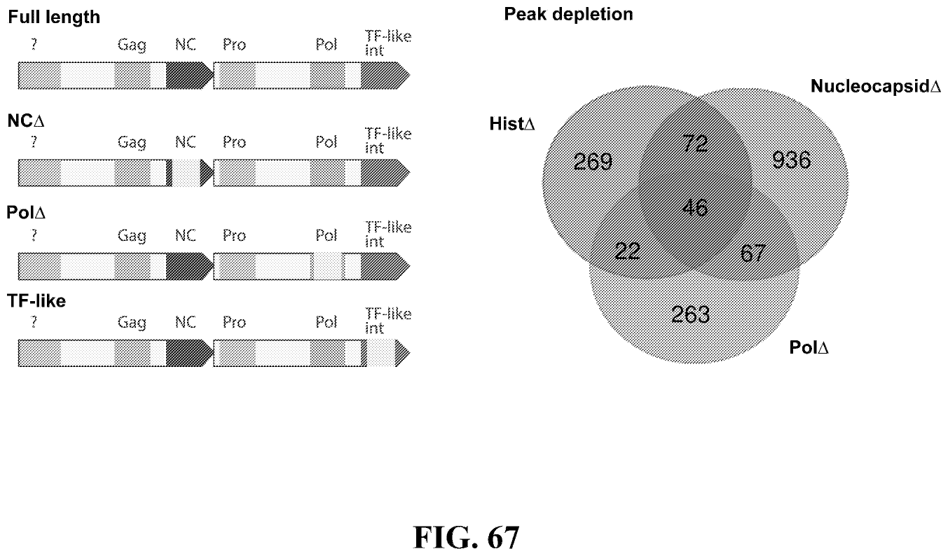

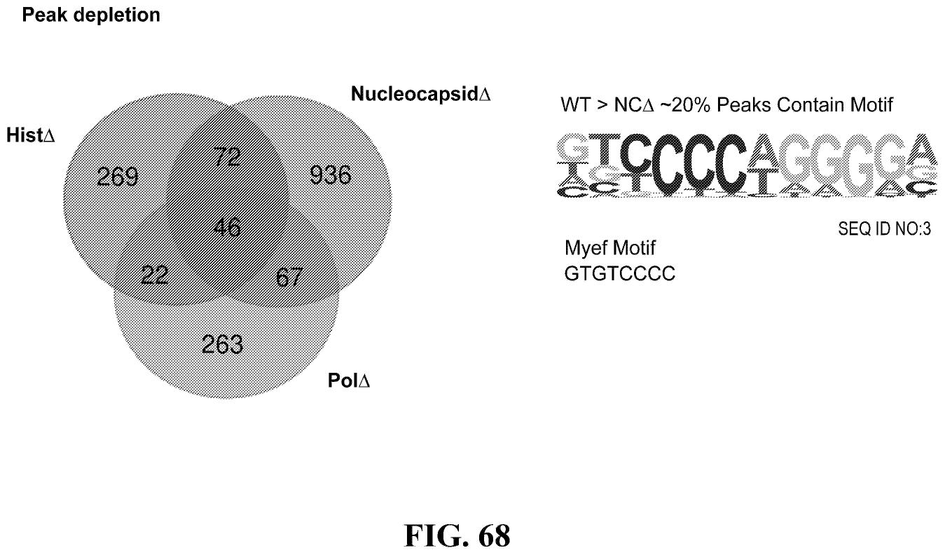

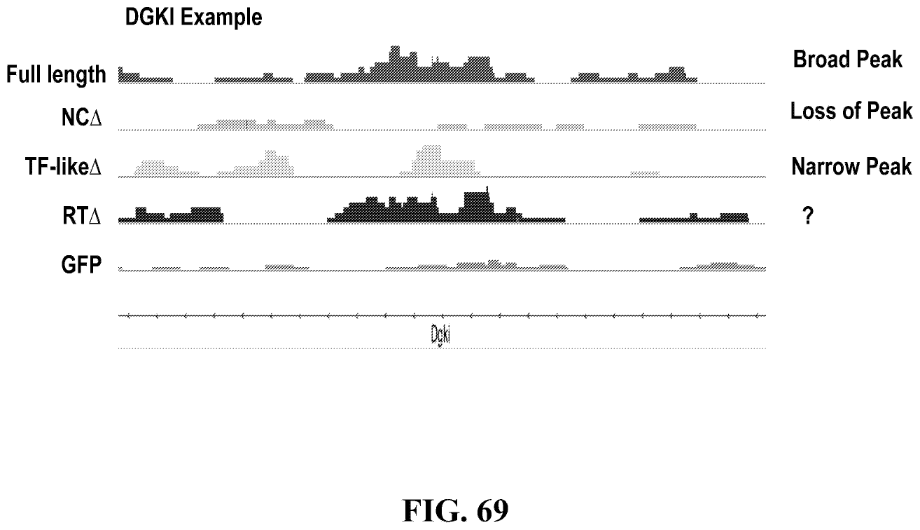

As any person of skill in the art would appreciate, any of the systems described herein can be further engineered to a minimal set of components and be applied to any suitable endogenous element. As described in Examples 3 and 4 and FIGS. 56-70, use of PEG10 is just an example approach that can be followed with any other endogenous element.

In some embodiments, the gag protein or other gag associated protein is genetically recoded. In some embodiments, the gag protein or other gag associated protein is genetically recoded such that packaging and/or delivery of a cargo is increased. See also Working Example 6.

In some embodiments, the gag protein is PEG10. In some embodiments, the PEG10 comprises a RT and HIST (putative histone interacting) domains. In some embodiments, the PEG10 comprises a mutation in a histone interacting domain.

In some embodiments, the PEG10 is a mutant that has increased oligomerization activity, increased cargo packaging activity, increased cargo delivery, or a combination thereof as compared to a suitable control PEG10. It will be appreciated that such mutants, are encoded by corresponding engineered polynucleotides, which are within the scope of this disclosure.

In some embodiments, as discussed elsewhere herein, the cargo can include one or more components of an RNA guided nuclease system, such as a CRISPR-Cas system or IscB system. In some embodiments, a guide polynucleotide of such a system can be included and packaged along with a polynucleotide encoding an RNA guided nuclease. As demonstrated in the Working Examples herein, a polynucleotide encoding a Cas can be co-packaged with a gRNA. In some embodiments, the delivery vesicle generation system that includes PEG10 can include a polynucleotide encoding an RNA guided nuclease (e.g., a Cas polypeptide) and a gRNA as cargo polynucleotides.

### Expanded Example Cargo Molecules

The delivery vesicles described herein may be used and further comprise a number of different cargo molecules for delivery. Representative cargo molecules may include, but are not limited to, nucleic acids, polynucleotides, proteins, polypeptides, polynucleotide/polypeptide complexes, small molecules, sugars, or a combination thereof. Cargos that can be delivered in accordance with the systems and methods described herein include, but are not necessarily limited to, biologically active agents, including, but not limited to, therapeutic agents, imaging agents, and monitoring agents. A cargo may be an exogenous material or an endogenous material.

### Polynucleotides

In some embodiments, the cargo is a cargo polynucleotide. As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “deoxyribonucleic acid (DNA)” and “ribonucleic acid (RNA)” can generally refer to any polyribonucleotide or polydeoxribonucleotide, which may be unmodified RNA or DNA or modified RNA or DNA. RNA can be in the form of non-coding RNA, including but not limited to, tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA), or coding mRNA (messenger RNA).

In some embodiments, the cargo polynucleotide is DNA. In some embodiments, the cargo polynucleotide is RNA. In some embodiments, the cargo polynucleotide is a polynucleotide (a DNA or an RNA) that encodes an RNA and/or a polypeptide. As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

### Polynucleotide Modifications

In some embodiments, the cargo polynucleotides include one or more modifications capable of modifying the e.g., functionality, packaging ability, stability, degredation localization, increase expression lifetime, resistance to degradation, or any combination thereof, of the at least one or more cargo polynucleotides. Modifications can be sequence modifications (e.g., mutations), chemical modifications, or other modifications, such as complexing to a lipid, polymer, etc. In some embodiments, the cargo polynucleotide is modified to protect it against degradation, by e.g., nucleases or otherwise prevent its degredation.

In some embodiments, one or more polynucleotides in the engineered polynucleotide are modified. In some embodiments, the engineered polynucleotide includes one or more non-naturally occurring nucleotides, which can be the result of modifying a naturally occurring nucleotide. In some embodiments, the modification is selected independently for each polynucleotide modified. In some embodiments, the modification(s) increase or decrease the stability of the polynucleotide, reduce the immunogenicity of the polynucleotide, increase or decrease the rate of transcription and/or translation, or any combination thereof. Non-naturally occurring nucleotides and/or nucleotide analogs may be modified at the ribose, phosphate, and/or base moiety.

Suitable modifications include, without limitation, methylpseudouridine, a phosphorothioate linkage, a locked nucleic acid (LNA) nucleotides comprising a methylene bridge between the 2′ and 4′ carbons of the ribose ring, or bridged nucleic acids (BNA), 2′-O-methyl analogs, 2′-deoxy analogs, or 2′-fluoro analogs, 2-aminopurine, 5-bromo-uridine, pseudouridine, (T), N1-methylpseudouridine (melΨ), 5-methoxyuridine(5moU), inosine, 7-methylguanosine, inosine, 7-methylguanosine. Examples of RNA, including but not limited to guide RNA, chemical modifications include, without limitation, incorporation of 2′-O-methyl (M), 2′-O-methyl 3′phosphorothioate (MS), S-constrained ethyl(cEt), or 2′-O-methyl 3′thioPACE (MSP) at one or more terminal nucleotides.

In some embodiments, the polynucleotide (DNA and/or RNA) is modified with a 5′- and/or 3′-cap structure. In some embodiments, the 5′ cap structure is cap0, cap1, ARCA, inosine, N1-methyl-guanosine, 2′-fluoro-guanosine, 7-deaza-guanosine, 8-oxo-guanosine, 2-amino-guanosine, LNA-guanosine, or 2-azido-guanosine. In some embodiments, the 5′terminal cap is 7mG(5′)ppp(5′)NlmpNp, m7GpppG cap, N′-methylguanine. In some embodiments, the 3′terminal cap is a 3′-O-methyl-m7GpppG, 2′Fluoro bases, inverted dT and dTTs, phosphorylation of the 3′ end nucleotide, a C3 spacer. Exemplary 5′- and/or 3′ that protect against degradation are described in e.g., Gagliardi and Dziembowski. Philosophical transactions of the Royal Society B. 2018. 313(1762). https://doi.org/10.1098/rstb.2018.0160; Boo and Kim. Experimental & Molecular Medicine volume 52, pages 400-408 (2020); and Adachai et al., 2021. Biomedicines 2021, 9, 550. https://doi.org/10.3390/biomedicines9050550.

In some embodiments, the 5′-UTR comprises a Kozak sequence.

In some embodiments, the polynucleotide can be modified with a tailing sequence may range from absent to 500 nucleotides in length (e.g., at least 60, 70, 80, 90, 120, 140, 160, 180, 200, 250, 300, 350, 400, 450, or 500 nucleotides). In some embodiments, the tailing region is or includes a polyA tail. Where the tailing region is a polyA tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional. In some embodiments, the poly-A tail is at least 160 nucleotides in length.

In some embodiments, about 10%, 15%, 20%, 24%, 30%, 35%, 40%, 45%, 50%, 55%, 60%, 65%, 70%, 75%, 80%, 85%, 90%, 95%, to/or about 100% of the uracils in of a polynucleotide of the present invention have a chemical modification, In some embodiments, about 10%, 15%, 20%, 24%, 30%, 35%, 40%, 45%, 50%, 55%, 60%, 65%, 70%, 75%, 80%, 85%, 90%, 95%, to/or about 100% of the uracils of a polynucleotide of the present invention have a N1-methyl pseudouridine in the 5-position of the uracil.

In some embodiments, the polynucleotide, optionally an RNA (e.g., an mRNA) includes a stabilization element. In some embodiments, the stabilization element is a histone stem-loop. In some embodiments, the stabilization element is a nucleic acid sequence having increased GC content relative to wild type sequence.

In some embodiments, a polynucleotide of the present invention includes a sequence encoding a self-cleaving peptide. The self-cleaving peptide may be, but is not limited to, a 2A peptide. In one embodiment, this sequence may be used to separate the coding regions of two or more polypeptides.

In some embodiments, the polynucleotides (e.g., mRNAs) are linear. In yet another embodiment, the polynucleotides of the present invention that are circular are known as “circular polynucleotides” or “circP.” As used herein, “circular polynucleotides” or “circP” means a single stranded circular polynucleotide which acts substantially like, and has the properties of, an R A. The term “circular” is also meant to encompass any secondary or tertiary configuration of the circP.

Other RNA modifications, such as mRNA modifications, that can be incorporated into a polynucleotide of the present invention include, but are not limited to, any one or more of those described e.g., U.S. Pat. Nos. 8,278,036, 8,691,966, 8,748,089, 9,750,824, 10,232,055, 10,703,789, 10,702,600, 10,577,403, 10,442,756, 10,266,485, 10,064,959, 9,868,692, 10,064,959, 10,272,150; U.S. Publications, US20130197068, US20170043037, US20130261172, US20200030460, US20150038558, US20190274968, US20180303925, US20200276300; International Patent Application Publication Nos. WO/2018/081638A1, WO/2016/176330A1, which are incorporated herein by reference and can be adapted for use with the present invention.

### Signaling and Localization Molecules

In some embodiments, the polynucleotide includes a signaling and/or localization molecule (e.g., a polynucleotide that is a signaling or localization molecule or a polynucleotide that encodes a signaling or localization peptide or polypeptide).

In some embodiments, the signaling or localization molecule directs a function (e.g. secretion, folding, etc.) and/or trafficking to a particular location within a cell (e.g., nucleus, Golgi, lysosome, peroxisome, cytoplasm, membrane, chloroplast, vacuole, mitochondria, etc.). In some embodiments, the signaling or localization molecule(s) is/are positioned at the 3′ and/or 5′ end of a polynucleotide of the present invention, such as a cargo polynucleotide. In some embodiments, the signaling or localization molecule(s) is/are located at one or more positions between the 3′ and 5′ end of a polynucleotide of the present invention. In some embodiments, the signaling or localization molecule(s) are located at the 3′ and/or 5′ end of a polynucleotide of the present invention and at one or more positions between the 3′ and 5′ end of a polynucleotide of the present invention. In some embodiments, the signaling and/or localization molecule(s) is/are incorporated in a polynucleotide, such as a cargo polynucleotide, such that it is at the C-terminus, N-terminus, or one or more positions between the C-terminus and N-terminus of a polypeptide encoded by the polynucleotide.

In some embodiments, a polynucleotide of the present invention includes a polynucleotide sequence that is or encodes one or more signal peptides, leucine rich repeat (LRR) sequences, nuclear localization signals, a Type IX secretion system (T9SS) substrate, secretion signal peptide, an amino acid sequence capable of directing clearance from a cell or organism, an Fc receptor directing binding to a dendritic cell, and/or directing antigen processing, an F-box domain or polypeptide, a subcellular localization sequence, a TOM70, TOM20, or TOM22 binding polypeptide, a stromal import sequence, a thylakoid targeting sequence, a peroxisome targeting signal 1 sequence, a peroxisome targeting signal 2 sequence, an endoplasmic reticulum signaling sequence.

Exemplary nuclear localization molecules are described in e.g., Lu et al., Cell Communication and Signaling. 2021. 19(60): 1-10 (particularly at Table 1 therein), which can be adapted for use with the present invention. Exemplary signal peptides are described in e.g., Owji et al., European J Cell Biol. 2018. 97(6):422-441, which can be adapted for use with the present invention. Exemplary peroxisome targeting sequences are described in e.g., Baerends et al., 2000. FEMS Microbiol Rev. 24(3): 291-301, which can be adapted for use with the present invention. Exemplary endoplasmic reticulum signaling molecules are described in e.g., Walter et al., J Cell Biol. 1981. 91(2 Pt. 1):545-50 doi:10.1083/jcb.91.2.545, which can be adapted for use with the present invention. Exemplary lysosomal and endosomal signaling molecules are described in e.g., Bonifacino and Traub. 2003. Ann. Rev. Biochem. 72:395-447, which can be adapted for use with the present invention. Exemplary endoplasmic reticulum signaling sequences are described in e.g., J Cell Biol. 1996 Jul. 2; 134(2): 269-278, which can be adapted for use with the present invention. Exemplary Golgi signaling sequences are described in e.g., Gleeson et al., 1994. Glycoconjugat J. 11:381-394, which can be adapted for use with the present invention.

### Interference RNAs

In certain example embodiments, the one or more polynucleotides may encode one or more interference RNAs. Interference RNAs are RNA molecules capable of suppressing gene expressions. Example types of interference RNAs include small interfering RNA (siRNA), microRNA (miRNA), and short hairpin RNA (shRNA).

In certain example embodiments, the interference RNA may be a siRNAs. Small interfering RNA (siRNA) molecules are capable of inhibiting target gene expression by interfering RNA. siRNAs may be chemically synthesized, or may be obtained by in vitro transcription, or may be synthesized in vivo in target cell. sRNAs may comprise double-stranded RNA from 15 to 40 nucleotides in length and can contain a protuberant region 3′ and/or 5′ from 1 to 6 nucleotides in length. Length of protuberant region is independent from total length of siRNA molecule. siRNAs may act by post-transcriptional degradation or silencing of target messenger. In some cases, the exogenous polynucleotides encode shRNAs. In shRNAs, the antiparallel strands that form siRNA are connected by a loop or hairpin region.

The interference RNA (e.g., siRNA) may suppress expression of genes to promote long term survival and functionality of cells after transplanted to a subject. In some examples, the interference RNAs suppress genes in TGFβ pathway, e.g., TGFβ, TGFβ receptors, and SMAD proteins. In some examples, the interference RNAs suppress genes in colony-stimulating factor 1 (CSF1) pathway, e.g., CSF1 and CSF1 receptors. In certain embodiments, the one or more interference RNAs suppress genes in both the CSF1 pathway and the TGFβ pathway. TGFβ pathway genes may comprise one or more of ACVR1, ACVR1C, ACVR2A, ACVR2B, ACVRL1, AMH, AMHR2, BMP2, BMP4, BMP5, BMP6, BMP7, BMP8A, BMP8B, BMPR1A, BMPR1B, BMPR2, CDKN2B, CHRD, COMP, CREBBP, CUL1, DCN, E2F4, E2F5, EP300, FST, GDF5, GDF6, GDF7, ID1, ID2, ID3, ID4, IFNG, INHBA, INHBB, INHBC, INHBE, LEFTY1, LEFTY2, LOC728622, LTBP1, MAPK1, MAPK3, MYC, NODAL, NOG, PITX2, PPP2CA, PPP2CB, PPP2R1A, PPP2R1B, RBL1, RBL2, RBX1, RHOA, ROCK1, ROCK2, RPS6KB1, RPS6KB2, SKP1, SMAD1, SMAD2, SMAD3, SMAD4, SMAD5, SMAD6, SMAD7, SMAD9, SMURF1, SMURF2, SP1, TFDP1, TGFB1, TGFB2, TGFB3, TGFBR1, TGFBR2, THBS1, THBS2, THBS3, THBS4, TNF, ZFYVE16, and/or ZFYVE9.

In some embodiments, the cargo polynucleotide is an RNAi molecule, antisense molecule, and/or a gene silencing oligonucleotide or a polynucleotide that encodes an RNAi molecule, antisense molecule, and/or gene silencing oligonucleotide.

As used herein, “gene silencing oligonucleotide” refers to any oligonucleotide that can alone or with other gene silencing oligonucleotides utilize a cell's endogenous mechanisms, molecules, proteins, enzymes, and/or other cell machinery or exogenous molecule, agent, protein, enzyme, and/or polynucleotide to cause a global or specific reduction or elimination in gene expression, RNA level(s), RNA translation, RNA transcription, that can lead to a reduction or effective loss of a protein expression and/or function of a non-coding RNA as compared to wild-type or a suitable control. This is synonymous with the phrase “gene knockdown” Reduction in gene expression, RNA level(s), RNA translation, RNA transcription, and/or protein expression can range from about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, to 1% or less reduction. “Gene silencing oligonucleotides” include, but are not limited to, any antisense oligonucleotide, ribozyme, any oligonucleotide (single or double stranded) used to stimulate the RNA interference (RNAi) pathway in a cell (collectively RNAi oligonucleotides), small interfering RNA (siRNA), microRNA, and short-hairpin RNA (shRNA). Commercially available programs and tools are available to design the nucleotide sequence of gene silencing oligonucleotides for a desired gene, based on the gene sequence and other information available to one of ordinary skill in the art.

In some embodiments a cargo polynucleotide, such as an encoding polynucleotide, is flanked by at least an (e.g., endogenous) LTR retroelement polypeptide (such as a retroviral gag protein or gag homolog) 3′ UTR or portion thereof, such as the proximal region of about 500 base pairs of the 3′ UTR. In some embodiments a cargo polynucleotide, such as an encoding polynucleotide, is flanked by an (e.g., endogenous) LTR retroelement polypeptide (such as a retroviral gag protein or gag homolog) 5′ UTR. In some embodiments a cargo polynucleotide, such as an encoding polynucleotide, is flanked by an (e.g., endogenous) LTR retroelement (such as a retroviral gag protein or gag homolog) 5′ and 3′ UTR. In some embodiments, the flanking (e.g., endogenous) LTR retroelement polypeptide UTR(s) are from PEG10. In some embodiments, the inclusion of the 3′ UTR, the 5′UTR, or both can increase packaging and/or delivery of the cargo that they flank. These and other packaging elements are described in greater detail elsewhere herein.

### Therapeutic Polynucleotides

In some embodiments, the cargo molecule is a therapeutic polynucleotide. Therapeutic polynucleotides are those that provide a therapeutic effect when delivered to a recipient cell. The polynucleotide can be a toxic polynucleotide (a polynucleotide that when transcribed or translated results in the death of the cell) or polynucleotide that encodes a lytic peptide or protein. In embodiments, delivery vesicles having a toxic polynucleotide as a cargo molecule can act as an antimicrobial or antibiotic. This is discussed in greater detail elsewhere herein. In some embodiments, the cargo molecule can be exogenous to the producer cell and/or a first cell. In some embodiments, the cargo molecule can be endogenous to the producer cell and/or a first cell. In some embodiments, the cargo molecule can be exogenous to the recipient cell and/or a second cell. In some embodiments, the cargo molecule can be endogenous to the recipient cell and/or second cell.

As described herein the cargo polynucleotide can be any polynucleotide endogenous or exogenous to the eukaryotic cell. For example, the cargo polynucleotide can be a polynucleotide residing in the nucleus of the eukaryotic cell. The cargo polynucleotide can be a sequence coding a gene product (e.g., a protein) or a non-coding sequence (e.g., a regulatory polynucleotide).

In some embodiments, the cargo polynucleotide is a DNA or RNA (e.g., a mRNA) vaccine.

### Aptamers

In certain example embodiments, the polynucleotide may be an aptamer. In certain embodiments, the one or more agents is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or “ligated” to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 93 tilizeition of pyrimidines, and 8 position of purines, U.S. Pat. No. 5,756,703 which describes oligonucleotides containing various 2′-modified pyrimidines, and U.S. Pat. No. ‘580,737’which describes highly specific nucleic acid ligands containing one or more nucleotides modified with 2′-amino (2′-NH2), 2′-fluoro (2′-F), and/or 2′-O-methyl (2′-OMe) substituents. Modifications of aptamers may also include modifications at exocyclic amines, substitution of 4-thiouridine, substitution of 5-bromo or 5-iodo-uracil; backbone modifications, phosphorothioate or allyl phosphate modifications, methylations' and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, 0-allyl, S-alkyl, S-allyl, or halo grIethods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include but are not limited to those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colorado). In certain embodiments, the present invention may utilize any aptamer containing any modification as described herein.

In certain other example embodiments, the polynucleotide may be a ribozyme or other enzymatically active polynucleotide.

### Biologically Active Agents

In some embodiments, the cargo is a biologically active agent. Biologically active agents include any molecule that induces, directly or indirectly, an effect in a cell. Biologically active agents may be a protein, a nucleic acid, a small molecule, a carbohydrate, and a lipid. When the cargo is or comprises a nucleic acid, the nucleic acid may be a separate entity from the DNA-based carrier. In these embodiments, the DNA-based carrier is not itself the cargo. In other embodiments, the DNA-based carrier may itself comprise a nucleic aciIgo. Therapeutic agents include, without limitation, chemotherapeutic agents, anti-oncogenic agents, anti-angiogenic agents, tumor suppressor agents, anti-microbial agents, enzyme replacement agents, gene expression modulating agents and expression constructs comprising a nucleic acid encoding a therapeutic protein or nucleic acid, and vaccines. Therapeutic agents may be peptides, proteins (including enzymes, antibodies and peptidic hormones), ligands of cytoskeleton, nucleic acid, small molecules, non-peptidic hormones and the like. To increase affinity for the nucleus, agents may be conjugated to a nuclear localization sequence. Nucleic acids that may be delivered by the method of the invention include synthetic and natural nucleic acid material, including DNA, RNA, transposon DNA, antisense nucleic acids, dsRNA, siRNAs, transcription RNA, messenger RNA, ribosomal RNA, small nucleolar RNA, microRNA, ribozymes, plasmids, expression constructs, etc.

Imaging agents include contrast agents, such as ferrofluid-based MRI contrast agents and gadolinium agents for PET scans, fluorescein isothiocyanate and 6-TAMARA. Monitoring agents include reporter probes, biosensors, green fluorescent protein and the like. Reporter probes include photo-emitting compounds, such as phosphors, radioactive moieties and fluorescent moieties, such as rare earth chelates (e.g., europium chelates), Texas Red, rhodamine, fluorescein, FITC, fluo-3, 5 hexadecanoyl fluorescein, Cy2, fluor X, Cy3, Cy3.5, Cy5, Cy5.5, Cy7, dansyl, phycocrytherin, phycocyanin, spectrum orange, spectrum green, and/or derivatives of any one or more of the above. Biosensors are molecules that detect and transmit information regarding a physiological change or process, for instance, by detecting the presence or change in the presence of a chemical. The information obtained by the biosensor typically activates a signal that is detected with a transducer. The transducer typically converts the biological response into an electrical signal. Examples of biosensors include enzymes, antibodies, DNA, receptors and regulator proteins used as recognition elements, which can be used either in whole cells or isolated and used independently (D'Souza, 2001, Biosensors and Bioelectronics 16:337-353).

One or two or more different cargoes may be delivered by the delivery particles described herein.

In some embodiments, the cargo may be linked to one or more envelope proteins by a linker, as described elsewhere herein. A suitable linker may include, but is not necessarily limited to, a glycine-serine linker. In some embodiments, the glycine-serine linker is (GGS)₃ (SEQ ID NO: 1).

In some embodiments, the cargo comprises a ribonucleoprotein. In specific embodiments, the cargo comprises a genetic modulating agent.

As used herein the term “altered expression” may particularly denote altered production of the recited gene products by a cell. As used herein, the term “gene product(s)” includes RNA transcribed from a gene (e.g., mRNA), or a polypeptide encoded by a gene or translated from RNA.

Also, “altered expression” as intended herein may encompass modulating the activity of one or more endogenous gene products. Accordingly, “altered expression”, “altering expression”, “modulating expression”, or “detecting expression” or similar may be used interchangeably with respectively “altered expression or activity”, “altering expression or activity”, “modulating expression or activity”, or “detecting expression or activity” or similar terms. As used herein, “modulating” or “to modulate” generally means either reducing or inhibiting the activity of a target or antigen, or alternatively increasing the activity of the target or antigen, as measured using a suitable in vitro, cellular or in vivo assay. In particular, “modulating” or “to modulate” can mean either reducing or inhibiting the (relevant or intended) activity of, or alternatively increasing the (relevant or intended) biological activity of the target or antigen, as measured using a suitable in vitro, cellular or in vivo assay (which will usually depend on the target or antigen involved), by at least 5%, at least 10%, at least 25%, at least 50%, at least 60%, at least 70%, at least 80%, or 90% or more, compared to activity of the target or antigen in the same assay under the same conditions but without the presence of the inhibitor/antagonist agents or activator/agonist agents described herein.

As will be clear to the skilled person, “modulating” can also involve effecting a change (which can either be an increase or a decrease) in affinity, avidity, specificity and/or selectivity of a target or antigen, for one or more of its targets compared to the same conditions but without the presence of a modulating agent. Again, this can be determined in any suitable manner and/or using any suitable assay known per se, depending on the target. In particular, an action as an inhibitor/antagonist or activator/agonist can be such that an intended biological or physiological activity is increased or decreased, respectively, by at least 5%, at least 10%, at least 25%, at least 50%, at least 60%, at least 70%, at least 80%, or 90% or more, compared to the biological or physiological activity in the same assay under the same conditions but without the presence of the inhibitor/antagonist agent or activator/agonist agent. Modulating can also involve activating the target or antigen or the mechanism or pathway in which it is involved.

### Gene Modifying Systems

In some embodiments, the cargo is a polynucleotide modifying system or component(s) thereof. In some embodiments the polynucleotide modifying system is a gene modifying system. In some embodiments, the gene modifying system is or is composed of a gene modulating agent. In some embodiments, the genetic modulating agent may comprise one or more components of a polynucleotide modification system (e.g., a gene editing system) and/or polynucleotides encoding thereof.

In some embodiments, the gene editing system may be an RNA-guided system or other programmable nuclease system. In some embodiments, the gene editing system is an IscB system. In some embodiments, the gene editing system may be a CRISPR-Cas system.

### CRISPR-Cas Systems

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA)(chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

### Class 1 Systems

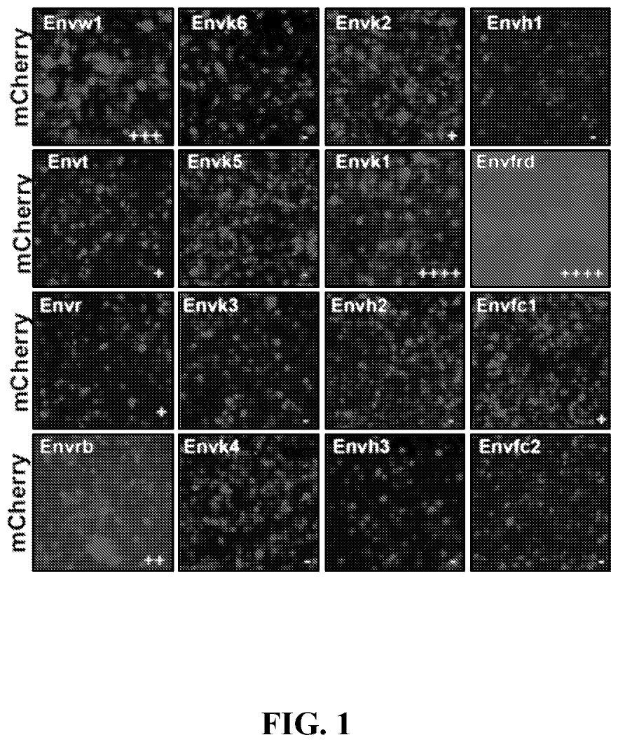

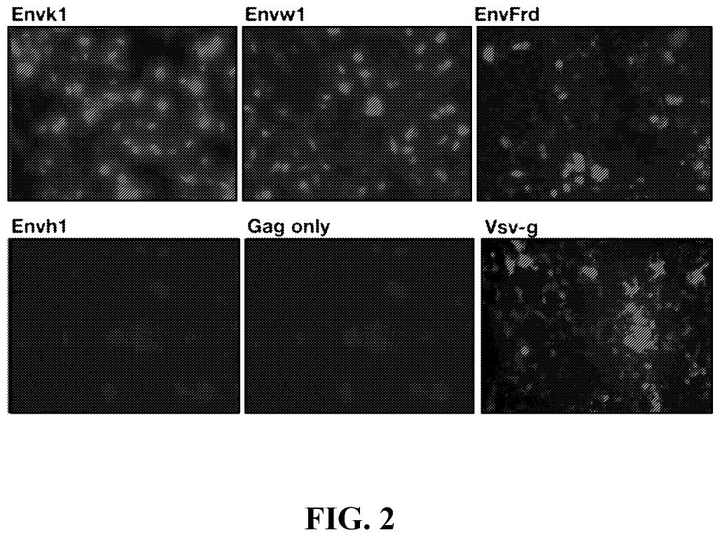

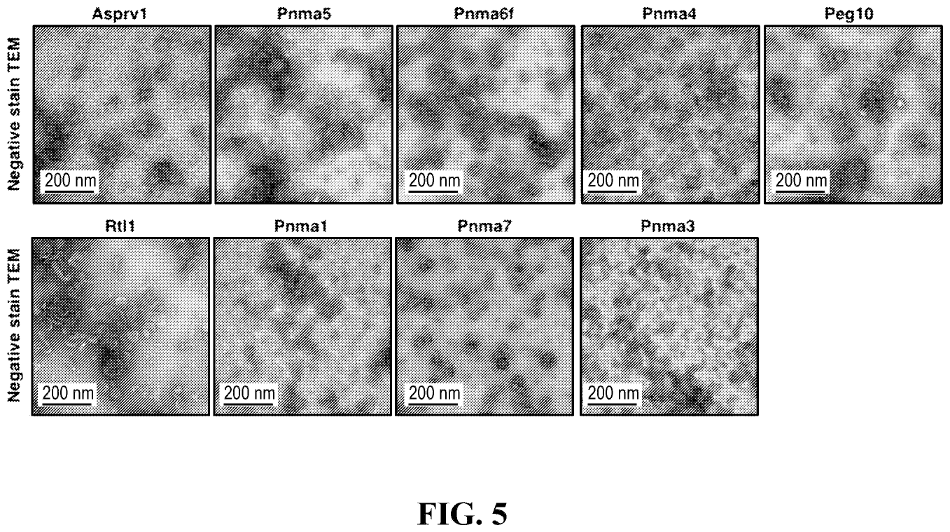

The methods, systems, and tools provided herein may be designed for use with Class 1 CRISPR proteins. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020)., incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g. Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g., Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin EV, Makarova KS. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade in particular Class 1 proteins can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprises one or more Cas5 subunits and two or more Cas7 subunits. Class 1 subtypes include Type I-A, I-B, I-C, I-U, I-D, I-E, and I-F, Type IV-A and IV-B, and Type III-A, III-D, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n 5, FIG. 5.

### Class 2 Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type VI systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), Cas12d (CasY), Cas12e (CasX), Cas14, and/or CasD.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Guide Molecules

The CRISPR-Cas or Cas-Based system described herein can, in some embodiments, include one or more guide molecules. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9(1981), 133-148). Another example folding algorithm is the online webserver RNAfold, developed at Institute for Theoretical Chemistry at the University of Vienna, using the centroid structure prediction algorithm (see e.g., A.R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 2 (from Gleditzsch et al. 2019) below shows several Cas polypeptides and the PAM sequence they recognize.

TABLE 2
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCasI3b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 29) or PKKKRKVEAS (SEQ ID NO: 30); the NLS from nucleoplasmin (e.g., the nucleoplasmin bipartite NLS with the sequence KRPAATKKAGQAKKKK (SEQ ID NO: 31)); the c-myc NLS having the amino acid sequence PAAKRVKLD (SEQ ID NO: 32) or RQRRNELKRSP (SEQ ID NO: 33); the hRNPAI M9 NLS having the sequence NQSSNFGPMKGGNFGGRSSGPYGGGGQYFAKPRNQGGY (SEQ ID NO: 34); the sequence RMRIZFKNKGKDTAELRRRRVEVSVELRKAKKDEQILKRRNV (SEQ ID NO: 35) of the IBB domain from importin-alpha; the sequences VSRKRPRP (SEQ ID NO: 36) and PPKKARED (SEQ ID NO: 37) of the myoma T protein; the sequence PQPKKKPL (SEQ ID NO: 38) of human p53; the sequence SALIKKKKKMAP (SEQ ID NO: 39) of mouse c-abl IV; the sequences DRLRR (SEQ ID NO: 40) and PKQKKRK (SEQ ID NO: 41) of the influenza virus NS1; the sequence RKLKKKIKKL (SEQ ID NO: 42) of the Hepatitis virus delta antigen; the sequence REKKKFLKRR (SEQ ID NO: 43) of the mouse Mx1 protein; the sequence KRKGDEVDGVDEVAKKKSKK (SEQ ID NO: 44) of the human poly(ADP-ribose) polymerase; and the sequence RKCLQAGMNLEARKTKK (SEQ ID NO: 45) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g., aptamers) for adapter proteins, which may be linked to or fused to a nucleotide deaminase or catalytic domain thereof. When such a guide forms a CRISPR complex (e.g., CRISPR-Cas protein binding to guide and target), the adapter proteins bind and the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

It will be appreciated that NLS and NES described herein with respect to Cas proteins can be used with other cargos, in particularly, gene modifying agents herein, and other proteins that can benefit from translocation in or out of a nuclease of a cell, such as a target cell.

### Donor Templates

In some embodiments, the composition for engineering cells comprise a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include a sequence that corresponds to both, a first site on the target sequence that is cleaved in a first Cas protein mediated event, and a second site on the target sequence that is cleaved in a second Cas protein mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template ‘nucle’c acid can include a sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include a sequence which results in a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain, embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149), which can be adapted for use with the present invention.

### Specialized Cas-based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4× domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and International Patent Publication WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system. See e.g., Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion and combinations thereof. Generally, a prime editing system, as exemplified by PE1, PE2, and PE3 (Id.), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

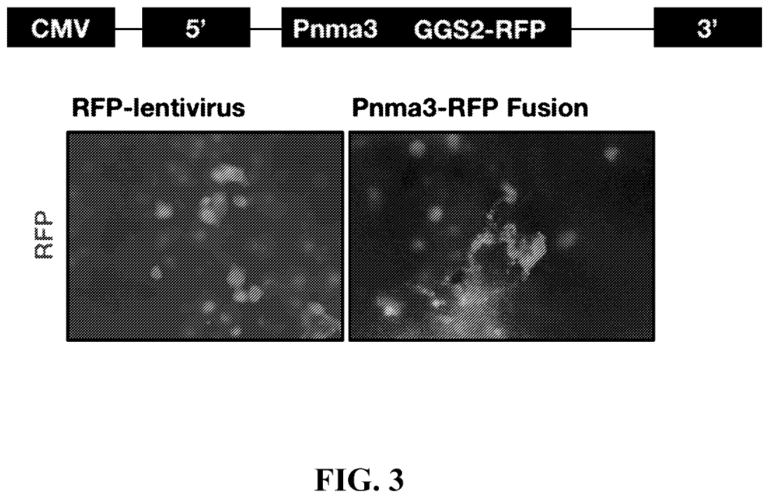

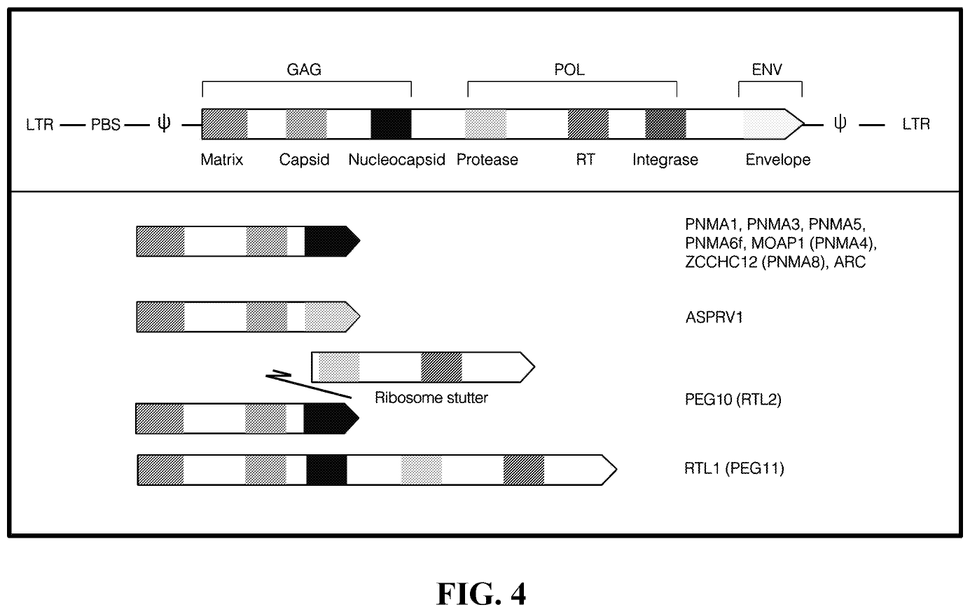

In some embodiments, the prime editing system can be a PE1 system or variant thereof, a PE2 system or variant thereof, or a PE3 (e.g., PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4.

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, the cargo is a CAST system or component thereof. In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class 1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### IscBs

In some embodiments, the nucleic acid-guided nucleases herein may be IscB proteins. In some embodiments a cargo can be an IscB protein, system or component thereof. An IscB protein may comprise an X domain and a Y domain as described herein. In some examples, the IscB proteins may form a complex with one or more guide molecules. In some cases, the IscB proteins may form a complex with one or more hRNA molecules which serve as a scaffold molecule and comprise guide sequences. In some examples, the IscB proteins are CRISPR-associated proteins, e.g., the loci of the nucleases are associated with an CRISPR array. In some examples, the IscB proteins are not CRISPR-associated.

In some examples, the IscB protein may be homolog or ortholog of IscB proteins described in Kapitonov V V et al., ISC, a Novel Group of Bacterial and Archaeal DNA Transposons That Encode Cas9 Homologs, J Bacteriol. 2015 Dec. 28; 198(5):797-807. doi: 10.1128/JB.00783-15, which is incorporated by reference herein in its entirety.

In some embodiments, the IscBs may comprise one or more domains, e.g., one or more of a X domain (e.g., at N-terminus), a RuvC domain, a Bridge Helix domain, and a Y domain (e.g., at C-terminus). In some examples, the nucleic-acid guided nuclease comprises an N-terminal X domain, a RuvC domain (e.g., including a RuvC-I, RuvC-II, and RuvC-III subdomains), a Bridge Helix domain, and a C-terminal Y domain. In some examples, the nucleic-acid guided nuclease comprises an N-terminal X domain, a RuvC domain (e.g., including a RuvC-I, RuvC-II, and RuvC-III subdomains), a Bridge Helix domain, an HNH domain, and a C-terminal Y domain.

In some embodiments, the nucleic acid-guided nucleases may have a small size. For example, the nucleic acid-guided nucleases may be no more than 50, no more than 100, no more than 150, no more than 200, no more than 250, no more than 300, no more than 350, no more than 400, no more than 450, no more than 500, no more than 550, no more than 600, no more than 650, no more than 700, no more than 750, no more than 800, no more than 850, no more than 900, no more than 950, or no more than 1000 amino acids in length.

In some examples, the IscB protein shares at least 80%, at least 85%, at least 90%, at least 95%, at least 99%, or 100% sequence identity with a IscB protein selected from Tables 3A-3B.

TABLE 3A

| No. | Proteins | Sequences |
|---|---|---|
| 1 | IscB(−HNH) | MSTDATLIRTTPSHAEADATDTLVATPLMPPRRVISPWPGPGE |
| | EFH81386 | GQSLMRIPVVDIRGMALMPCTPAKARHLLKSGNARPKRNKL |
| | | GLFYVQLSYEQEPDNQSLVAGVDPGSKFEGLSVVGTKDTVL |
| | | NLMVEAPDHVKGAVQTRRTMRRARRQRKWRRPKRFHNRLN |
| | | RMQRIPPSTRSRWEAKARIVAHLRTILPFTDVVVEDVQAVTR |
| | | KGKGGTWNGSFSPVQVGKEHLYRLLRAMGLTLHLREGWQT |
| | | KELREQHGLKKTKSKSKQSFESHAVDSWVLAASISGAEHPTC |
| | | TRLWYMVPAILHRRQLHRLQASKGGVRKPYGGTRSLGVKRG |
| | | TLVEHKKYGRCTVGGVDRKRNTISLHEYRTNTRLTQAAKV |
| | | ETCRVLTWLSWRSWLLRGKRTSSKGKGSHSS (SEQ ID NO: 46) |
| 2 | IscB(+HNH) | MQPAKQQNWVFQINGDKQPLDMINPGRCRELQNRGKLASFR |
| | TAE54104.1 | RFPYVVIQQQTIENPQTKEYILKIDPGSQWTGFAIQCGNDILFR |
| | | AELNHRGEAIKFDLVKRAWFRRGRRSRNLRYRKKRLNRAKP |
| | | EGWLAPSIRHRVLTVETWIKRFMRYCPIAWIEIEQVRFDTQKL |
| | | ANPEIDGVEYQQGELQGYEVREYLLQKWGRKCAYCGTENVP |
| | | LEVEHIQSKSKGGSSRIGNLTLACHVCNVKKGNLDVRDFLAK |
| | | SPDILNQVLENSTKPLKDAAAVNSTRYAIVKMAKSICENVKC |
| | | SSGARTKMNRVRQGLEKTHSLDAACVGESGASIRVLTDRPLL |
| | | ITCKGHGSRQSIRVNASGFPAVKNAKTVFTHIAAGDVVRFTIG |
| | | KDRKKAQAGTYTARVKTPTPKGFEVLIDGARISLSTMSNVVF |
| | | VHRSDGYGYEL (SEQ ID NO: 47) |
| 3 | IscB(+HNH) | MAVFVIDKHKRPLMPCSEKRARLLLERGRAVVHRQVPFV |
| | WP_038093640.1 | IRLKDRTVQHSAVQPLRVALDPGSRATGMALVREKNTVD |
| | | TGTGEVYRERIALNLFELVHRGHRIREQLDQRRNFRRRRR |
| | | GANLRYRAPRFDNRRRPPGWLAPSLQHRVDTTMAWVRR |
| | | LCRWAPASAIGIETVRFDTQRLQNPEISGVEYQQGALAGC |
| | | EVREYLLEKWGRKCAYCGAENVPLEIEHIVPKSRGGSDRV |
| | | SNLALACRACNQAKGNRDVRAFLADQPERLARILAQAKA |
| | | PLKDAAAVNATRWALYRALVDTGLPVEAGTGGRTKWNR |
| | | TRLGLPKTHALDALCVGQVDQVRHWRVPVLGIRCAGRGS |
| | | YRRTRLTRHGFPRGYLTRNKSAFGFQTGDLIRAVVTKGK |
| | | KAGTYLGRIAIRASGSFNIQTPMGVVQGIHHRFCTLLQR |
| | | ADGYGYFVQPKPTEAALSSPRLKAGVSSAGN (SEQ ID NO: 48) |
| 4 | IscB(+HNH) | MTTNVVFVIDTNQKPLQPCSAAVARKLLLRGKAAMFRRY |
| | WP_052490348.1 | PAVIILKKEVDSVGKPKIELRIDPGSKYTGFALVDSKDNAD |
| | | FIIWGTELEHRGAAICKELTKRSAIRRSRRNRKTRYRKKRF |
| | | ERRKPEGWLAPSLQHRVDTTLTWVKRICKFVPIMSISVEQ |
| | | VKFDLQKLENSDIQGIEYQQGTLAGYTLREALLEHWGRK |
| | | CAYCDVENVFLEIEHIYPKSKGGSDKFSNLTLACHKCNIN |
| | | KGNKSIDEFLLSDHKRLEQIKLHQKKTLKDAAAVNATRK |
| | | KLVTTLQEKTFLNVLVSDGASTKMTRLSSSLAKRHWIDA |
| | | GCVNTTLIVILKTLQPLQVKCNGHGNKQFVTMDAYGFPR |
| | | KSYEPKKVRKDWKAGDIIRVTKKDGTMLMGRVKKAAKK |
| | | LVYIPFGGKEASFSSENAKAIHRSDGYRYSFAAIDSELLQK |
| | | MAT (SEQ ID NO: 49) |
| 5 | IscB(+HNH) | MPNKYAFVLDSKGKLLDPTKSKKAWYLIRKGKASLVEEY |
| | WP_015325818.1 | PLIIKLKREVPKDQVNSDKLILGIDDGTKKVGFALVQKCQ |
| | | TKNKVLFKAVMEQRQDVSKKMEERRGYRRYRRSHKRYR |
| | | PARFDNRSSSKRKGRIPPSILQKKQAILRVVNKLKKYIRID |
| | | KIVLEDVSIDIRKLTEGRELYNWEYQESNRLDENLRKATL |
| | | YRDDCTCQLCGTTETMLHAHHIMPRRDGGADSIYNLITLC |
| | | KACHKDKVDNNEYQYKDQFLAIIDSKELSDLKSASHVMQ |
| | | GKTWLRDKLSKIAQLEITSGGNTANKRIDYEIEKSHSNDAI |
| | | CTTGLLPVDNIDDIKEYYIKPLRKKSKAKIKELKCFRQRDL |
| | | VKYTKRNGETYTGYITSLRIKNNKYNSKVCNFSTLKGKIF |
| | | RGYGFRNLTLLNRPKGLMIV (SEQ ID NO: 50) |
| 6 | sp\|G3ECR1\| | MLFNKCIIISINLDFSNKEKCMTKPYSIGLDIGTNSVGWAVI |
| | CAS9_STRTR | TDNYKVPSKKMKVLGNTSKKYIKKNLLGVLLFDSGITAE |
| | | GRRLKRTARRRYTRRRNRILYLQEIFSTEMATLDDAFFQR |
| | | LDDSFLVPDDKRDSKYPIFGNLVEEKVYHDEFPTIYHLRK |
| | | YLADSTKKADLRLVYLALAHMIKYRGHFLIEGEFNSKNN |
| | | DIQKNFQDFLDTYNAIFESDLSLENSKQLEEIVKDKISKLE |
| | | KKDRILKLFPGEKNSGIFSEFLKLIVGNQADFRKCFNLDEK |
| | | ASLHFSKESYDEDLETLLGYIGDDYSDVFLKAKKLYDAIL |
| | | LSGFLTVTDNETEAPLSSAMIKRYNEHKEDLALLKEYIRNI |
| | | SLKTYNEVFKDDTKNGYAGYIDGKTNQEDFYVYLKNLLA |
| | | EFEGADYFLEKIDREDFLRKQRTFDNGSIPYQIHLQEMRAI |
| | | LDKQAKFYPFLAKNKERIEKILTFRIPYYVGPLARGNSDFA |
| | | WSIRKRNEKITPWNFEDVIDKESSAEAFINRMTSFDLYLPE |
| | | EKVLPKHSLLYETFNVYNELTKVRFIAESMRDYQFLDSKQ |
| | | KKDIVRLYFKDKRKVTDKDIIEYLHAIYGYDGIELKGIEKQ |
| | | FNSSLSTYHDLLNIINDKEFLDDSSNEAIIEEIIHTLTIFEDRE |
| | | MIKQRLSKFENIFDKSVLKKLSRRHYTGWGKLSAKLINGI |
| | | RDEKSGNTIDYLIDDGISNRNFMQLIHDDALSFKKKIQKAQ |
| | | IIGDEDKGNIKEVVKSLPGSPAIKKGILQSIKIVDELVKVMG |
| | | GRKPESIVVEMARENQYTNQGKSNSQQRLKRLEKSLKEL |
| | | GSKILKENIPAKLSKIDNNALQNDRLYLYYLQNGKDMYT |
| | | GDDLDIDRLSNYDIDHIIPQAFLKDNSIDNKVLVSSASNRG |
| | | KSDDFPSLEVVKKRKTFWYQLLKSKLISQRKFDNLTKAER |
| | | GGLLPEDKAGFIQRQLVETRQITKHVARLLDEKFNNKKDE |
| | | NNRAVRTVKIITLKSTLVSQFRKDFELYKVREINDFHHAH |
| | | DAYLNAVIASALLKKYPKLEPEFVYGDYPKYNSFRERKSA |
| | | TEKVYFYSNIMNIFKKSISLADGRVIERPLIEVNEETGESV |
| | | WNKESDLATVRRVLSYPQVNVVKKVEEQNHGLDRGKPK |
| | | GLFNANLSSKPKPNSNENLVGAKEYLDPKKYGGYAGISNS |
| | | FAVLVKGTIEKGAKKKITNVLEFQGISILDRINYRKDKLNF |
| | | LLEKGYKDIELIIELPKYSLFELSDGSRRMLASILSTNNKRG |
| | | EIHKGNQIFLSQKFVKLLYHAKRISNTINENHRKYVENHK |
| | | KEFEELFYYILEFNENYVGAKKNGKLLNSAFQSWQNHSID |
| | | ELCSSFIGPTGSERKGLFELTSRGSAADFEFLGVKIPRYRDY |
| | | TPSSLLKDATLIHQSVTGLYETRIDLAKLGEG |
| | | (SEQ ID NO: 51) |
| 7 | sp\|J7RUA5\| | MKRNYILGLDIGITSVGYGIIDYETRDVIDAGVRLFKEANV |
| | CAS9_STAAU | ENNEGRRSKRGARRLKRRRRHRIQRVKKLLFDYNLLTDH |
| | | SELSGINPYEARVKGLSQKLSEEEFSAALLHLAKRRGVHN |
| | | VNEVEEDTGNELSTKEQISRNSKALEEKYVAELQLERLKK |
| | | DGEVRGSINRFKTSDYVKEAKQLLKVQKAYHQLDQSFID |
| | | TYIDLLETRRTYYEGPGEGSPFGWKDIKEWYEMLMGHCT |
| | | YFPEELRSVKYAYNADLYNALNDLNNLVITRDENEKLEY |
| | | YEKFQIIENVFKQKKKPTLKQIAKEILVNEEDIKGYRVTST |
| | | GKPEFTNLKVYHDIKDITARKEIIENAELLDQIAKILTIYQS |
| | | SEDIQEELTNLNSELTQEEIEQISNLKGYTGTHNLSLKAINL |
| | | ILDELWHTNDNQIAIFNRLKLVPKKVDLSQQKEIPTTLVDD |
| | | FILSPVVKRSFIQSIKVINAIIKKYGLPNDIIIELAREKNSKDA |
| | | QKMINEMQKRNRQTNERIEEIIRTTGKENAKYLIEKIKLHD |
| | | MQEGKCLYSLEAIPLEDLLNNPFNYEVDHIIPRSVSFDNSF |
| | | NNKVLVKQEENSKKGNRTPFQYLSSSDSKISYETFKKHIL |
| | | NLAKGKGRISKTKKEYLLEERDINRFSVQKDFINRNLVDT |
| | | RYATRGLMNLLRSYFRVNNLDVKVKSINGGFTSFLRRKW |
| | | KFKKERNKGYKHHAEDALIIANADFIFKEWKKLDKAKKV |
| | | MENQMFEEKQAESMPEIETEQEYKEIFITPHQIKHIKDFKD |
| | | YKYSHRVDKKPNRELINDTLYSTRKDDKGNTLIVNNLNG |
| | | LYDKDNDKLKKLINKSPEKLLMYHHDPQTYQKLKLIMEQ |
| | | YGDEKNPLYKYYEETGNYLTKYSKKDNGPVIKKIKYYGN |
| | | KLNAHLDITDDYPNSRNKVVKLSLKPYRFDVYLDNGVYK |
| | | FVTVKNLDVIKKENYYEVNSKCYEEAKKLKKISNQAEFIA |
| | | SFYNNDLIKINGELYRVIGVNNDLLNRIEVNMIDITYREYL |
| | | ENMNDKRPPRIIKTIASKTQSIKKYSTDILGNLYEVKSKKH |
| | | PQIIKKG (SEQ ID NO: 52) |
| 8 | Streptococcus_ | KYSIGLDIGTNSVGWAVITDEYKVPSKKFKVLGNTDRHSI |
| | pyogenes_ | KKNLIGALLFDSGETAEATRLKRTARRRYTRRKNRICYLQ |
| | SF370 | EIFSNEMAKVDDSFFHRLEESFLVEEDKKHERHPIFGNIVD |
| | | EVAYHEKYPTIYHLRKKLVDSTDKADLRLIYLALAHMIKF |
| | | RGHFLIEGDLNPDNSDVDKLFIQLVQTYNQLFEENPINASG |
| | | VDAKAILSARLSKSRRLENLIAQLPGEKKNGLFGNLIALSL |
| | | GLTPNFKSNFDLAEDAKLQLSKDTYDDDLDNLLAQIGDQ |
| | | YADLFLAAKNLSDAILLSDILRVNTEITKAPLSASMIKRYD |
| | | EHHQDLTLLKALVRQQLPEKYKEIFFDQSKNGYAGYIDG |
| | | GASQEEFYKFIKPILEKMDGTEELLVKLNREDLLRKQRTF |
| | | DNGSIPHQIHLGELHAILRRQEDFYPFLKDNREKIEKILTFR |
| | | IPYYVGPLARGNSRFAWMTRKSEETITPWNFEEVVDKGAS |
| | | AQSFIERMTNFDKNLPNEKVLPKHSLLYEYFTVYNELTKV |
| | | KYVTEGMRKPAFLSGEQKKAIVDLLFKTNRKVTVKQLKE |
| | | DYFKKIECFDSVEISGVEDRFNASLGTYHDLLKIIKDKDFL |
| | | DNEENEDILEDIVLTLTLFEDREMIEERLKTYAHLFDDKV |
| | | MKQLKRRRYTGWGRLSRKLINGIRDKQSGKTILDFLKSDG |
| | | FANRNFMQLIHDDSLTFKEDIQKAQVSGQGDSLHEHIANL |
| | | AGSPAIKKGILQTVKVVDELVKVMGRHKPENIVIEMAREN |
| | | QTTQKGQKNSRERMKRIEEGIKELGSQILKEHPVENTQLQ |
| | | NEKLYLYYLQNGRDMYVDQELDINRLSDYDVDHIVPQSF |
| | | LKDDSIDNKVLTRSDKNRGKSDNVPSEEVVKKMKNYWR |
| | | QLLNAKLITQRKFDNLTKAERGGLSELDKAGFIKRQLVET |
| | | RQITKHVAQILDSRMNTKYDENDKLIREVKVITLKSKLVS |
| | | DFRKDFQFYKVREINNYHHAHDAYLNAVVGTALIKKYPK |
| | | LESEFVYGDYKVYDVRKMIAKSEQEIGKATAKYFFYSNIM |
| | | NFFKTEITLANGEIRKRPLIETNGETGEIVWDKGRDFATVR |
| | | KVLSMPQVNIVKKTEVQTGGFSKESILPKRNSDKLIARKK |
| | | DWDPKKYGGFDSPTVAYSVLVVAKVEKGKSKKLKSVKE |
| | | LLGITIMERSSFEKNPIDFLEAKGYKEVKKDLIIKLPKYSLF |
| | | ELENGRKRMLASAGELQKGNELALPSKYVNFLYLASHYE |
| | | KLKGSPEDNEQKQLFVEQHKHYLDEIIEQISEFSKRVILAD |
| | | ANLDKVLSAYNKHRDKPIREQAENIIHLFTLTNLGAPAAF |
| | | KYFDTTIDRKRYTSTKEVLDATLIHQSITGLYETRIDLSQL |
| | | GGD (SEQ ID NO: 53) |

TABLE 3B

| No. | Proteins | Domains and amino acid positions |
|---|---|---|
| 1 | IscB(−HNH) EFH81386 | X domain: 51-97 |
| | | RuvC-I: 104-118 |
| | | Bridge Helix: 140-160 |
| | | RuvC-II: 169-212 |
| | | RuvC-III: 226-278 |
| 2 | IscB(+HNH) TAE54104.1 | X domain: 11-56 |
| | | RuvC-I: 63-77 |
| | | Bridge Helix: 100-121 |
| | | RuvC-II: 129-172 |
| | | HNH: 211-243 |
| | | RuvC-III: 279-321 |
| 3 | IscB(+HNH) | X domain: 4-50 |
| | WP_038093640.1 | RuvC-I: 57-71 |
| | | Bridge Helix: 108-129 |
| | | RuvC-II: 138-181 |
| | | HNH: 220-252 |
| | | RuvC-III: 288-330 |
| 4 | IscB(+HNH) | X domain: 7-52 |
| | WP_052490348.1 | RuvC-I: 59-73 |
| | | Bridge Helix: 100-121 |
| | | RuvC-II: 129-172 |
| | | HNH: 211-243 |
| | | RuvC-III: 279-322 |
| 5 | IscB(+HNH) | X domain: 7-52 |
| | WP_015325818.1 | RuvC-I: 61-75 |
| | | Bridge Helix: 101-121 |
| | | RuvC-II: 132-175 |
| | | HNH: 215-247 |
| | | RuvC-III: 284-327 |
| 6 | sp\|G3ECR1\|CAS9_STRTR | RuvC-I: 28-42 |
| | | Bridge Helix: 85-108 |
| | | Rec: 118-736 |
| | | RuvC-II: 750-799 |
| | | HNH: 864-896 |
| | | RuvC-III: 957-1019 |
| | | PAM Interaction (PI): 1119-1409 |
| 7 | sp\|J7RUA5\|CAS9_STAAU | RuvC-I: 7-21 |
| | | Bridge Helix: 49-72 |
| | | Rec: 80-433 |
| | | RuvC-II: 445-493 |
| | | HNH: 553-585 |
| | | RuvC-III: 654-709 |
| | | PAM Interaction (PI): 789-1053 |
| 8 | Streptococcus_pyogenes_ | RuvC-I: 4-18 |
| | SF370 | Bridge Helix: 61-84 |
| | | Rec: 94-718 |
| | | RuvC-II: 725-774 |
| | | HNH: 833-865 |
| | | RuvC-III: 926-988 |
| | | PAM Interaction (PI): 1099-1365 |

### X Domains

In some embodiments, the IscB proteins comprise an X domain, e.g., at its N-terminal.

In certain embodiments, the X domain include the X domains in Tables 3A-3B. Examples of the X domains also include any polypeptides a structural similarity and/or sequence similarity to a X domain described in the art. In some examples, the X domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with X domains in Tables 3A-3B.

In some examples, the X domain may be no more than 10, no more than 20, no more than 30, no more than 40, no more than 50, no more than 60, no more than 70, no more than 80, no more than 90, or no more than 100 amino acids in length. For example, the X domain may be no more than 50 amino acids in length, such as comprising 2 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

### Y Domain

In some embodiments, the IscB proteins comprise a Y domain, e.g., at its C-terminal.

In certain embodiments, the X domain include Y domains in Tables 3A-3B. Examples of the Y domain also include any polypeptides a structural similarity and/or sequence similarity to a Y domain described in the art. In some examples, the Y domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with Y domains in Table 3.

### RuvC Domain

In some embodiments, the IscB proteins comprises at least one nuclease domain. In certain embodiments, the IscB proteins comprise at least two nuclease domains. In certain embodiments, the one or more nuclease domains are only active upon presence of a cofactor. In certain embodiments, the cofactor is Magnesium (Mg). In embodiments where more than one nuclease domain is present and the substrate is a double-strand polynucleotide, the nuclease domains each cleave a different strand of the double-strand polynucleotide. In certain embodiments, the nuclease domain is a RuvC domain.

The IscB proteins may comprise a RuvC domain. The RuvC domain may comprise multiple subdomains, e.g., RuvC-I, RuvC-II and RuvC-III. The subdomains may be separated by interval sequences on the amino acid sequence of the protein.

In certain embodiments, examples of the RuvC domain include those in Tables 3A-3B. Examples of the RuvC domain also include any polypeptides a structural similarity and/or sequence similarity to a RuvC domain described in the art. For example, the RuvC domain may share a structural similarity and/or sequence similarity to a RuvC of Cas9. In some examples, the RuvC domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with RuvC domains in Tables 3A-3B.

### Bridge Helix

The IscB proteins comprise a bridge helix (BH) domain. The bridge helix domain refers to a helix and arginine rich polypeptide. The bridge helix domain may be located next to anyone of the amino acid domains in the nucleic-acid guided nuclease. In some embodiments, the bridge helix domain is next to a RuvC domain, e.g., next to RuvC-I, RuvC-II, or RuvC-III subdomain. In one example, the bridge helix domain is between a RuvC-1 and RuvC2 subdomains.

The bridge helix domain may be from 10 to 100, from 20 to 60, from 30 to 50, e.g., 35, 36, 37, 38, 39, 40, 41, 42, 43, 44, 45, 46 or 47, 48, 49, or 50 amino acids in length. Examples of bridge helix includes the polypeptide of amino acids 60-93 of the sequence of S. pyogenes Cas9.

In certain embodiments, examples of the BH domain include those in Tables 3A-3B. Examples of the BH domain also include any polypeptides a structural similarity and/or sequence similarity to a BH domain described in the art. For example, the BH domain may share a structural similarity and/or sequence similarity to a BH domain of Cas9. In some examples, the BH domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with BH domains in Tables 3A-3B.

### HNH Domain

The IscB proteins comprise an HNH domain. In certain embodiments, at least one nuclease domain shares a substantial structural similarity or sequence similarity to a HNH domain described in the art.

In some examples, the nucleic acid-guided nuclease comprises a HNH domain and a RuvC domain. In the cases where the RuvC domain comprises RuvC-I, RuvC-II, and RuvC-III domain, the HNH domain may be located between the Ruv C II and RuvC III subdomains of the RuvC domain.

In certain embodiments, examples of the HNH domain include those in Tables 3A-3B. Examples of the HNH domain also include any polypeptides a structural similarity and/or sequence similarity to a HNH domain described in the art. For example, the HNH domain may share a structural similarity and/or sequence similarity to a HNH domain of Cas9. In some examples, the HNH domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with HNH domains in Tables 3A-3B.

hRNA

In some examples, the IscB proteins capable of forming a complex with one or more hRNA molecules. The hRNA complex can comprise a guide sequence and a scaffold that interacts with the IscB polypeptide. An hRNA molecules may form a complex with an IscB polypeptide nuclease or IscB polypeptide and direct the complex to bind with a target sequence. In certain example embodiments, the hRNA molecule is a single molecule comprising a scaffold sequence and a spacer sequence. In certain example embodiments, the spacer is 5′ of the scaffold sequence. In certain example embodiments, the hRNA molecule may further comprise a conserved nucleic acid sequence between the scaffold and spacer portions.

As used herein, a heterologous hRNA molecule is an hRNA molecule that is not derived from the same species as the IscB polypeptide nuclease, or comprises a portion of the molecule, e.g., spacer, that is not derived from the same species as the IscB polypeptide nuclease, e.g., IscB protein. For example, a heterologous hRNA molecule of a IscB polypeptide nuclease derived from species A comprises a polynucleotide derived from a species different from species A, or an artificial polynucleotide.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁—(X₁₂X₁₃)—X₁₄₋₃₃ or ₃₄ or ₃₅, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁—(X₁₂X₁₃)—X₁₄₋₃₃ or ₃₄ or ₃₅)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 54)
M D P I R S R T P S P A R E L L S G P Q P D G V
Q P T A D R G V S P P A G G P L D G L P A R R T
M S R T R L P S P P A P S P A F S A D S F S D L
L R Q F D P S L F N T S L F D S L P P F G A H H
T E A A T G E W D E V Q S G L R A A D A P P P T
M R V A V T A A R P P R A K P A P R R R A A Q P
S D A S P A A Q V D L R T L G Y S Q Q Q Q E K I
K P K V R S T V A Q H H E A L V G H G F T H A H
I V A L S Q H P A A L G T V A V K Y Q D M I A A
L P E A T H E A I V G V G K Q W S G A R A L E A
L L T V A G E L R G P P L Q L D T G Q L L K I A
K R G G V T A V E A V H A W R N A L T G A P L N

An exemplary amino acid sequence of a C-terminal capping region is:

(SEQ ID NO: 55)
R P A L E S I V A Q L S R P D P A L A A L T N D
H L V A L A C L G G R P A L D A V K K G L P H A
P A L I K R T N R R I P E R T S H R V A D H A Q
V V R V L G F F Q C H S H P A Q A F D D A M T Q
F G M S R H G L L Q L F R R V G V T E L E A R S
G T L P P A S Q R W D R I L Q A S G M K R A K P
S P T S T Q T P D Q A S L H A F A D S L E R D L
D A P S P M H E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full-length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4× domain or a Kruppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

### Zinc Finger Nucleases

Zinc Finger proteins can comprise a functional domain. The first synthetic zinc finger nucleases (ZFNs) were developed by fusing a ZF protein to the catalytic domain of the Type US restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated herein by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Polypeptides

In certain example embodiments, the cargo molecule may one or more polypeptides. The polypeptide may be a full-length protein or a functional fragment or functional domain thereof, that is a fragment or domain that maintains the desired functionality of the full-length protein. As used within this section “protein” is meant to refer to full-length proteins and functional fragments and domains thereof. A wide array of polypeptides may be delivered using the engineered delivery vesicles described herein, including but not limited to, secretory proteins, immunomodulatory proteins, anti-fibrotic proteins, proteins that promote tissue regeneration and/or transplant survival functions, hormones, anti-microbial proteins, anti-fibrillating polypeptides, and antibodies. The one or more polypeptides may also comprise combinations of the aforementioned example classes of polypeptides. It will be appreciated that any of the polypeptides described herein can also be delivered via the engineered delivery vesicles and systems described herein via delivery of the corresponding encoding polynucleotide.

### Secretor Proteins

In certain example embodiments, the one or more polypeptides may comprise one or more secretory proteins. A secretory is a protein that is actively transported out of the cell, for example, the protein, whether it be endocrine or exocrine, is secreted by a cell. Secretory pathways have been shown conserved from yeast to mammals, and both conventional and unconventional protein secretion pathways have been demonstrated in plants. Chung et al., “An Overview of Protein Secretion in Plant Cells,” MIMB, 1662:19-32, Sep. 1, 2017. Accordingly, identification of secretory proteins in which one or more polynucleotides may be inserted can be identified for particular cells and applications. In embodiments, one of skill in the art can identify secretory proteins based on the presence of a signal peptide, which consists of a short hydrophobic N-terminal sequence.

In embodiments, the protein is secreted by the secretory pathway. In embodiments, the proteins are exocrine secretion proteins or peptides, comprising enzymes in the digestive tract. In embodiments the protein is endocrine secretion protein or peptide, for example, insulin and other hormones released into the blood stream. In other embodiments, the protein is involved in signaling between or within cells via secreted signaling molecules, for example, paracrine, autocrine, endocrine or neuroendocrine. In embodiments, the secretory protein is selected from the group of cytokines, kinases, hormones and growth factors that bind to receptors on the surface of target cells.

As described, secretory proteins include hormones, enzymes, toxins, and antimicrobial peptides. Examples of secretory proteins include serine proteases (e.g., pepsins, trypsin, chymotrypsin, elastase and plasminogen activators), amylases, lipases, nucleases (e.g. deoxyribonucleases and ribonucleases), peptidases enzyme inhibitors such as serpins (e.g., al-antitrypsin and plasminogen activator inhibitors), cell attachment proteins such as collagen, fibronectin and laminin, hormones and growth factors such as insulin, growth hormone, prolactin platelet-derived growth factor, epidermal growth factor, fibroblast growth factors, interleukins, interferons, apolipoproteins, and carrier proteins such as transferrin and albumins. In some examples, the secretory protein is insulin or a fragment thereof. In one example, the secretory protein is a precursor of insulin or a fragment thereof. In certain examples, the secretory protein is c-peptide. In a preferred embodiment, the one or more polynucleotides is inserted in the middle of the c-peptide. In some embodiments, the secretory protein is GLP-1, glucagon, betatrophin, pancreatic amylase, pancreatic lipase, carboxypeptidase, secretin, CCK, a PPAR (e.g., PPAR-alpha, PPAR-gamma, PPAR-delta or a precursor thereof (e.g., preprotein or preproprotein). In aspects, the secretory protein is fibronectin, a clotting factor protein (e.g., Factor VII, VIII, IX, etc.), α2-macroglobulin, α1-antitrypsin, antithrombin III, protein S, protein C, plasminogen, α2-antiplasmin, complement components (e.g., complement component C1-9), albumin, ceruloplasmin, transcortin, haptoglobin, hemopexin, IGF binding protein, retinol binding protein, transferrin, vitamin-D binding protein, transthyretin, IGF-1, thrombopoietin, hepcidin, angiotensinogen, or a precursor protein thereof. In aspects, the secretory protein is pepsinogen, gastric lipase, sucrase, gastrin, lactase, maltase, peptidase, or a precursor thereof. In aspects, the secretory protein is renin, erythropoietin, angiotensin, adrenocorticotropic hormone (ACTH), amylin, atrial natriuretic peptide (ANP), calcitonin, ghrelin, growth hormone (GH), leptin, melanocyte-stimulating hormone (MSH), oxytocin, prolactin, follicle-stimulating hormone (FSH), thyroid stimulating hormone (TSH), thyrotropin-releasing hormone (TRH), vasopressin, vasoactive intestinal peptide, or a precursor thereof.

### Immunomodulatory Polypeptides

In certain example embodiments, the one or more polypeptides may comprise one or more immunomodulatory protein. In certain embodiments, the present invention provides for modulating immune states. The immune state can be modulated by modulating T cell function or dysfunction. In particular embodiments, the immune state is modulated by expression and secretion of IL-10 and/or other cytokines as described elsewhere herein. In certain embodiments, T cells can affect the overall immune state, such as other immune cell” in proximity.

The polynucleotides may encode one or more immunomodulatory proteins, including immunosuppressive proteins. The term “immunosuppressive” means that immune response in an organism is reduced or depressed. An immunosuppressive protein may suppress, reduce, or mask the immune system or degree of response of the subject being treated. For example, an immunosuppressive protein may suppress cytokine production, downregulate or suppress self-antigen expression, or mask the MHC antigens. As used herein, the term “immune response” refers to a response by a cell of the immune system, such as a B cell, T cell (CD4+ or CD8+), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”) and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. In some cases, the immunosuppressive proteins may exert pleiotropic functions. In some cases, the immunomodulatory proteins may maintain proper regulatory T cells versus effector T cells (Treg/Teff) balance. For examples, the immunomodulatory proteins may expand and/or activate the Tregs and blocks the actions of Teffs, thus providing immunoregulation without global immunosuppression. Target genes associated with immune suppression include, for example, checkpoint inhibitors such PD1, Tim3, Lag3, TIGIT, CTLA-4, and combinations thereof.

The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thap, CD4⁺, CD8⁺, effector Th, memory Th, regulatory Th, CD4⁺/CD8⁺ thymocytes, CD4−/CD8− thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

T cell response refers more specifically to an immune response in which T cells directly or indirectly mediate or otherwise contribute to an immune response in a subject. T cell-mediated response may be associated with cell mediated effects, cytokine mediated effects, and even effects associated with B cells if the B cells are stimulated, for example, by cytokines secreted by T cells. By means of an example but without limitation, effector functions of MHC class I restricted Cytotoxic T lymphocytes (CTLs), may include cytokine and/or cytolytic capabilities, such as lysis of target cells presenting an antigen peptide recognized by the T cell receptor (naturally-occurring TCR or genetically engineered TCR, e.g., chimeric antigen receptor, CAR), secretion of cytokines, preferably IFN gamma, TNF alpha and/or or more immunostimulatory cytokines, such as IL-2, and/or antigen peptide-induced secretion of cytotoxic effector molecules, such as granzymes, perforins or granulysin. By means of example but without limitation, for MHC class II restricted T helper (Th) cells, effector functions may be antigen peptide-induced secretion of cytokines, preferably, IFN gamma, TNF alpha, IL-4, IL5, IL-10, and/or IL-2. By means of example but without limitation, for T regulatory (Treg) cells, effector functions may be antigen peptide-induced secretion of cytokines, preferably, IL-10, IL-35, and/or TGF-beta. B cell response refers more specifically to an immune response in which B cells directly or indirectly mediate or otherwise contribute to an immune response in a subject. Effector functions of B cells may include in particular production and secretion of antigen-specific antibodies by B cells (e.g., polyclonal B cell response to a plurality of the epitopes of an antigen (antigen-specific any response)), antigen presentation, and/or cytokine secretion.

During persistent immune activation, such as during uncontrolled tumor growth or chronic infections, subpopulations of immune cells, particularly of CD8+ or CD4+ T cells, become compromised to different extents with respect to their cytokine and/or cytolytic capabilities. Such immune cells, particularly CD8+ or CD4+ T cells, are commonly referred to as “dysfunctional” or as “functionally exhausted” or “exhausted”. As used herein, the term “dysfunctional” or “functional exhaustion” refer to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation (e.g., in response to a cytokine, such as IFN-gamma) or cell division, entrance into the cell cycle, cytokine production, cytotoxicity, migration and trafficking, phagocytotic activity, or any combination thereof. Normal input signals can include, but are not limited to, stimulation via a receptor (e.g., T cell receptor, B cell receptor, co-stimulatory receptor). Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type. In some particular embodiments of the aspects described herein, a cell that is dysfunctional is a CD8+ T cell that expresses the CD8+ cell surface marker. Such CD8+ cells normally proliferate and produce cell killing enzymes, e.g., they can release the cytotoxins perforin, granzymes, and granulysin. However, exhausted/dysfunctional T cells do not respond adequately to TCR stimulation, and display poor effector function, sustained expression of inhibitory receptors and a transcriptional state distinct from that of functional effector or memory T cells. Dysfunction/exhaustion of T cells thus prevents optimal control of infection and tumors. Exhausted/dysfunctional immune cells, such as T cells, such as CD8+ T cells, may produce reduced amounts of IFN-gamma, TNF-alpha and/or one or more immunostimulatory cytokines, such as IL-2, compared to functional immune cells. Exhausted/dysfunctional immune cells, such as T cells, such as CD8+ T cells, may further produce (increased amounts of) one or more immunosuppressive transcription factors or cytokines, such as IL-10 and/or Foxp3, compared to functional immune cells, thereby contributing to local immunosuppression. Dysfunctional CD8+ T cells can be both protective and detrimental against disease control. As used hI, a “dysfunctional immune state” refers to an overall suppressive immune state in a subject or microenvironment of the subject (e.g., tumor microenvironment). For example, increased IL-10 production leads to suppression of other immune cells in a population of immune cells.

CD8+ T cell function is associated with their cytokine profiles. It has been reported that effector CD8+ T cells with the ability to simultaneously produce multiple cytokines (polyfunctional CD8+ T cells) are associated with protective immunity in patients with controlled chronic viral infections as well as cancer patients responsive to immune therapy (Spranger et al., 2014, J. Immunother. Cancer, vol. 2, 3). In the presence of persistent antigen CD8+ T cells were found to have lost cytolytic activity completely over time (Moskophidis et al., 1993, Nature, vol. 362, 758-761). It was subsequently found that dysfunctional T cells can differentially produce IL-2, TNFa and IFNg in a hierarchical order (Wherry et al., 2003, J. Virol., vol. 77, 4911-4927). Decoupled dysfunctional and activated Cell states have also been described (see, e.g., Singer, et al. (2016). A Distinct Gene Module for Dysfunction Uncoupled from Activation in Tumor-Infiltrating T Cells. Cell 166, 1500-1511 e1509; WO/2017/075478; and WO/2018/049025).

The invention provides compositions and methods for modulating T cell balance. The invention provides T cell modulating agents that modulate T cell balance. For example, in some embodiments, the invention provides T cell modulating agents and methods of using these T cell modulating agents to regulate, influence or otherwise impact the level of and/or balance between T cell types, e.g., between Th17 and other T cell types, for example, Th1-like cells. For example, in some embodiments, the invention provides T cell modulating agents and methods of using these T cell modulating agents to regulate, influence or otherwise impact the level of and/or balance between Th17 activity and inflammatory potential. As used herein, terms such as “Th17 cell” and/or “Th17 phenotype” and all grammatical variations thereof refer to a differentiated T helper cell that expresses one or more cytokines selected from the group the consisting of interleukin 17A (IL-17A), interleukin 17F (IL-17F), and interleukin 17A/F heterodimer (IL17-AF). As used herein, terms such as “Th1 cell” and/or “Th1 phenotype” and all grammatical variations thereof refer to a differentiated T helper cell that expresses interferon gamma (IFNγ). As used herein, terms such as “Th2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses Foxp3.

In some examples, immunomodulatory proteins mI immunosuppressive cytokines. In general, cytokines are small proteins and include interleukins, lymphokines and cell signal molecules, such as tumor necrosis factor and the interferons, which regulate inflammation, hematopoiesis, and response to infections. Examples of immunosuppressive cytokines include interleukin 10 (IL-10), TGF-β, IL-Ra, IL-18Ra, IL-2, IL-3, IL-4, IL-5, IL-6, IL-7, IL-8, IL-9, IL-11, IL-12, IL-13, IL-14, IL-15, IL-16, IL-17, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, PGE2, SCF, G-CSF, CSF-1R, M-CSF, GM-CSF, IFN-α, IFN-β, IFN-γ, IFN-λ, bFGF, CCL2, CXCL1, CXCL8, CXCL12, CX3CL1, CXCR4, TNF-α and VEGF. Examples of immunosuppressive proteins may further include FOXP3, AHR, TRP53, IKZF3, IRF4, IRF1, and SMAD3. In one example, the immunosuppressive protein is IL-10. In one example, the immunosuppressive protein is IL-6. In one example, the immunosuppressive protein is IL-2.

### Anti-Fibrotic Proteins

In certain example embodiments, the one or more polypeptides may comprise an anti-fibrotic protein. Examples of anti-fibrotic proteins include any protein that reduces or inhibits the production of extracellular matrix components, fibronectin, proteoglycan, collagen, elastin, TGIFs, and SMAD7. In embodiments, the anti-fibrotic protein is a peroxisome proliferator-activated receptor (PPAR) or may include one or more PPARs. In some embodiments, the protein is PPARα, PPAR γ is a dual PPARα/γ. Derosa et al., “The role of various peroxisome proliferator-activated receptors and their ligands in clinical practice” Jan. 18, 2017 J. Cell. Phys. 223:1 153-161.

Proteins that Promote Tissue Regeneration and/or Transplant Survival Functions

In certain example embodiments, the one or more polypeptides may comprise a proteins that proteins that promote tissue regeneration and/or transplant survival functions. In some cases, such proteins may induce and/or up-regulate the expression of genes for pancreatic β cell regeneration. In some cases, the proteins that promote transplant survival and functions include the products of genes for pancreatic β cell regeneration. Such genes may include proislet peptides that are proteins or peptides derived from such proteins that stimulate islet cell neogenesis. Examples of genes for pancreatic β cell regeneration include Reg1, Reg2, Reg3, Reg4, human proislet peptide, parathyroid hormone-related peptide (1-36), glucagon-like peptide-1 (GLP-1), extendin-4, prolactin, Hgf, Igf-1, Gip-1, adipsin, resistin, leptin, IL-6, IL-10, Pdx1, Ptfa1, Mafa, Pax6, Pax4, Nkx6.1, Nkx2.2, PDGF, vglycin, placental lactogens (somatomammotropins, e.g. CSH1, CHS2), isoforms thereof, homologs thereof, and orthologs thereof. In certain embodiments, the protein promoting pancreatic B cell regeneration is a cytokine, myokine, and/or adipokine.

### Hormones

In certain embodiments, the one or more polynucleotides may comprise one or more hormones. The term “hormone” refers to polypeptide hormones, which are generally secreted by glandular organs with ducts. Hormones include proteins from natural sources or from recombinant cell culture and biologically active equivalents of the native sequence hormone, including synthetically produced small-molecule entities and pharmaceutically acceptable derivatives and salts thereof. Included among the hormones are, for example, growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH),144tilizes144stimulating hormone (TSH), and luteinizing hormone (LH); prolactin, placental lactogen, mouse gonadotropin-associated peptide, inhibin; activin; mullerian-inhibiting substance; and thrombopoietin, growth hormone (GH), adrenocorticotropic hormone (ACTH), dehydroepiandrosterone (DHEA), cortisol, epinephrine, thyroid hormone, estrogen, progesterone, placental lactogens (somatomammotropins, e.g. CSH1, CHS2), testosterone. and neuroendocrine hormones. In certain examples, the hormone is secreted from pancreas, e.g., insulin, glucagon, somatostatin, pancreatic polypeptide and ghrelin. In some examples, the hormone is insulin.

Hormones herein may also include growth factors, e.g., fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoiet 144 tilizes 144 izatios (HeGFs), platelet-derived endothelial cell growth factor (PD-ECGF), angiopoietin, vascular endothelial growth factor (VEGF) family, and glucocorticoidds. In a particular embodiment, the hormone is insulin or incretins such as exenatide, GLP-1.

### Neurohormones

In embodiments, the secreted peptide is a neurohormone, a hormone produced and released by neuroendocrine cells. Example neurohormones include Thyrotropin-releasing hormone, Corticotropin-releasing hormone, Histamine, Growth hormone-releasing hormone, Somatostatin, Gonadotropin-releasing hormone, Serotonin, Dopamine, Neurotensin, Oxytocin, Vasopressin, Epinephrine, and Norepinephrine.

### Anti-Microbial Proteins

In some embodiments, the one or more polypeptides may comprise one or more anti-microbial proteins. In embodiments where the cell is mammalian cell, human host defense antimicrobial peptides and proteins (AMPs) play a critical role in warding off invading microbial pathogens. In certain embodiments, the anti-microbial is α-defensin HD-6, HNP-1 and ρ-defensin hBD-3, lysozyme, cathelcidin LL-37, C-type lectin RegIIIalpha, for example. See, e.g., Wang, “Human Antimicrobial Peptide and Proteins” Pharma, May 2014, 7(5): 545-594, incorporated herein by reference.

### Anti-Fibrillating Proteins

In certain example embodiments, the one or more polypeptides may comprise one or more anti-fibrillating polypeptides. The anti-fibrillating polypeptide can be the secreted polypeptide. In some embodiments, the anti-fibrillating polypeptide is co-expressed with one or more other polynucleotides and/or polypeptides described elsewhere herein. The anti-fibrillating agent can be secreted and act to inhibit the fibrillation and/or aggregation of endogenous proteins and/or exogenous proteins that it may be co-expressed with. In some aspects, the anti-fibrillating agent is P4 (VITYF (SEQ ID NO: 56)), P5 (VVVVV (SEQ ID NO: 57)), KR7 (KPWWPRR (SEQ ID NO: 58)), NK9 (NIVNVSLVK (SEQ ID NO: 59)), iAb5p (Leu-Pro-Phe-Phe-Asp (SEQ ID NO: 60)), KLVF (SEQ ID NO: 61) and derivatives thereof, indolicidin, carnosine, a hexapeptide as set forth in Wang et al. 2014. ACS Chem Neurosci. 5:972-981, alpha sheet peptides having alternating D-amino acids and L-amino acids as set forth in Hopping et al. 2014. Elife 3:e01681, D-(PGKLVYA), RI-OR2-TAT, cyclo(17, 21)-(Lys17, Asp21)A_(1-28), SEN304, SEN1576, D3, R8-AP(25-35), human yD-crystallin (HGD), poly-lysine, heparin, poly-Asp, polyGl, poly-L-lysine, poly-L-glutamic acid, LVEALYL (SEQ ID NO: 62), RGFFYT (SEQ ID NO: 63), a peptide set forth or as designed/generated by the method set forth in U.S. Pat. No. 8,754,034, and combinations thereof. In aspects, the anti-fibrillating agent is a D-peptide. In aspects, the anti-fibrillating agent is an L-peptide. In aspects, the anti-fibrillating agent is a retro-inverso modified peptide. Retro-inverso modified peptides are derived from peptides by substituting the L-amino acids for their D-counterparts and reversing the sequence to mimic the original peptide since they retain the same spatial positioning of the side chains and 3D structure. In aspects, the retro-inverso modified peptide is derived from a natural or synthetic Aβ peptide. In some embodiments, the polynucleotide encodes a fibrillation resistant protein. In some embodiments, the fibrillation resistant protein is a modified insulin, see e.g. U.S. Pat. No. 8,343,914.

Antibodies

In certain embodiments, the one or more polypeptides may comprise one or more antibodies. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scF″ and/or Fv fragments. As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g., the IgG1, IgG2, IgG3, and IgG4 subclasses of IgG” obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG—IgG1, IgG2, IgG3, and IgG4 that have seen identified in humans and higher mammals by the heavy chains of the immunoglobulins, VI—γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of “constant” domain, or the significant variation within the domains of various class “embers” n the case of a “variable” domain. Antibody or polypeptide “domains” are often referred to interchangeably in the ar “a” antibody or polypeptide “regions” “The” constant domains of an antibody light chain are referred to interchangeably as “light chain constant regions”, “light chain constant domains”, “CL” regions or “CL” domains” The “constant domains of an antibody heavy chain are referred to interchangeably as “heavy chain constant regions”, “heavy chain constant domains”, “CH” regions or “CH” domains)” The “variable” domains of an antibody light chain are referred to interchangeably as “light chain variable regions”, “light chain variable domains”, “VL” regions or “VL” domains)” The variable domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase light (or heavy) chain conformation refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a sma149tiliza. 58 residues) and robust, disulphide-crosslinked serine protease inhibitor, typically of human origin (e.g., LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins—harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin “peptides (Kolmar” Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity, but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunogloblins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L}domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92 (6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-321 4 (1998); Yoon et al., J. Immunol. 160(7):3170-3179 (1998); Pr at et al., J. Cell. Sci. III (Pt2):237-247 (1998); Pitard e t al., J. Immunol. Methods 205 (2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### Protease Cleavage Sites

The one or more cargo polypeptides, as exemplified above, may comprise one or more protease cleavage sites, i.e., amino acid sequences that can be recognized and cleaved by a protease. The protease cleavage sites may be used for generating desired gene products (e.g., intact gene products without any tags or portion of other proteins). The protease cleavage site may be one end or both ends of the protein. Examples of protease cleavage sites that can be used herein include an enterokinase cleavage site, a thrombin cleavage site, a Factor Xa cleavage site, a human rhinovirus 3C protease cleavage site, a tobacco etch virus (TEV) protease cleavage site, a dipeptidyl aminopeptidase cleavage site and a small ubiquitin-like modifier (SUMO)/ubiquitin-like protein-1(ULP-1) protease cleavage site. In certain examples, the protease cleavage site comprises Lys-Arg.

### Small Molecules

In some embodiments, the engineered delivery vesicle can deliver one or more small molecule compounds. Thus, in some embodiments, the cargo molecule is a small molecule. In some embodiments, the small molecule compound(s) can be linked or directly attached to a polynucleotide that can bind a polynucleotide binding protein that can be included in the engineered delivery system polynucleotide. In some embodiments, the engineered delivery system polynucleotide can include a small molecule binding protein (e.g., a receptor for the small molecule) that, like the polynucleotide binding protein discussed elsewhere herein, can be incorporated into the engineered delivery vesicle.

In some embodiments, the small molecule compound(s) can be linked or directly attached to a polynucleotide that can bind a polynucleotide binding protein that can be included in the engineered delivery system polynucleotide or delivery vesicle. In some embodiments, the engineered delivery system polynucleotide or delivery vesicle can include a small molecule binding protein (e.g., a receptor for the small molecule) that, like the polynucleotide binding protein discussed elsewhere herein, can be incorporated into the engineered delivery system polynucleotide or delivery vesicle.

Suitable hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-154tilizes 154iza hormone), eicosanoids (e.g., arachidonic acid, lipoxins, and prostaglandins), and steroid hormones (e.g., estradiol, testosterone, tetrahydro testosteron Cortisol). Suitable immunomodulators include, but are not limited to, prednisone, azathioprine, 6-MP, cyclosporine, tacrolimus, methotrexate, interleukins (e.g., IL-2, IL-7, and IL-12), cytokines (e.g., interferons (e.g., IFN-α, IFN-β, IFN-ε, IFN-K, IFN-ω, and IFN-γ), granulocyte colony-stimulating factor, and imiquimod), chemokines (e.g., CCL3, CCL26 and CXCL7), cytosine phosphate-guanosine, oligodeoxynucleotides, glucans, antibodies, and aptamers).

Suitable antipyretics include, but are not limited to, non 154tilizes 154il anti-inflammants (e.g., ibuprofen, naproxen, ketoprofen, and nimesulide), aspirin and related salicylates (e.g., choline salicylate, magnesium salicylae, and sodium salicaylate), paracetamol/acetaminophen, metamizole, nabumetone, phenazone, and quinine.

Suitable anxiolytics include, but are not limited to, benzodiazepines (e.g., alprazolam, bromazepam, chlordiazepoxide, clonazepam, clorazepate, diazepam, flurazepam, lorazepam, oxazepam, temazepam, triazolam, and tofisopam), serotenergic antidepressants (e.g., selective serotonin reuptake inhibitors, tricyclic antidepresents, and monoamine oxidase inhibitors), mebicar, afobazole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipaperone, timip 154tilizes 154izpirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melper 154 tilizes 154 iapride, olanzapine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammants (e.g., ibuprofen, naproxen, ketoprofen, and nimesulide), COX-2 inhibitors (e.g., rofecoxib, celecoxib, and etoricoxib), opioids (e.g., morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene. Suitable anti-inflammatories include, but are not limited to, prednisone, non-steroidal anti-inflammants (e.g., ibuprofen, naproxen, ketoprofen, and nimesulide), COX-2 inhibitors (e.g., rofecoxib, celecoxib, and etoricoxib), and immune selective anti-inflammatory derivatives (e.g., submandibular gland peptide-T and its derivatives).

Suitable anti-histamines include, but are not limited to, H1-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, 155 tilizes 155 pheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, rafitidine, and roxatidine), tritoqualine, catechin, cromoglicate, nedocromil, and p2-adrenergic agonists.

Suitable anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrante 155 tilizes 155 izale, ivermectin, praziquantel, abendazole, thiabendazole, oxamniquine), antifungals (e.g., azole antifungals (e.g., itraconazole, fluconazole, posaconazole, ketoconazole, clotrimazole, miconazole, and voriconazole), echinocandins (e.g., caspofungin, anidulafungin, and micafungin), griseofulvin, terbinafine, flucytosine, and polyenes (e.g. nystatin, and amphotericin b), antimalarial agents (e.g., pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g., aminosalicylates (e.g., aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g., doripenem, meropenem, ertapenem, and cilastatin/mipenem), cephalosporins (e.g., cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g., vancomycin, dalbavancin, oritavancin, and telvancin), glycylcyclines (e.g., tigecycline), leprostatics (e.g., clofazimine and th156tilizes156iza lincomycin and derivatives thereof (e.g., clindamycin and lincomycin), macrolides and derivatives thereof (e.g., telithromycin, fidaxomi 156tilizes156iomycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, o 156 tilizes 156 izacloxacillin, and nafcillin), quinolones (e.g., lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfasoxazole), tetracyclines (e.g., doxycycline, demeclocycline, minocycline, doxycyc 157tilizes 157iyclic acid, doxycycline/omega-3 polyunsaturated fatty acids, and tetracycline), and urinary anti-infectives (e.g., nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daun 157 tilizes 157 izlofarabine, cabozantinib, dactinomycin, ramucirumab, cytarabine, Cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, as 157 tilizes 157 izawinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thio 157 tilize (tioguanine), dabrafenib, erlotinib, bexarotene, temozolomide, thiotepa, thalidomi 157tilizes157izmsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

### Formulations

Described in certain example embodiments are formulations comprising an engineered delivery vesicle generation system as described herein; and a buffer optimized for RNA binding and/or encapsidation. In certain example embodiments, the buffer comprises an optimized concentration of a salt, optionally NaCl, and an optimized concentration of ZnSO₄.

In certain example embodiments, the optimized concentration of NaCl ranges from 0 mM to 1 M. In some embodiments, the optimized concentration of NaCl is about 0 mM or M, 0.01 mM or M, 0.02 mM or M, 0.03 mM or M, 0.04 mM or M, 0.05 mM or M, 0.06 mM or M, 0.07 mM or M, 0.08 mM or M, 0.09 mM or M, 0.1 mM or M, 0.11 mM or M, 0.12 mM or M, 0.13 mM or M, 0.14 mM or M, 0.15 mM or M, 0.16 mM or M, 0.17 mM or M, 0.18 mM or M, 0.19 mM or M, 0.2 mM or M, 0.21 mM or M, 0.22 mM or M, 0.23 mM or M, 0.24 mM or M, 0.25 mM or M, 0.26 mM or M, 0.27 mM or M, 0.28 mM or M, 0.29 mM or M, 0.3 mM or M, 0.31 mM or M, 0.32 mM or M, 0.33 mM or M, 0.34 mM or M, 0.35 mM or M, 0.36 mM or M, 0.37 mM or M, 0.38 mM or M, 0.39 mM or M, 0.4 mM or M, 0.41 mM or M, 0.42 mM or M, 0.43 mM or M, 0.44 mM or M, 0.45 mM or M, 0.46 mM or M, 0.47 mM or M, 0.48 mM or M, 0.49 mM or M, 0.5 mM or M, 0.51 mM or M, 0.52 mM or M, 0.53 mM or M, 0.54 mM or M, 0.55 mM or M, 0.56 mM or M, 0.57 mM or M, 0.58 mM or M, 0.59 mM or M, 0.6 mM or M, 0.61 mM or M, 0.62 mM or M, 0.63 mM or M, 0.64 mM or M, 0.65 mM or M, 0.66 mM or M, 0.67 mM or M, 0.68 mM or M, 0.69 mM or M, 0.7 mM or M, 0.71 mM or M, 0.72 mM or M, 0.73 mM or M, 0.74 mM or M, 0.75 mM or M, 0.76 mM or M, 0.77 mM or M, 0.78 mM or M, 0.79 mM or M, 0.8 mM or M, 0.81 mM or M, 0.82 mM or M, 0.83 mM or M, 0.84 mM or M, 0.85 mM or M, 0.86 mM or M, 0.87 mM or M, 0.88 mM or M, 0.89 mM or M, 0.9 mM or M, 0.91 mM or M, 0.92 mM or M, 0.93 mM or M, 0.94 mM or M, 0.95 mM or M, 0.96 mM or M, 0.97 mM or M, 0.98 mM or M, 0.99 mM or M, 1 mM or M, or any numerical value or range of values therein.

In certain example embodiments, the optimized concentration of ZnSO₄ ranges from 0 μM to 1 mM. In some embodiments, the optimized concentration of ZnSO₄ is about 0 μM, mM, or M, 0.01 μM, mM, or M, 0.02 μM, mM, or M, 0.03 μM, mM, or M, 0.04 μM, mM, or M, 0.05 μM, mM, or M, 0.06 μM, mM, or M, 0.07 μM, mM, or M, 0.08 μM, mM, or M, 0.09 μM, mM, or M, 0.1 μM, mM, or M, 0.11 μM, mM, or M, 0.12 μM, mM, or M, 0.13 μM, mM, or M, 0.14 μM, mM, or M, 0.15 μM, mM, or M, 0.16 μM, mM, or M, 0.17 μM, mM, or M, 0.18 μM, mM, or M, 0.19 μM, mM, or M, 0.2 μM, mM, or M, 0.21 μM, mM, or M, 0.22 μM, mM, or M, 0.23 μM, mM, or M, 0.24 μM, mM, or M, 0.25 μM, mM, or M, 0.26 μM, mM, or M, 0.27 μM, mM, or M, 0.28 μM, mM, or M, 0.29 μM, mM, or M, 0.3 μM, mM, or M, 0.31 μM, mM, or M, 0.32 μM, mM, or M, 0.33 μM, mM, or M, 0.34 μM, mM, or M, 0.35 μM, mM, or M, 0.36 μM, mM, or M, 0.37 μM, mM, or M, 0.38 μM, mM, or M, 0.39 μM, mM, or M, 0.4 μM, mM, or M, 0.41 μM, mM, or M, 0.42 μM, mM, or M, 0.43 μM, mM, or M, 0.44 μM, mM, or M, 0.45 μM, mM, or M, 0.46 μM, mM, or M, 0.47 μM, mM, or M, 0.48 μM, mM, or M, 0.49 μM, mM, or M, 0.5 μM, mM, or M, 0.51 μM, mM, or M, 0.52 μM, mM, or M, 0.53 μM, mM, or M, 0.54 μM, mM, or M, 0.55 μM, mM, or M, 0.56 μM, mM, or M, 0.57 μM, mM, or M, 0.58 μM, mM, or M, 0.59 μM, mM, or M, 0.6 μM, mM, or M, 0.61 μM, mM, or M, 0.62 μM, mM, or M, 0.63 μM, mM, or M, 0.64 μM, mM, or M, 0.65 μM, mM, or M, 0.66 μM, mM, or M, 0.67 μM, mM, or M, 0.68 μM, mM, or M, 0.69 μM, mM, or M, 0.7 μM, mM, or M, 0.71 μM, mM, or M, 0.72 μM, mM, or M, 0.73 μM, mM, or M, 0.74 μM, mM, or M, 0.75 μM, mM, or M, 0.76 μM, mM, or M, 0.77 μM, mM, or M, 0.78 μM, mM, or M, 0.79 μM, mM, or M, 0.8 μM, mM, or M, 0.81 μM, mM, or M, 0.82 μM, mM, or M, 0.83 μM, mM, or M, 0.84 μM, mM, or M, 0.85 μM, mM, or M, 0.86 μM, mM, or M, 0.87 μM, mM, or M, 0.88 μM, mM, or M, 0.89 μM, mM, or M, 0.9 μM, mM, or M, 0.91 μM, mM, or M, 0.92 μM, mM, or M, 0.93 μM, mM, or M, 0.94 μM, mM, or M, 0.95 μM, mM, or M, 0.96 μM, mM, or M, 0.97 μM, mM, or M, 0.98 μM, mM, or M, 0.99 μM, mM, or M, 1 μM, mM, or M, or any numerical value or range of values therein.

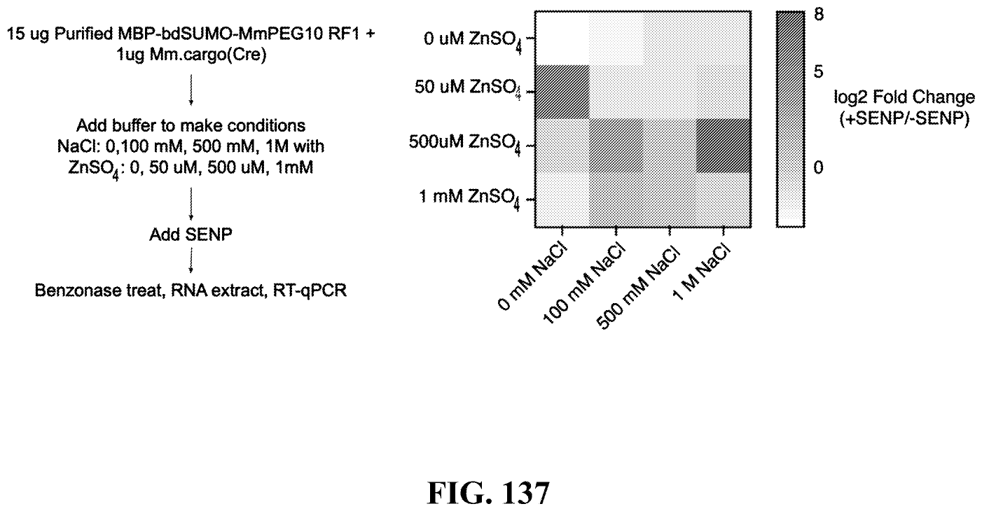

In certain example embodiments, the optimized concentration of NaCl is about 1 M and the optimized concentration of ZnSO₄ is about 0.5 mM. In certain example embodiments, the optimized concentration of NaCl is about 0 M and the optimized concentration of ZnSO₄ ranges from about 0.05 mM to about 0.5 mM. In certain example embodiments, the optimized concentration of ZnSO₄ is about 0.05 mM or about 0.5 mM. In certain example embodiments, the formulation further comprises a pharmaceutically acceptable carrier. Further exemplary buffers are described at least in the Working Examples elsewhere herein. See also e.g., FIG. 137.

### Delivery Vesicles

Also envisioned within the scope of the invention is a delivery vesicle generated from the engineered delivery system described herein. Described in several embodiments herein are delivery vesicles comprising an (e.g., endogenous) LTR retroelement polypeptide and a non-heterologous cargo molecule, the (e.g., endogenous) LTR retroelement polypeptide forming the delivery vesicle and encapsulating the non-heterologous cargo molecule. As used herein “non-heterologous” is used to refer to cargo molecules not normally packaged by the delivery vesicle. For example, in the context of PEG10 which can package its own mRNA, a non-heterologous cargo molecule would exclude a naturally occurring PEG10 delivery vesicle comprising its own naturally occurring mRNA. In some embodiments, the delivery vesicle elicits a poor immune response, as described elsewhere herein.

In some embodiments, the vesicle further comprises a reverse transcriptase.

### Engineered Cells

Described herein are various aspects of engineered cells that can include one or more of the engineered delivery system polynucleotides, polypeptides, vectors, and/or vector systems, and/or engineered delivery vesicles (e.g., those produced from an engineered delivery system polynucleotide and/or vector(s)) described elsewhere herein. In some embodiments, the engineered cells can express one or more of the engineered delivery system polynucleotides and/or can produce one or more engineered delivery vesicles, which are described in greater detail herein. Such cells are also referred to herein as “producer cells” or donor cells, depending on the context. It will be appreciated that these engineered cells are different from “modified cells” described elsewhere herein in that the modified cells are not necessarily producer or donor cells (e.g., they do not make engineered delivery vesicles) unless they include one or more of the engineered delivery system molecules or vectors described herein that render the cells capable of producing an engineered delivery vesicle. Modified cells can be recipient cells of an engineered delivery vesicle and can, in some embodiments, be said to be modified by the engineered delivery vesicles and/or a cargo present in the engineered delivery vesicle that is delivered to the recipient cell. The term “modification” can be used in connection with modification of a cell that is not dependent on being a recipient cell. For example, isolated cells can be modified prior to receiving an engineered delivery system or engineered delivery vesicle and/or cargo.

In an aspect, the invention provides a non-human eukaryotic organism; for example, a multicellular eukaryotic organism, including a eukaryotic host cell containing one or more components of an engineered delivery system described herein according to any of the described embodiments. In other aspects, the invention provides a eukaryotic organism; preferably a multicellular eukaryotic organism, comprising a eukaryotic host cell containing one or more components of an engineered delivery system described herein according to any of the described embodiments. In some embodiments, the organism is a host of AAV.

The engineered cell can be any eukaryotic cell, including but not limited to, human, non-human animal, plant, algae, and the like.

The engineered cell can be a prokaryotic cell. The prokaryotic cell can be bacterial cell. The prokaryotic cell can be an archaea cell. The bacterial cell can be any suitable bacterial cell. Suitable bacterial cells can be from the genus Escherichia, Bacillus, Lactobacillus, Rhodococcus, Rodhobacter, Synechococcus, Synechoystis, Pseudomonas, Psedoaltermonas, Stenotrophamonas, and Streptomyces Suitable bacterial cells include, but are not limited to Escherichia coli cells, Caulobacter crescentus cells, Rodhobacter sphaeroides cells, Psedoaltermonas haloplanktis cells. Suitable strains of bacterial include, but are not limited to BL21(DE3), DL21(DE3)-pLysS, BL21 Star-pLysS, BL21-SI, BL21-AI, Tuner, Tuner pLysS, Origami, Origami B pLysS, Rosetta, Rosetta pLysS, Rosetta-gami-pLysS, BL21 CodonPlus, AD494, BL2trxB, HMS174, NovaBlue(DE3), BLR, C41(DE3), C43(DE3), Lemo21(DE3), Shuffle T7, ArcticExpress and ArticExpress (DE3).

The engineered cell can be a eukaryotic cell. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, the engineered cell can be a cell line. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CiR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, Bl16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see e.g., the American Type Culture Collection (ATCC) (Manassas, Va.)).

Further, the engineered cell may be a fungus cell. As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastomycota, fungal cells may include yeasts, molds, and filamentous fungi. In some embodiments, the fungal cell is a yeast cell.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In some embodiments, the yeast cell is an S. cerevisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientali, a.k.a. Pichia kudriavzevii and Candida acidothermophilum). In some embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus (e.g., Aspergillus niger), Trichoderma spp. (e.g., Trichoderma reesei), Rhizopus spp. (e.”., Rhizopus oryza”), and Mortierella spp. (e.g., Mortierella isabellina).

In some embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Example” of industrial strains can include, without limitation, JAY270 and ATCC4124.

In some embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest.

In some embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In some embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

In some embodiments, the engineered cell is a cell obtained from a subject. In some embodiments, the subject is a healthy or non-diseased subject. In some embodiments, the subject is a subject with a desired physiological and/or biological characteristic such that when an engineered delivery vesicle is produced it can package one or more molecules that are within the producer cell that can be related to the desired physiological and/or biological characteristic. In this context, the cargo molecules incorporated into the delivery vesicles can be capable of transferring the desired characteristic to a recipient cell.

In some embodiments, a cell can be obtained from a subject, modified such that it is an engineered delivery vesicle producer cell, and administered back to the subject from which it was obtained (autologous) or delivered to an allogenic subject. In other words, a producer cell described herein can be used in an autologous or allogenic context, such as in a cell therapy. In these embodiments, the cells can deliver a cargo, such as a therapeutic cargo or a cargo that can manipulate a cellular microenvironment within the subject.

Conventional viral and non-viral based gene transfer methods can be used to introduce nucleic acids (e.g., such as one or more of the polynucleotides of the engineered delivery system described herein) in cells or target tissues. Such methods can be used to administer nucleic acids encoding components of a nucleic acid-targeting system to cells in culture, or in a host organism. In some embodiments, a delivery is via a polynucleotide molecule (e.g., a DNA or RNA molecule) not contained in a vector. In some embodiments, delivery is via a vector. In some embodiments, delivery, is via viral particles. In aspects delivery is via a particle, (e.g., a nanoparticle) carrying one or more engineered delivery system polynucleotides, vectors, or viral particles. Particles, including nanoparticles, are discussed in greater detail elsewhere herein.

Vector delivery can be appropriate in some embodiments, where in vivo expression is envisaged. It will be appreciated that the engineered cells can be generated in vitro, ex vivo, in situ, or in vivo by delivery of one or more components of the engineered delivery systems as described elsewhere herein.

Suitable conventional viral and non-viral based methods of engineering cells to contain and/or express the engineered delivery system polynucleotides and/or vectors described herein are generally known in the art and/or described elsewhere herein.

### Formulations

Component(s) of the engineered delivery system, engineered cells, engineered delivery vesicles, or combinations thereof can be included in a formulation that can be delivered to a subject or cell. In some embodiments, the formulation is a pharmaceutical formulation. One or more of the polypeptides, polynucleotides, vectors, cells, and combinations thereof described herein can be provided to a subject in need thereof or a cell alone or as an active ingredient, such as in a pharmaceutical formulation. As such, also described herein are pharmaceutical formulations containing an amount of one or more of the polypeptides, polynucleotides, vectors, cells, and combinations thereof described herein. In some embodiments, the pharmaceutical formulation can contain an effective amount of the one or more of the polypeptides, polynucleotides, vectors, cells, and combinations thereof described herein. The pharmaceutical formulations described herein can be administered to a subject in need thereof or a cell.

In some embodiments, the amount of the one or more of the polypeptides, polynucleotides, vectors, cells, virus particles, nanoparticles, other delivery particles, and combinations thereof described herein contained in the pharmaceutical formulation can range from about 1 pg/kg to about 10 mg/kg based upon the bodyweight of the subject in need thereof or average bodyweight of the specific patient population to which the pharmaceutical formulation can be administered. The amount of the one or more of the polypeptides, polynucleotides, vectors, cells, and combinations thereof described herein in the pharmaceutical formulation can range from about 1 pg to about 10 g, from about 10 nL to about 10 ml. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells. In aspects where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells per nL, μL, mL, or L.

### Pharmaceutically Acceptable Carriers and Auxiliary Ingredients and Agents

In aspects, the pharmaceutical formulation containing an amount of one or more of the polypeptides, polynucleotides, vectors, cells, virus particles, nanoparticles, other delivery particles, and combinations thereof described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In addition to an amount of one or more of the polypeptides, polynucleotides, vectors, cells, engineered delivery vesicles, nanoparticles, other delivery particles, and combinations thereof described herein, the pharmaceutical formulation can also include an effective amount of an auxiliary active agent, including but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

In embodiments where there is an auxiliary active agent contained in the pharmaceutical formulation in addition to the one or more of the polypeptides, polynucleotides, CRISPR-Cas complexes, vectors, cells, virus particles, nanoparticles, other delivery particles, and combinations thereof described herein, amount, such as an effective amount, of the auxiliary active agent will vary depending on the auxiliary active agent. In some embodiments, the amount of the auxiliary active agent ranges from 0.001 micrograms to about 1 milligram. In other embodiments, the amount of the auxiliary active agent ranges from about 0.01 IU to about 1000 IU. In further embodiments, the amount of the auxiliary active agent ranges from 0.001 mL to about 1 mL. In yet other embodiments, the amount of the auxiliary active agent ranges from about 1% w/w to about 50% w/w of the total pharmaceutical formulation. In additional embodiments, the amount of the auxiliary active agent ranges from about 1% v/v to about 50% v/v of the total pharmaceutical formulation. In still other embodiments, the amount of the auxiliary active agent ranges from about 1% w/v to about 50% w/v of the total pharmaceutical formulation.

### Dosage Forms

In some embodiments, the pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to 1000 g of a pharmaceutical formulation containing a therapeutically effective amount or an appropriate fraction thereof of the targeted effector fusion protein and/or complex thereof or composition containing the one or more of the polypeptides, polynucleotides, vectors, cells, and combinations thereof described herein. The oral dosage form can be administered to a subject in need thereof.

Where appropriate, the dosage forms described herein can be microencapsulated.

The dosage form can also be prepared to prolong or sustain the release of any ingredient. In some embodiments, the one or more of the polypeptides, polynucleotides, vectors, cells, and combinations thereof described herein can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax” gels, and the like. Delayed release dosage formulations can b” prepared -s described in standard references such as “Pharmaceutical dosage form tablets,” eds. Liberman et. al. (New York, Marcel Dekker, Inc., 1989), “Remington—The silence and practice of pharmacy”, 20th ed., Lippincott Williams & Wilkins, Baltimore, M D, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the one or more of the polypeptides, polynucleotides, vectors, cells, and combinations thereof described herein can be formulated with a paraffinic or water-miscible ointment base. In some embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the one or more of the polypeptides, polynucleotides, vectors, cells, and combinations thereof described herein is contained in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g., the one or more of the polypeptides, polynucleotides, vectors, cells, and combinations thereof described herein and/or auxiliary active agent), which may be generated by various types of metered dose pressurized aerosols, nebulizers, or insufflators.

In some embodiments, the dosage forms can be aerosol formulations suitable for administration by inhalation. In some of these embodiments, the aerosol formulation can contain a solution or fine suspension of the one or more of the polypeptides, polynucleotides, vectors, cells, and combinations thereof described herein and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of one or more of the polypeptides, polynucleotides, vectors, cells, and combinations thereof described herein. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the one or more of the polypeptides, polynucleotides, vectors, cells, and combinations thereof described herein, an auxili169tilizesive ingredient, and/or pharmaceutically acceptable salt thereof, such a dosage form can contain a powder base such as lactose, glucose, trehalose, manitol, and/or starch. In some of these embodiments, the one or more of the polypeptides, polynucleotides, vectors, cells, and combinations thereof described herein is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol dosage forms can be arranged so that each metered dose of aerosol contains a predetermined amount of an active ingredient, such as the one or more of the one or more of the polypeptides, polynucleotides, vectors, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas.

Dosage forms adapted for parenteral administration and/or adapted for any type of injection (e.g., intravenous, intraperitoneal, subcutaneous, intramuscular, intradermal, intraosseous, epidural, intracardiac, intraarticular, intracavernous, gingival, subginigival, intrathecal, intravireal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

Dosage forms adapted for ocular administration can include aqueous and/or nonaqueous sterile solutions that can optionally be adapted for injection, and which can optionally contain anti-oxidants, buffers, bacteriostats, solutes that render the composition isotonic with the eye or fluid contained therein or around the eye of the subject, and aqueous and nonaqueous sterile suspensions, which can include suspending agents and thickening agents.

For some embodiments, the dosage form contains a predetermined amount of the one or more of the polypeptides, polynucleotides, vectors, cells, and combinations thereof described herein per unit dose. In some embodiments, the predetermined amount of the Such unit doses may therefore be administered once or more than once a day. Such pharmaceutical formulations may be prepared by any of the methods well known in the art.

### Methods of Loading Cargo Molecules in Delivery Vesicle Systems

The cargo, which is of a size sufficiently small to be enclosed in the delivery vesicle, e.g., nucleic acids and/or polypeptides, can be introduced to cells by transduction by a viral or pseudoviral particle. Methods of packaging the cargos in viral particles can be accomplished using any suitable vector systems. Such vector systems are described in greater detail elsewhere herein. As used in this context herein “transduction” refers to the process by which foreign nucleic acids and/or proteins are introduced to a cell (prokaryote or eukaryote) by a viral or pseudo viral particle. Cargo-loaded delivery vesicles of the present invention can be exposed to cells (e.g., in vitro, ex vivo, or in vivo) where the delivery vesicles deliver the cargo to the target cell, for example, by transduction. Delivery vesicles can be optionally concentrated prior to exposure to target cells.

One approach for packaging cargo inside vesicles involves the use of one or more “bioreactors” which produce and subsequently secrete one or more cargo-carrying vesicles. Bioreactors may comprise cells, microorganisms, or acellular systems. A bioreactor cell is generated by administering to a cell one or more polynucleotides encoding one or more (e.g., endogenous) LTR retroelement polypeptides for forming a delivery vesicle and one or more capture moieties for packaging a cargo within the delivery vesicle. Accordingly, the bioreactor may be capable of producing cargo-carrying vesicles that not only deliver the biologically active RNA molecule(s) to the extracellular matrix, but also to specific cells and tissues. Cells suitable for being bioreactor cells for producing engineered delivery system polynucleotides, polypeptides, and/or engineered delivery vesicles (loaded with a cargo(s) or not) are described elsewhere herein including but not limited to the section “Engineered cells”.

In certain example embodiments, the one or more bioreactors are one or more cells, optionally one or more eukaryotic cells or prokaryotic cells. Exemplary eukaryotic and prokaryotic cells that are suitable bioreactors are described elsewhere herein.

Described in certain example embodiments herein are methods of generating engineered delivery vesicles loaded with one or more cargo polynucleotides, comprising delivering to and/or incubating a delivery vesicle generation system as described herein in one or more bioreactors; and isolating generated engineered delivery vesicles from the one or more bioreactors. The producer cells (or bioreactors) can secrete engineered delivery vesicles, including loaded engineered delivery vesicles with a packaged cargo, into a suitable media formulation that the bioreactors are cultured in. The media can be collected, and the delivery vesicles can be harvested, isolated, and/or purified from the cell culture media. Isolation and purification techniques can include, without limitation, size separation methods (e.g., size exclusion chromatography methods), chromatography (e.g., HPLC, UHPLC), centrifugation (e.g., ultracentrifugation), optionally over a sucrose gradient, affinity chromatography, immunoseparation, and any combination thereof. In some embodiments, a protocol for producing a viral particle, such as a lentiviral particle, can be used and adapted for production of the engineered delivery vesicles described herein. Exemplary techniques for viral particle production, including those carrying an exogenous cargo, are described elsewhere herein and in Brown et al., STAR Protocols 1, 100152, Dec. 18, 2020. https://doi.org/10.1016/j.xpro.2020.100152, Roldao et al., 2017. Comprehen. Biotech. 2017: 633-656, which are incorporated by reference herein and can be adapted for use with the present invention.

In certain example embodiments, the cells are cultured in suspension during incubation. In some embodiments the cells are cultured adherent to plates or other culture vessels. In some embodiments, cells cultured in suspension produce more desirable (e.g., improved characteristics, cargo loading/packaging, and/or functionalities) engineered delivery vesicles than on cells cultured on plates. In certain example embodiments, the method further comprises concentrating the isolated and/or purified engineered delivery vesicles, optionally 1-5000×. In some embodiments concentration is achieved using a centrifugation methods, such as ultracentrifugation, optionally over a sucrose cushion. In some embodiments, the engineered delivery vesicles are concentrated about 1×, 10×, 20×, 30×, 40×, 50×, 60×, 70×, 80×, 90×, 100×, 110×, 120×, 130×, 140×, 150×, 160×, 170×, 180×, 190×, 200×, 210×, 220×, 230×, 240×, 250×, 260×, 270×, 280×, 290×, 300×, 310×, 320×, 330×, 340×, 350×, 360×, 370×, 380×, 390×, 400×, 410×, 420×, 430×, 440×, 450×, 460×, 470×, 480×, 490×, 500×, 510×, 520×, 530×, 540×, 550×, 560×, 570×, 580×, 590×, 600×, 610×, 620×, 630×, 640×, 650×, 660×, 670×, 680×, 690×, 700×, 710×, 720×, 730×, 740×, 750×, 760×, 770×, 780×, 790×, 800×, 810×, 820×, 830×, 840×, 850×, 860×, 870×, 880×, 890×, 900×, 910×, 920×, 930×, 940×, 950×, 960×, 970×, 980×, 990×, 1000×, 1010×, 1020×, 1030×, 1040×, 1050×, 1060×, 1070×, 1080×, 1090×, 1100×, 1110×, 1120×, 1130×, 1140×, 1150×, 1160×, 1170×, 1180×, 1190×, 1200×, 1210×, 1220×, 1230×, 1240×, 1250×, 1260×, 1270×, 1280×, 1290×, 1300×, 1310×, 1320×, 1330×, 1340×, 1350×, 1360×, 1370×, 1380×, 1390×, 1400×, 1410×, 1420×, 1430×, 1440×, 1450×, 1460×, 1470×, 1480×, 1490×, 1500×, 1510×, 1520×, 1530×, 1540×, 1550×, 1560×, 1570×, 1580×, 1590×, 1600×, 1610×, 1620×, 1630×, 1640×, 1650×, 1660×, 1670×, 1680×, 1690×, 1700×, 1710×, 1720×, 1730×, 1740×, 1750×, 1760×, 1770×, 1780×, 1790×, 1800×, 1810×, 1820×, 1830×, 1840×, 1850×, 1860×, 1870×, 1880×, 1890×, 1900×, 1910×, 1920×, 1930×, 1940×, 1950×, 1960×, 1970×, 1980×, 1990×, 2000×, 2010×, 2020×, 2030×, 2040×, 2050×, 2060×, 2070×, 2080×, 2090×, 2100×, 2110×, 2120×, 2130×, 2140×, 2150×, 2160×, 2170×, 2180×, 2190×, 2200×, 2210×, 2220×, 2230×, 2240×, 2250×, 2260×, 2270×, 2280×, 2290×, 2300×, 2310×, 2320×, 2330×, 2340×, 2350×, 2360×, 2370×, 2380×, 2390×, 2400×, 2410×, 2420×, 2430×, 2440×, 2450×, 2460×, 2470×, 2480×, 2490×, 2500×, 2510×, 2520×, 2530×, 2540×, 2550×, 2560×, 2570×, 2580×, 2590×, 2600×, 2610×, 2620×, 2630×, 2640×, 2650×, 2660×, 2670×, 2680×, 2690×, 2700×, 2710×, 2720×, 2730×, 2740×, 2750×, 2760×, 2770×, 2780×, 2790×, 2800×, 2810×, 2820×, 2830×, 2840×, 2850×, 2860×, 2870×, 2880×, 2890×, 2900×, 2910×, 2920×, 2930×, 2940×, 2950×, 2960×, 2970×, 2980×, 2990×, 3000×, 3010×, 3020×, 3030×, 3040×, 3050×, 3060×, 3070×, 3080×, 3090×, 3100×, 3110×, 3120×, 3130×, 3140×, 3150×, 3160×, 3170×, 3180×, 3190×, 3200×, 3210×, 3220×, 3230×, 3240×, 3250×, 3260×, 3270×, 3280×, 3290×, 3300×, 3310×, 3320×, 3330×, 3340×, 3350×, 3360×, 3370×, 3380×, 3390×, 3400×, 3410×, 3420×, 3430×, 3440×, 3450×, 3460×, 3470×, 3480×, 3490×, 3500×, 3510×, 3520×, 3530×, 3540×, 3550×, 3560×, 3570×, 3580×, 3590×, 3600×, 3610×, 3620×, 3630×, 3640×, 3650×, 3660×, 3670×, 3680×, 3690×, 3700×, 3710×, 3720×, 3730×, 3740×, 3750×, 3760×, 3770×, 3780×, 3790×, 3800×, 3810×, 3820×, 3830×, 3840×, 3850×, 3860×, 3870×, 3880×, 3890×, 3900×, 3910×, 3920×, 3930×, 3940×, 3950×, 3960×, 3970×, 3980×, 3990×, 4000×, 4010×, 4020×, 4030×, 4040×, 4050×, 4060×, 4070×, 4080×, 4090×, 4100×, 4110×, 4120×, 4130×, 4140×, 4150×, 4160×, 4170×, 4180×, 4190×, 4200×, 4210×, 4220×, 4230×, 4240×, 4250×, 4260×, 4270×, 4280×, 4290×, 4300×, 4310×, 4320×, 4330×, 4340×, 4350×, 4360×, 4370×, 4380×, 4390×, 4400×, 4410×, 4420×, 4430×, 4440×, 4450×, 4460×, 4470×, 4480×, 4490×, 4500×, 4510×, 4520×, 4530×, 4540×, 4550×, 4560×, 4570×, 4580×, 4590×, 4600×, 4610×, 4620×, 4630×, 4640×, 4650×, 4660×, 4670×, 4680×, 4690×, 4700×, 4710×, 4720×, 4730×, 4740×, 4750×, 4760×, 4770×, 4780×, 4790×, 4800×, 4810×, 4820×, 4830×, 4840×, 4850×, 4860×, 4870×, 4880×, 4890×, 4900×, 4910×, 4920×, 4930×, 4940×, 4950×, 4960×, 4970×, 4980×, 4990×, to/or about 5000×.

In some embodiments, the culture conditions for producer cells during engineered delivery vesicle generation are optimized to reduce or eliminate serum inactivation of the produced engineered delivery vesicles. In some embodiments, the producer cells are cultured in a suitable serum-free media during one or more steps of production of the engineered delivery vesicles. Other parameters that may be changed is culture system (e.g., in suspension, adherent), cells used, transfection method or reagents used to deliver the engineered delivery vesicle generation system to a producer cell, isolation and/or purification method, and/or the like. Exemplary serum free media is described in e.g., Li et al., Front. Bioeng. Biotechnol., 15 Mar. 2021|https://doi.org/10.3389/fbioe.2021.646363 particularly at Table 1. Other suitable medias will be appreciated in view of the disclosure herein.

In some embodiments, the cargo molecule can be a polynucleotide or polypeptide that can alone or when delivered as part of a system, whether or not delivered with other components of the system, operate to modify the genome, epigenome, and/or transcriptome of a cell to which it is delivered. Such systems include, but are not limited to, CRISPR-Cas systems. Other gene modification systems, e.g., TALENs, Zinc Finger nucleases, Cre-Lox, morpholinos, etc. are other non-limiting examples of gene modification systems whose one or more components can be delivered by the engineered AAV particles described herein.

The present invention provides nucleic acid molecules, specifically polynucleotides which, in some embodiments, encode one or more peptides or polypeptides of interest. The term “nucleic acid,” in its broadest sense, includes any compound and/or substance that comprise a polymer of nucleotides. These polymers are often referred to as polynucleotides.

Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or hybrids or combinations thereof.

In some embodiments, the polynucleotides of the present invention may be circular. As used herein, “circular polynucleotides” means a single stranded circular polynucleotide which acts substantially like, and has the properties of, an RNA. The term “circular” is also meant to encompass any secondary or tertiary configuration of the circular polynucleotide.

In some embodiments, the polynucleotide includes from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000).

Delivery vesicles formed from the bioreactors described herein may be isolated by any suitable method known in the art. For example, vesicles may include a tag that may bind an antibody or an aptamer. Vesicles may also be isolated and sorted by fluorescence-activated cell sorting (FACS) or by use of size exclusion methods. Vesicles may be isolated by any suitable size, charge or other physical property exclusion or separation methods (chromatography, centrifugation, filtration (e.g., tangential flow filtration, dialysis, combinations thereof, and the like). Vesicles can be affinity purified, which may be enhanced or facilitated by a selectable marker or tag that, in some embodiments, is displayed on the surface of the vesicles.

In some embodiments, the producer cell line is engineered to reduce the immunogenicity of the engineered delivery particles that it produces. In some embodiments, the producer cells can be engineered to produce engineered delivery particles lacking one or more proteins, e.g., immunogenic proteins, from the surface of the engineered delivery particles. In some embodiments, the producer cells are engineered to express one or more proteases that are specific to an immunogenic protein that is produced on the surface of an engineered delivery vesicle.

In some embodiments, the producer cell line is optimized to improve production, such as improving cell viability (e.g., by overexpression of various transcription factors such as BCL2, XIAP, AVEN, MCL1) and pro-proliferative genes see e.g., S. Fischer, R. Handrick, K. Otte. The art of CHO cell engineering: a comprehensive retrospect and future perspectives. Biotechnol Adv, 33 (8) (2015), pp. 1878-1896, 10.1016/j.biotechadv.2015.10.015). In some embodiments, the producer line is modified to include miRNA overexpression (e.g., miR-2861, miR-23, miR-17) or knock-down by antagomiRs, or via viral vectors, sponge and tough decoy vectors (e.g. miR-7, miR-106b, miR-14 and many others) to adapt cells to stressful environment, temperature changes and to enhance protein production (S. Fischer, A. J. Paul, A. Wagner, S. Mathias, M. Geiss, F. Schandock, et al. miR-2861 as novel HDAC5 inhibitor in CHO cells enhances productivity while maintaining product quality. Biotechnol Bioeng, 112 (10) (2015), pp. 2142-2153, 10.1002/bit.25626; V. Jadhav, M. Hackl, G. Klanert, J. A. Hernandez Bort, R. Kunert, J. Grillari, et al. Stable overexpression of miR-17 enhances recombinant protein production of CHO cells. J Biotechnol, 175 (2014), pp. 38-44; Kelly et al., Biotechnol J, 10 (7) (2015), pp. 1029-1040; Coleman et al., J Proteomics, 195 (2019), pp. 23-32; and Xu et al., Appl Microbiol Biotechnol, 103 (17) (2019), pp. 7085-709. In some embodiments, the producer cell is modified to have reduced or eliminated expression of metabolic (e.g., LDHA), pro-apoptotic (BAX, BAK) and anti-proliferative genes, and cell cycle checkpoint kinases (ATR). Tihanyi and Nyitray Drug. Disc. today: TEhc. Volume 38, December 2020, Pages 25-34 describe additional modifications to producer lines which can be adapted for use with the producer cells described herein. Other suitable exemplary cargos are described elsewhere herein.

### Methods for Delivery of Cargo Using Delivery Vesicles

Also envisioned within the scope of the invention is a method for delivering cargo to one or more cells using the delivery vesicles described herein. As described, the engineered delivery vesicle may deliver the cargo to one or more cells of a subject.

In certain example embodiments, the fusogenic polypeptide may provide trophism for a specific cell. In other example embodiments, the delivery vesicles described herein may comprise one or more targeting moieties that are capable of specifically binding to a target cell. Such targeting moieties may include, but are not necessarily limited to membrane fusion proteins, antibodies, peptides, cyclic peptides, small molecules or related molecular structure capable of being directed through its binding to a target, including non-immunoglobulin scaffolds, including fibronectin, lipocalin, protein A, ankyrin, thioredoxin, and the like. In some embodiments, a membrane fusion protein may include, but is not necessarily limited to, the G envelope protein of vesicular stomatitis virus (VSV-G), herpes simplex virus 1 gB (HSV-1 gB), ebolavirus glycoprotein, members of the SNARE family of proteins, and members of the syncytin family of proteins.

In some embodiments, the cargo may comprise a therapeutic agent. The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

Target cells may include, but are not necessarily limited to, mammalian cells, cancer cells, cells that are infected with a pathogen, such as a virus, bacterium, fungus, or parasite. In some embodiments, the invention comprises delivery of cargo across the blood brain barrier. As one of skill in the art may appreciate, vesicles can be engineered to have tropism to any particular desired cell type.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Ird, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

It will be appreciated that administration of therapeutic entities in accordance with the invention may be in the presence of suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, PA (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman WN “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “in need of treatment”, or “in need thereof” as used herein refers to a judgment made by a caregiver (e.g., physician, nurse, nurse practitioner, or individual in the case of humans; veterinarian in the case of animals, including non-human animals) that a subject requires or will benefit from treatment. This judgment is made based on a variety of factors that are in the realm of a caregiver's experience, but that include the knowledge that the subject is ill, or will be ill, as the result of a condition that is treatable by the compounds of the invention.

As used in this context, to “treat” means to cure, ameliorate, stabilize, prevent, or reduce the severity of at least one symptom or a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

The administration of compositions, agents, cells, or populations of cells, as disclosed herein may be carried out in any convenient manner including by aerosol inhalation, injection, ingestion, transfusion, implantation or transplantation. The composition may be administered to a patient subcutaneously, intradermally, intratumorally, intranodally, intramedullary, intramuscularly, intrathecally, by intravenous or intralymphatic injection, or intraperitoneally.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

Methods of administering the pharmacological compositions, including agonists, antagonists, antibodies or fragments thereof, to an individual include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

The amount of the agents which will be effective in the treatment of a particular disorder or condition will depend on the nature of the disorder or condition, and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. In general, the daily dose range lies within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases. In certain embodiments, suitable dosage ranges for intravenous administration of the agent are generally about 5-500 micrograms (μg) of active compound per kilogram (Kg) body weight. Suitable dosage ranges for intranasal administration are generally about 0.01 pg/kg body weight to 1 mg/kg body weight. In certain embodiments, a composition containing an agent of the present invention is subcutaneously injected in adult patients with dose ranges of approximately 5 to 5000 μg/human and preferably approximately 5 to 500 μg/human as a single dose. It is desirable to administer this dosage 1 to 3 times daily. Effective doses may be extrapolated from dose-response curves derived from in vitro or animal model test systems. Suppositories generally contain active ingredient in the range of 0.5% to 10% by weight; oral formulations preferably contain 10% to 95% active ingredient. Ultimately the attending physician will decide on the appropriate duration of therapy using compositions of the present invention. Dosage will also vary according to the age, weight and response of the individual patient.

Preferably, the therapeutic agent may be administered in a therapeutically effective amount of the active components. The term “therapeutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of a disease or condition being treated.

In some embodiments, the therapeutic agent may comprise one or more components of a gene editing system and/or polynucleotide encoding thereof.

### Methods of Use

### General Discussion

The engineered delivery system polynucleotides, molecule(s), vector(s), engineered cells, engineered delivery vesicles can be used generally to package and/or deliver one or more cargo molecules to a recipient cell. In some embodiments, engineered delivery system polynucleotides and/or engineered delivery vesicles produced therefrom can be administered to a subject or cell and directly mediate the transfer of cargo to from the engineered delivery vesicle to a recipient cell (such as a target cell). In other embodiments, engineered cells capable of producing engineered delivery vesicles can be generated from engineered delivery system polynucleotides and/or vector(s). In some embodiments, the engineered delivery system polynucleotides, vector(s), engineered delivery system vesicles, and/or formulations thereof can be delivered to a subject (such as a cell, tissue, organ, or whole organism). When delivered to a subject, they engineered delivery system polynucleotide(s) and/or vector(s) can transform one or more of a subject's cells to produce an engineered cell that can be capable of making an engineered delivery vesicles (i.e., become a producer cell), which can be released from the engineered cell and deliver cargo molecule(s) to a recipient cell that is in immediate or distant proximity to the producer cell. Delivery can be ex vivo, in vitro, or in vivo. Thus, production of engineered delivery vesicles can be ex vivo, in vitro, or in vivo. Engineered delivery vesicle producing cells can be used in a cell therapy, such as an autologous or allogenic cell therapy, by administering such cells to a subject in need thereof. In some embodiments, an engineered cell can be delivered to a subject (e.g., a human or non-human animal or plant), where it can release produced engineered delivery vesicles such that they can then deliver a cargo molecule(s) to a recipient cell. These general processes can be used in a variety of ways to treat and/or prevent disease or a symptom thereof in a subject, generate model cells, generate modified organisms, provide cell selection and screening assays, in bioproduction, and in other various applications.

The engineered delivery systems and vesicles produced therefrom described herein can also be used in various culture systems such as co-cultures for a variety of experimental, therapeutic, and/or industrial applications.

### Co-Culture Systems

Described in several exemplary embodiments herein are co-culture systems comprising two or more cell types, where at least one, all, or a sub-combination of cell types comprise an engineered delivery system as described in greater detail elsewhere herein, wherein the engineered delivery system is capable of generating one or more delivery vesicles. In general, a co-culture as the term is used herein, is a cell culture system in which two or more different populations of cells are grown with some degree of contact between the two or more different populations. Cell populations within the co-culture can differ in cell type, state, origin, lineage, passage, species of origin, and the like.

In some embodiments, the engineered delivery system in a given cell population within the co-culture includes a cargo and thus can produce a delivery vesicle comprising a cargo. The delivery vesicle can be released by the cell which produced it into the co-culture where it can then deliver its cargo to another cell, such as a cell of another cell population within the co-culture. This can drive, for example, the development of synthetic interactions between cells of the co-culture, formation of synthetic ecologies, or other complex interactions within the co-culture.

The co-cultures can be used for studying and/or engineering complex multicellular populations and synthetic systems. In some embodiments, the co-cultures described herein can be configured and used for culturing one or more cell populations, such as traditionally difficult to culture cell populations. In some embodiments, the co-cultures described herein can be configured and used for establishing synthetic interactions between populations. In some embodiments, the co-cultures described herein can be configured and used for studying natural interactions such as infections and creating model systems and biomimetic environments of natural systems, such as artificial tissues or organs. Such systems can be used in screening assays to study complex reactions to agents of interest, such as therapeutic agents, pathogens, and/or toxins. Additional applications for the co-cultures containing at least one cell population containing an engineered delivery system and capable of generating engineered delivery vesicles therefrom are described in e.g., Goers et al., 2014. J R. Soc. Interface 11:20140065; http://dx.doi.org/10.1098/rsif.20140065.

### Methods of Treatment

The engineered delivery system polynucleotides and vector(s), engineered cells, engineered delivery vesicles described herein, formulations thereof, or a combination thereof can be delivered to a subject (e.g., a cell, tissue, organ, or organism) as a treatment or prevention of a disease, condition or disorder. Delivery can be in vitro, in vivo, or ex vivo and be by any suitable administration method or technique. In some embodiments, the cargo(s) to be delivered by the engineered delivery vesicles herein are therapeutic and can treat and/or prevent a disease or disorder once delivered by the engineered delivery vesicles. In other embodiments, the producer cells can be delivered as an adoptive cell therapy to facilitate cargo delivery and subsequent treatment or prevention mediated by the cargo(s). In some embodiments, a cell to which the delivery vesicles deliver a cargo to are infected with a pathogen. In some embodiments, the pathogen may be a virus or bacterial pathogen.

### Adoptive Cell Therapies

Generally speaking, adoptive cell transfer involves the transfer of cells (autologous, allogeneic, and/or xenogeneic) to a subject. The cells may or may not be modified and/or otherwise manipulated prior to delivery to the subject.

In some embodiments, an engineered cell, such as one containing an engineered delivery vesicle generation system and/or engineered delivery vesicles described herein, as described herein can be included in an adoptive cell transfer therapy. In some embodiments, an engineered cell as described herein can be delivered to a subject in need thereof. In some embodiments, the cell can be isolated from a subject, manipulated in vitro such that it is capable of generating an engineered delivery vesicles described herein to produce an engineered cell and delivered back to the subject in an autologous manner or to a different subject in an allogeneic or xenogeneic manner. The cell isolated, manipulated, and/or delivered can be a eukaryotic cell. The cell isolated, manipulated, and/or delivered can be a stem cell. The cell isolated, manipulated, and/or delivered can be a differentiated cell. The cell isolated, manipulated, and/or delivered can be an immune cell, a blood cell, an endocrine cell, a renal cell, an exocrine cell, a nervous system cell, a vascular cell, a muscle cell, a urinary system cell, a bone cell, a soft tissue cell, a cardiac cell, a neuron, or an integumentary system cell. Other specific cell types will instantly be appreciated by one of ordinary skill in the art.

In some embodiments, the isolated cell can be manipulated such that it becomes an engineered cell as described elsewhere herein (e.g., contain and/or express one or more engineered delivery system molecules or vectors described elsewhere herein). Methods of making such engineered cells are described in greater detail elsewhere herein. In some embodiments, the engineered cell can be engineered to be capable of packaging molecules endogenous to the isolated cell into the engineered delivery vesicles. Once delivered to a subject, the engineered cell can produce engineered delivery vesicles whose cargo is one or more molecules endogenous to the isolated (now engineered cell). The engineered delivery vesicles can be released from the engineered cell and circulate within the subject and deliver the molecule(s) endogenous to the isolated cell to another cell (the recipient cell) within the subject. In some embodiments, the recipient cell is the same type of cell as the isolated cell. In some embodiments, the recipient cell is a different type of cell than the donor cell. In some embodiments, the engineered cell can be engineered to be capable of packaging molecules exogenous to the isolated cell into engineered delivery vesicles. Once delivered to a subject, the engineered cell can produce engineered delivery vesicles whose cargo is one or more molecules exogenous to the isolated (now engineered cell). The engineered delivery vesicles can be released from the engineered cell and circulate within the subject and deliver the molecule(s) exogenous to the isolated cell to another cell (the recipient cell) within the subject. In some embodiments, the recipient cell is the same type of cell as the isolated cell. In some embodiments, the recipient cell is a different type of cell than the donor cell.

The administration of the cells or population of cells according to the present invention may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can be or involve the administration of 10⁴-10⁹ cells per kg body weight including all integer values of cell numbers within those ranges. In some embodiments, 10⁵ to 10⁶ cells/kg are delivered Dosing in adoptive cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The adminition can be an intravenous administration. The administration can be directly done by injection within a tissue. In some embodiments, the tissue can be a tumor.

To guard against possible adverse reactions, engineered cells can be equipped with a transgenic safety switch, in the form of a transgene that renders the cells vulnerable to exposure to a specific signal. For example, the herpes simplex viral thymidine kinase (TK) gene may be used in this way, for example by introduction into the engineered cell similar to that discussed in Greco, et al., Improving the safety of cell therapy with the TK-suicide gene. Front. Pharmacol. 2015; 6: 95. In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

Methods of modifying isolated cells to obtain the engineered cells with the desired properties are described elsewhere herein. In some embodiments, the methods can include genome editing using a CRISPR-Cas system to modify the cell. This can be in addition to introduction of an engineered delivery system molecule describe herein.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1;112(12):4746-54). Thus, to prevent rejection of allogeneic cells, the host's immune system usually has to be suppressed to some extent. However, in the case 186tilizptive cell transfer the use of immunosuppressive drugs also have a detrimental effect on the introduced therapeutic cells, such as engineered cells described herien. Therefore, to effectively use an adoptive immunotherapy approach in these conditions, the introduced cells would need to be resistant to the immunosuppressive treatment. Thus, in a particular embodiment, the present invention further comprises a step of modifying the engineered cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to engineered cells for adoptive cell therapy by inactivating the target of the immunosuppressive agent in engineered cells. As non-limiting examples, targets for an immunosuppressive agent can be a receptor for an immunosuppressive agent such as: CD52, glucocorticoid receptor (GR), a FKBP family gene member and a cyclophilin family gene member.

Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT1 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1 or TIM-3. In some embodiments, the gene locus involved in the expression of PD-1 or CTLA-4 genes is targeted. In some embodiments, combinations of genes are targeted, such as but not limited to PD-1 and TIGIT.

In some embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ.

Whether prior to or after genetic or other modification of the engineered cells (such as engineered T cells (e.g., the isolated cell is a T cell), the engineered cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. The engineered cells can be expanded in vitro or in vivo.

### EXAMPLES

### Example 1—Pseudotyping Lentiviruses with Endogenous Retroviral Envelope Proteins

Expression of various individual env proteins was tested in HEK293T cells (FIG. 1). Best expression was achieved with Envw1, Envk1, and Envfrd (Envw2). The glycoprotein of vesicular stomatitis virus (VSV-G) is able to mediate cell attachment and induce direct fusion between membranes. Applicants compared pseudotyping efficiencies of different env proteins with lentivirus DNA. Efficient particle formation was observed with Envk1, Envw1, and Envfrd (FIG. 2).

To see if the gag homology protein Pnma3 is expressed in neuronal cells, Applicants fused it to a red fluorescent reporter protein (RFP) and tested its expression in mouse and rat neurons. Results showed that expression of this fusion protein was comparable to a control RFP-lentivirus construct (FIG. 3).

### Example 2—Screening of Endogenous Gag Protein Candidates for their Ability to Form Capsids, Secrete Proteins, and Transfer Information

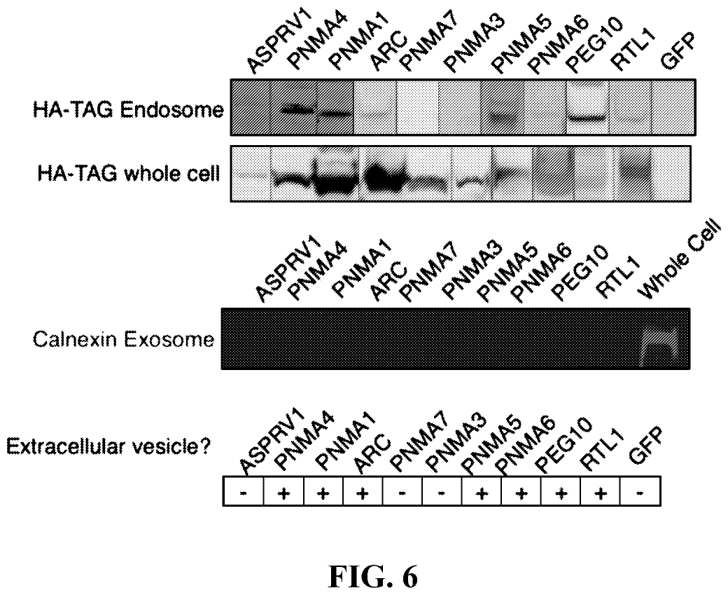

Nine endogenous gag protein candidates were identified and screened for their ability to form vesicles in vitro (FIGS. 4-5). Of the candidates tested, all except Asprv1 were able to form vesicles (FIG. 5 and Table 4). However, only six were able to be secreted from cells (Table 5, FIG. 6).

TABLE 4
Ability of gag protein candidates to form vesicles

| | Capsid forming in vitro? |
|---|---|
| Asprv1 | − |
| Pnma1 | + |
| Pnma3 | + |
| Pnma4 | + |
| Pnma5 | + |
| Pnma6 | + |
| Pnma7 | + |
| Peg10 | + |
| Rtl1 | + |

TABLE 5
Ability of gag protein candidates to be secreted from cells

| | Secreted Proteins? |
|---|---|
| Asprv1 | − |
| Pnma1 | + |
| Pnma3 | − |
| Pnma4 | + |
| Pnma5 | + |
| Pnma6 | + |
| Pnma7 | − |
| Peg10 | + |
| Rtl1 | + |

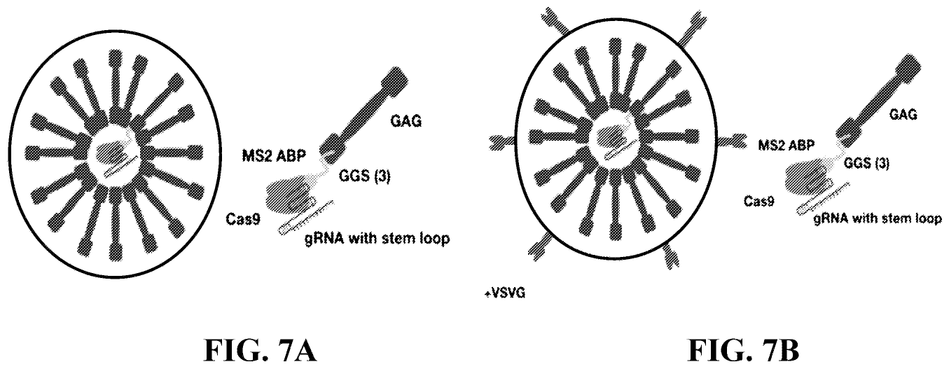

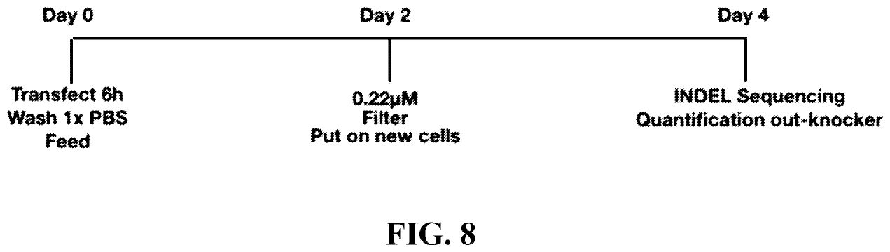

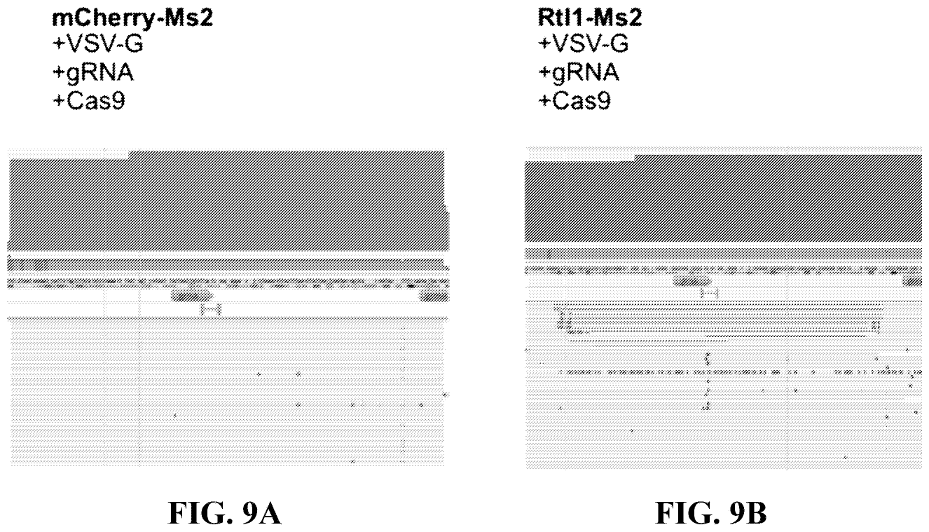

Applicants next tested the ability of the various gag protein candidates to transfer Cas9/gRNA complexes to another cell. In the absence of a membrane fusion protein (FIG. 7A), none of the candidates were able to successfully facilitate this process. However, the inclusion of VSV-G (FIG. 7B) was critical for achieving delivery of the complex to another cell (Table 6).

TABLE 6
Ability of gag protein candidates to deliver information to a new cell

| | Transfer? |
|---|---|
| Asprv1 | − |
| Pnma1 | − |
| Pnma3 | − |
| Pnma4 | + |
| Pnma5 | − |
| Pnma6 | − |
| Pnma7 | − |
| Peg10 | + |
| Rtl1 | + |

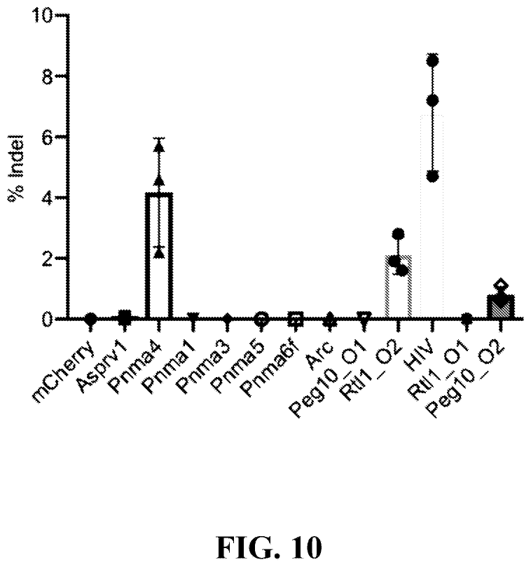

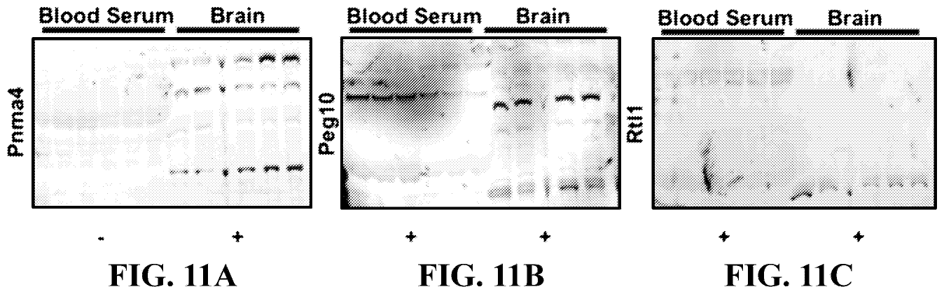

Vesicles formed using PNMA4 and RTL1 showed the highest ability to transfer gene editing complexes to new cells and induce formation of indels (FIG. 10).

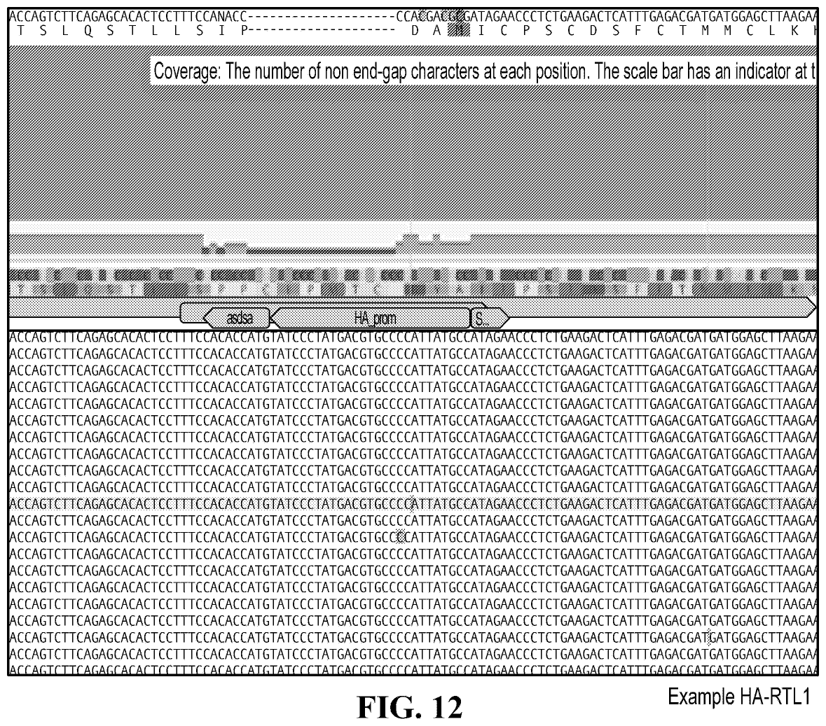

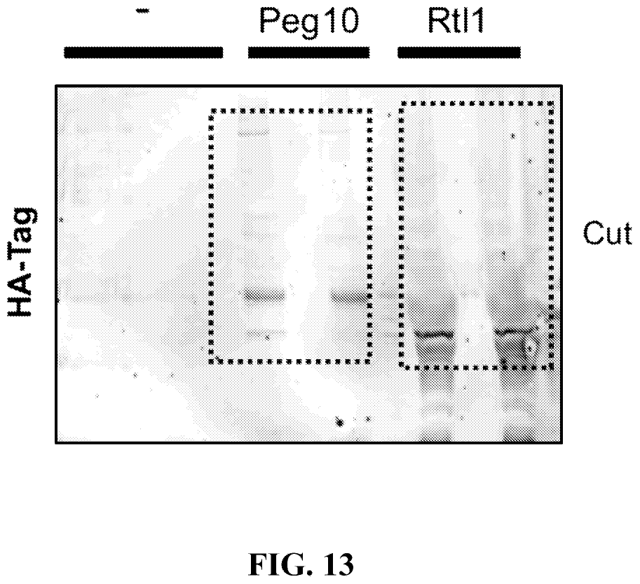

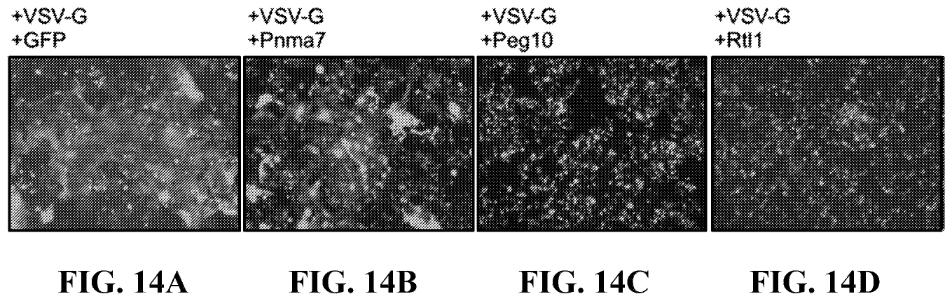

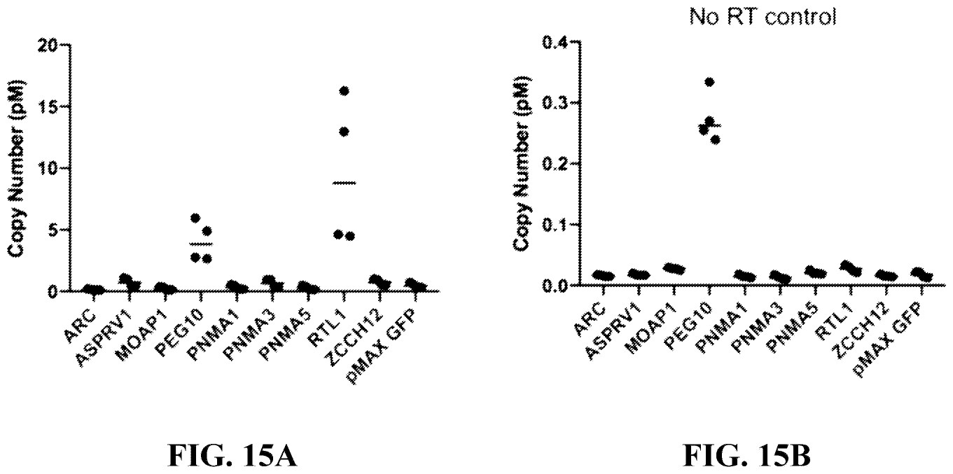

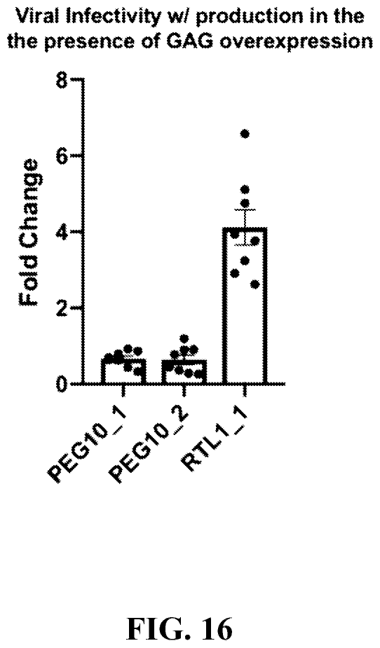

To evaluate whether gag candidates facilitated secretion from cells and subsequent transfer of information from one cell to another, Applicants also generated knock-in mice that expressed an HA-tag on endogenous gag proteins. DNA sequences encoding an exemplary HA-tagged RTL1 protein are shown in FIG. 12.

### Example 3—Engineering an Endogenous Vector for Gene Therapy

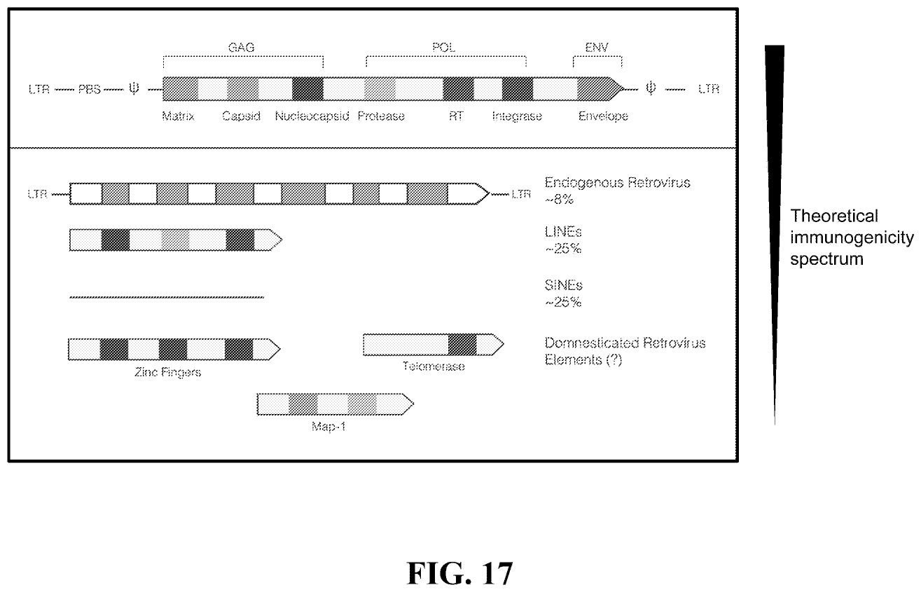

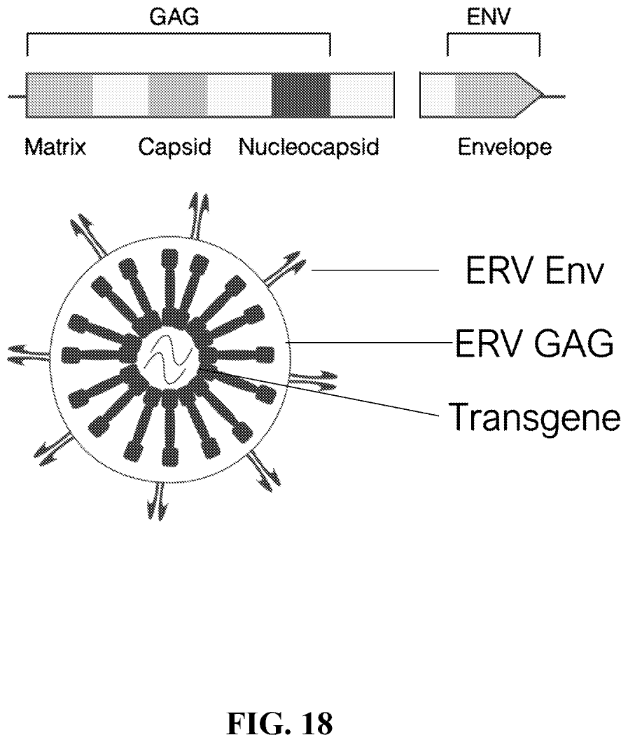

Applicants set out to create a non-immunogenic vector that can efficiently deliver gene therapies in vivo. While viral vectors are highly efficient, they can potentially be immunogenic, eliciting unwanted immune responses in target cells against the vector itself, thus rendering the therapeutic agent contained therein ineffective. Lipid nanoparticles (LNPs) are easy to produce but they have limited tropism and are typically only able to deliver about 2% of their encoded payload. Exosomes are potentially non-immunogenic but have a complicated biology and their efficacy is unclear. Applicants wanted to explore endogenous signaling systems for their potential to mediate intercellular gene transfer. For example, there are at least 40,000 coding GAGs in the human genome, with varying immunogenic potential (FIG. 17). Some highly expressed endogenous GAGs are shown in FIG. 4.

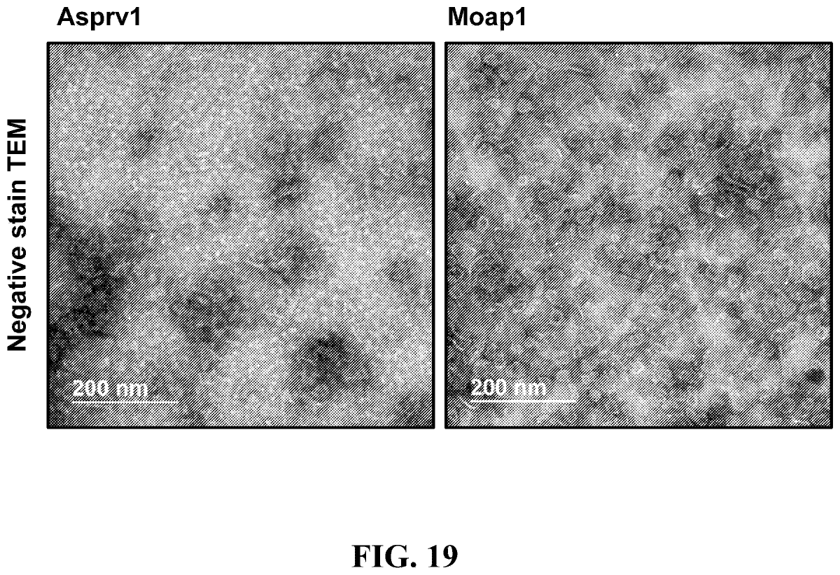

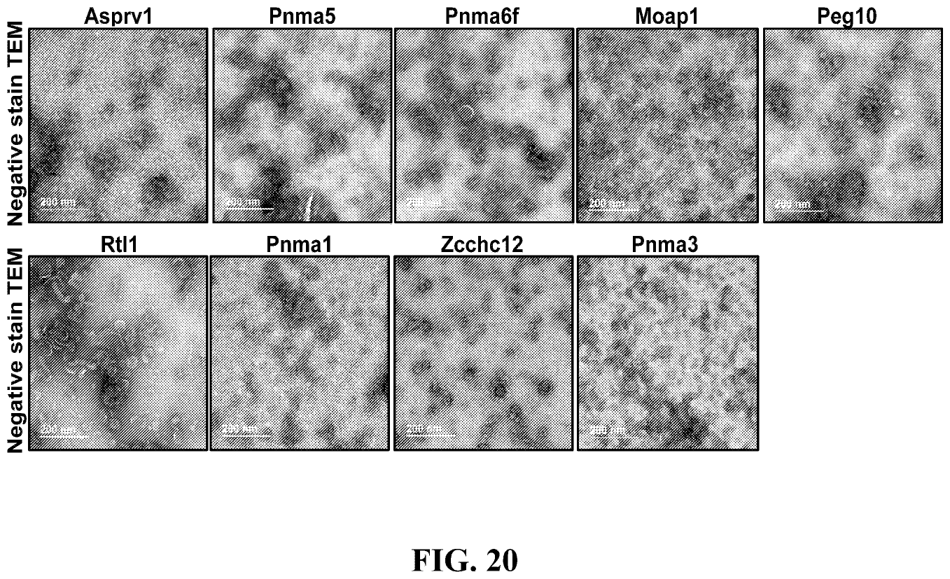

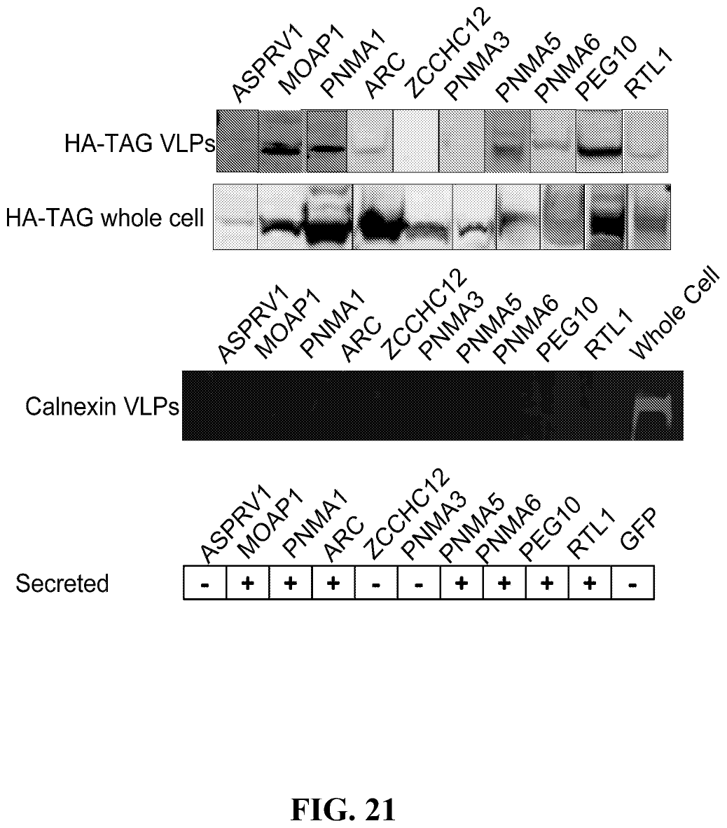

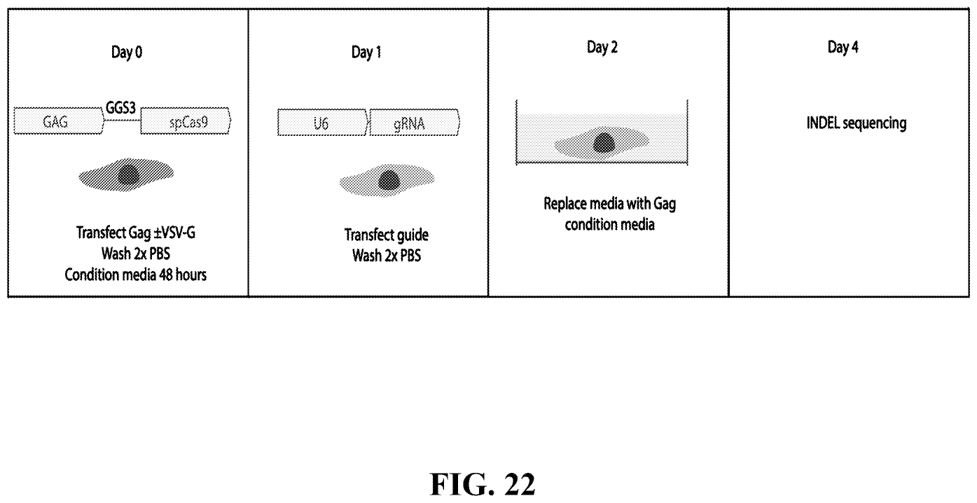

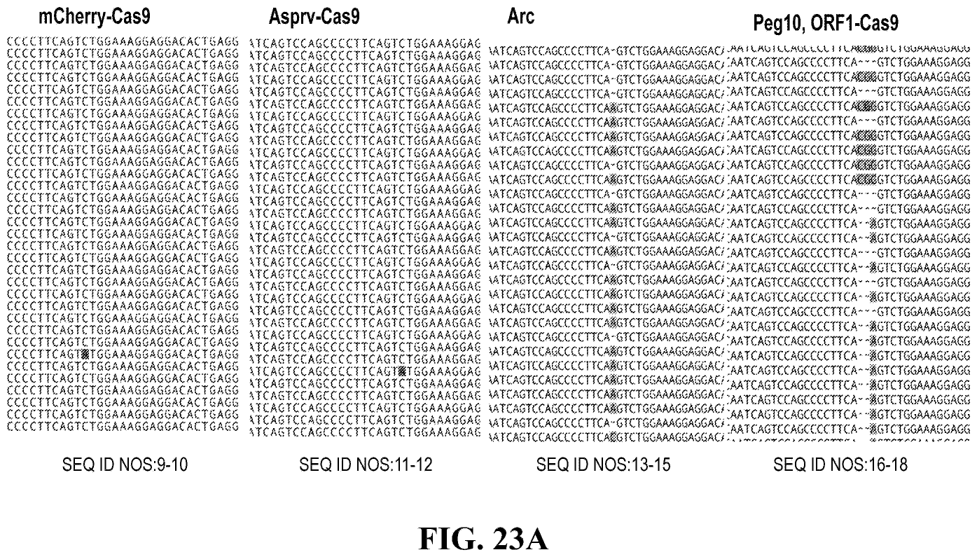

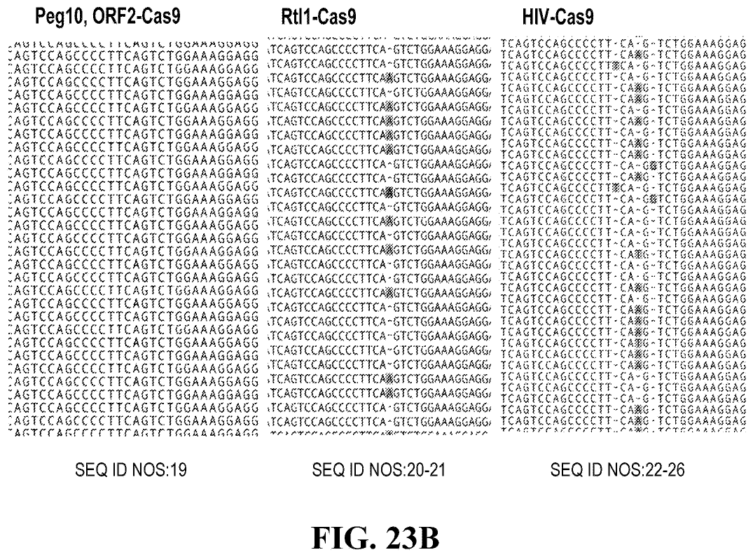

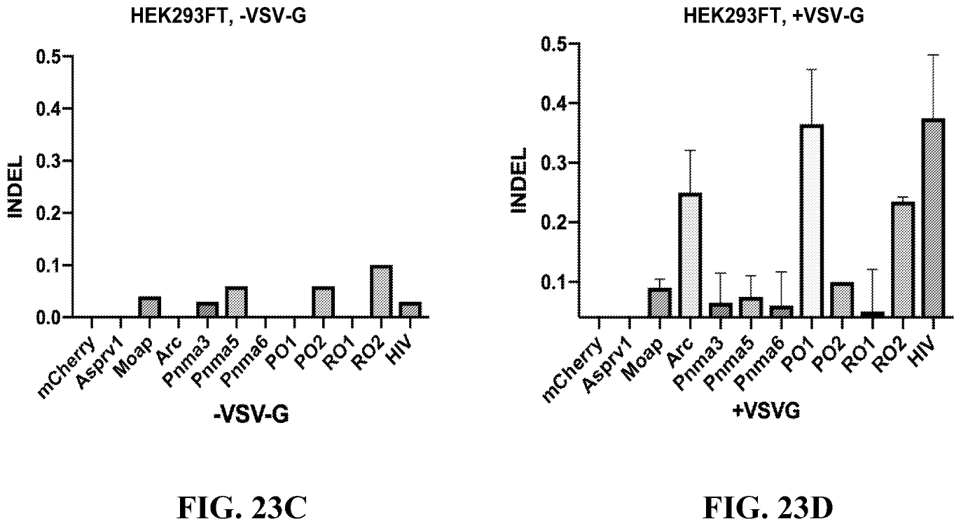

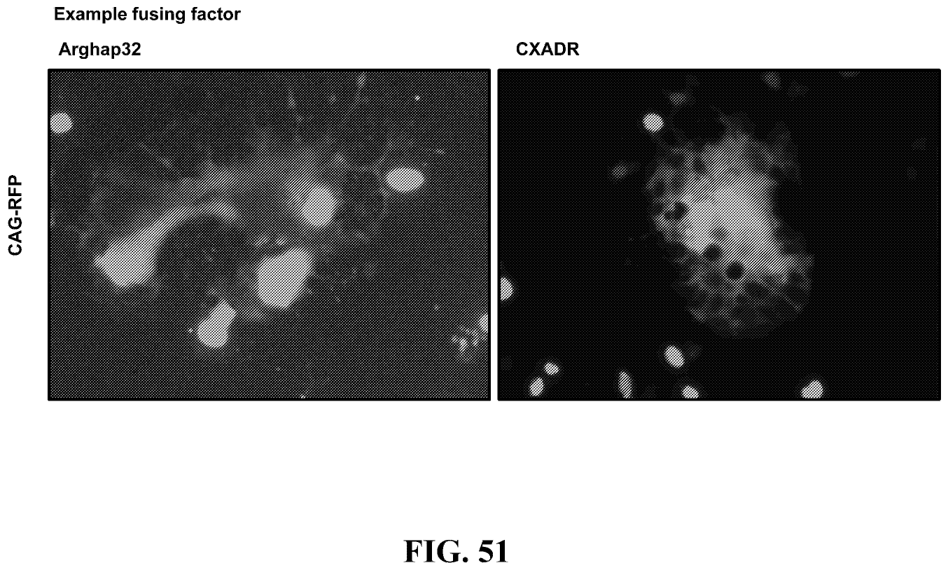

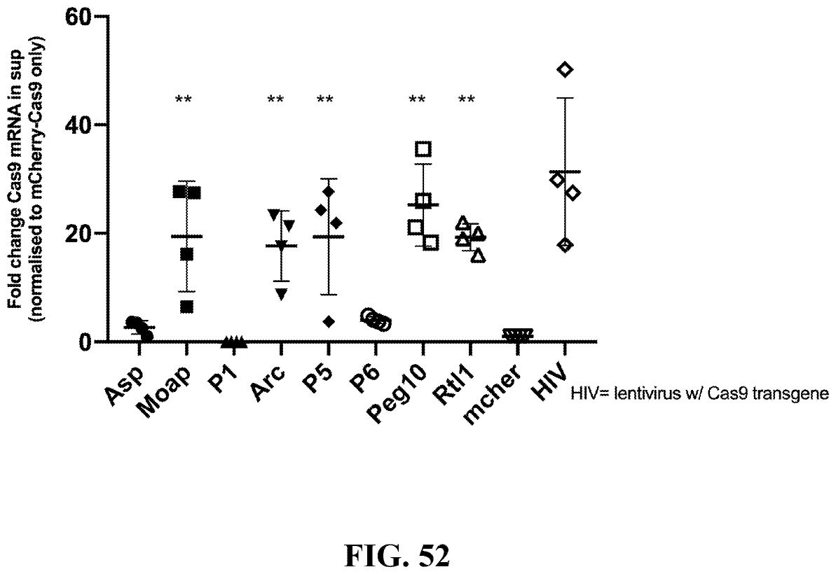

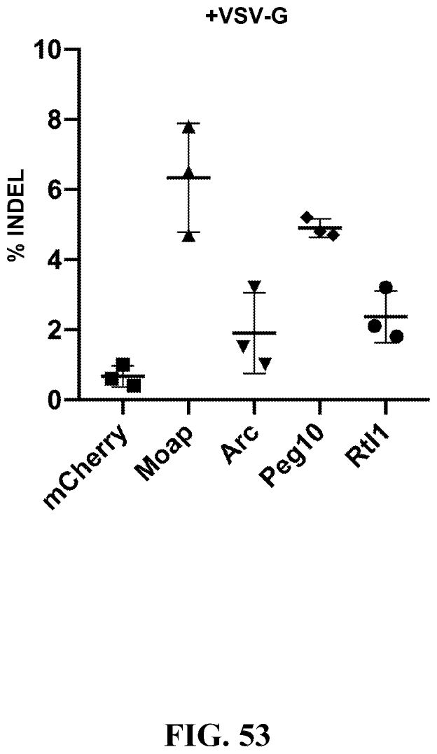

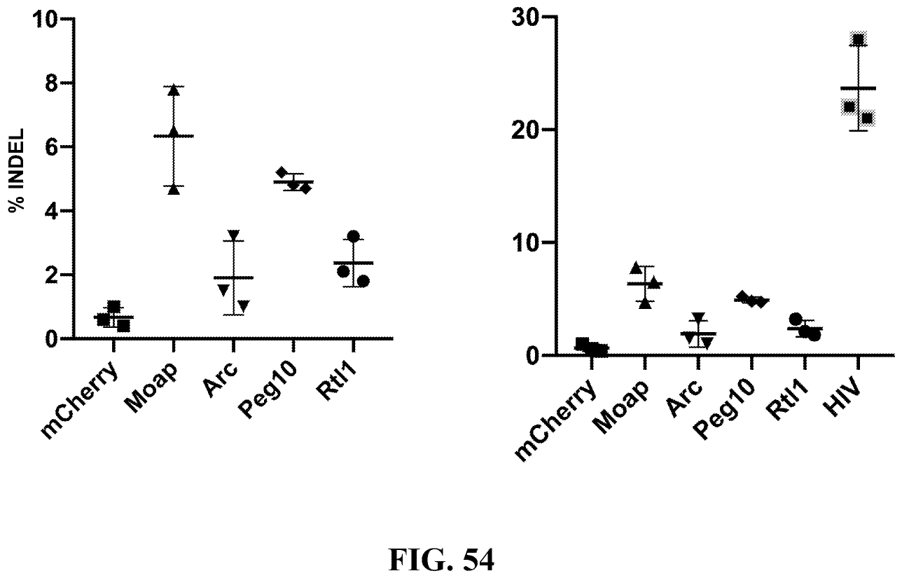

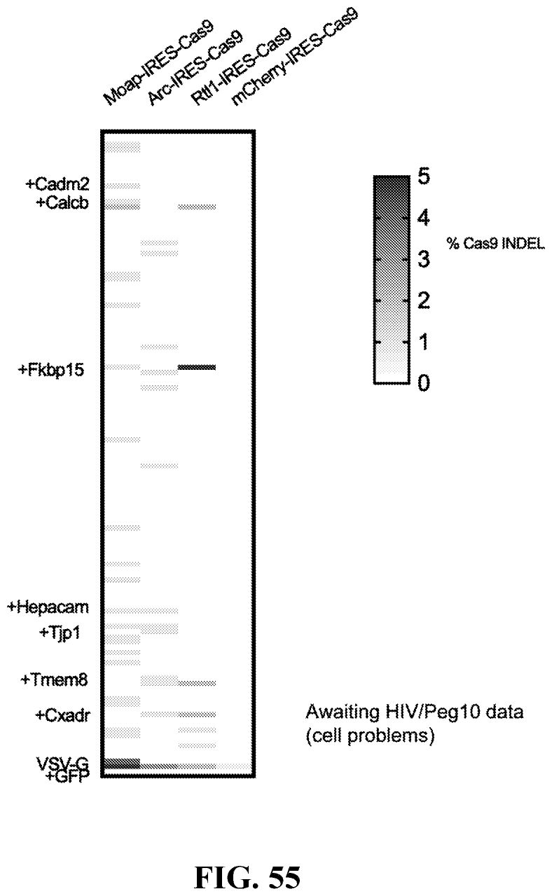

Applicants analyzed several GAGs for their ability to spontaneously form vesicles (FIGS. 19, 20). To determine which GAGs can form vesicles, HA-tagged GAGs were over-expressed in HEK cells and the supernatant was collected. The VLP fraction was centrifuged using PEG (FIG. 21). Applicants found that addition of VSV-G fusogen both improved uptake of secreted GAGs by target cells and boosted generation of INDELs (FIGS. 23A-23D, 24, 52, and 53).

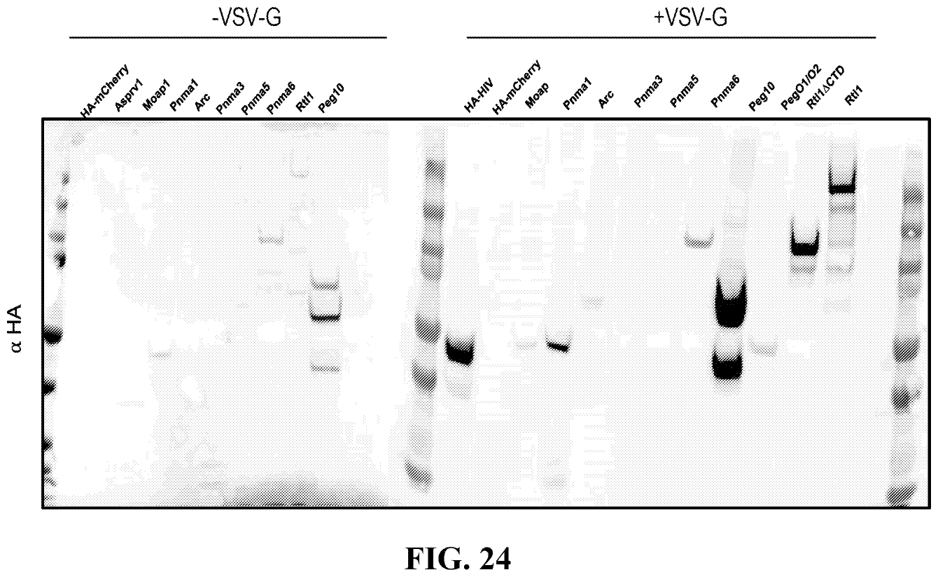

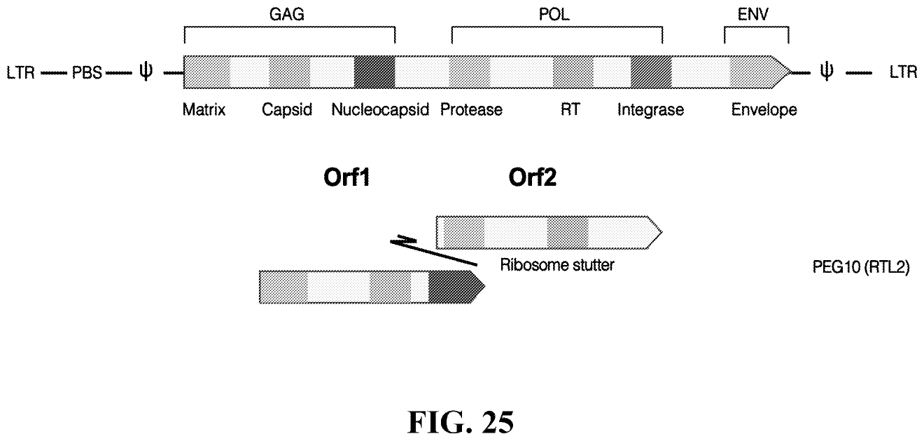

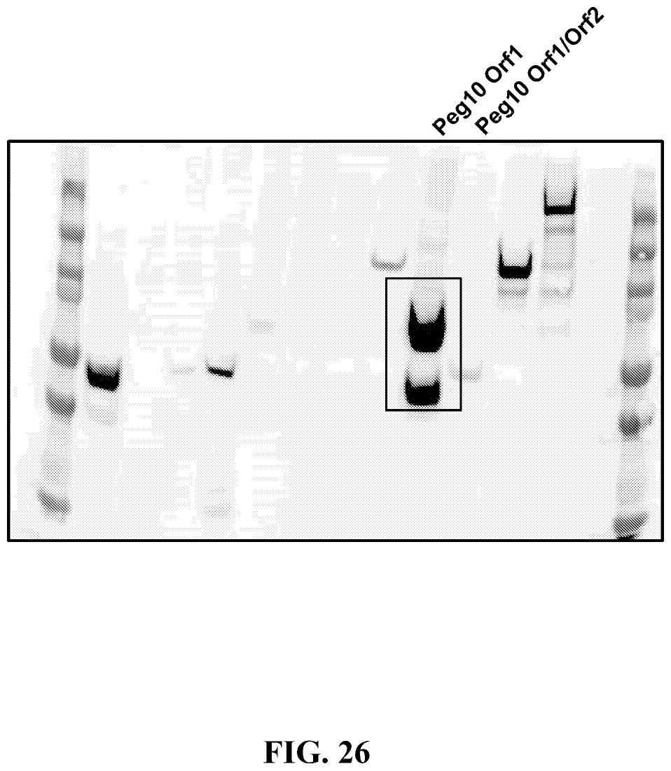

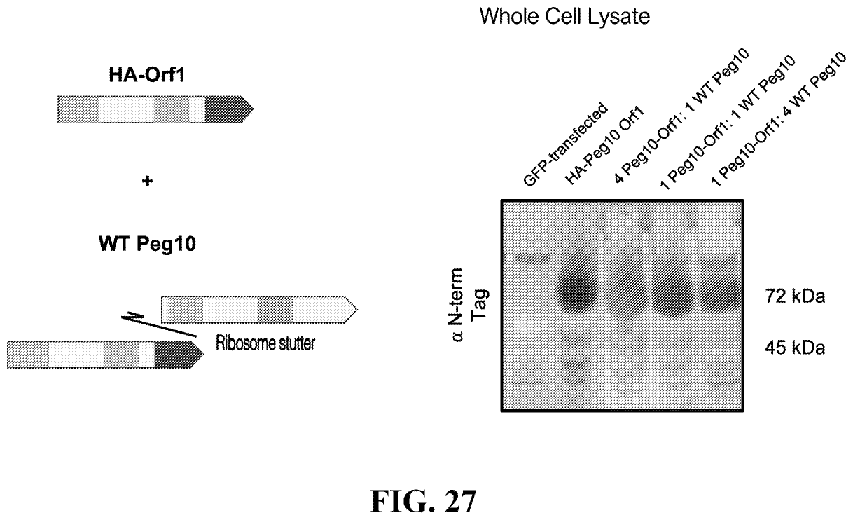

Out of all the GAGs tested, Applicants determined that PEG10 was the best candidate to mediate transfer and generate VLPs on par with HIV lentiviruses (FIG. 24). To optimize PEG10 for delivery, Applicants wanted to understand the precise biological function of PEG10, as well as the extent to which PEG10 can be reprogrammed. The gene for PEG10 includes two overlapping reading frames of the same transcript encoding distinct isoforms. The shorter isoform has a CCHC-type zinc finger motif containing a sequence characteristic of gag proteins of most retroviruses and some retrotransposons, and it functions in part by interacting with members of the TGF-beta receptor family. The longer isoform has the active-site DSG consensus sequence of the protease domain of pol proteins. The longer isoform is the result of −1 translational frameshifting that is also seen in some retroviruses (FIGS. 25, 26).

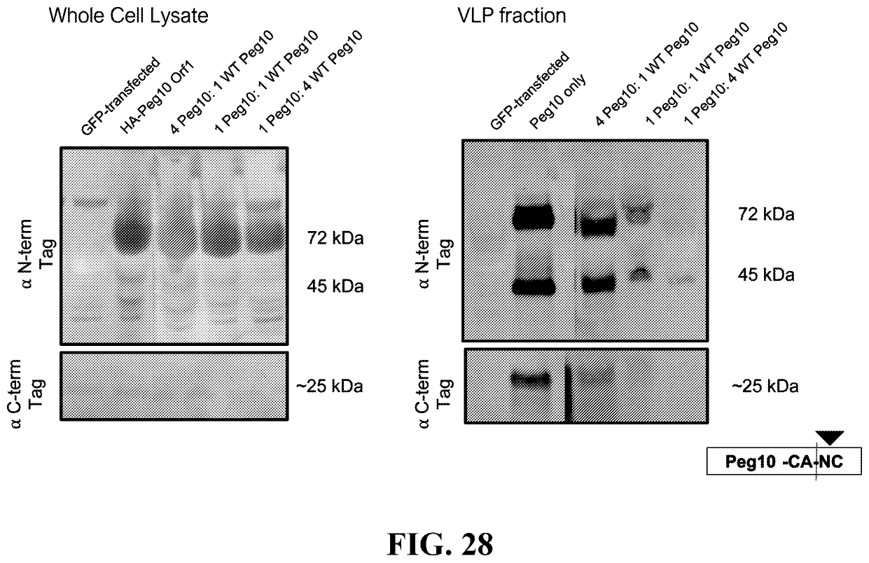

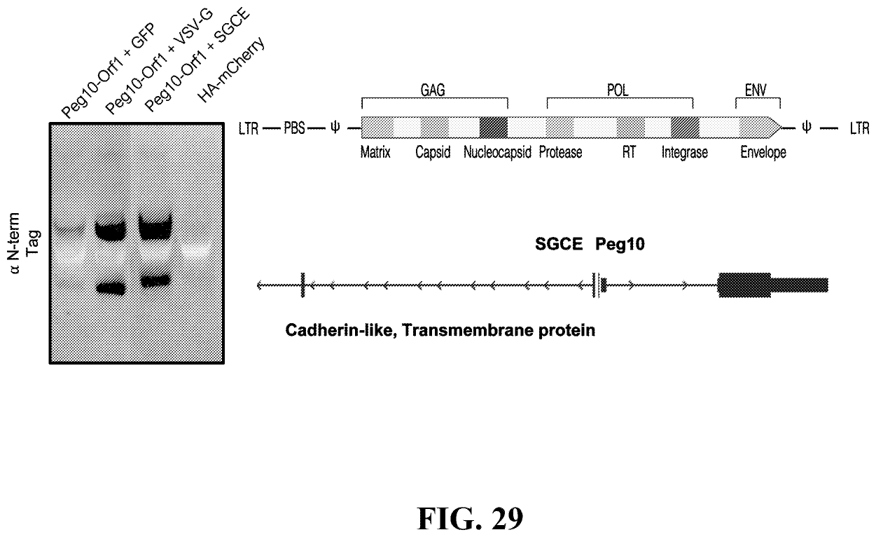

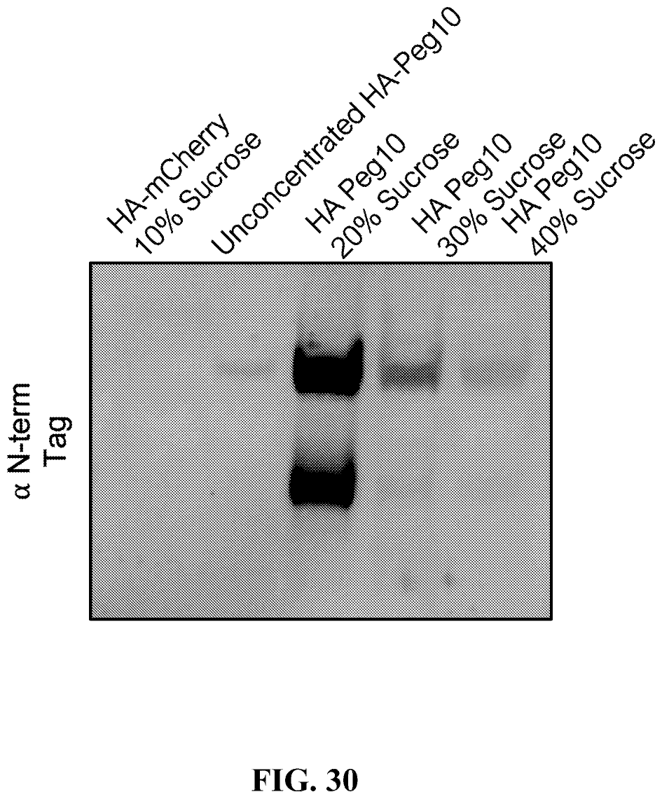

Applicants transfected cells with various PEG10 constructs and analyzed whole cell lysates and VLP fractions by immunoprecipitation. Results showed that PEG10 VLPs are processed but the protease domain is not required for this processing to occur (FIG. 28). Applicants also found that addition of VSV-G boosts PEG10 secretion and enables uptake in target cells (FIG. 29).

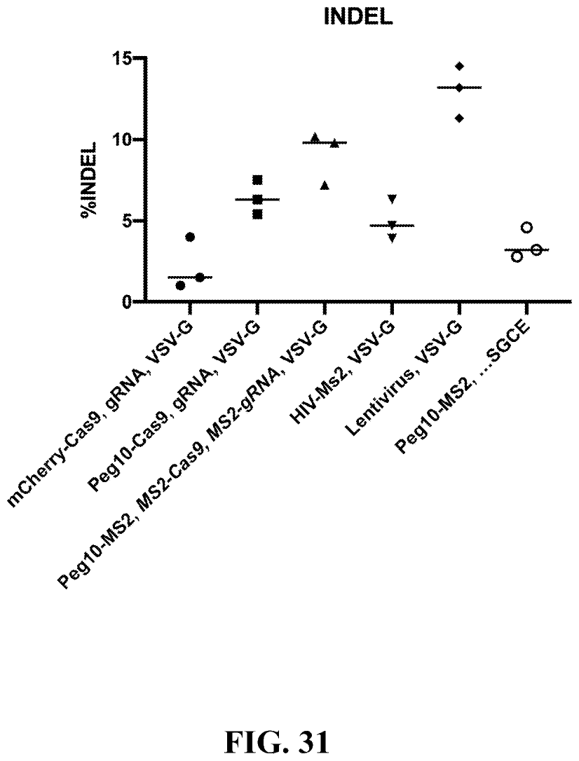

To boost efficiency of delivery, Applicants cultured HEK293T cells in T225 flasks. Cells were transfected with various delivery components, filtered with a 45 μm filter, and ultracentrifuged with a 20% sucrose cushion. VLPs were resuspended in 250 μL of PBS and 10 μL aliquots of the suspension were added to 20E3 cells. INDELs were then detected 48 hours later by Next Generation Sequencing (FIG. 31). These experiments revealed that PEG10 is a secreted, capsid-forming protein, and that VSV-G enables PEG10 to deliver Cas9 to target cells and mediate generation of INDELs. PEG10 VLPs are likely processed at the C-terminal domain. Applicants also found that addition of SGCE boosts PEG10 secretion but does not help boost entry (at least in HEK cells).

Applicants compiled and cloned a list of an additional 165 genes that could act as potential fusogens (Tables 7 and 8). Each of these will be evaluated individually with HIV, PEG10, Arc, and Rtl1 GAGs.

TABLE 7

| | | | | |
|---|---|---|---|---|
| Adam 10 | IZUMO2 | FRMD5 | CDH3 | SPECC1 |
| ADAMDEC1 | IZUMO4 | FRMD6 | CDHR3 | Spock |
| ADGRF3 | Klrb1c | FZD6 | CLDND1 | SPOCK2 |
| AGRN | LDLRAD4 | GALNT14 | COL7A1 | ST3GAL4 |
| AGTR2 | LIMS2 | GAP43 | COPB1 | TAL1 |
| AHCY | LRP6 | GDAP1 | CXDAR | TBC1D16 |
| ALDH3B1 | LRPAP1 | GHITM | DNAJC8 | THSD4 |
| AOC3 | MAMDC2 | GHR | DOC2A | TJP2 |
| APBB1 | NCKAP1L | GNAO1 | DUSP10 | TM9SF2 |
| Arhgap32 | Nectin1 | GPM6A | DUSP15 | TMEM140 |
| ARHGAP45 | NEDD4L | GPR161 | EDA2R | Tmem68 |
| ARMCX5 | Negr1 | GRIA4 | EDNRB | Tmem8 |
| ATF5 | NLGN1 | GRID1 | EMILIN1 | TMPRSS11E |
| ATP2C2 | Nrcam | GUCY1A1 | ENPP1 | TMTC4 |
| B3GALNT1 | NRN1L | HEPACAM2 | EPHA2 | TPSAB1 |
| BAIAP2L1 | NRXN3 | IL1RAPL1 | EPHB6 | tspan11 |
| Begain | ODC1 | CD37 | ESR2 | TSPAN4 |
| TEL1L | OLFM4 | CD40 | EXTL2 | TSPAN8 |
| BIN2 | OR2T29 | CD53 | FAM198B | tspan9 |
| CACNG2 | OR5K4 | CD59 | FKBP1a | VIPR2 |
| CADM2 | OR8K1 | CD63 | FKBP15 | VOPP1 |
| CALCB | PAFAH1B1 | CD69 | FKBP2 | ZSWIM8 |
| CCDC77 | PCDH11X | CD7 | PRSS27 | SDK2 |
| CCL4L2 | PCDHGA5 | CD82 | RCAN2 | SEMA4A |
| CD160 | PDE6B | Cd9 | RCC1L | SIGLEC15 |
| CD164 | PIGV | CDC14B | RGL4 | SLA |
| CD200R1 | PIP4P1 | CDC20B | RIMS1 | SLC34A3 |
| CD247 | PLCD1 | CDC42BPB | RTBDN | SLC4A11 |
| CD248 | PLEKHB1 | CDC42SE1 | Scamp3 | SLITRK4 |
| CD2AP | PLPPR1 | CDH1 | SGCE | Sntal |
| CD2BP2 | POMGNT1 | CDH20 | DLK1 | SNX11 |
| CD300LF | PPFIBP1 | CDH23 | CASD1 | |

TABLE 8

| | | | | |
|---|---|---|---|---|
| ADGRE5 | FBLN2 | MFAP2 | SLC27A6 | TMEM164 |
| ANXA5 | Frdm3 | MTMR4 | SLC32A1 | TMEM18 |
| ARHGAP20 | GABRA1 | NECTIN4 | SLC38A2 | tmem255a |
| ARHGAP8 | GNA11 | OLFML2B | SLC39A14 | TMEM54 |
| ATP1B1 | GNAS | OPCML | SLC4A2 | TRAF4 |
| balap3 | HTR7 | OR5B17 | SNTA1 | ZCCHC14 |
| cd63 | IL27RA | OSBPL6 | SOBP | Pnma6 |
| Cldn1 | IRS4 | Pianp | SPATA13 | |
| CLDN5 | izumo2 | PIGQ | ST3GAL4 | |
| clmp | JCAD | PLA2G12A | st8sia4 | |
| cpn2 | KIR2DL3 | PMEPA1 | TCIRG1 | |
| CRISPLD2 | KLHDC10 | PTPRB | TESC | |
| Egflam | LY6K | SLC13A5 | TGFBR3 | |
| EML6 | LYNX1 | SLC14A1 | THSD4 | |
| EXTL3 | LYPD5 | SLC22A3 | TMED8 | |

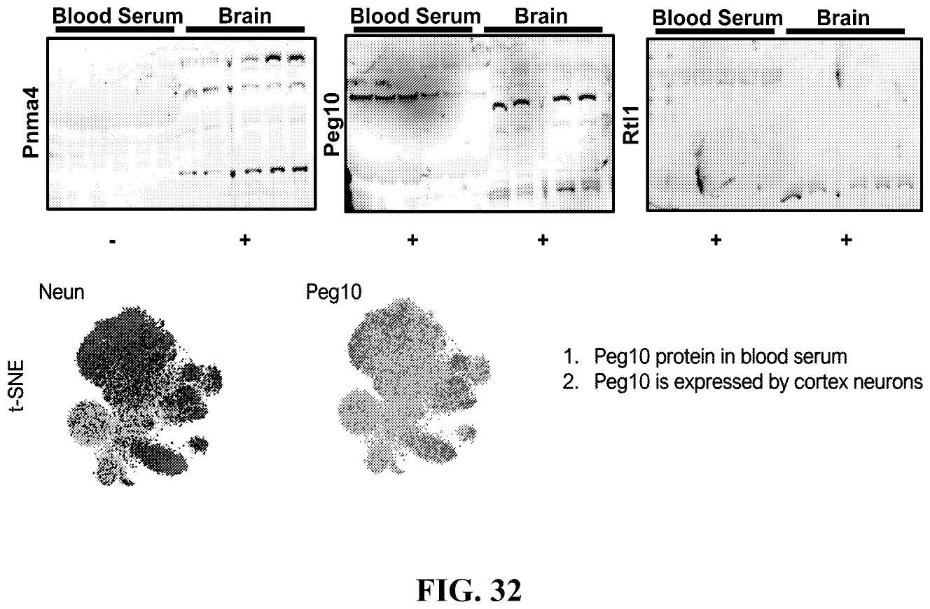

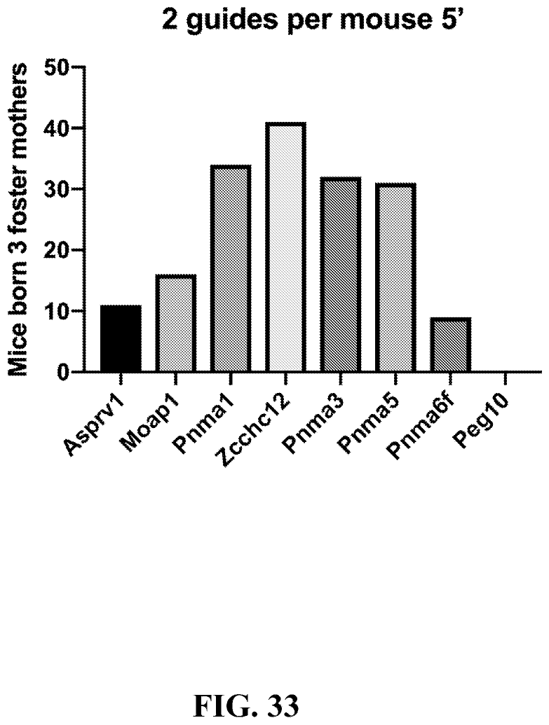

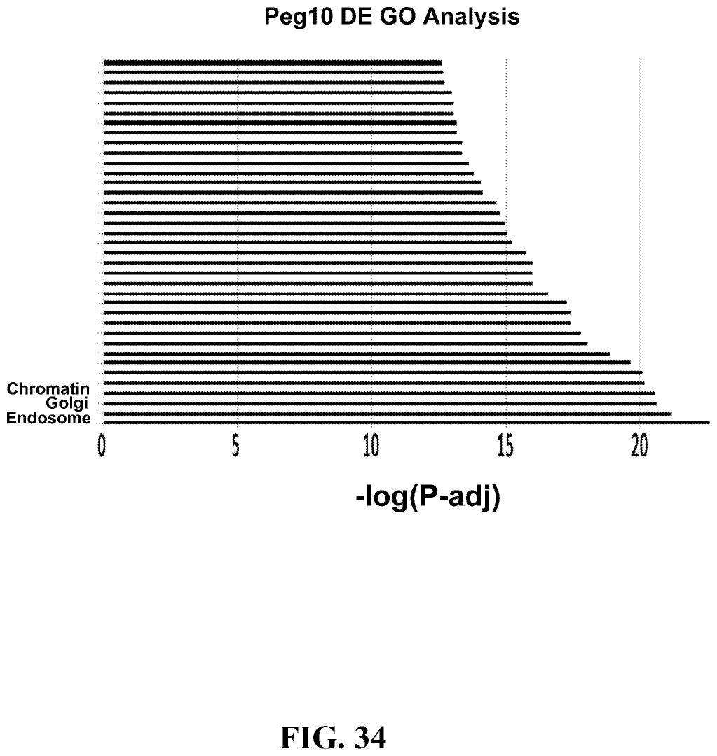

Applicants next determined that PEG10 can be found in both the blood serum and the cortex neurons in the brain (FIG. 32). Consistent with previous reports, knockout mice lacking PEG10 show early embryonic lethality, indicating the importance of this gene in embryonic development (FIG. 33). Gene ontology analysis of primary mouse neurons revealed three groups of differentially expressed genes: 1) genes involved in chromatin remodeling, 2) genes involved in the trans-Golgi network and exocytosis, and 3) SNAREs and other genes coding for endosomal and transmembrane proteins.

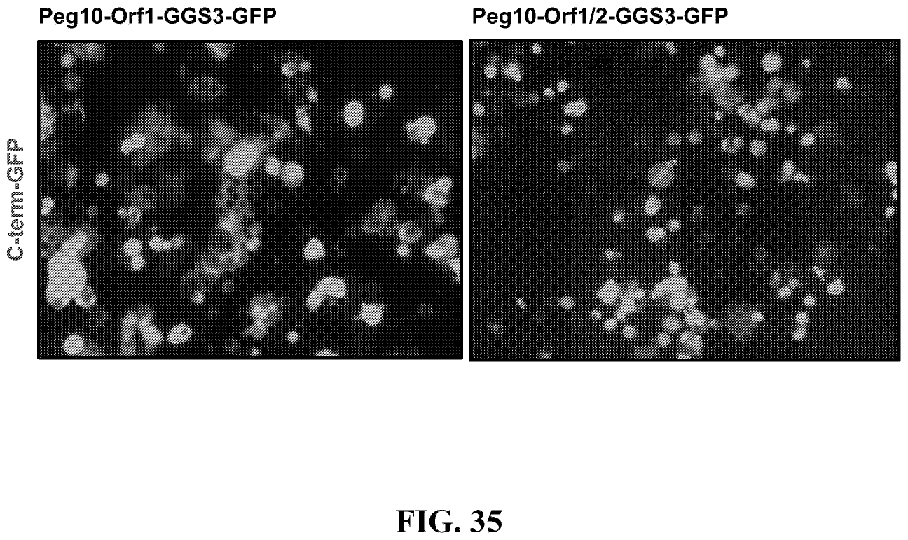

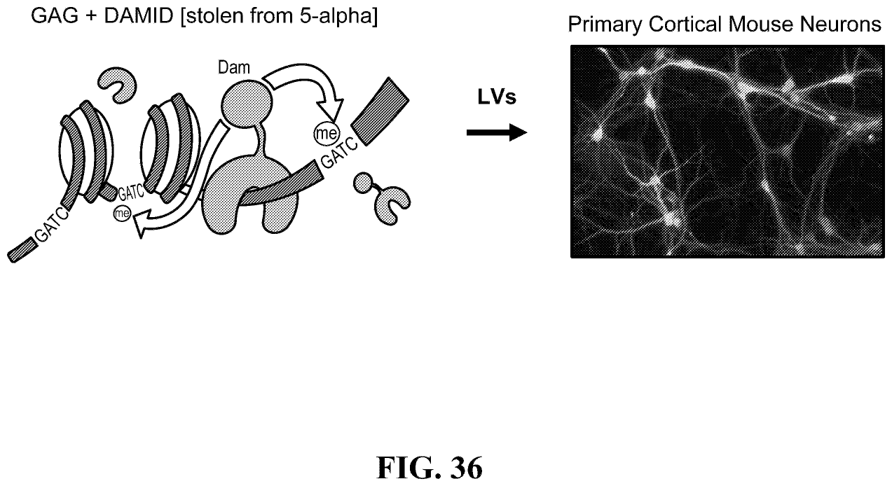

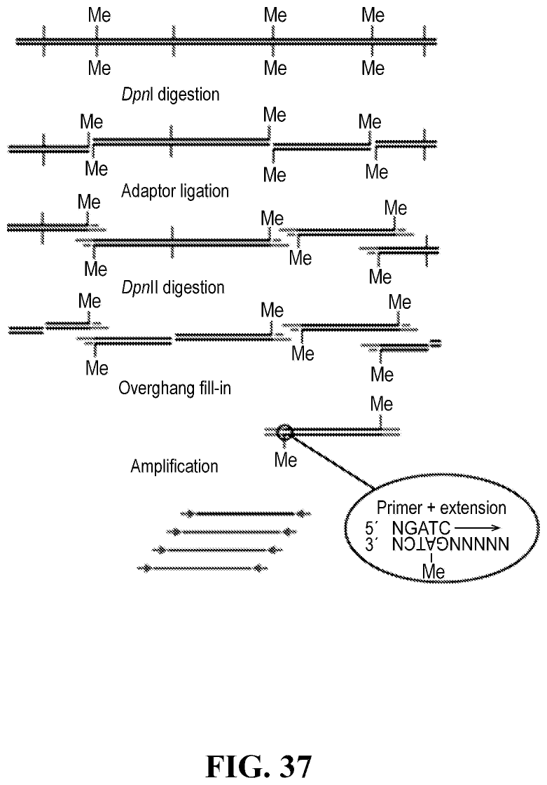

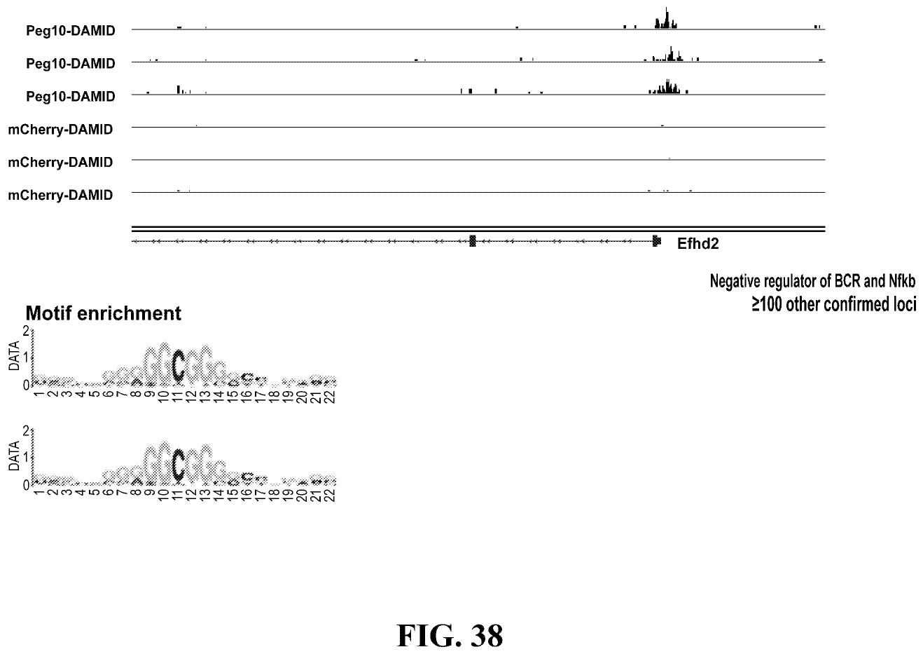

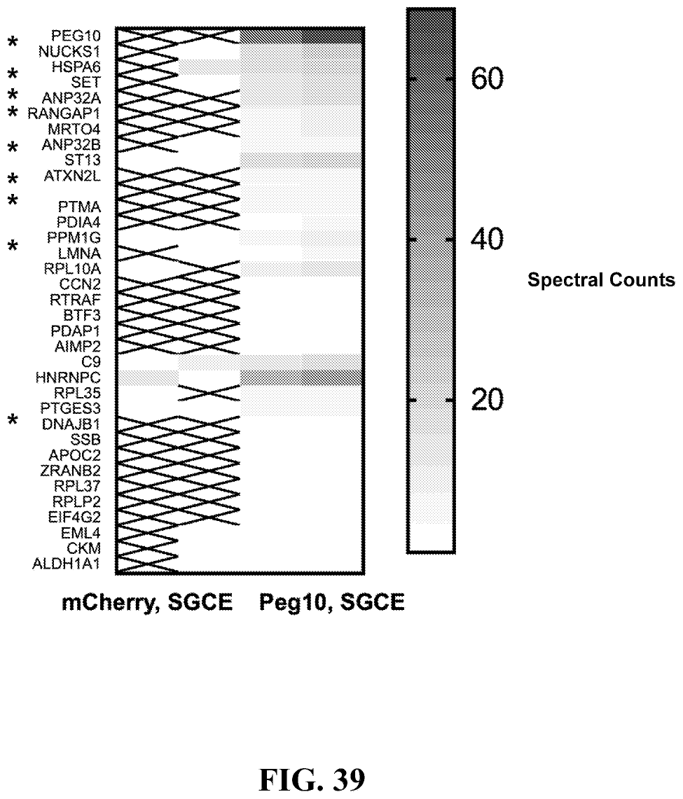

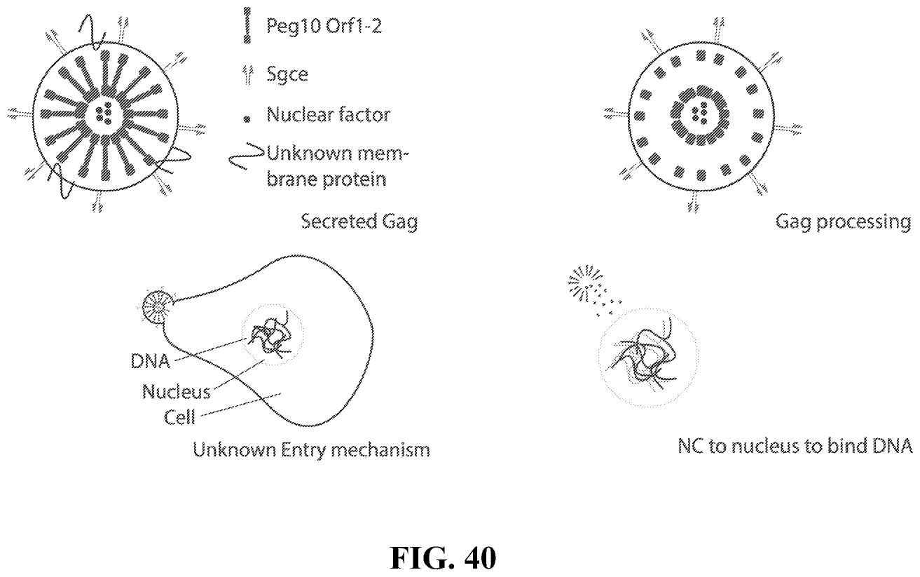

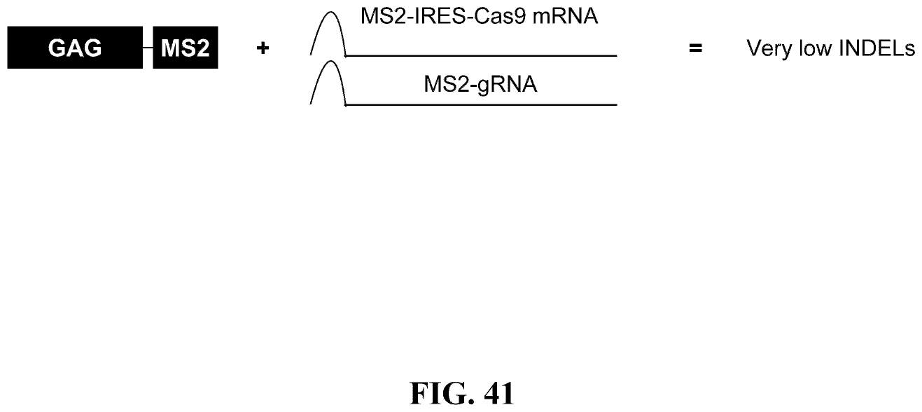

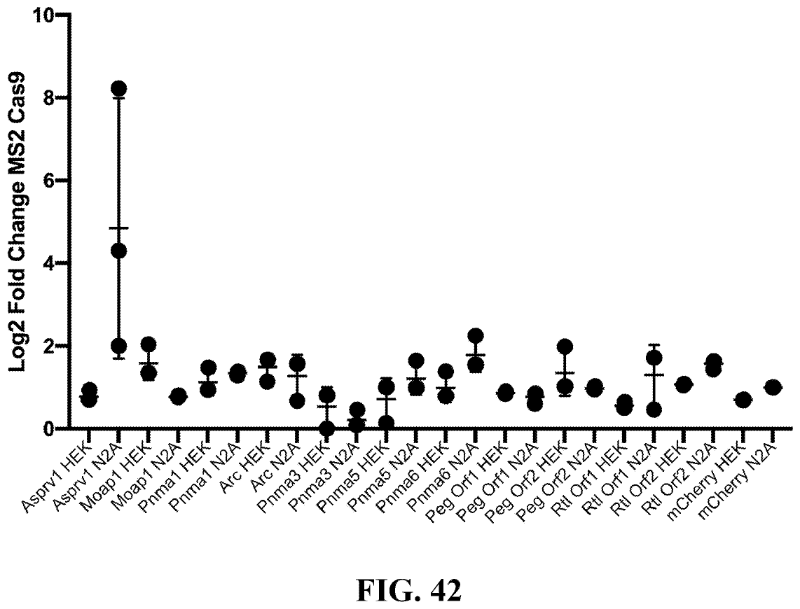

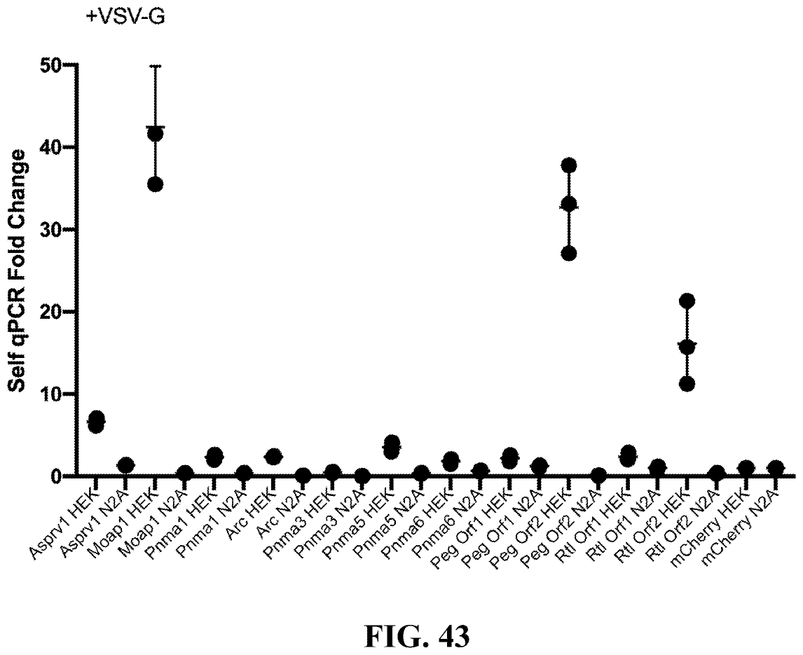

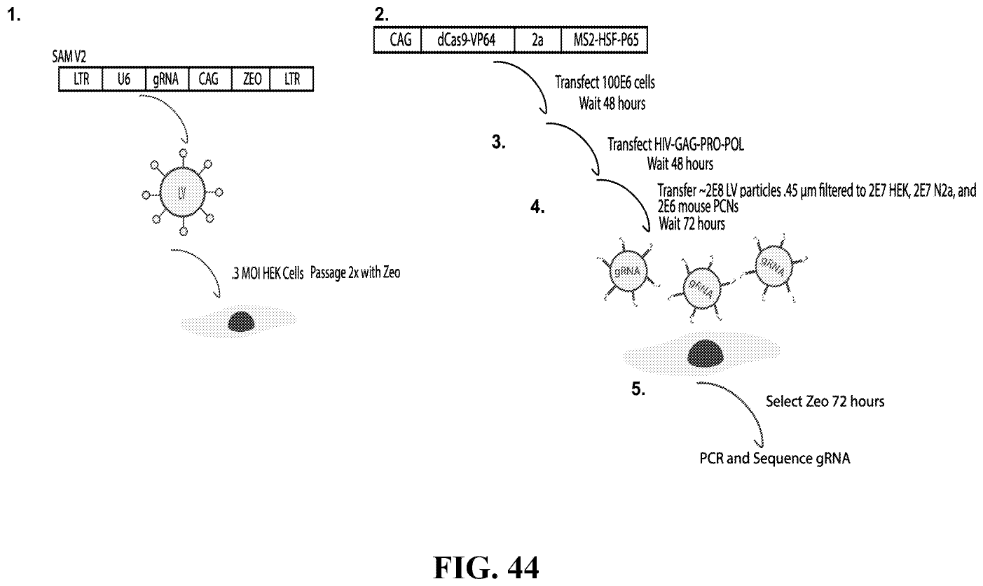

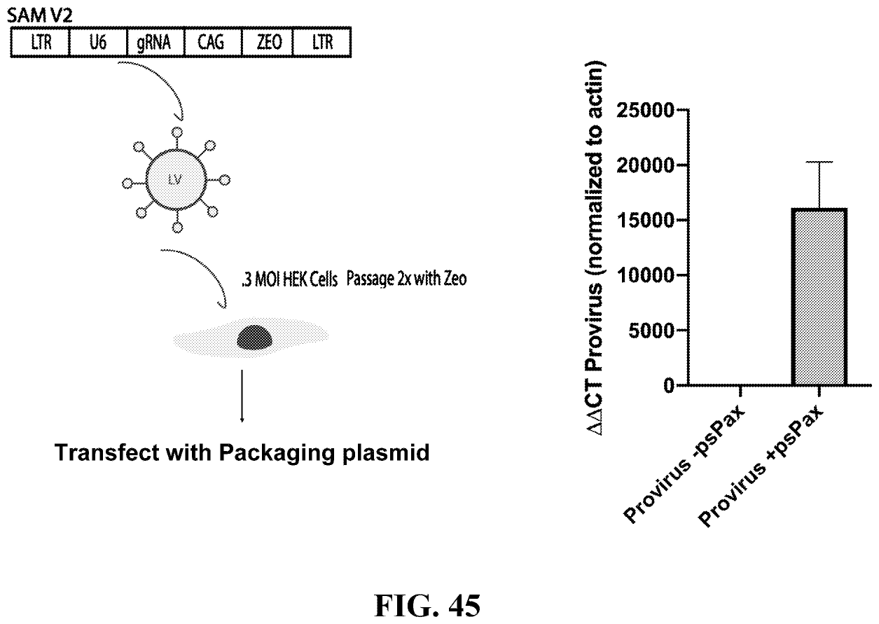

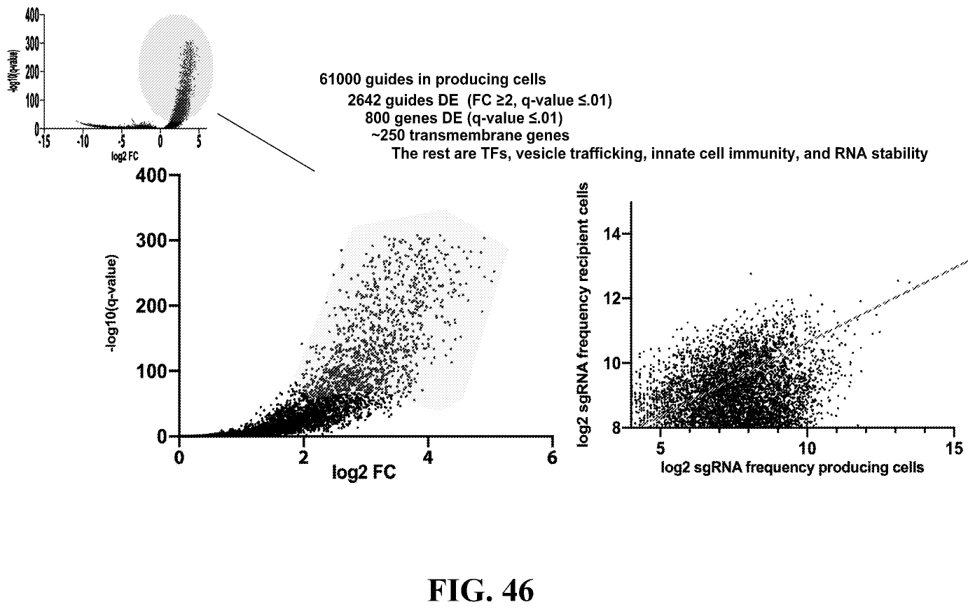

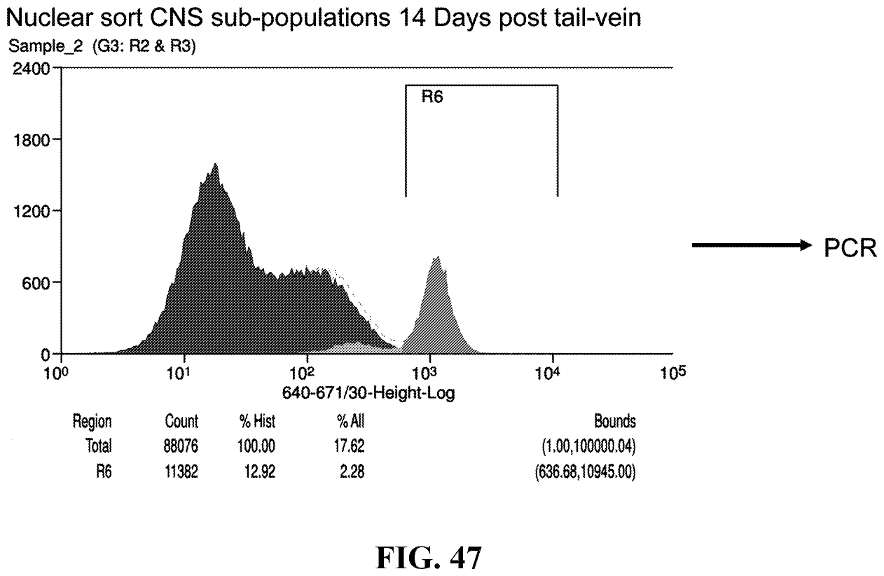

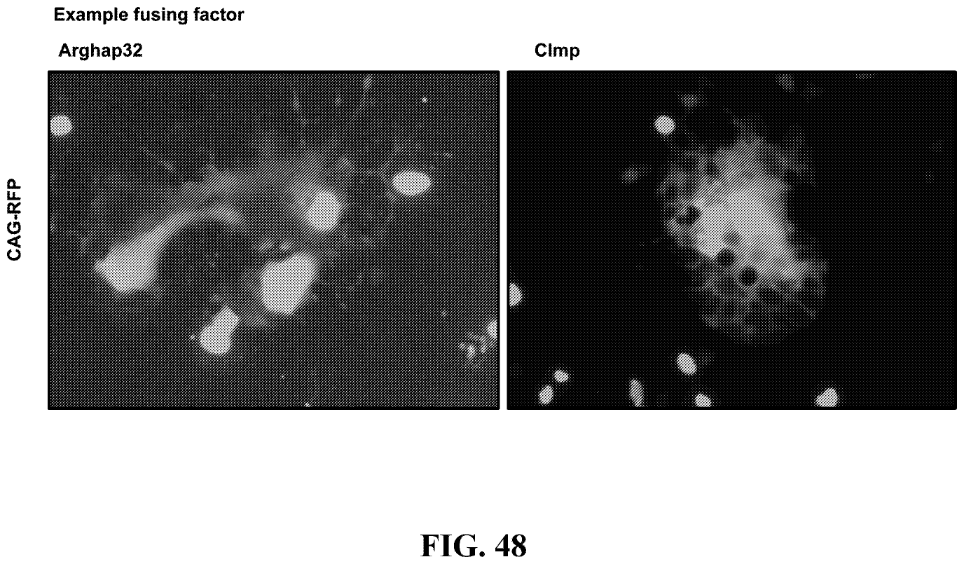

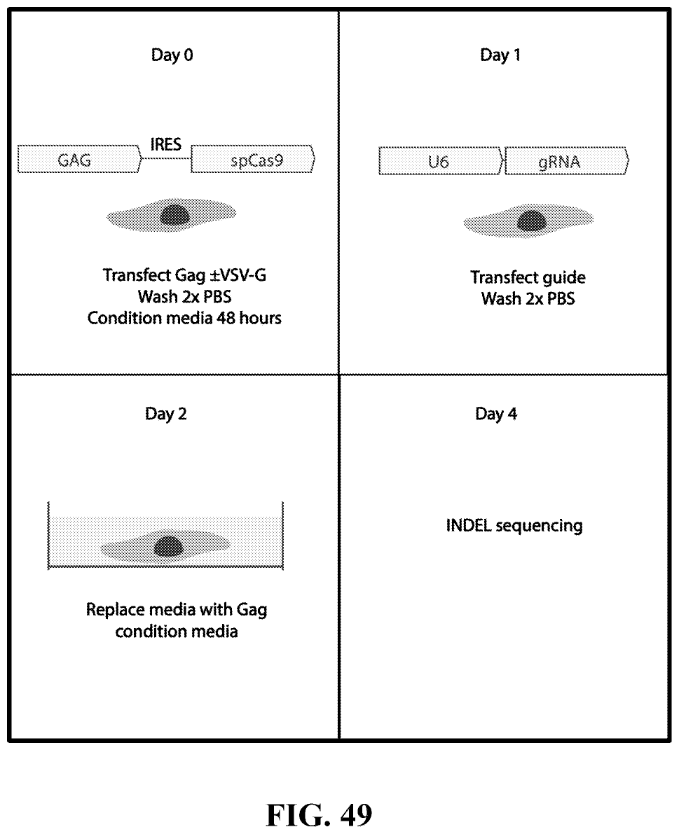

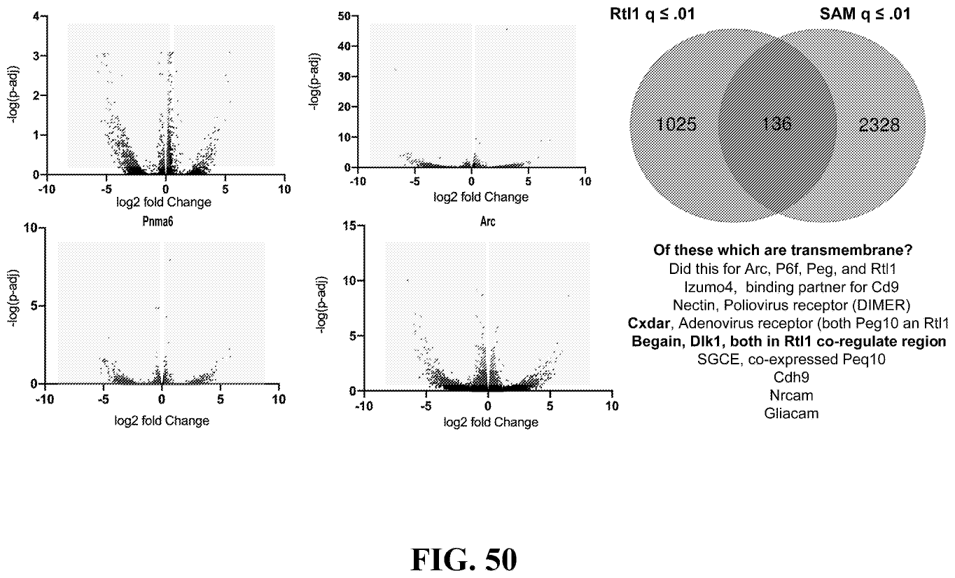

To figure out whether secreted GAGs are chromatin modifiers that bind DNA but not RNA, Applicants carried out DNA adenine methyltransferase identification (DamID), a protocol used to map the binding sites of DNA- and chromatin-binding protein in eukaryotes. DamID identifies binding sites by expressing the proposed DNA-binding protein as a fusion protein with DNA methyltransferase. Binding of the protein of interest to DNA localizes the methyltransferase in the region of the binding site. Adenosine methylation does not occur naturally in eukaryotes and therefore adenine methylation in any region can be concluded to have been caused by the fusion protein, implying the region is located near the binding site (FIG. 36). To carry out this protocol, Applicants digested the genome with DpnI, which cuts only methylated GATCs. Double-stranded adapters with a known sequence were then ligated to the ends generated by DpnI. Ligation products were digested using DpnII, which cuts non-methylated GATCs, ensuring that only fragments flanked by consecutive methylated GATCs were amplified in the subsequent PCR. A PCR with primers matching the adaptors was then carried out, leading to the specific amplification of genomic fragments flanked by methylated GATCs (FIG. 37). The data obtained by DamID mapping were then cross-referenced with ATAC-sequencing data (FIG. 38). Applicants overexpressed PEG10 and SGCE in N2A cells, ultracentrifuged the VLP fraction and analyzed precipitated proteins by mass-spectrometry. Various proteins, including RNA turnover factors, transcription factors, and chromatin remodelers were found to be enriched in this fraction (FIG. 39). Applicants concluded that PEG10 is efficiently secreted from cells and that it can mediate delivery of large macromolecules. Because PEG10 is found across the body, it likely binds DNA and may itself be what is delivered to cells, entering a cell and directly binding DNA (FIG. 40).

### Example 4—Processing of PEG10 and Functional Properties of Processed Domains

The ability of PEG10 to form vesicles led to two central questions. 1) How is PEG10 processed, and, 2) what do each of the functional domains do? To answer the first question, Applicants overexpressed N- and C-terminal HA-tagged mouse PEG10 in HEK293FT cells, immunoprecipitated PEG10 using HA magnetic beads, and analyzed bands by Western blotting. Corresponding commassie-dyed bands were analyzed by mass-spectrometry. Results showed that the protein is cleaved into all the respective predicted domains (FIGS. 56, 57A-57F, 58A, and 58B).

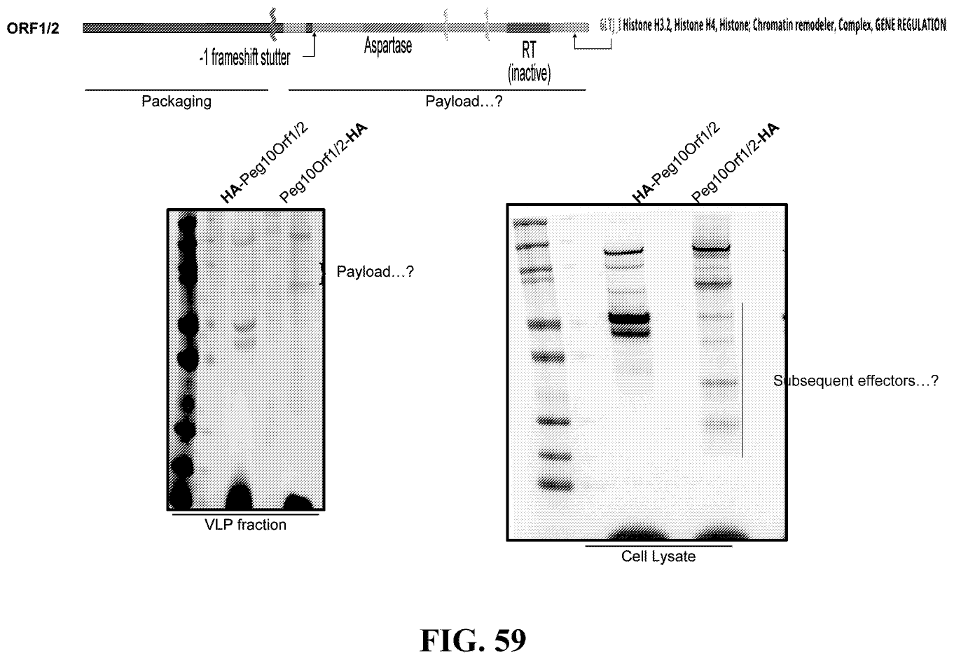

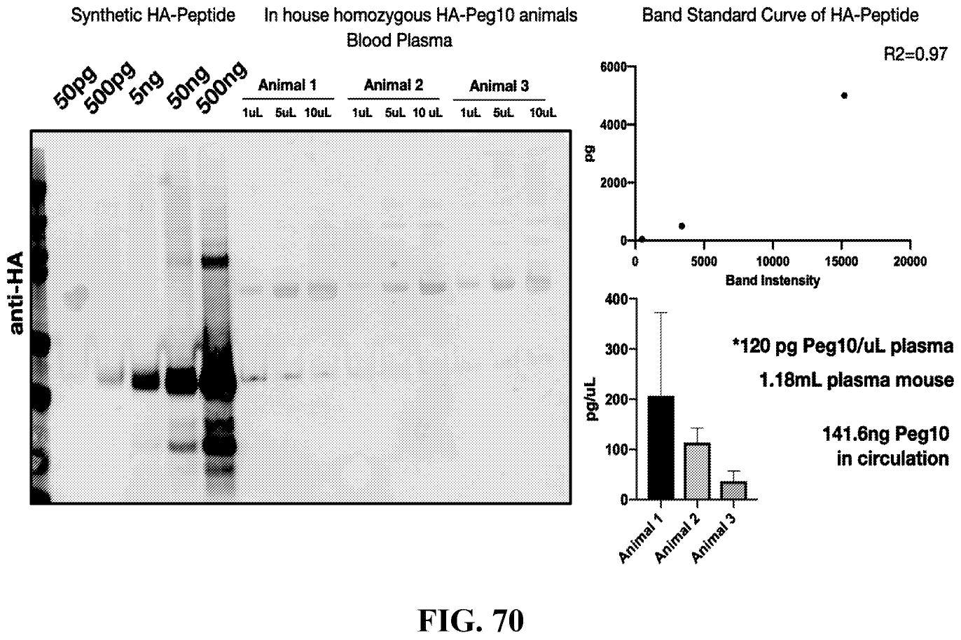

To answer the second question, Applicants compared PEG10 to a previously-identified protein known as MYEF, a DNA-binding protein that binds a very specific 10-basepair sequence in a 3× repeat (shown on the right side of FIG. 59). Applicants determined that PEG10 binds the exact same sequence, so they attempted to package particles that express the DNA sequence. When PEG10 was overexpressed with a plasmid DNA containing this sequence, Applicants noted that PEG10 preferentially packages and encapsulates that 10-basepair DNA sequence and secretes the plasmid carrying the sequence. To quantify how much PEG10 circulates in the blood, Applicants engineered mice with a PEG10 antibody receptor tag and determined that PEG10 is expressed at about 120 pg/μL of blood plasma in mice (FIG. 70).

### Example 5—Adoption of PEG10 Capsids for Efficient and Specific Transfer of mRNA into Cells

More than 40% of the human genome is composed of sequence derived from retroelements, including retroviruses, that have integrated into mammalian genomes throughout evolution (1, 2). Most of the endogenous retrovirus genomes have lost their original function, but some of the virus genes have been recruited for diverse roles in normal mammalian physiology. In particular, the fusogenic syncytins evolved from retroviral Env proteins and the capsid-like PEG10 evolved from Gag are involved in mammalian placenta formation and are essential for mammalian embryonic development (3-5). Arc, a gag homolog expressed in neurons, forms secreted capsid-like structures that seem to be involved in memory consolidation in the brain and in the regulation of inflammation in the skin (6-8). Given the diversity of the domesticated virus-derived genes in mammals, it appears likely that presently uncharacterized virus gene homologs perform fundamental roles in mammalian physiology and disease, through the export of nucleic acids from cells, and could provide a programmable mechanism for intercellular communication.

To explore the diversity of the endogenous retrovirus derived genes, efforts focused on the major capsid protein, known as Gag (after group-specific antigen) in the case of retroviruses, the core structural protein that forms the capsid within the infectious retrovirus virion. The capsid protects the viral genome and enables efficient transfer of virus genomes between host cells (9). Previous genome analyses have identified many endogenous gag homologs in mammalian genomes (10). Nevertheless, given that substantially improved genome assembly and annotation of the human and mouse genomes became available since then, we mined the human genome for Gag homologs. This analysis resulted in the identification of 51 gag-derived genes in the human genome and 85 gag homologs in the mouse genome; for 37 genes, one to one orthologous relationships between human and mouse was readily traced, whereas the remaining ones appeared to be species-specific (Table 9). Table 9 shows a summary of orthologous groups of proteins with domains homologous to Gag capsid protein in human and mouse based on sequence alignments. Most orthologous groups with proteins from both species include one human sequence and a few mouse sequences, in most cases these mouse sequences in one group correspond to the same gene but differ mostly by truncation or point mutations. In two groups, namely with human proteins NP_776159.1 and NP_001299820.1 the mouse proteins are clearly paralogous but are practically equally close to human group members. Overall, there are 19 orthologous groups with proteins from both species, two of which are with obvious mouse paralogs, and 15 human and 48 mouse singletons.

TABLE 9

| Candidates (Homo sapiens) | | | Orthologous candidates (Mus musculus) | |
|---|---|---|---|---|
| Identified protein | Domain | Accession | Identified protein | Accession |
| endogenous retrovirus | Gag_p24 | XP_011511643.1 | — | — |
| group K member 5 Gag | | | | |
| prolyprotein | | | | |
| endogenous retrovirus | Gag_p30 | XP_016863109.1 | — | — |
| group K member 7 Gag | | | | |
| polyprotein | | | | |
| uncharacterized protein | Gag_p30 | XP_016883063.1 | — | — |
| LOC107985332 | | | | |
| paraneoplastic antigen- | PNMA | NP_001096620.1 | — | — |
| like protein 5 | | | | |
| retrotransposon-derived | PNMA | NP_001165908.1 | Retrotransposon-derived | sp\|Q7TN75 |
| protein PEG10 isoform 3 | | | protein PEG10 | |
| retrotransposon Gag-like | DUF4939 | NP_001127793.1 | paraneoplastic antigen-like | NP_001007570.1 |
| protein 8A isoform 2 | | | protein 8A | |
| retrotransposon Gag-like | DUF4939 | NP_001071639.1 | — | — |
| protein 8B | | | | |
| retrotransposon Gag-like | DUF4939 | NP_001071641.1 | — | — |
| protein 8B | | | | |
| retrotransposon Gag-like | DUF4939 | NP_001071640.1 | — | — |
| protein 8A isoform 1 | | | | |
| paraneoplastic antigen | PNMA | NP_001269464.1 | unnamed protein product, | BAE24735.1 |
| Ma3 isoform 2 | | | partial | |
| | | | paraneoplastic antigen Ma3 | NP_694809.1 |
| | | | homolog | |
| paraneoplastic antigen- | PNMA | NP_116271.3 | mCG1032934 | EDL29902.1 |
| like protein 6A | | | | |
| paraneoplastic antigen- | PNMA | NP_443158.1 | — | — |
| like protein 5 | | | | |
| paraneoplastic antigen | PNMA | NP_006020.4 | unnamed protein product | BAC25885.1 |
| Ma1 | | | paraneoplastic antigen Ma1 | NP_081714.2 |
| | | | homolog | |
| modulator of apoptosis 1 | PNMA | NP_071434.2 | Modulator of apoptosis 1 | AAH55374.1 |
| | | | modulator of apoptosis 1 | NP_001136409.1 |
| paraneoplastic antigen | PNMA | NP_009188.1 | mKIAA0883 protein, partial | BAD90245.1 |
| Ma2 | | | paraneoplastic antigen MA2, | EDL35986.1 |
| | | | isoform CRA_a, partial | |
| | | | paraneoplastic antigen MA2, | EDL35987.1 |
| | | | isoform CRA_b | |
| | | | paraneoplastic antigen Ma2 | NP_780707.1 |
| | | | homolog | |
| paraneoplastic antigen- | PNMA | NP_065760.1 | unnamed protein product, | BAE24315.1 |
| like protein 8B | | | partial | |
| | | | unnamed protein product, | BAE25295.1 |
| | | | partial | |
| | | | PNMA-like protein 2 | NP_001093106.1 |
| zinc finger CCHC | PNMA | NP_001137450.1 | zinc finger, CCHC domain | EDL23888.1 |
| domain-containing | | | containing 18, isoform | |
| protein 18 | | | CRA_a, partial | |
| | | | zinc finger CCHC domain- | NP_001030586.1 |
| | | | containing protein 18 | |
| zinc finger CCHC | PNMA | NP_776159.1 | zinc finger CCHC domain- | NP_001345405.1 |
| domain-containing | | | containing protein 12 | |
| protein 12 | | | unnamed protein product | BAB23950.1 |
| protein Bop | DUF4939 | NP_078903.3 | — | — |
| retrotransposon Gag-like | DUF4939 | NP_689907.1 | retrotransposon Gag-like | NP_955762.1 |
| protein 3 | | | protein 3 | |
| retrotransposon Gag-like | DUF4939 | NP_001019626.1 | retrotransposon Gag-like | NP_001265463.1 |
| protein 5 | | | protein 5 | |
| | | | mKIAA2001 protein, partial | BAD90267.1 |
| retrotransposon-like | PNMA | NP_001128360.1 | — | — |
| protein 1 | | | | |
| retrotransposon Gag-like | DUF4939 | NP_115663.2 | retrotransposon Gag-like | NP_808298.2 |
| protein 6 | | | protein 6 | |
| | | | unnamed protein product | BAC39047.1 |
| protein LDOC1 | DUF4939 | NP_036449.1 | — | — |
| zinc finger CCHC | PNMA | NP_001299820.1 | zinc finger CCHC domain- | NP_001345405.1 |
| domain-containing | | | containing protein 12 | |
| protein 12 | | | unnamed protein product | BAB23950.1 |
| zinc finger CCHC | PNMA | XP_011529314.1 | zinc finger, CCHC domain | EDL23888.1 |
| domain-containing | | | containing 18, isoform | |
| protein 18 isoform X1 | | | CRA_a, partial | |
| | | | zinc finger CCHC domain- | NP_001030586.1 |
| | | | containing protein 18 | |
| retrotransposon Gag-like | DUF4939 | NP_065820.1 | mKIAA1318 protein, partial | BAD90439.1 |
| protein 9 | | | mCG7581 | EDL14737.1 |
| | | | retrotransposon Gag-like | NP_001035524.2 |
| | | | protein 9 | |
| | | | retrotransposon Gag-like | sp\|Q32KG4.1 |
| | | | protein 9 | |
| activity-regulated | Arc2 | NP_056008.1 | activity-regulated | NP_001263613.1 |
| cytoskeleton-associated | | | cytoskeleton-associated | |
| protein | | | protein | |
| retrotransposon Gag-like | PNMA | NP_001004308.2 | — | — |
| protein 4 | | | | |
| endogenous retrovirus | Gag_p24 | XP_011526763.1 | — | — |
| group K member 8 Gag | | | | |
| polyprotein-like | | | | |
| paraneoplastic antigen- | PNMA | NP_001171853.1 | mKIAA1934 protein, partial | BAD90475.1 |
| like protein 5 | | | paraneoplastic antigen-like | NP_001093931.1 |
| | | | protein 5 | |
| retroviral-like aspartic | DUF4939 | NP_690005.2 | — | — |
| protease 1 | | | | |
| paraneoplastic antigen | PNMA | NP_001341909.1 | mCG1032934 | EDL29902.1 |
| Ma6F | | | | |

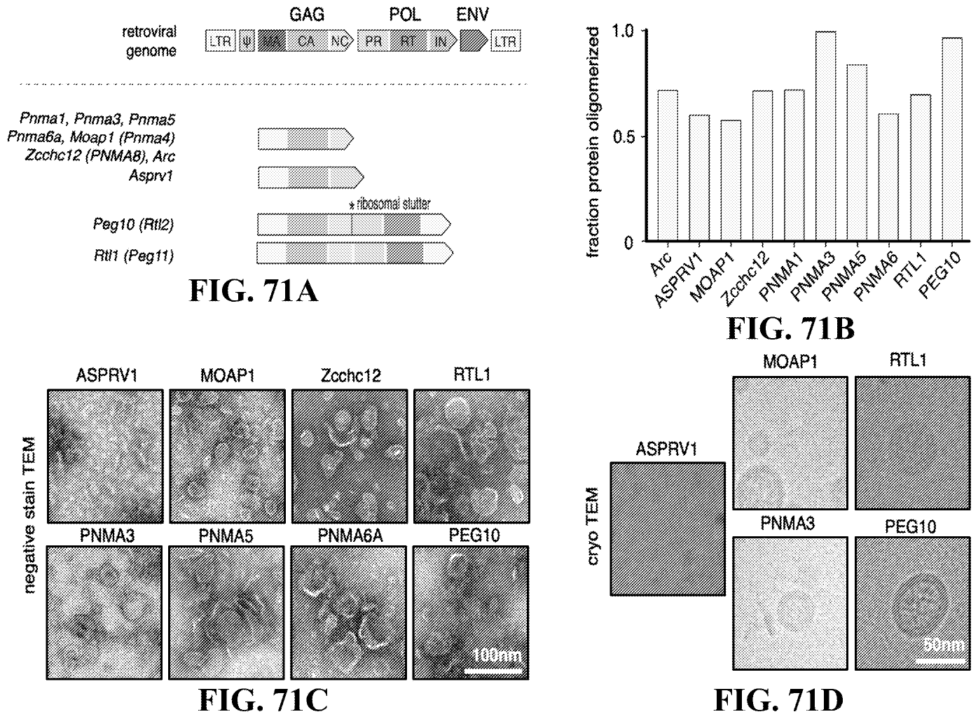

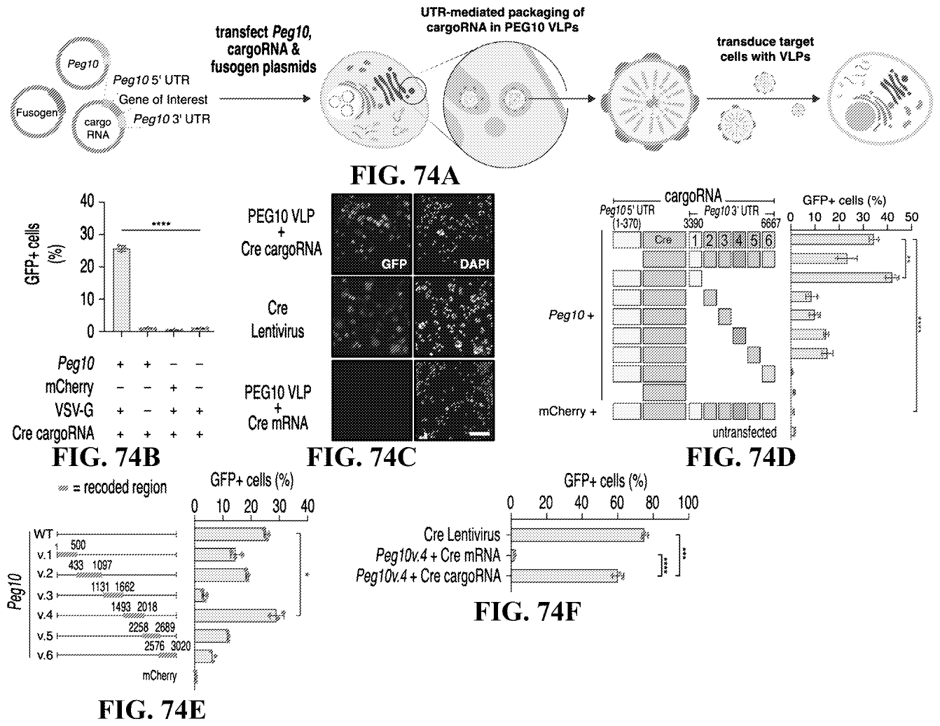

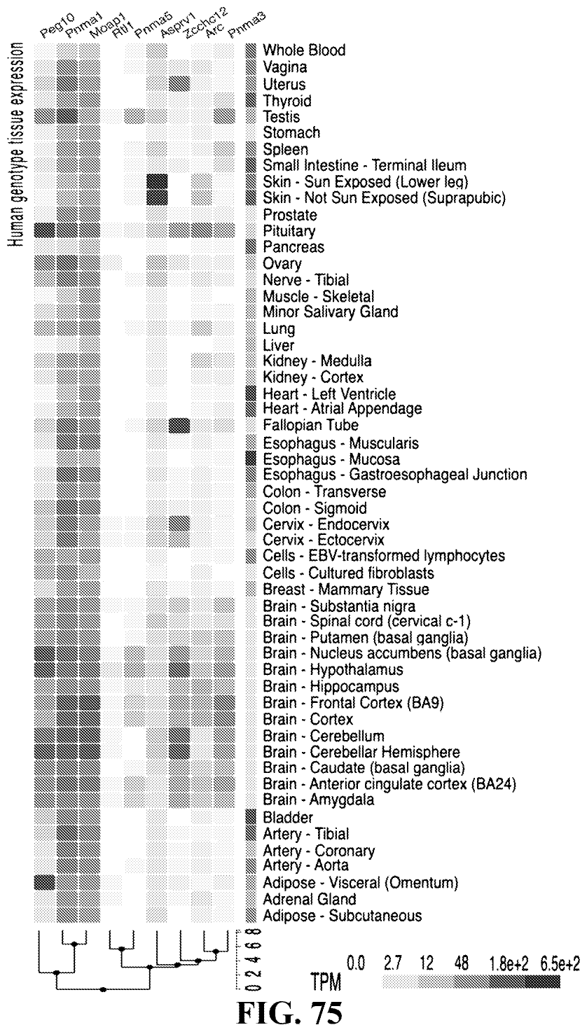

The canonical retroviral genome encodes a long polyprotein that consists of the matrix (MA), capsid (CA), nucleocapsid (NC, Zn finger), protease (PRO), reverse transcriptase (RT), and integrase (IN) domains. Apart from the CA domain, the Gag homologs encoded in the human and mouse genomes encompass several distinct combinations of retrovirus-derived domains, which could underlie the specific functions of these proteins in mammals (FIG. 71A). Some families of endogenous Gag proteins, such as the PNMA family, contain only the CA and NC domains, whereas others, such as RTL1 and PEG10 (also known as RTL2 or Mart2), additionally contain PRO and RT domains. To narrow down the scope of the search, efforts were focused on those Gag proteins that, first, are conserved between human and mouse, and second, have detectable levels of mRNA in adult human tissues, reasoning that such CA proteins were most likely to be co-opted for important physiological roles in mammals (FIG. 75).

To validate the genome mining results, the mouse versions of the selected CA proteins were produced in E. coli and found that a number of these formed higher molecular weight oligomers identified by size exclusion (FIG. 71B and FIG. 76A), as previously noted for some of these proteins, such as Arc (8). Electron microscopy of these aggregated proteins shows that several of them, including MOAP1, ZCCHC12, RTL1, PNMA3, PNMA5, PNMA6a, and PEG10, self-assemble into capsid-like structures, many of which appear spherical (FIGS. 71C, 71D, and FIGS. 76B-76C). PEG10 and ARC have previously been shown to form capsids (8, 11, 12), but the other proteins have been less well characterized.

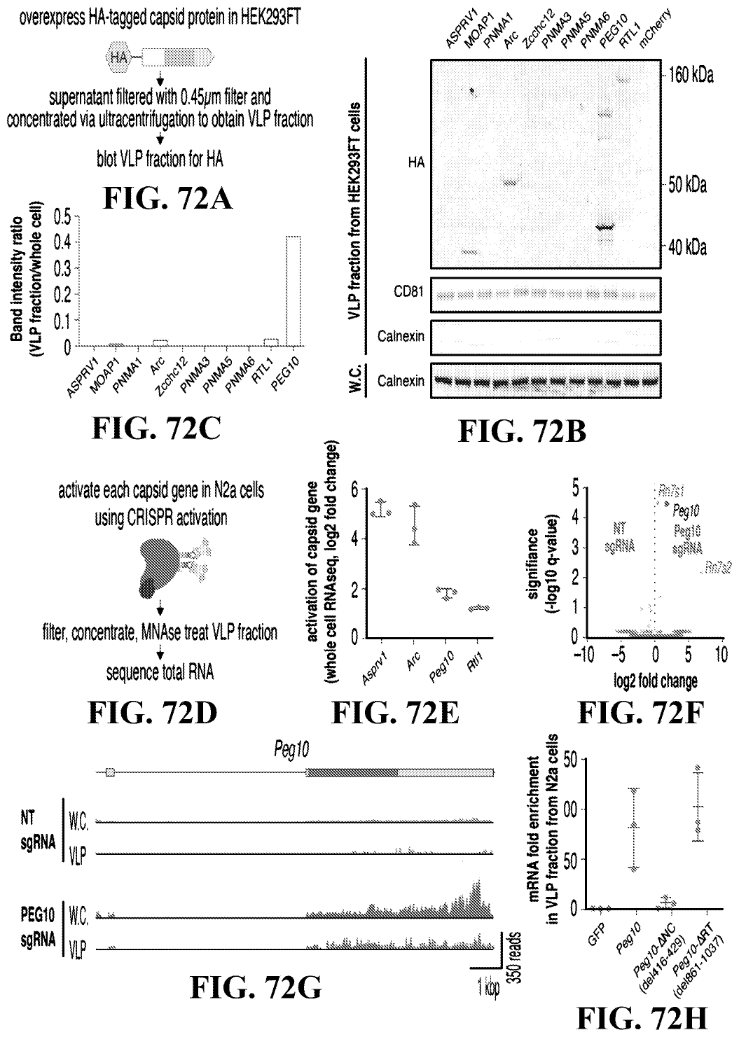

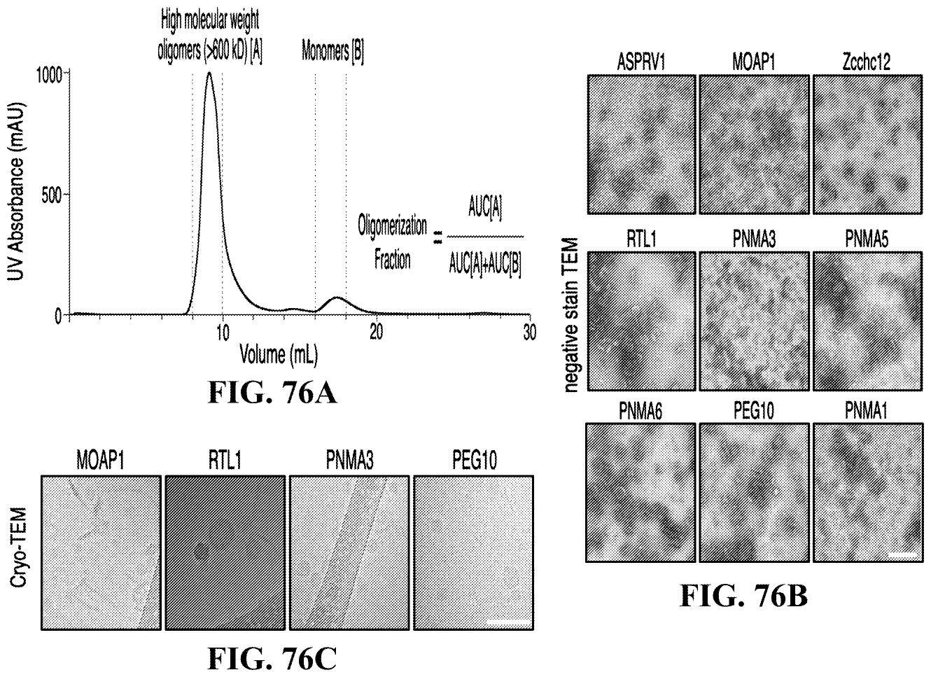

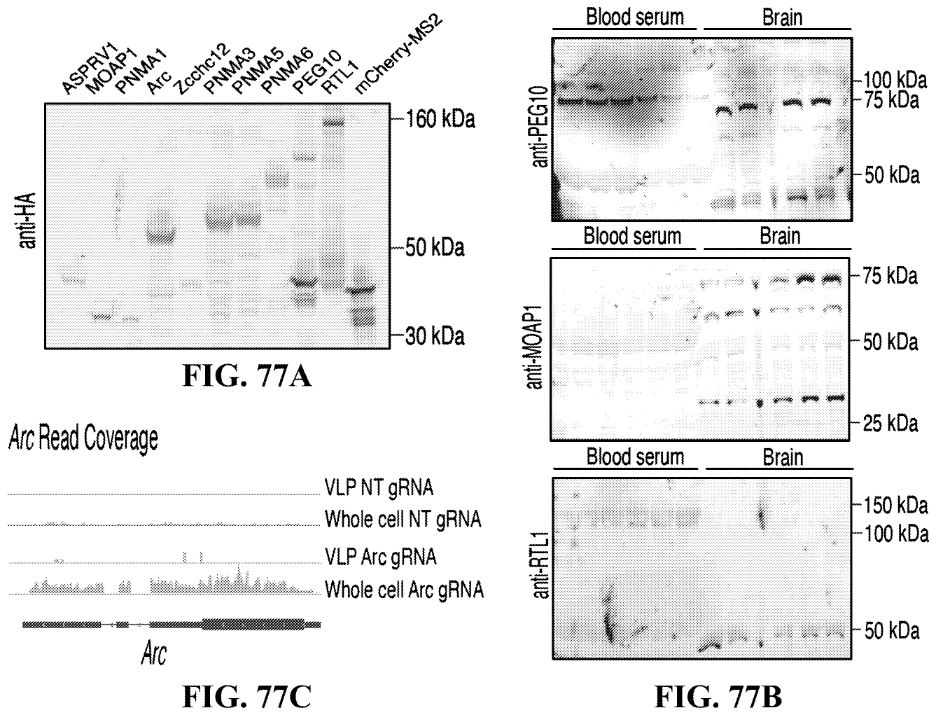

Given that these proteins spontaneously form viral capsid-like structures in vitro (FIGS. 71B to 71D), and secretion of Arc capsids by mammalian neurons has been reported (8), it was hypothesized that such capsids are released by cells in a process similar to retrovirus egress. To test this hypothesis, the mouse orthologue of each CA-containing gene was transfected into HEK293FT cells and both the whole cell lysate and the virus-like particle (VLP) fraction was harvested by clarification and ultracentrifugation of the culture media (FIGS. 72A and 77A). It was found that MOAP1, ARC, PEG10, and RTL1 were all present in the VLP fraction (FIG. 72B), but PEG10 was by far the most abundantly secreted protein (FIG. 72C). Additionally, PEG10, but not MOAP1 or RTL 1, were readily detectable in cell-free adult mouse serum (FIG. 77B).

It was next tested whether any of the secreted capsid-like particles contained specific mRNAs. To avoid sequencing contamination from plasmid transfection, CRISPR activation (13) was used to induce expression of each endogenous gene in mouse N2a cells (FIGS. 72D and 72E). mRNA sequencing was performed on whole cells and the VLP fraction to identify any RNA species that might be actively secreted with the CA-containing proteins. The VLP fraction was nuclease treated to remove any residual, un-encapsidated RNA. Among the transcriptionally activated genes, Peg10 was the only one for which enrichment for a specific mRNA species in the VLP fraction was observed. Indeed, Peg10 transcriptional activation led to accumulation of significant substantial amounts of Peg10 mRNA in the VLP fraction (FIG. 72F), whereas activation of the other genes, including Arc, showed no enrichment for self in this fraction (FIG. 77C). Moreover, reads from the VLP fraction covered the entire length of the Peg10 transcript (FIG. 72G), including the 5′ and 3′ untranslated regions (UTRs), showing that the enrichment for this mRNA reflected its bona fide secretion, rather than spurious contamination. To further validate these results, overexpression plasmids of UTR-containing Peg10 mutants were transiently transfected into N2a cells and it was found that mRNA secretion depended on the PEG10 NC, because removing the nucleic acid-binding zinc finger CCHC motif from the PEG10 NC substantially reduced secretion of its mRNA (FIG. 72H). Given PEG10 expression in adult tissues, together with the ability of this protein to form capsids and secrete its own mRNA, the roles of different domains of PEG10 were investigated in further detail in order to elucidate its properties and potential functions and to determine if it would be a suitable candidate for reprogramming as an mRNA delivery vehicle.

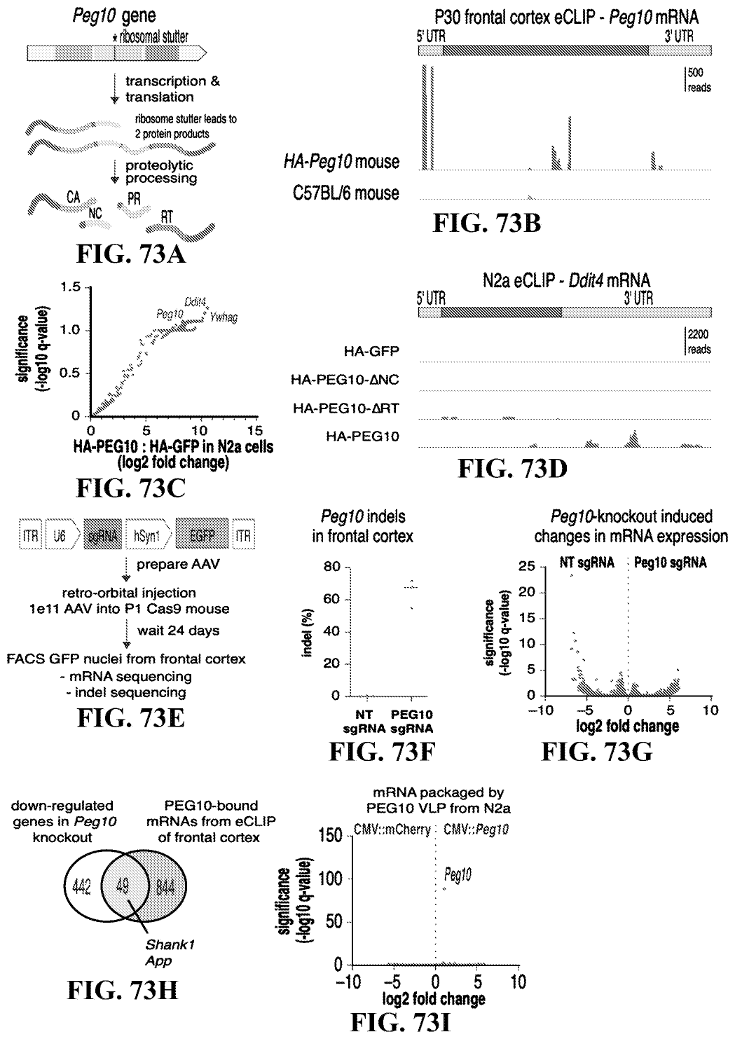

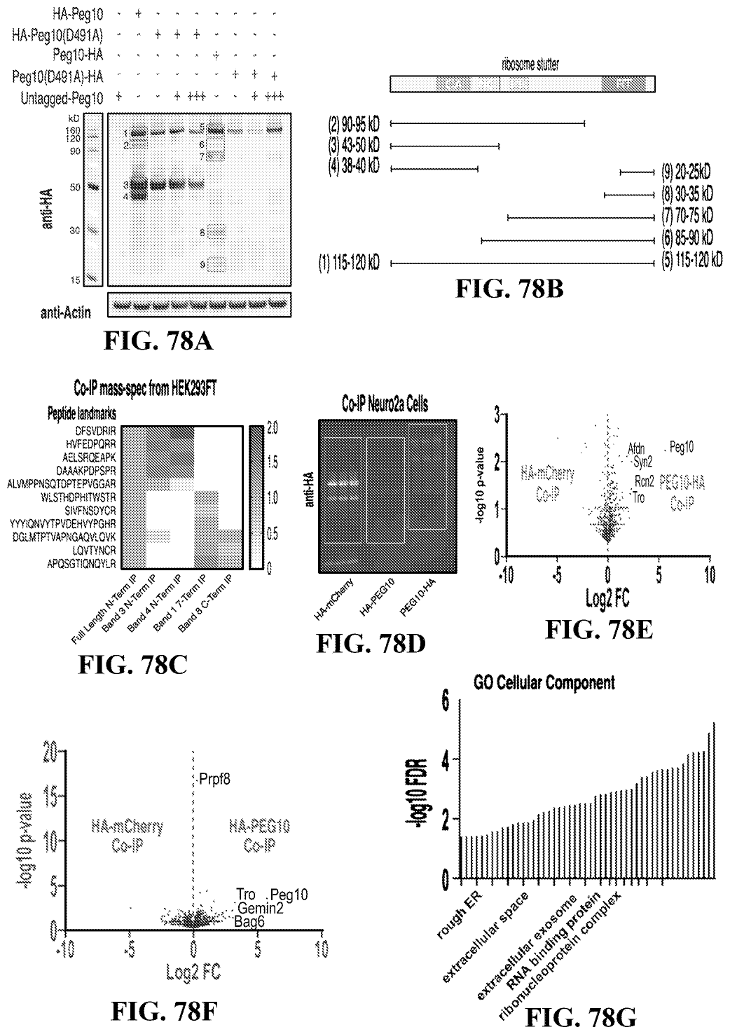

The full length PEG10 polyprotein encodes four conserved retroviral domains: CA, NC, PRO, and a catalytically inactive RT (14, 15). Like some retroelements, the transcript is translated in two protein isoforms, namely, reading frame 1 (RF1), which encodes only CA and NC, and reading frame 2 (RF2), which is generated via a −1 ribosomal frameshift (16) and encodes all four domains (FIG. 73A). The PEG10 polyprotein is then processed in cis by the protease into individual proteins as shown previously (17) and confirmed by Western blotting (FIG. 78A). Inactivation of the protease by mutating the catalytic aspartate inhibits self-processing (FIG. 78A). The band sizes on the Western blot correspond to the sizes of the predicted functional domains of PEG10 (FIG. 78B), as confirmed by examination of the peptide signatures from these bands by mass spectroscopy (FIG. 78C).

To reprogram PEG10 for delivery, first the mode of its secretion, and second, which nucleic acids it bound in the cell were determined. To characterize the interactions that might be relevant for the functions of PEG10, Co-IP mass spectroscopy was performed on N and C-terminally HA-tagged Peg10 that was transiently transfected into N2a cells (FIGS. 78D-78F) and found that PEG10 associated with many rough ER proteins, such as RCN2, as well as proteins associated with RNA processing, such as MOV10 and GEMIN2 (FIG. 78G). These findings suggest that PEG10 is translated in the rough ER, where it is bound for secretion, and also might be involved in splicing and processing of bound mRNA.

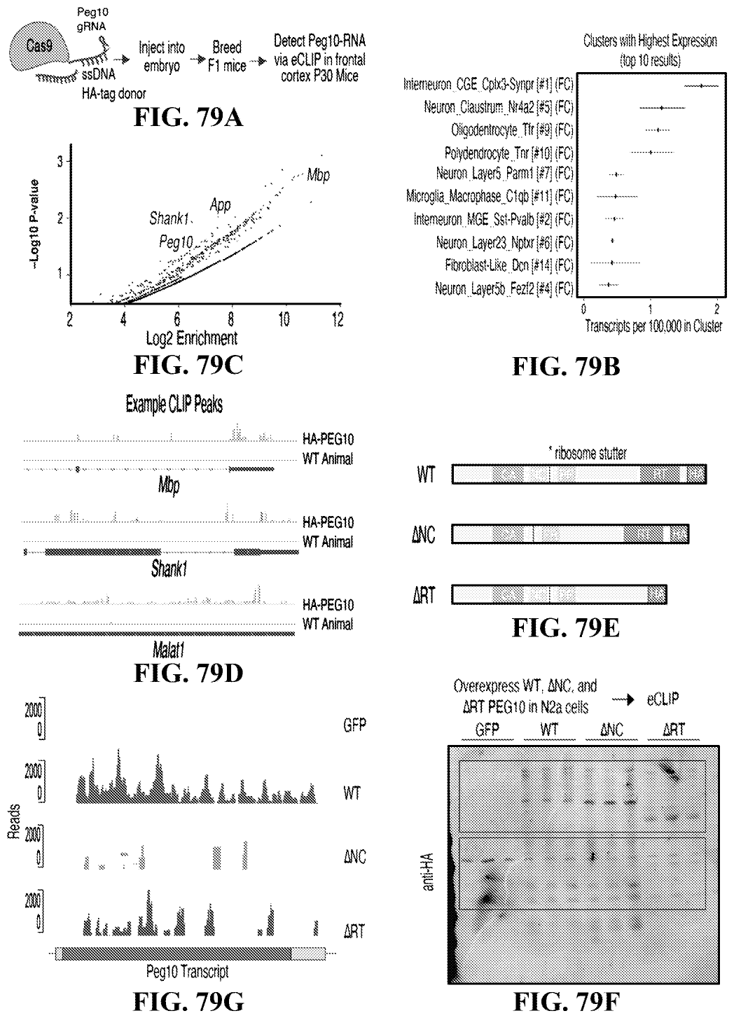

To investigate potential RNA binding by PEG10 in the native context, we generated knock-in mice carrying an N-terminal HA-tag on the endogenous PEG10 protein (FIG. 79A). Expression of Peg10 in cortical neurons has been demonstrated previously (18) and validated here (FIG. 79B). Using eCLIP to detect PEG10-RNA complexes in the frontal cortex of P30 HA-PEG10 mice, it was found that PEG10 binds many mRNAs in the mouse brain, including mRNAs for Shank1, App, and Mbp, key genes involved in neurodevelopment (FIGS. 79C-79D) (19, 20). PEG10 also was found to bind its own mRNA, where strong binding in the 5′ UTR was detected. Some additional binding near the boundary between the nucleocapsid and protease coding sequences and in the beginning of the 3′ UTR (FIG. 73B) was detected and was in agreement with previous PEG10 eCLIP datasets (11). To assess the roles of the nucleic acid binding domains of PEG10 in RNA binding, eCLIP in N2a cells was performed after transient transfection with HA-tagged Peg10, as well as the previously described zinc finger and reverse transcriptase mutants (FIGS. 79E-79F). Compared to the control, PEG10 strongly bound a number of mRNAs in N2a cells including its own mRNA (FIG. 73C), as well as Ddit4, a gene known to inhibit neuronal differentiation and myelination (21), and Ywhag, a gene encoding a 14-3-3 family protein, a key regulator of protein interactions, which also plays a role in neuronal migration (22). Both the NC and the RT domains are required for the binding of these mRNAs by PEG10 (FIGS. 73D, and 79G).

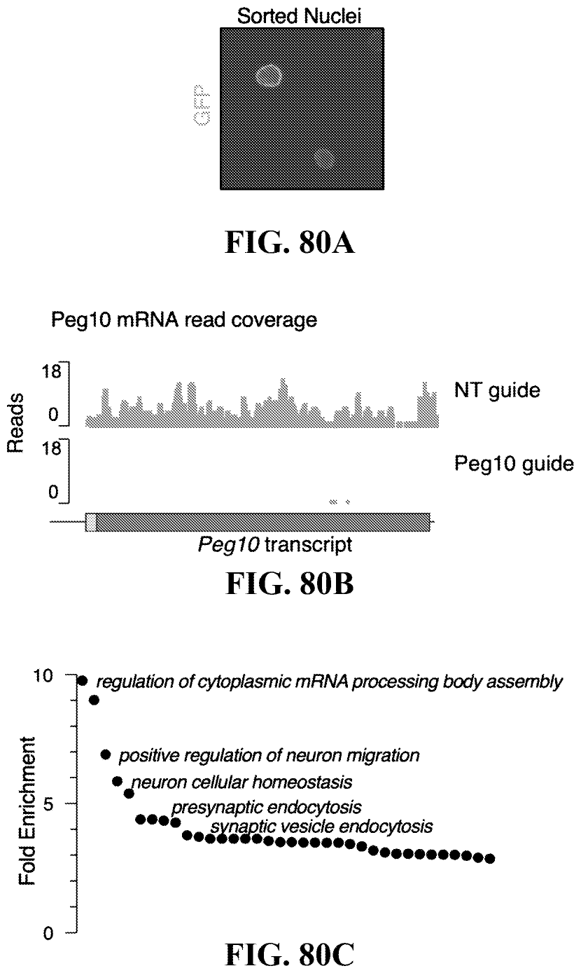

To investigate the role of PEG10 in the native context, Peg10 gene expression in the postnatal mouse brain was perturbed and expression changes of PEG10 bound transcripts was assessed. To this end, we injected AAV-PHP.eB encoding a neuron specific expression of nuclear membrane GFP protein (GFP-KASH) and sgRNAs against Peg10 into P1 Cas9 mice (FIG. 73E). After 24 days, cells were harvested and FACS for GFP+ nuclei was performed (FIG. 80A) from the frontal cortex of the Peg10 knockout mice and non-target control mice. Next generation sequencing for indels in the Peg10 locus confirmed robust gene knockout with 55-70% INDELs (FIG. 73F), and mRNA sequencing showed a near complete loss of reads mapping to the Peg10 locus (FIG. 80B). In cortical neurons, many genes are significantly downregulated and a few genes are upregulated in PEG10 knockout nuclei compared to the control sorted nuclei (FIG. 73G). Pathway analysis of these genes shows significant enrichment of downregulated genes in pathways responsible for cytoplasmic RNA processing, as well as pathways involved in neuron migration, homeostasis, and synaptic endocytosis (FIG. 80C).

By comparing the mRNA sequencing to in vivo eCLIP dataset, it was found that the mRNAs of 49 genes downregulated in the brain upon Peg10 knockdown are bound to PEG10 in the similarly aged mouse brain (FIG. 73H), in agreement with the hypothesis that one of the functions of PEG10 is to bind and stabilize mRNAs. Finally, to determine whether PEG10 mediates secretion of any of these bound mRNAs, RNA sequencing was performed on VLPs from transiently transfected N2a cells. The results of this experiment confirmed the CRISPR activation results by showing that PEG10 VLPs only contain detectable amounts of the Peg10 mRNA (FIG. 73I).

Based on the results of the dissection of the PEG10 polypeptide, a specific role for each domain was proposed. The capsid domain is responsible for forming the capsid-like protein shell around the encapsidated mRNA, which is bound and packaged by the nucleocapsid domain. The protease is responsible for auto-processing of the PEG10 polyprotein, whereas the RT domain that is not required for PEG10 mRNA packaging is responsible for binding, and, possibly, stabilization of other mRNAs. Based on these similarities to commonly used viral vectors, it was surmised that PEG10 could be reprogrammed to deliver cargo mRNAs other than its own mRNA.

Given that PEG10 specifically binds its own mRNA, primarily in the 5′ and 3′ UTRs (FIG. 73B), and thus mediates its efficient secretion (FIG. 73I), we tested whether a cargo mRNA (cargoRNA) consisting of both the 5′ and 3′ UTR of PEG10 flanking a gene of interest would be efficiently packaged, secreted, delivered, and translated in the target cells. As previously shown, this approach is efficient for the Ty3 retroelement (FIG. 74A)(23). The Cre recombinase coding sequence was first flanked with the mouse PEG10 UTRs and mouse PEG10 was co-transfected with this Cre packaging plasmid with and without the Vesicular Stomatitis Virus envelope protein, VSV-G. It was found that PEG10 VLPs pseudotyped with VSV-G were able to mediate transfer of Cre mRNA into target loxP-GFP reporter N2a cells in a VSV-G and PEG10 UTR dependent manner (FIGS. 74B-74C). This result suggests that addition of the UTRs to a cargo mRNA enables packaging of that mRNA into VLPs, and that VLP depends on an appropriate fusogenic protein for cell entry.

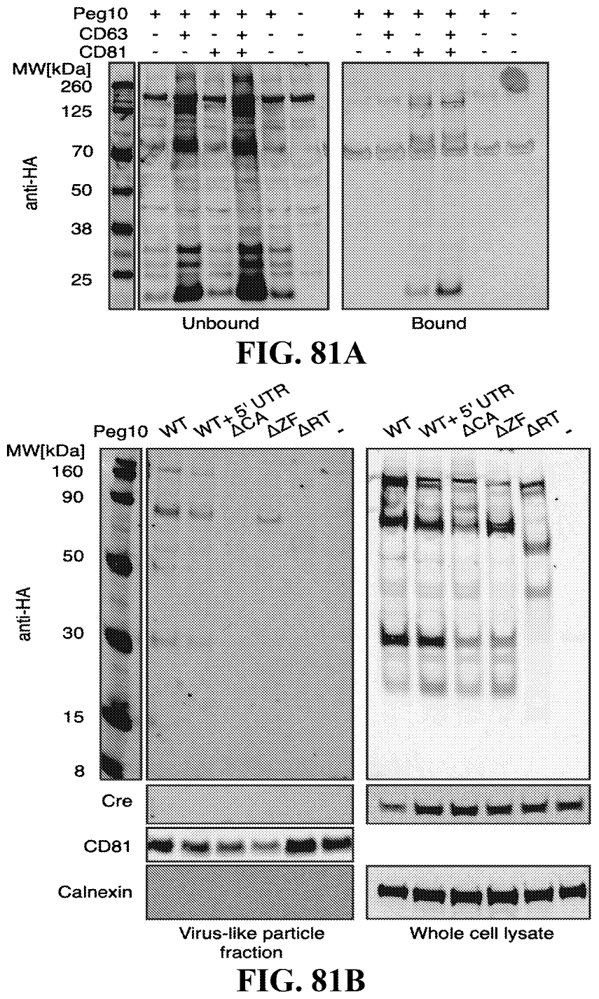

Additionally, the role observed for a fusogen in the Example implies that PEG10 VLPs are membrane bound, similar to retroviral virions. The membrane association of PEG10 was further supported by immunoprecipitation of PEG10 VLPs with anti-CD63 microbeads, a common membrane marker for extracellular vesicles, which showed the presence of PEG10-HA in the membrane-bound exosome fraction (FIG. 81A). This phenomenon was observed only when cells were co-transfected with mouse CD63 and CD81. Tetraspanins, such as CD63 and CD81, facilitate the exit of some viruses, such as HIV, from the host cell (24), and are likely to exert the same effect on PEG10 VLPs. Additionally, to ensure that PEG10 VLPs transferred mRNA rather than protein, we blotted the VLPs for HA as well as Cre recombinase, calnexin, and CD81 (FIG. 81B). The VLP fraction contained HA tagged PEG10 and CD81, but not Cre recombinase or calnexin, a marker of cell contamination. In contrast, Calnexin, Cre, and PEG10 were readily detectable in the whole cell lysate.

The 3′ UTR of Peg10 is approximately 4 kB in length, but eCLIP data showed that only portions of the 3′ UTR were actually bound by PEG10. Constructs containing the Peg10 5′ UTR, Cre, and 500 bp segments of the PEG10 3′ UTR were created to determine if there is a minimal UTR packaging signal for mediating efficient packaging and functional transfer. It was found that the proximal 500 bp of the PEG10 3′ UTR are necessary and sufficient for efficient functional transfer of Cre mRNA into target reporter cells (FIGS. 74C-74D). Interestingly, although the 5′ UTR seems to be the portion of the transcript most strongly bound (FIG. 73B), it appears to only marginally increase functional transfer of the mRNA, from ˜25% to ˜35% (FIG. 74D). Importantly, PEG10 mRNA packaging is highly specific to RNAs containing the Peg10 UTRs. No efficient functional mRNA transfer was observed for non-UTR flanked Cre or for Cre without the proximal 500 bp of the 3′ UTR (FIGS. 74C-74D).

Although both UTRs of the Peg10 mRNA are bound by the PEG10 protein, eCLIP data indicates that there is also binding inside the Peg10 coding sequence (FIG. 73B). It was hypothesized that this affinity led to decoy binding and packaging of the Peg10 mRNA self-transcript from our overexpression constructs rather than of the Cre cargoRNA, thereby reducing the efficiency of transfer. Six Peg10 overexpression constructs were generated, each with an overlapping 500-700 bp of the sequence with codons swapped to eliminate any potential PEG10-binding mRNA sequences in the mRNA. Functional transfer was increased as a result of codon swapping in the portion of the coding sequence on the boundary between the nucleocapsid and the protease domain (FIG. 74E), which corresponds to the PEG10-bound region in the eCLIP experiments. In contrast, codon swapping in other portions of the transcript reduced functional transfer (FIG. 74E), suggesting that additional elements of the mRNA sequence might affect packaging and/or expression.

Combining the parameters that were found to enhance the transfer of functional mRNA, we produced VLPs with Peg10v.4 (FIG. 74E) and an optimized packaging plasmid and detected a substantial, up to 60%, increase in functional transfer (FIG. 74F). This result suggests that, by reducing self-packaging and providing optimal packaging sequences, an endogenous Gag system, such as Peg10, can be engineered for efficient mRNA delivery. When compared with a VSV-G pseudotyped lentivirus delivering Cre recombinase under the same promoter, we observed similar levels of reporter cell activation between the lentivirus and PEG10 VLPs (FIG. 74F).

mRNA sequencing from neuronal nuclei harvested from the cortex identified about 500 genes differentially expressed (q-value ≤0.10) in animals in which Peg10 has been depleted (FIG. 74G). Of the genes significantly downregulated in Peg10 depleted neurons, many are significantly bound by PEG10 in the brain as demonstrated by the eCLIP data (FIG. 74H). mRNA sequencing of PEG10 VLPs generated by transient transfection in N2 a cells confirmed that the only Peg10 is packaged inside the VLP (FIG. 74I).

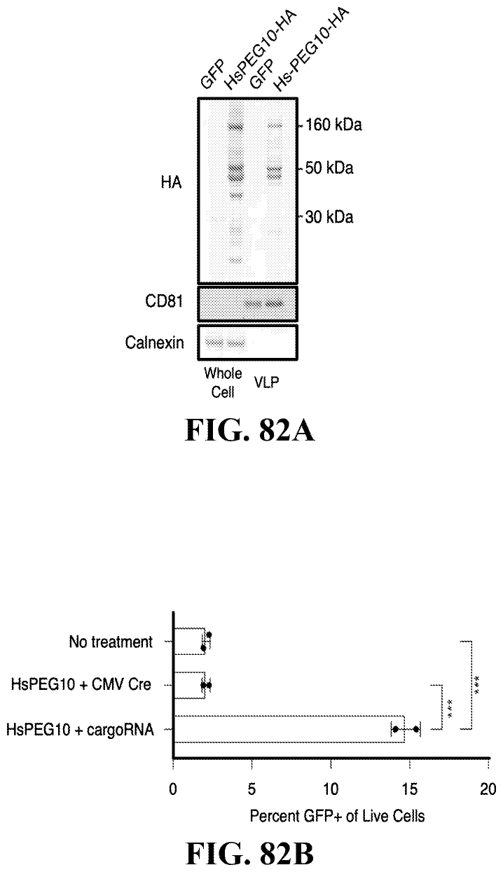

Finally, these findings were recapitulated with the human Peg10. Similar to its mouse orthologue, human PEG10 is an abundantly secreted protein (FIG. 82A). Using the same approach, the mouse PEG10 was employed and up to 18% functional transfer was achieved with the human PEG10. Similarly, the human system is highly specific, requiring PEG10 UTR flanks for mRNA packaging whereas non-flanked Cre produced only minimal reporter cell activity (FIG. 82B).

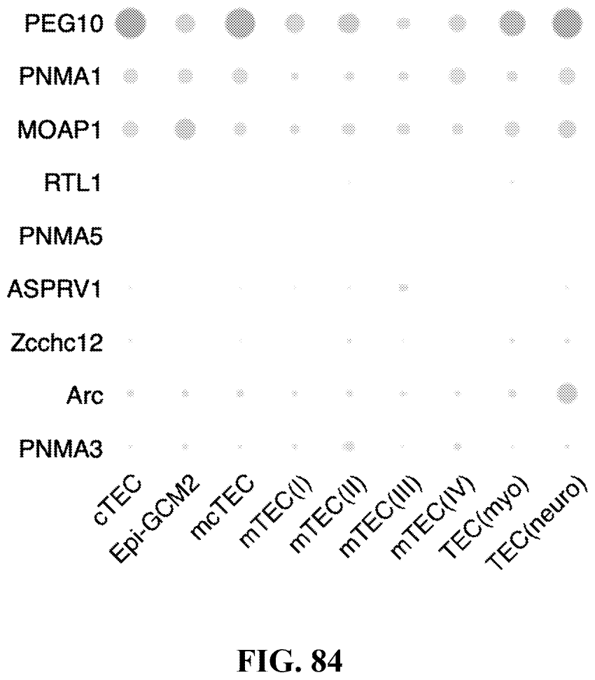

In summary, the diversity of endogenous retrovirus-derived CA-domain containing proteins in the human and mouse genomes was explored and it was found that several such proteins produced secreted VLPs, of which PEG10 was by far the most abundant. It was further shown that the PEG10 VLPs specifically encapsidated PEG10 mRNA and that the encapsidation of this mRNA depended on its UTRs. The roles of each of the PEG10 domains in VLP formation and mRNA encapsidation was identified. This information was harnessed to re-engineer PEG10 and develop a system called Selective Endogenous eNcapsidation for cellular 1Delivery (SEND) that enables functional and efficient transfer of cargoRNA. The PEG10 VLPs are likely to have reduced immunogenicity compared to the currently available viral vectors due to the use of endogenous Gag proteins. Exploration of gene expression in the developing human thymus shows that Peg10 is highly expressed compared to other CA-containing genes in the thymic epithelium (FIG. 84), which is responsible for T cell tolerance induction. Thus, PEG10-specific T cells might be selected against in the human thymus although further studies are needed to investigate the immunogenicity of the PEG10 VLPs.

The physiological function of the PEG10 VLPs that, similarly to an actual virus, specifically encapsidate the Peg10 mRNA itself remains to be investigated. The observation that PEG10 binds a variety of mRNAs with important functions in neurons and might stabilize these molecules seems to suggest a major regulatory role for PEG10, in agreement with the embryonic lethality of the Peg10 knockouts. The VLPs can serve to deliver PEG10 mRNA to cells that do not actively express this gene. It was showed that functional mRNA transfer by PEG10 VLPs requires a fusogenic protein. Conceivably, fusogens can be readily swapped to direct particle tropism. Further studies will investigate the biodistribution, nucleic acid cargo capacities, and immunogenicity of systemically delivered Peg10 VLPs.

### Materials and Methods

### Cell Culture

Human embryonic kidney cells (HEK-293FT, ThermoFisher R70007) were maintained at 37 C with 5% CO₂ in DMEM+GlutaMAX (Gibco) supplemented with 10% fetal bovine serum (VWR Seradigm 1500-500) and 1% penicillin/streptomycin (Gibco). Neuro-2A (N2a, ATCC #CRL-131) cells were maintained similarly to HEK293FT cells except in the absence of penicillin/streptomycin. HEK and N2a cells at 80-90% confluence were transfected with Lipofectamine 3000 (ThermoFisher, L3000001) according to manufacturer's guidelines. Mouse primary cortical neurons (ThermoFisher, A15585) were cultured according to manufacturer's guidelines.

### Plasmids

A list of relevant plasmids can be found in Supplementary Table S3. All coding sequences of mouse orthologs were amplified from a mouse embryonic stem cell cDNA library with PCR using gene-specific primers designed from the mm10 annotation. The cDNA library was prepared from a P30 mouse brain using the Protoscript II First Strand cDNA synthesis kit (NEB, E6300S). RNA from mouse brains were purified using the Directzol RNA Miniprep kit (Zymo, R2061). Plasmids were cloned using PCR amplification with Phusion Flash 2× Master Mix (Thermo Fisher Scientific F548S) and assembled with Gibson Assembly 2× Master Mix (NEB, E2611L), or blunt-end ligated with KLD Enzyme Mix (NEB, M0554S).

### Western Blot

Whole cell lysate of cultured HEK293FT and N2a cells were harvested using M-PER Mammalian Protein Extraction Reagent (ThermoFisher, 78501) with Halt Protease Inhibitor Cocktail (ThermoFisher, 78429). Harvested mouse brain tissue was homogenized using a glass dounce homogenizer in M-PER containing Protease Inhibitor Cocktail. Harvested mouse plasma was prepared from blood collected by cardiac puncture and spun with 5 μL of 0.5M EDTA (ThermoFisher, AM9260G). Plasma particles were precipitated using the Total Exosome Isolation Kit, according to the manufacturer's guidelines (ThermoFisher, 4484450). Protein concentrations were analyzed with the Pierce 660 nm Protein Assay Reagent (ThermoFisher, 22660) by Nanodrop. Protein samples were loaded onto 4-12% Bis-Tris gels (ThermoFisher, NWO4125) and electrophoresis was performed according to manufacturer's guidelines. Proteins were transferred to PVDF membranes using the iBlot Transfer Stack (ThermoFisher, 1B401031). Blots were blocked for 1 hour in Intercept (TBS) Blocking Buffer (LI-COR, 92760001), incubated with primary antibodies in Intercept (TBS) Blocking Buffer overnight at 4° C., washed 3× in 1×TBST, incubated with secondary antibodies in Intercept (TBS) Blocking Buffer for 1 h at room temperature, washed 3× in 1×TBST, then imaged with BioRad ChemiDoc XRS+. Quantification was performed using ImageJ Western blot analysis toolkit.

### RT-qECR

For all RT-qPCR experiments, total RNA was extracted with TRI Reagent (Zymo Research, R2050-1-200) and purified using the Direct-zol RNA Microprep Kit (Zymo Research, R2061). Total RNA was reverse transcribed using the Agilent cDNA qPCR Synthesis Kit (Agilent, 600559) according to manufacturer's guidelines. Gene-specific primers used for RT-qPCR are shown in Table 12. PCR was performed using Fast SYBR Green Master Mix (ThermoFisher, 4385610) on a Roche Lightcycler 480 according to manufacturer's guidelines.

Purification of VLPs from Cell Culture Media

Unless otherwise noted, cell culture media containing extracellular VLPs were harvested, clarified by low centrifugation at 2000 g for 10 minutes, filtered through a 0.45 μm Durapore PVDF filter (EMD Millipore, #SE1M003M00), and ultracentrifuged at 120,000×g g for 2 h at 4 C in a Beckman Coulter Optima XPN-80 ultracentrifuge. The supernatant was decanted, and the remaining pellet was resuspended up to 100 uL 1×PBS. To remove DNA and RNA not protected by a vesicle, purified VLPs were treated with 2.5 μL Micrococcal Nuclease (NEB, M0247S) in 1× Micrococcal Nuclease buffer and 100 ug/mL BSA for 2 h at 37° C. Micrococcal nuclease was then inactivated by the addition of EDTA to a final concentration of 10 mM. RNA was extracted with Tri reagent using the Direct-zol RNA Microprep Kit.

### Mice

Wildtype C57BL/6J-Elite mice were obtained from Charles River Laboratories. Cas9 mice were obtained from a breeding colony maintained by our group (25). All housing, breeding, and procedures were performed according to protocols approved by the Institutional Animal Care and Use Committees (IACUC) of the Broad Institute of MIT and Harvard.

All animal line generation was performed at Harvard Genome Modification Facility (GMF). Briefly, Cas9 RNPs and ssDNA donors with the HA tag and 130 bp of homology were injected into wild type C57BL/6J embryos. Neonates were genotyped for correct insertions using NGS on DNA isolated from ear clippings (Lucigen; QE09050).

### Homology-Based Mining of Retroviral Proteins

In order to obtain a comprehensive census of Gag homologs encoded in human and mouse genomes, we ran HHpred search with a multiple alignment of mammalian orthologs of the human Arc protein employed as the query (https://toolkit.tuebingen.mpg.de/tools/hhpred (PMID: 33315308). In addition to the highly significant hits in the human proteome, this search retrieved the homologous domains from the Pfam database: Gag-p24, Gag-p30, gag-gag2, SCAN, PNMA, DUF1759, and DUF4219. Although all these domains are homologous, they belong to two different Pfam clans, namely, clan GAG-polyprotein (CL0523) and clan Viral_Gag (CL0148). HHpred searches were repeated using multiple alignments of each of these Pfam domains as queries. These seed alignments were also used to build HMM profiles (PMID: 22039361). These profiles and the profile for mammalian Arc orthologs were used to search the human proteome (ANNOTATION_RELEASE_109) and the mouse proteome (ANNOTATION_RELEASE_106), from NCBI ftp site https://www.ncbi.nlm.nih.gov/genome/annotation_euk/Mus_musculus/106 and https://www.ncbi.nlm.nih.gov/genome/annotation_euk/Homo_sapiens/109/]. In order to confirm the significant hits produced by these search as Gag homologs they were used as queries for reciprocal searches of the Pfam (PMID: 30357350), SMART (PMID: 33104802), and CDD (PMID: 31777944) databases. The confirmed Gag homologs encoded in the human and mouse genomes with apparent orthologous relationships among them are listed in Table 9. Additional confirmed Gag homologs encoded in the mouse genomes are listed in Table 10. Importantly, retroviruses integrated into mammalian genomes in multiple waves because of which one to one orthologous relationships can be demonstrated only for a subset of retrovirus-derived genes.

TABLE 10
Summary of additional proteins with domains homologous to Gag capsid protein
in mouse based on sequence alignments. In addition to those listed in Table 9, 48 additional
proteins with domains homologous to Gag capsid protein are identified in mouse.
Candidates (Mus musculus)

| Identified protein | Domain | Accession |
|---|---|---|
| protein LDOC1 | DUF4939 | NP_001018097.1 |
| PREDICTED: agouti-signaling protein isoform X1 | Gag_p24 | XP_011237991.1 |
| gag protein | Gag_p24 | AAC12789.1 |
| Gag | Gag_p24 | AAC52922.1 |
| BC005685 protein, partial | Gag_p24 | AAH05685.1 |
| unnamed protein product | Gag_p24 | BAC38137.1 |
| gag | Gag_p24 | BAC79170.1 |
| gag | Gag_p24 | BAF81988.1 |
| TPA_exp: gag protein | Gag_p24 | DAA01924.1 |
| TPA_exp: gag protein | Gag_p24 | DAA01925.1 |
| TPA_exp: gag protein | Gag_p24 | DAA01928.1 |
| mCG142377, partial | Gag_p24 | EDL00544.1 |
| PREDICTED: uncharacterized protein LOC108167332 | Gag_p24 | XP_011239845.1 |
| IgE-binding protein | Gag_p24 | sp\|P03975.1\|IGEB_ |
| | | MOUSE |
| mCG1044120, partial | Gag_p24 | EDL07694.1 |
| PREDICTED: endogenous retrovirus group K member | Gag_p24 | XP_011245081.1 |
| 24 | | |
| PREDICTED: endogenous retrovirus group K member 8 | Gag_p24 | XP_017167946.1 |
| Gag polyprotein-like | | |
| gag-myb protein, partial | Gag_p30 | AAA39784.1 |
| putative | Gag_p30 | AAA51041.1 |
| Gag-Pol polyprotein | Gag_p30 | AAB06450.1 |
| gag protein | Gag_p30 | AAN46638.1 |
| truncated polyprotein | Gag_p30 | AAY27069.1 |
| gag po211tiliztein pr65 | Gag_p30 | ABD14432.1 |
| gag-pro-pol polyprotein | Gag_p30 | ABD14433.1 |
| gag polyprotein pr65 | Gag_p30 | ABD14435.1 |
| gag-pro-pol polyprotein | Gag_p30 | ABD14436.1 |
| glyco-gag polyprotein | Gag_p30 | AID54952.1 |
| gag polyprotein | Gag_p30 | AID54953.1 |
| gag-pro-pol polyprotein | Gag_p30 | AID54954.1 |
| gag, partial | Gag_p30 | AMK48512.1 |
| putative gag-pro-pol polyprotein | Gag_p30 | ARB03507.1 |
| unnamed protein product | Gag_p30 | BAC41106.1 |
| unnamed protein product | Gag_p30 | BAC41107.1 |
| truncated gag-pro-pol polyprotein | Gag_p30 | CCD57102.1 |
| gag-pro-pol polyprotein | Gag_p30 | CCD57104.1 |
| gag protein | Gag_p30 | CCD57105.1 |
| mCG144922, isoform CRA_b, partial | Gag_p30 | EDL00999.1 |
| LOC72520 protein, partial | Gag_p30 | AAH21868.1 |
| BC040756 protein, partial | Gag_p30 | AAH40756.1 |
| LOC72520 protein, partial | Gag_p30 | AAH44668.2 |
| PREDICTED: uncharacterized protein LOC108167440 | Gag_p30 | XP_017167935.1 |
| isoform X1 | | |
| PREDICTED: uncharacterized protein LOC108167440 | Gag_p30 | XP_017167936.1 |
| isoform X2 | | |
| PREDICTED: uncharacterized protein LOC108167440 | Gag_p30 | XP_017167937.1 |
| isoform X3 | | |
| unnamed protein product, partial | PNMA | BAC37719.1 |
| mCG1050067, isoform CRA_a | PNMA | EDL42061.1 |
| coiled-coil domain-containing protein 8 homolog | PNMA | NP_001095005.1 |
| predicted gene, 42372 | PNMA | NP_001357780.1 |
| PREDICTED: paraneoplastic antigen Ma2 homolog | PNMA | XP_011249051.1 |

Protein Production. Purification and Size-Exclusion Chromatography

Gag proteins cloned from mouse cDNA were subcloned into a TwinStrep-SUMO expression backbone and transformed into Rosetta™ 2(DE3) (Millipore Sigma, 71400). Overnight cultures were inoculated with single colonies and grown. The following day 1 mL of overnight culture was used to inoculate 50 mL of Terrific Broth (TB) medium. Cultures were grown at 37 C until O.D. 0.6-0.7 and induced with 0.3 mM IPTG at 25 C for 16-18 hours. Cultures were spun down and resuspended in 50 mM Tris-HCl, pH 8.0 (Invitrogen), 500 mM NaCl (Millipore Sigma), 1 mM DTT (Millipore Sigma) with the addition of Complete™ Protease Inhibitor Cocktail (Roche). Bacteria were lysed with 25 kpsi of pressure on an LM-20 Microfluidizer (Microfluidics International Corporation). Insoluble components were spun at 20,000×g for 30 mins at 4 C and the supernatant was decanted and 0.45 um filtered. Protein was isolated by flowing over Strep Tactin Superflow Plus resin (Qiagen, 30004) followed by 5 C.V. washing with 50 mM Tris-HCl, pH 8.0, 500 mM NaCl. Protein was eluted with 50 mM Tris-HCl, 500 mM NaCl, 2.5 mM desthiobiotin. The resulting proteins were cleaved overnight with homemade SUMO protease to remove tags and size excluded into 50 mM Tris-HCl, 150 mM NaCl on an ÅKTA Pure system using a Superdex 200 Increase 10/300 GL column (Cytiva).

### Electron Microscopy

All electron microscopy experiments were performed at MIT's Koch Nanotechnology Materials Core Facility. In sample preparation for cryo-electron microscopy, 3 uL of sample and buffer containing solution was dropped on a lacey copper grid coated with a continuous carbon film and blotted to remove excess sample without damaging the carbon layer by Gatan Cryo Plunge III. Grid was mounted on a Gatan 626 single tilt cryo-holder equipped in the TEM column. The specimen and holder tip were cooled down by liquid-nitrogen, which keeps maintaining during transfer into the microscope and subsequent imaging. Imaging on a JEOL 2100 FEG microscope was done using a minimum dose method that were essential to avoid sample damage under the electron beam. The microscope was operated at 200 kV and with a magnification in the range of 10,000-60,000 for assessing particle size and distribution. All images were recorded on a Gatan 2k×2k UltraScan CCD camera.

In sample preparation for negative stain-electron microscopy, 7 uL of sample and buffer containing solution was dropped on a 200 meshes copper grid coated with a continuous carbon film and waited for 60 seconds and removed excess solution by touching the grid with a kimwipes and then 10 uL of negative staining solution, phosphotungstic acid, 1% aqueous solution was dropped on the TEM grid and immediately removed it by kimwipes and 10 uL of the stain is then applied to the grid and after 40 seconds, the excess stain is removed by touching the edge with kimwipes. Finally, dried the grid at room temperature. After that, the grid was mounted on a JEOL single tilt holder equipped in the TEM column. The specimens were cooled down by liquid-nitrogen and imaging on an JEOL 2100 FEG microscope was done using minimum dose methods that were essential to avoid sample damage under the electron beam. The microscope was operated at 200 kV and with a magnification in the range of 10,000-60,000 for assessing particle size and distribution. All images were recorded on a Gatan 2k×2k UltraScan CCD camera.

### CRISPRa of CA-Containing-Like Genes in N2a Cells

Guides were selected from the mouse SAM V1 guide library (13) for each of the screened CA-containing genes (Table 11). Guides were annealed and cloned via golden gate into the UniSAM addgene plasmid (Addgene, 99866) as covered in previous publications from our lab (13).

Plasmids were transfected into N2a cells at 80% confluency in 1× T225 flasks per replicate per condition. Cell culture media was replaced with fresh media 5 h post-transfection. VLPs were harvested by filtration and ultracentrifugation 48 hours after transfection as indicated above, treated with micrococcal nuclease, and lysed in trizol, as covered in the previous section.

TABLE 11
List of reagents used in Example 5.
Antibodies

| Items | Vendor | Dilution (for WB) |
|---|---|---|
| anti HA (mouse) | Cell Signaling, #2367 | 1:1000 |
| anti HA (rabbit) | Abcam, ab910 | 1:1000 |
| anti CD81 | Santa Cruz, sc-166029 | 1:500 |
| anti beta-Actin | Abcam, ab8227 | 1:5000 |
| anti Moap1 | Sigma, SAB1411249 | 1:1000 |
| anti Peg10 | Proteintech, A4412 | 1:1000 |
| anti Rtl1 | ThermoFisher, PA566887 | 1:1000 |
| IRDye 680RD | LI-COR 926-68072 | 1:10,000 |
| Donkey anti- | | |
| Mouse IgG | | |
| (H + L) | | |
| IRDye 680RD | LI-COR 926-68073 | 1:10,000 |
| Donkey anti- | | |
| Rabbit IgG | | |
| (H + L) | | |
| IRDye® 800CW | LI-COR 926-32211 | 1:10,000 |
| Goat anti-Rabbit | | |
| IgG Secondary | | |
| Antibody | | |

Plasmids

| Items | Source | Application |
|---|---|---|
| PB-UniSAM | Addgene, 99866 | CRISPR A |
| pUCmini-iCAP- | Addgene, 103005 | CRISPR KO |
| PHP.eB | | |
| CAG-GFP-IRES- | Addgene, 48201 | Cre cargoRNA cloning |
| CRE | | |
| pHelper | pHelper was a gift from CRUK | AAV production |
| PX552 | Addgene, 60958 | AAV production |
| pMD2.G | Addgene, 12259 | Lentivirus |
| psPAX | Addgene, 12260 | Lentivirus |
| RV-Cag-Dio- | Addgene, 87662 | Cre reporter N2a cell |
| GFP | | line |

Oligos

| Target | Sequence | Application |
|---|---|---|
| Asprv1 | F: AGGAACCCTGGGGGCCCA (SEQ ID NO: 64) | Gibson assembly of |
| | R: GTGGGAGCCCTCCGGTGC (SEQ ID NO: 65) | the coding sequence |
| Moap1 | F: ACACTGAGACTTCTAGAAGACTGG (SEQ ID NO: 66) | from species-specific |
| | R: AGTGCAATAGCCTTCTAATTCG (SEQ ID NO: 67) | cDNA library into |
| Pnma1 | F: GCTATGACACTATTGGAAGACTGGTGC (SEQ ID NO: 68) | mammalian |
| | R: GAAGTGCCCCTCCAGGCC (SEQ ID NO: 69) | overexpression vectors |
| Arc | F: GAGCTGGACCATATGACCACC (SEQ ID NO: 70 | |
| | R: TTCAGGCTGGGTCCTGTCACT (SEQ ID NO: 71) | |
| Zcchc12 | F: GCTAGCATCCTTTCACGTTTGG (SEQ ID NO: 72) | |
| | R: CTGTGGTTCAGATAGGCCAATG (SEQ ID NO: 73) | |
| Pnma3 | F: ATGAAACAGCGAAGGAAGCCTC (SEQ ID NO: 74) | |
| | R: ATGTGCTGGATGCAGTGGCT (SEQ ID NO: 75) | |
| Pnma5 | F: GCCGTGGCTCTATTAGATGA (SEQ ID NO: 76) | |
| | R: CTCACGAAAGGACTCAAGGG (SEQ ID NO: 77) | |
| Pnma6 | F: GTTATCACATTCCTCCAGGACG (SEQ ID NO: 78) | |
| | R: ATGGCGGTGACCATGCTG (SEQ ID NO: 79) | |
| Peg10 | F: GCTGCTGCGGGTGGTTCC (SEQ ID NO: 80) | |
| | R: CGCAGCACTGCAGGATGA (SEQ ID NO: 81) | |
| Rtl1 | F: GATAGAACCCTCTGAAGACT (SEQ ID NO: 82) | |
| | R: GTCAAGTTCATCATCTGAGT (SEQ ID NO: 83) | |
| PEG10 | F: GCTGCTGCGGGTGGTTCC (SEQ ID NO: 84) | |
| | R: CGCAGCACTGCAGGATGA (SEQ ID NO: 85) | |
| Peg10 | F: GCAGCCCCTATCCCAAACTT (SEQ ID NO: 86) | RT-qPCR |
| | R: CGATCAGCATGCTTGTCACG (SEQ ID NO: 87) | |
| Peg10 CDS | F: GCTTCTGGTGCATCTGGCAAC (SEQ ID NO: 88) | Mutagenesis and |
| A1468C | R: AATCATAGCTCGGACAAACAGGGT (SEQ ID NO: 89) | cloning via KLD |
| (for PEG10 | | enzyme mix |
| D491A) | | |
| Peg10 ΔNC | F: CCAGCGAAAGCCTCCAAG (SEQ ID NO: 90) | |
| | R: CAAATTCATTTTGCGGCGTCTC (SEQ ID NO: 91) | |
| Peg10 ΔRT | F: TGTGCCTGTTGTAATCACCTGGTCT (SEQ ID NO: 92) | |
| | R: CATGTGGTAGAAGAATGGTGGCTG (SEQ ID NO: 93) | |
| Peg10 | F: TGTTTACAGTGCCACAACCGAATT (SEQ ID NO: 94) | Indel sequencing |
| | R: AGATGCTCATGCTGATCTGGAG (SEQ ID NO: 95) | |

CRISPR-mediated perturbations

| | Sequence | |
|---|---|---|
| Target | (of spacers unless noted otherwise) | Application |
| Asprv1 | AGGTGTCCCGTAGGTACTGA (SEQ ID NO: 96) | CRISPRa |
| Asprv1 | GGGTGGAGCTTCTAGAACAA (SEQ ID NO: 97) | |
| Arc | GCGAGTAGGCGCGGAAGGCG (SEQ ID NO: 98) | |
| Arc | GGCCCGTGGGCGGCAGCTCG (SEQ ID NO: 99) | |
| Peg10 | AGCGTGCTTCGCGAGCAGCG (SEQ ID NO: 100) | |
| Peg10 | CGCTGCTCGCGAAGCACGCT (SEQ ID NO: 101) | |
| Rtl1 | GGGCGCGGCATGCACTGCTT (SEQ ID NO: 102) | |
| Rtl1 | AGCAATTTAGGTTCTCAAGA (SEQ ID NO: 103) | |
| Peg10 | TGCAGATGCTGATGCATATG (SEQ ID NO: 104) | CRISPR KO |
| Peg10 | TCTGTATCCGGTTATGCACC (SEQ ID NO: 105) | |
| Non-targeting | GCTTTCACGGAGGTTCGACG (SEQ ID NO: 106) | CRISPRa and CRISPR |
| Non-targeting | ATGTTGCAGTTCGGCTCGAT (SEQ ID NO: 107) | KO |
| Peg10 | AGAGGGGCTTCACTCCCCTG (SEQ ID NO: 108) | CRISPR Knock-in |
| ssDNA donor for | GCTAATAGCGACTGCTCTGAATGAATATGTT | |
| knock-in | GAATGTATGCTTCTGTTGTCATTTACAGGAAC | |
| | AGGCGGGTTTTAAGAACCAAAAGACGCCAAC | |
| | CACGAGGGTCCCAGGATCCAGGGCTCCCTCC | |
| | CCAGGCCACCATGTATCCCTATGACGTGCCC | |
| | GATTATGCCGCTGCTGCGGGTGGTTCCTCCA | |
| | ACTGCCCGCCCCCTCCCCCTCCCCCTCCTCCC | |
| | AACAACAACAACAACAACAACACCCCAAAG | |
| | AGCCCAGGCGTGCCTGACGCCGAAGATGATG | |
| | ATGAACGCAGACACG (SEQ ID NO: 109) | |

### CO-IP Mass Spectrometry

N2a cells cultured in 1× T225 flasks per replicate were transiently with lipofectamine 3000 with N-terminal and C-terminally HA tagged-Peg10 overexpression plasmids. Cells were lysed and PEG10 protein was immunoprecipitated using the Pierce™ HA-Tag Magnetic IP/Co-IP Kit (Thermofisher; 88838). Briefly, cells were lysed in the Pierce kit IP lysis buffer and rotated at room temperature for 30 minutes with 25 μL of Pierce HA magnetic beads. Protein was eluted from the beads using the kit's acidic elution buffer and neutralized.

### In-Gel Protein Digestion

Gel bands were cut out, reduced with 5 mM DTT and alkylated with 10 mM iodoacetamide and digested with either trypsin at 37 C essentially as described previously (26).

### Immunoprecipitation Protein Digestion

Beads were digested according to the S-Trap Micro Spin Column Digestion Protocol (www.protifi.com).

### LC-MS/MS Analysis for Gelbands

The dried peptide mix was reconstituted in a solution of 2% formic acid (FA) for MS analysis. Peptides were loaded with the autosampler directly onto a 50 cm EASY-Spray C18 column (ES803a, Thermo Scientific). Peptides were eluted from the column using a Dionex Ultimate 3000 Nano LC system with a 14.8 min gradient from 1% buffer B to 23% buffer B (100% acetonitrile, 0.1% formic acid), followed by a 0.2 min gradient to 80% B, and held constant for 0.5 min. Finally, the gradient was changed from 80% buffer B to 99% buffer A (100% water, 0.1% formic acid) over 0.5 min, and then held constant at 99% buffer A for 14 more minutes. The application of a 2.2 kV distal voltage electrosprayed the eluting peptides directly into the Thermo Exploris480 mass spectrometer equipped with an EASY-Spray source (Thermo Scientific). Mass spectrometer-scanning functions and HPLC gradients were controlled by the Xcalibur data system (Thermo Scientific). MS1 scans parameters were 60,000 resolution, AGC at 300%, IT at 25 ms. MS2 scan parameters were 30,000 resolution, isolation width at 1.2, HCD collision energy at 28%, AGC target at 100% and max IT at 55 ms. 15MS/mS scans were taken for each MS1 scan. Expected peptides for Retrotransposon-derived protein PEG10 GN=Peg10 were put into an inclusion list.

### LC-MS/MS Analysis for IPs

The dried peptide mix was reconstituted in a solution of 2% formic acid (FA) for MS analysis. Peptides were loaded with the autosampler directly onto a 50 cm EASY-Spray C18 column (ES803a, Thermo Scientific). Peptides were eluted from the column using a Dionex Ultimate 3000 Nano LC system with a 3 min gradient from 1% buffer B to 5% buffer B (100% acetonitrile, 0.1% formic acid), followed by a 36.8 min gradient to 25%, and a 10.2 min gradient to 35% B, followed by a 0.5 min gradient to 80% B, and held constant for 4.5 min. Finally, the gradient was changed from 80% buffer B to 99% buffer A (100% water, 0.1% formic acid) over 0.1 min, and then held constant at 99% buffer A for 19.9 more minutes. The application of a 2.2 kV distal voltage electrosprayed the eluting peptides directly into the Thermo Exploris480 mass spectrometer equipped with an EASY-Spray source (Thermo Scientific). Mass spectrometer-scanning functions and HPLC gradients were controlled by the Xcalibur data system (Thermo Scientific). MS1 scans parameters were 120,000 resolution, AGC at 300%, IT at 50 ms. MS2 scan parameters were 30,000 resolution, isolation width at 1.2, HCD collision energy at 28%, AGC target at 300% and IT set to Auto. Cycle time for MS2 was 3 sec for each MS1 scan.

### Database Search

Tandem mass spect220tilize searched with Sequest (Thermo Fisher Scientific, San Jose, CA, USA; version IseNode in Proteome Discoverer 2.5.0.400). Sequest was set up to search a mouse uniprot database (database version Mar. 21, 2020; 55699 entries containing common contaminants) assuming the digestion enzyme trypsin. Sequest was searched with a fragment ion mass tolerance of 0.02 Da and a parent ion tolerance of 10.0 PPM. Carbamidomethyl of cysteine was specified in Sequest as a fixed modification. Oxidation of methionine was specified in Sequest as a variable modification. Scaffold (version Scaffold_4.11.1, Proteome Software Inc., Portland, OR) was used to validate MS/MS based peptide and protein identifications. Peptide identifications were accepted if they could be established at greater than 95.0% probability by the Percolator posterior error probability calculation (27). Protein identifications were accepted if they could be established at greater than 99.0% probability and contained at least 2 identified peptides. Protein probabilities were assigned by the Protein Prophet algorithm (28). Proteins that contained similar peptides and could not be differentiated based on MS/MS analysis alone were grouped to satisfy the principles of parsimony. Proteins sharing significant peptide evidence were grouped into clusters.

Cloning of pEG10 Variants

Structural domains within PEG10 were identified via homology detection of the full Peg10 peptide sequence against known domains via HHPred with the HHsuite v3.0 release at default settings (database: PDB_mmCIF70) [Zimmermann 2018 paper]. The protease domain in PEG10 was made inactive by mutating the catalytic aspartic acid to alanine (D491A) using oligos listed in Table 11. In-frame deletions of the NC and the RT domains were made using oligos listed in Table 11. Peg10 codon optimization was performed using the IDT codon optimization tool for human and gene blocks were ordered from IDT.

### AAV PHP.eB Production and Delivery

AAV CRISPR guides were designed using Benchling online CRISPR gRNA design tool and validated for efficiency in mouse embryos and cloned with the previously described golden gate method into PX552 (Addgene, 60958). PHP.eB AAV vectors were generated as previously described (29). In brief, HEK293 cells were grown in T225 flasks plastic dishes and transfected with the pHelper plasmid, the PHP.eB capsid plasmid, and the Px552 transgene plasmid. Five days later, cells were lysed, virus was isolated using Optiprep density-gradient medium (Sigma; D1556), and ultra-centrifuged at 350,000 g. The viral layer was isolated and concentrated using Amicon Ultra-15 centrifugal filter units (Sigma; Z648043-24EA). AAVs titre was determined using SYBR Green qPCR. Two vectors with two separate Peg10 targeting gRNAs were produced in parallel and pooled in equal titer. For in vivo administration of the virus, P1 mice were anesthetized and 5E11 viral genomes for each virus were retro-orbitally injected into in-house generated Cas9 mice. Mice are publicly available (Jackson labs, 026179).

### FACS of Neuronal Nuclei

Prefrontal cortex of P25 mice was dissected and flash frozen in liquid nitrogen. Nuclei were prepared from fresh frozen brain using the Nuclei EZ Prep kit (Sigma, NUC101), as previously described (30). Briefly, nuclei were dounced exactly 20 times in 1 mL of Nuclei lysis buffer on ice immediately after removal from dry ice and washed twice in Nuclei lysis buffer. Following prep, nuclei were counterstained with DAPI and resuspended in PBS with 0.5% BSA (ThermoFisher, 15260037). For each condition, 20,000 neuronal nuclei were sorted based on DAPI and GFP on a Sony MA900 Cell Sorter directly into Tri reagent. RNA was extracted using the Direct-zol RNA Microprep Kit and mRNA sequencing library was prepared as listed below.

mRNA-Sequencing of Nuclei, Whole Cell RNA and EVs

N2a cells, purified EVs, and sorted neuronal nuclei were lysed with TRI Reagent (Zymo Research, R2050-1-200), and total RNA was extracted using Direct-zol RNA Microprep Kit (Zymo Research, R2061) and treated with DNase I (Zymo Research, E1010). Whole cell mRNA was enriched using the NEBNext Poly(A) mRNA Magnetic Isolation Module (NEB, E7490), and the multiplexed RNA sequencing library was prepared using NEBNext Ultra II RNA Library Prep Kit for Illumina (NEB, E7775). Libraries were sequenced on the Illumina NextSeq 550 with 50 cycles for read 1 (forward), 25 cycles for read 2 (reverse) for a read coverage of approximately 15-20 million reads per sample.

Pseudo alignments and differential gene expression analysis was performed using Kallisto and Sleuth (31, 32). Full read alignments were generated using STAR and converted to indexed BAM files with SAM tools to generate read tracks.

### Indel Sequencing of Sorted Neuronal Nuclei

The target region was amplified from genomic DNA by PCR with primers described in Table 8 and barcoded with indexed Illumina P5 and P7 primers for NGS. Libraries were purified with Ampure XP (Beckman Coulter), quantified with Qubit hsDNA reagent, and sequenced on an Illumina MiSeq system (300 bp read 1). Indels were quantified from the resulting library using OutKnocker, an online tool used to detect indels from NGS libraries (33).

In Vivo and In Vitro eCLIP

Female prefrontal cortex of P30-P32 HA-tagged mice generated and bred in house were dissected and flash frozen in liquid nitrogen. To prepare the CLIP library, 300 mg of brain was pulverized with a pestle on ice and immediately UV irradiated twice at 400 mJ/cm2 (34). The tissue was then processed using an adapted eCLIP protocol (35). Briefly, tissue was resuspended in the recommended CLIP lysis buffer and sonicated for 5 minutes at low intensity on a Bioruptor Pico sonicator (Diagenode, B01080010).

For in vitro eCLIP experiments, ˜2 million N2A cells were transiently transfected in a 6-well plate in the same manner as listed above. Three days following transfection, media was changed to PBS (ThermoFisher, 10010023) and UV irradiated once at 400 mj/cm2, resuspended in the CLIP lysis buffer and scraped from the plate.

Unless otherwise stated, all methods and components for the CLIP preparation were exactly as listed previously (35). Briefly, lysed samples were nuclease treated as recommended and immunoprecipitated using Pierce Anti-HA magnetic beads (ThermoFisher, 88836). As per the protocol, immunoprecipitated samples were washed and overnight ligated with the recommended CLIP 3′ RNA adaptor. Simultaneously, a subsample was blotted for HA to determine the efficacy of the precipitation and expected band size. Following overnight ligation, samples were loaded onto a NuPAGE gel (ThermoFisher, 223 tilizes 223) and transferred onto a nitrocellulose membrane (ThermoFisher, IB23001). Bands were excised with a scalpel on a sterile bioassay plate, transferred to an eppendorf tube, and sequentially digested in Proteinase K with urea, and then Acid-Phenol:Chloroform (ThermoFisher, AM9722) on an orbital thermoshaker at 37° C. RNA was extracted and prepared for sequencing as recommended from the CLIP protocol. The samples were run on the NextSeq 550 with 50 cycles for read 1 (forward), 25 cycles for read 2 (reverse). Sequencing reads were aligned with STAR, quantified using htseq-count, and differential expression analysis was performed with DESeq2.

### Cre-Reporter Cell Line Generation

A lentiviral lox-P GFP Cre reporter cassette was generated by subcloning the loxP-GFP cassette from RV-Cag-Dio-GFP (Addgene #87662) into a lentiviral transfer plasmid encoding a blasticidin resistance gene. To produce virus HEK293FT cells were seeded at 1e7 cells per 15 cm dish. 16 hours later cells were co-transfected with 5 ug psPAX2 (Addgene #12260), 4.7 ug pMD2.G (Addgene #12259), and 7.7 ug of the Cre reporter plasmid using PEI HCl MAX (Polysciences 24765-1), media was changed 4 hours post transfection. 48 hours later viral supernatant was harvested, spun at 2000×g for 10 mins to remove cell debris, 0.45 um filtered, aliquoted, and frozen at −80 for later use.

N2a reporter cell lines were created by seeding cells at 50% confluency in 6 well plates at day 0. 1 mL of clarified virus supernatant was added to the cells along with 8 ug/mL polybrene (TR1003G). A day later media was changed, and cells were selected for two weeks starting on day 3 with 10 ug/mL Blasticidin-HCl (Thermo Fisher Scientific A1113903). Successful reporter line generation was confirmed by transfection of a Cre encoding plasmid.

### Lentivirus and VLP Production Transfer.

Both lentivirus and Peg10 VLPs packaging Cre-encoding mRNA were produced and purified in an identical manner. HEK293FT cells were seeded at a density of 4e6 cells per 10 cm dish. The next day, cells were transfected with 2 ug each of i) [vector RNA name], ii) a Peg10 overexpression plasmid or psPAX2 (Addgene #12260), and iii) pMD2.G (Addgene #12259). Cells were washed with PBS and fresh medium 4 h post-transfection. 48 hours later, the culture supernatant was harvested, and particles were purified in the same manner as the purification of EVs from the CRISPRa experiment described above.

For all transfer experiments, Cre-reporter N2a cells were plated onto 96-well plates at 5e4 cells per well one hour before transfer. 20 ul of purified VLPs of lentivirus derived from a 10 cm dish were applied to N2a cells in triplicate. Cells were incubated for 72 hours before flow cytometry analysis.

### VLP Immunoprecipitation

PEG10 VLPs were produced by co-transfection of PEG10 overexpression plasmids, pMD2.G, with and without plasmids encoding the mouse tetraspanin CD63 and CD81. VLPs were harvested as described above. Immunoprecipitations were performed for CD63 using Exosome-Human CD63 Isolation/Detection Reagent (Thermo Fisher Scientific 10606D) according to manufacturer's instructions. Immunoprecipitated VLPs were eluted in SDS-PAGE buffer by boiling and western blotted as described above with a rabbit anti-HA antibody.

### Flow Cytometry

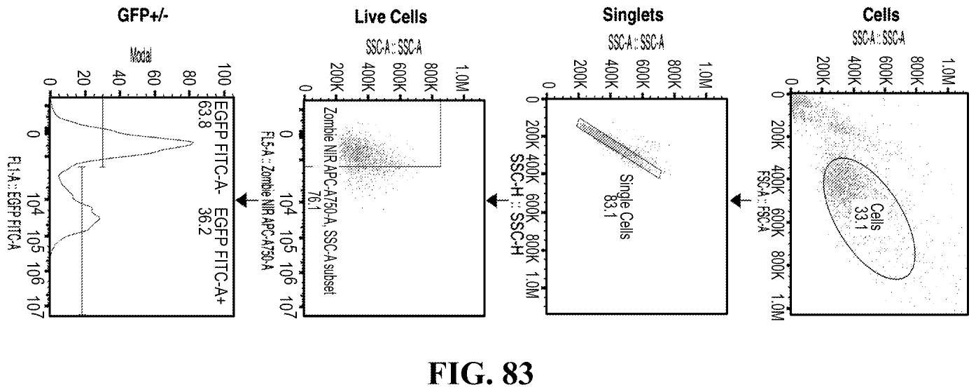

N2a cells in 96-well plates were washed once with 1×PBS, and dissociated with TrypLE (ThermoFisher, 12604013). Cells were resuspended with 2% FBS in PBS, centrifuged at 500 g for 5 min, and washed once in PBS. Cells were viability stained for 30 mins with Zombie NIR Fixable Viability Dye (Biolegend, 423105) in PBS at 4 C. Cells were washed twice in 2% FBS in PBS and resuspended in 2% FBS in PBS+2 mM EDTA for analysis on a Beckman Coulter Cytoflex S flow cytometer. Analysis was performed using FlowJo v10.7 (BD Biosciences). Representative gating schemes are shown in FIG. 83.

### Cell Imaging

N2a or HEK293FT cells were plated on glass bottom plates (Cellvis, P96-1.5H-N). Following VLP transfer experiments, cells were fixed with 4% PFA in 1×PBS for 15 min at room temperature, washed 3× with 1×PBS for 5 min each, permeabilized with 0.1% Tween in PBS for 5 min, and counterstained with DAPI (ThermoFisher, R37606). Confocal images were obtained using a Leica Stellaris 5 confocal microscope

### References for Example 5

1. J. L. Goodier, H. H. Kazazian Jr, Retrotransposons revisited: the restraint and rehabilitation of parasites. Cell. 135, 23-35 (2008).
2. P. N. Perrat, S. DasGupta, J. Wang, W. Theurkauf, Z. Weng, M. Rosbash, S. Waddell, Transposition-driven genomic heterogeneity in the Drosophila brain. Science. 340, 91-95 (2013).
3. R. Ono, K. Nakamura, K. Inoue, M. Naruse, T. Usami, N. Wakisaka-Saito, T. Hino, R. Suzuki-Migishima, N. Ogonuki, H. Miki, T. Kohda, A. Ogura, M. Yokoyama, T. Kaneko-Ishino, F. Ishino, Deletion of Peg10, an imprinted gene acquired from a retrotransposon, causes early embryonic lethality. Nat. Genet. 38, 101-106 (2006).
4. A. Dupressoir, C. Vernochet, O. Bawa, F. Harper, G. Pierron, P. Opolon, T. Heidmann, Syncytin-A knockout mice demonstrate the critical role in placentation of a fusogenic, endogenous retrovirus-derived, envelope gene. Proc. Natl. Acad. Sci. U.S.A 106, 12127-12132 (2009).
5. C. Henke, P. L. Strissel, M.-T. Schubert, M. M-chell, C. C. Stolt, F. Faschingbauer, M. W. Beckmann, R. Strick, Selective expression of sense and antisense transcripts of the sushi-ichi-related retrotransposon-derived family during mouse placentogenesis. Retrovirology. 12, 9 (2015).
6. P. Barragan-Iglesias, J. B. De La Pena, T. F. Lou, S. Loerch, Intercellular Arc signaling regulates vasodilation (2020) (available at https://papers.ssrn.com/sol3/papers.cfm?abstract_id=3684856).
7. J. Ashley, B. Cordy, D. Lucia, L. G. Fradkin, V. Budnik, T. Thomson, Retrovirus-like Gag Protein Arc1 Binds RNA and Traffics across Synaptic Boutons. Cell. 172, 262-274.e11 (2018).
8. E. D. Pastuzyn, C. E. Day, R. B. Kearns, M. Kyrke-Smith, A. V. Taibi, J. McCormick, N. Yoder, D. M. Belnap, S. Erlendsson, D. R. Morado, J. A. G. Briggs, C. Feschotte, J. D. Shepherd, The Neuronal Gene Arc Encodes a Repurposed Retrotransposon Gag Protein that Mediates Intercellular RNA Transfer. Cell. 173, 275 (2018).
9. M. Krupovic, E. V. Koonin, Multiple origins of viral capsid proteins from cellular ancestors. Proc. Natl. Acad. Sci. U.S.A 114, E2401-E2410 (2017).
10. M. Campillos, T. Doerks, P. K. Shah, P. Bork, Computational characterization of multiple Gag-like human proteins. Trends Genet. 22, 585-589 (2006).
11. M. Abed, E. Verschueren, H. Budayeva, P. Liu, D. S. Kirkpatrick, R. Reja, S. K. Kummerfeld, J. D. Webster, S. Gierke, M. Reichelt, K. R. Anderson, R. J. Newman, M. Roose-Girma, Z. Modrusan, H. Pektas, E. Maltepe, K. Newton, V. M. Dixit, The Gag protein PEG10 binds to RNA and regulates trophoblast stem cell lineage specification. PLoS One. 14, e0214110 (2019).
12. S. Erlendsson, D. R. Morado, H. B. Cullen, C. Feschotte, J. D. Shepherd, J. A. G. Briggs, Structures of virus-like capsids formed by the Drosophila neuronal Arc proteins. Nat. Neurosci. 23, 172-175 (2020).
13. S. Konermann, M. D. Brigham, A. E. Trevino, J. Joung, O. O. Abudayyeh, C. Barcena, P. D. Hsu, N. Habib, J. S. Gootenberg, H. Nishimasu, O. Nureki, F. Zhang, Genome-scale transcriptional activation by an engineered CRISPR-Cas9 complex. Nature. 517, 583-588 (2015).
14. R. Ono, S. Kobayashi, H. Wagatsuma, K. Aisaka, T. Kohda, T. Kaneko-Ishino, F. Ishino, A retrotransposon-derived gene, PEG10, is a novel imprinted gene located on human chromosome 7q21. Genomics. 73, 232-237 (2001).
15. J. Volff, C. Körting, M. Schartl, Ty3/Gypsy retrotransposon fossils in mammalian genomes: did they evolve into new cel226tilizes226ization Mol. Biol. Evol. 18, 266-270 (2001).
16. K. Shigem226tilizesBrennan, E. Walls, C. J. Watson, D. Stott, P. W. Rigby, A. D. Reith, Identification and characterisation of a developmentally regulated mammalian gene that utilises −1 programmed ribosomal frameshifting. Nucleic Acids Res. 29, 4079-4088 (2001).
17. M. Golda, J. A. Motyin, M. Mahdi, J. Tözsér, Functional Study of the Retrotransposon-Derived Human PEG10 Protease. Int. J. Mol. Sci. 21(2020), doi:10.3390/ijms21072424.
18. A. Saunders, E. Z. Macosko, A. Wysoker, M. Goldman, F. M. Krienen, H. de Rivera, E. Bien, M. Baum, L. Bortolin, S. Wang, A. Goeva, J. Nemesh, N. Kamitaki, S. Brumbaugh, D. Kulp, S. A. McCarroll, Molecular Diversity and Specializations among the Cells of the Adult Mouse Brain. Cell. 174 (2018), pp. 1015-1030.e16.
19. D. Sato, A. C. Lionel, C. S. Leblond, A. Prasad, D. Pinto, S. Walker, I. O'Connor, C. Russell, I. E. Drmic, F. F. Hamdan, J. L. Michaud, V. Endris, R. Roeth, R. Delorme, G. Huguet, M. Leboyer, M. Rastam, C. Gillberg, M. Lathrop, D. J. Stavropoulos, E. Anagnostou, R. Weksberg, E. Fombonne, L. Zwaigenbaum, B. A. Fernandez, W. Roberts, G. A. Rappold, C. R. Marshall, T. Bourgeron, P. Szatmari, S. W. Scherer, SHANK1 Deletions in Males with Autism Spectrum Disorder. Am. J. Hum. Genet. 90, 879-887 (2012).
20. I. Caillé, B. Allinquant, E. Dupont, C. Bouillot, A. Langer, U. Muller, A. Prochiantz, Soluble form of amyloid precursor protein regulates proliferation of progenitors in the adult subventricular zone. Development. 131, 2173-2181 (2004).
21. R. Noseda, S. Belin, F. Piguet, I. Vaccari, S. Scarlino, P. Brambilla, F. Martinelli Boneschi, M. L. Feltri, L. Wrabetz, A. Quattrini, E. Feinstein, R. L. Huganir, A. Bolino, DDIT4/REDD1/RTP801 is a novel negative regulator of Schwann cell myelination. J. Neurosci. 33, 15295-15305 (2013).
22. T. Wachi, B. Cornell, C. Marshall, V. Zhukarev, P. W. Baas, K. Toyo-oka, Ablation of the 14-3-3gamma Protein Results in Neuronal Migration Delay and Morphological Defects in the Developing Cerebral Cortex. Dev. Neurobiol. 76, 600-614 (2016).
23. K. Clemens, V. Bilanchone, N. Beliakova-Bethell, Sequence requirements for localization and packaging of Ty3 retroelement RNA. Virus Res. (2013) (available at https://www.sciencedirect.com/science/article/pii/S0168170212003784).
24. C. Jolly, Q. J. Sattentau, Human immunodeficiency virus type 1 assembly, budding, and cell-cell spread in T cells take place in tetraspanin-enriched plasma membrane domains. J. Virol. 81, 7873-7884 (2007).
25. R. J. Platt, S. Chen, Y. Zhou, M. J. Yim, L. Swiech, H. R. Kempton, J. E. Dahlman, O. Parnas, T. M. Eisenhaure, M. Jovanovic, D. B. Graham, S. Jhunjhunwala, M. Heidenreich, R. J. Xavier, R. Langer, D. G. Anderson, N. Hacohen, A. Regev, G. Feng, P. A. Sharp, F. Zhang, CRISPR-Cas9 knockin mice for genome editing and cancer modeling. Cell. 159, 440-455 (2014).
26. A. Shevchenko, O. N. Jensen, A. V. Podtelejnikov, F. Sagliocco, M. Wilm, O. Vorm, P. Mortensen, A. Shevchenko, H. Boucherie, M. Mann, Linking genome and proteome by mass spectrometry: large-scale identification of yeast proteins from two dimensional gels. Proc. Natl. Acad. Sci. U.S.A 93, 14440-14445 (1996).
27. L. Kill, J. D. Storey, W. S. Noble, Non-parametric estimation of posterior error probabilities associated with peptides identified by tandem mass spectrometry. Bioinformatics. 24, i42-8 (2008).
28. A. I. Nesvizhskii, A. Keller, E. Kolker, R. Aebersold, A statistical model for identifying proteins by tandem mass spectrometry. Anal. Chem. 75, 4646-4658 (2003).
29. R. C. Challis, S. R. Kumar, K. Y. Chan, C. Challis, M. J. Jang, P. S. Rajendran, J. D. Tompkins, K. Shivkumar, B. E. Deverman, V. Gradinaru, Widespread and targeted gene expression by systemic AAV vectors: Production, purification, and administration. Cold Spring Harbor Laboratory (2018), p. 246405.
30. N. Habib, I. Avraham-Davidi, A. Basu, T. Burks, K. Shekhar, M. Hofree, S. R. Choudhury, F. Aguet, E. Gelfand, K. Ardlie, D. A. Weitz, O. Rozenblatt-Rosen, F. Zhang, A. Regev, Massively parallel single-nucleus RNA-seq with DroNc-seq. Nature Methods. 14 (2017), pp. 955-958.
31. H. Pimentel, N. L. Bray, S. Puente, P. Melsted, L. Pachter, Differential analysis of RNA-seq incorporating quantification uncertainty. Nat. Methods. 14, 687-690 (2017).
32. N. L. Bray, H. Pimentel, P. Melsted, L. Pachter, Near-optimal probabilistic RNA-seq quantification. Nat. Biotechnol. 34, 525-527 (2016).
33. J. L. Schmid-Burgk, T. Schmidt, M. M. Gaidt, K. Pelka, E. Latz, T. S. Ebert, V. Hornung, OutKnocker: a web tool for rapid and simple genotyping of designer nuclease edited cell lines. Genome Res. 24, 1719-1723 (2014).
34. M. J. Moore, C. Zhang, E. C. Gantman, A. Mele, J. C. Darnell, R. B. Darnell, Mapping Argonaute and conventional RNA-binding protein interactions with RNA at single-nucleotide resolution using HITS-CLIP and CIMS analysis. Nature Protocols. 9 (2014), pp. 263-293.
35. E. L. Van Nostrand, T. B. Nguyen, C. Gelboin-Burkhart, R. Wang, S. M. Blue, G. A. Pratt, A. L. Louie, G. W. Yeo, Robust, Cost-Effective Profiling of RNA Binding Protein Targets with Single-end Enhanced Crosslinking and Immunoprecipitation (seCLIP). Methods Mol. Biol. 1648, 177-200 (2017).
36. J.-E. Park, R. A. Botting, C. Dominguez Conde, D.-M. Popescu, M. Lavaert, D. J. Kunz, I. Goh, E. Stephenson, R. Ragazzini, E. Tuck, A. Wilbrey-Clark, K. Roberts, V. R. Kedlian, J. R. Ferdinand, X. He, S. Webb, D. Maunder, N. Vandamme, K. T. Mahbubani, K. Polanski, L. Mamanova, L. Bolt, D. Crossland, F. de Rita, A. Fuller, A. Filby, G. Reynolds, D. Dixon, K. Saeb-Parsy, S. Lisgo, D. Henderson, R. Vento-Tormo, O. A. Bayraktar, R. A. Barker, K. B. Meyer, Y. Saeys, P. Bonfanti, S. Behjati, M. R. Clatworthy, T. Taghon, M. Haniffa, S. A. Teichmann, A cell atlas of human thymic development defines T cell repertoire formation. Science. 367 (2020), doi:10.1126/science.aay3224.

### Example 6—Effect of TSPANs on P10 Secretion

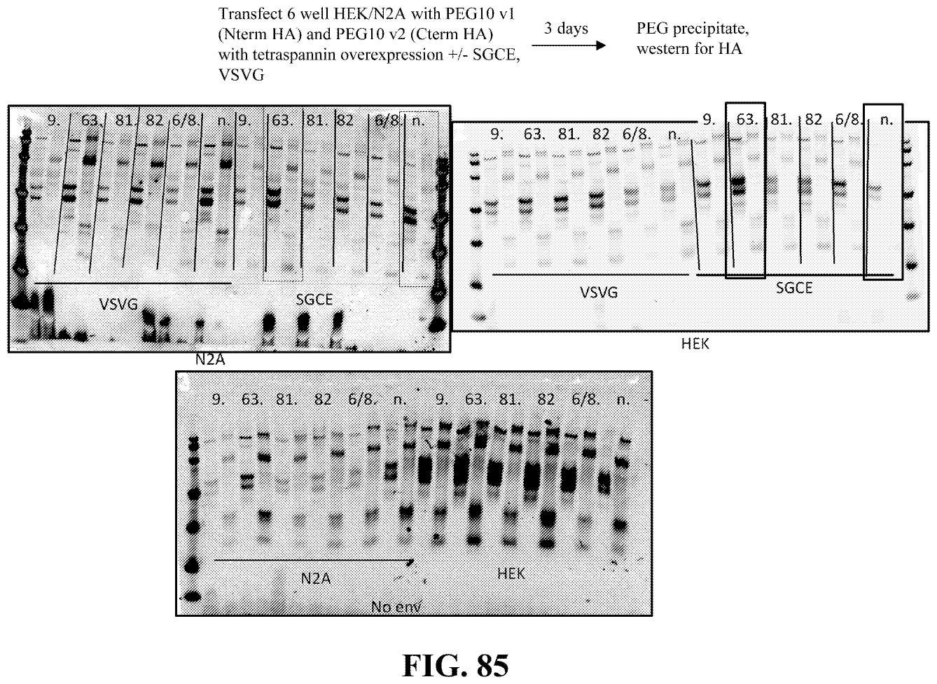

FIG. 85 shows the effect of overexpression of TSPANs on P10 secretion.

Experimental outline and results from overexpressing TSPANs in HEK/N2A cells to evaluate the effect of overexpression of TSPANs on P10 secretion.

### Example 7—PEG10 as an Exosome

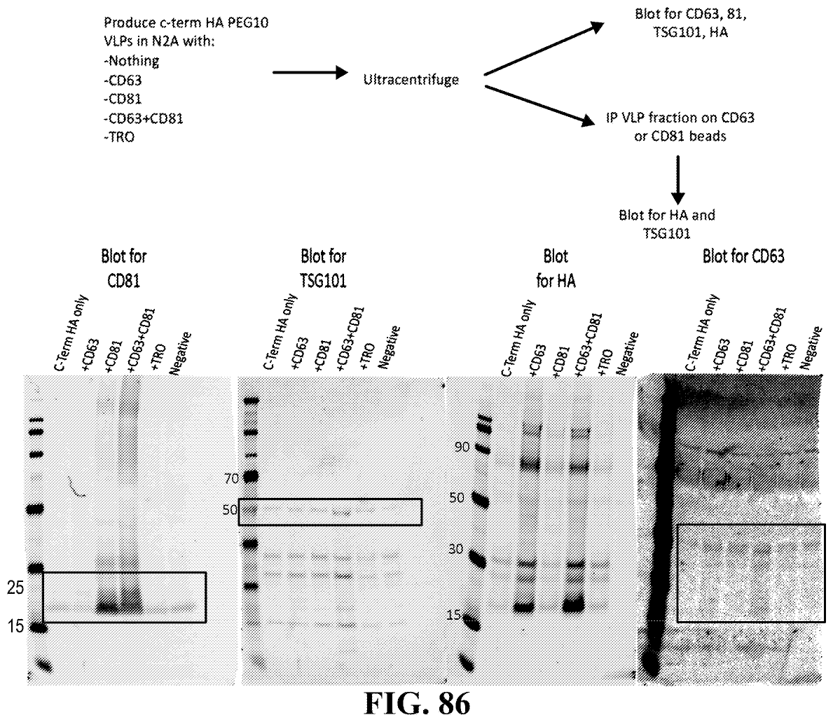

FIG. 86 shows a experimental outline and results determining if PEG10 is an exosome. C-terminal HA tagged PEG10 virus like particles (VLPs) were produced in N2As with the noted proteins and ability to produce exosomes was examined.

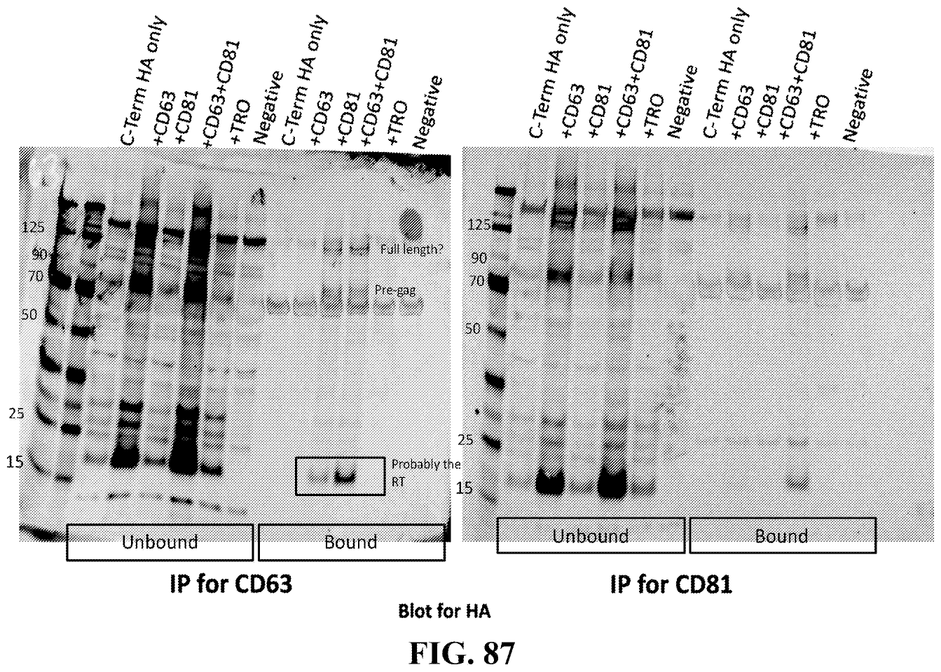

FIG. 87 shows further results determining if PEG10 is an exosome. As in FIG. 86, FIG. 87 presents further results from the experiment described in relation to FIG. 86 designed to determine if PEG10 is an exosome.

### Example 8—Oligomerization of PEG10 Gag Mutants

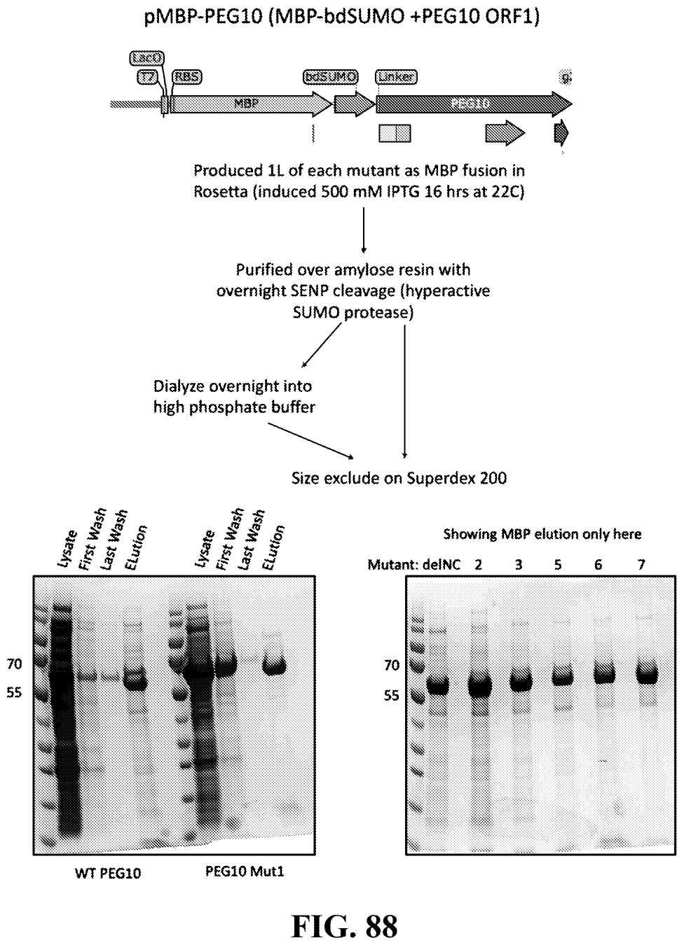

FIG. 88 shows a schematic of a production/purification strategy and results from production of Gag mutants. As shown in the schematic, gag mutants were expressed as fusion proteins with an MBP cleavable tag. The protein gels demonstrate that PEG10 Gag mutants produced well from the production and purification strategy.

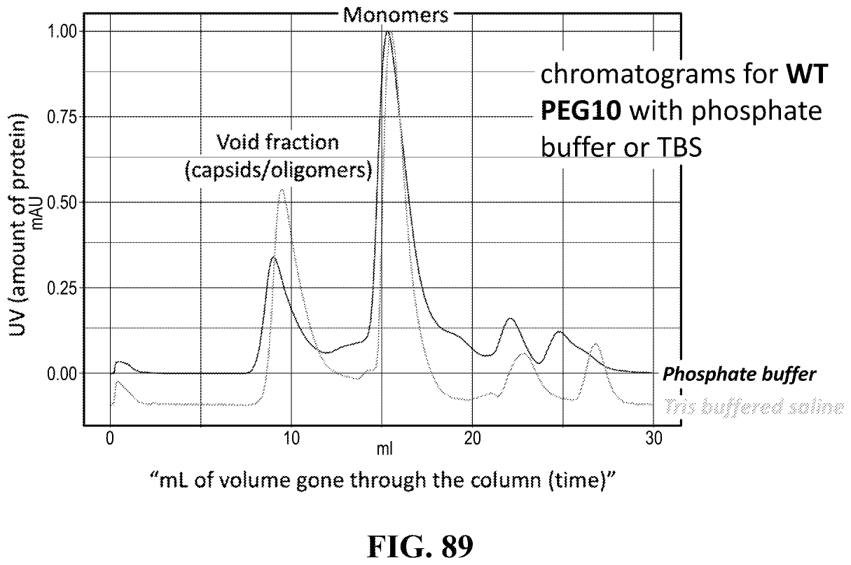

FIG. 89 shows chromatograms demonstrating an effect of phosphate on oligomerization of wild type (WT) PEG10. As demonstrated by the decrease in presence of capsid/oligomers in the presence of phosphate buffer can indicate that phosphate may be detrimental to oligomerization of PEG10.

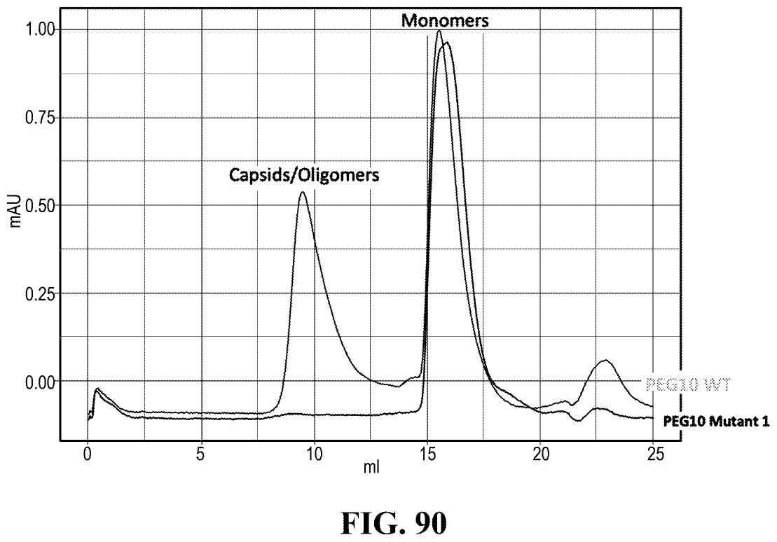

FIG. 90 shows a chromatogram of the Gag Mutant 1 that demonstrates a complete capsid loss and an inability to oligomerize. This chromatogram can demonstrate the mutated region in Gag1 mutant plays a role oligomerization of the polypeptide.

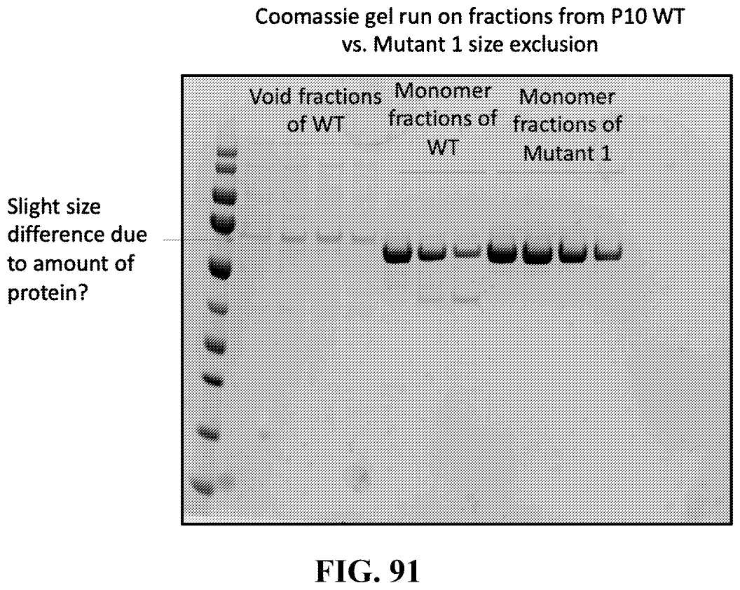

FIG. 91 shows a Coomassie stained protein gel demonstrating proteins in the void fraction from chromatography. As demonstrated by this gel the void fraction and monomers are the same protein.

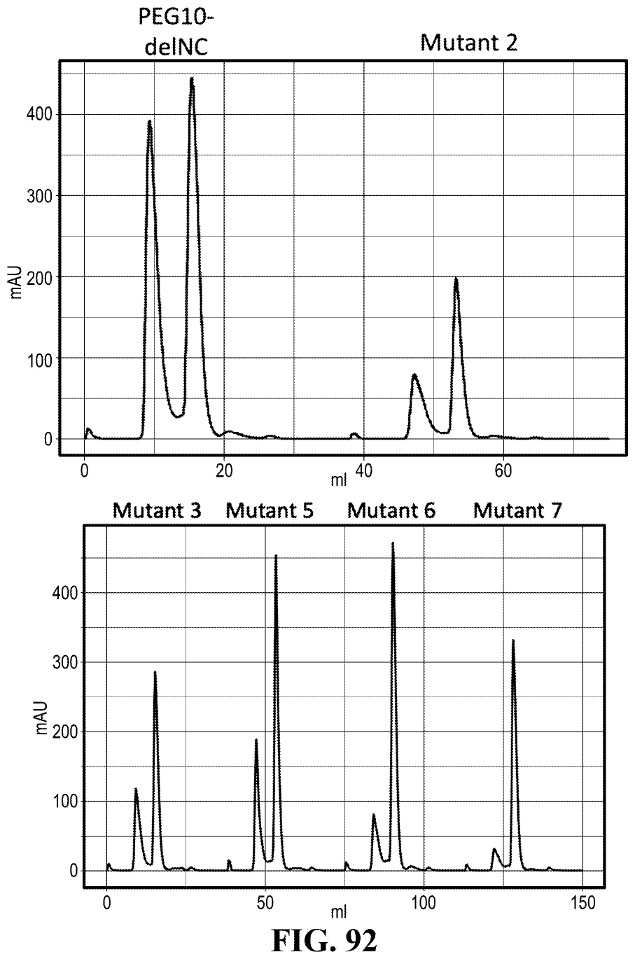

FIG. 92 shows chromatograms of other gag mutants produced. FIG. 92 can demonstrate that other gag mutants do not show as impaired of capsid assembly as compared to gag mutant 1.

### Example 9—PEG10 Capsid Formation and Effect of Co-Transfection with Fusogenic Protein SynA

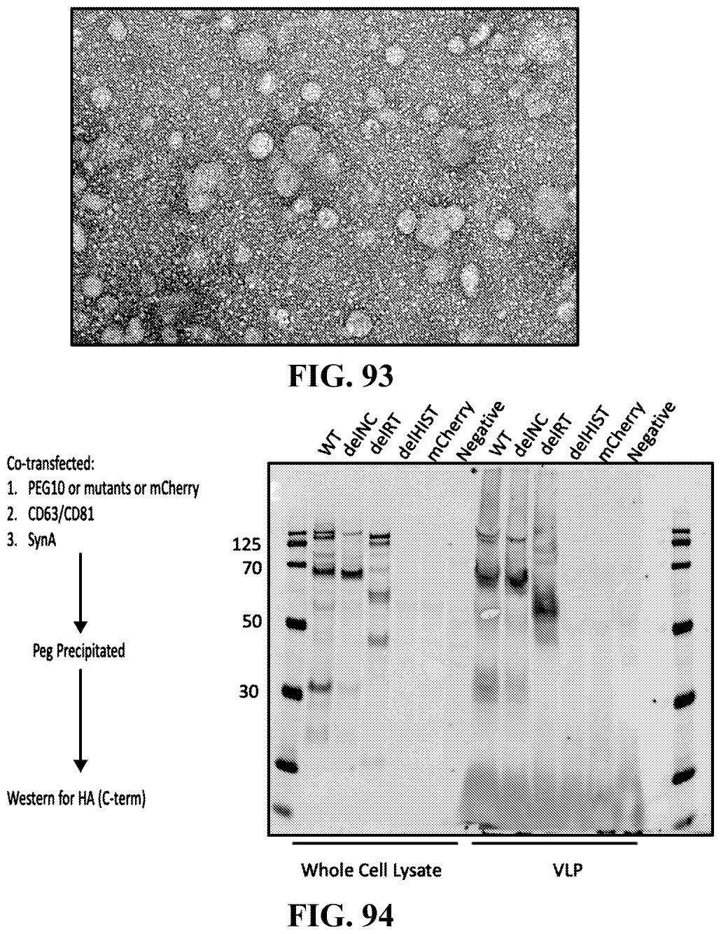

FIG. 93 shows a photomicrographic image demonstrating that PEG10 protein produced from E. coli forms capsids. Capsids produced had an average diameter of about 20-30 nm.

FIG. 94 shows an experimental strategy and protein gel demonstrating effect of co-transfection with a membrane fusion protein on secretion. As demonstrated by FIG. 94, PEG10 is still secreted when co-transfected with SynA.

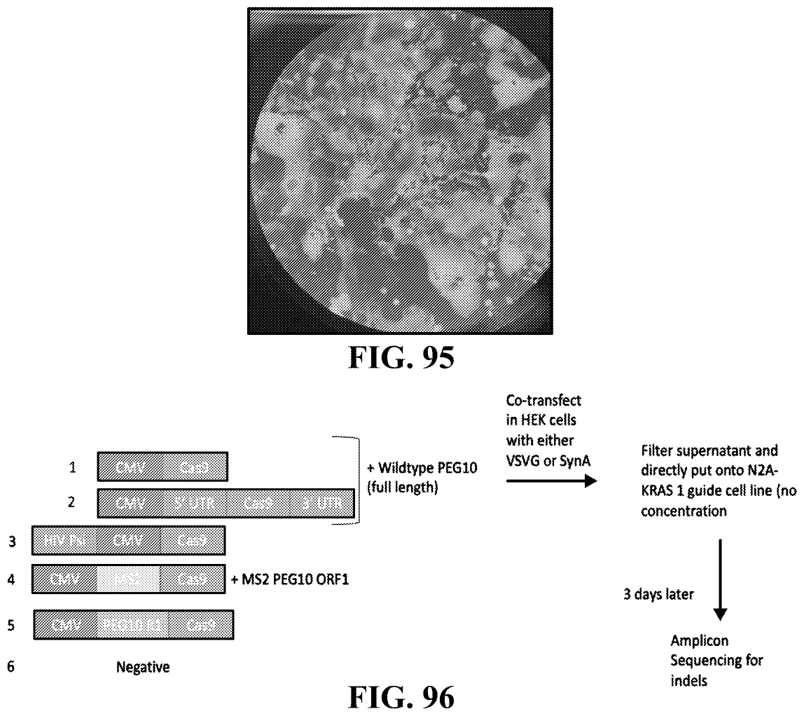

FIG. 95 shows a fluorescent microscopy image demonstrating that SynA induces substantial membrane fusion in HEK cells. SynA was co-transfected with CMV-GFP in HEK 293FT cells which demonstrated that SynA can induce substantial membrane fusion.

### Example 10—Effect of UTRs on PEG10 Packaging and Delivery

FIG. 96 shows construct maps for evaluating a trans packaging strategy with PEG10 untranslated regions (UTRs).

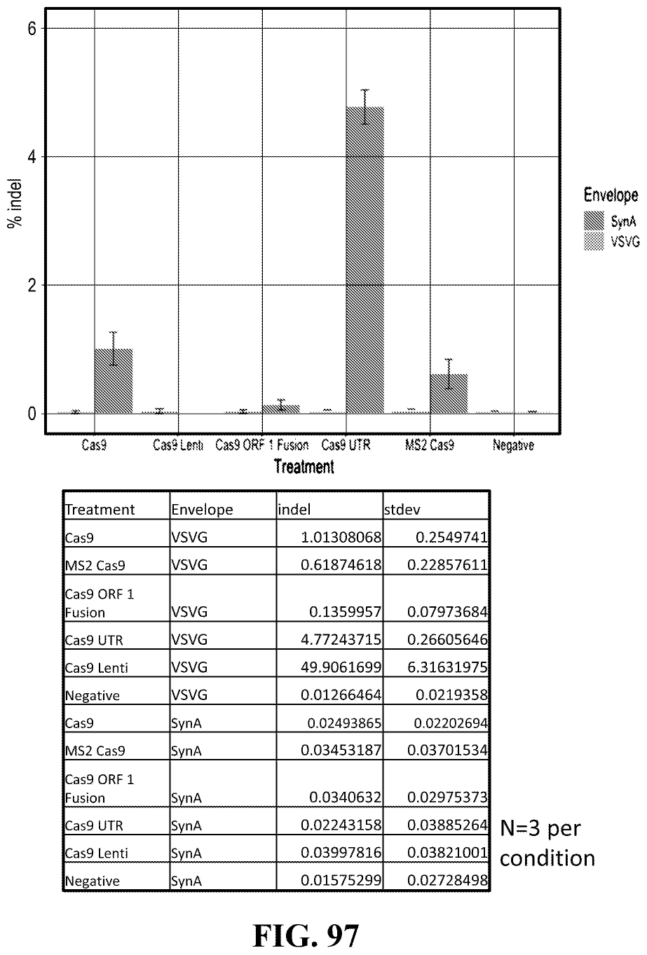

FIG. 97 shows results that can at least demonstrate that the addition of UTRs to a cargo can allows for packaging and delivery of the cargo. As per the strategy shown in FIG. 96, Cas9, when flanked with PEG10 UTRs, demonstrated higher activity (as measured by % indels generated) as compared to when UTRs were not flanking the Cas9.

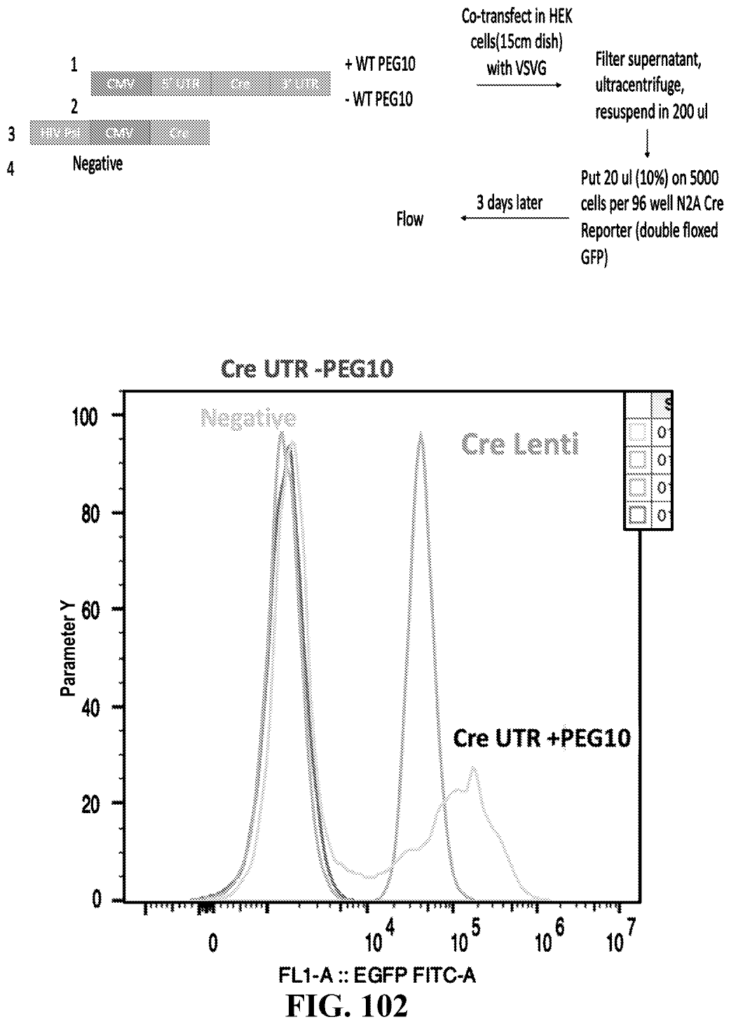

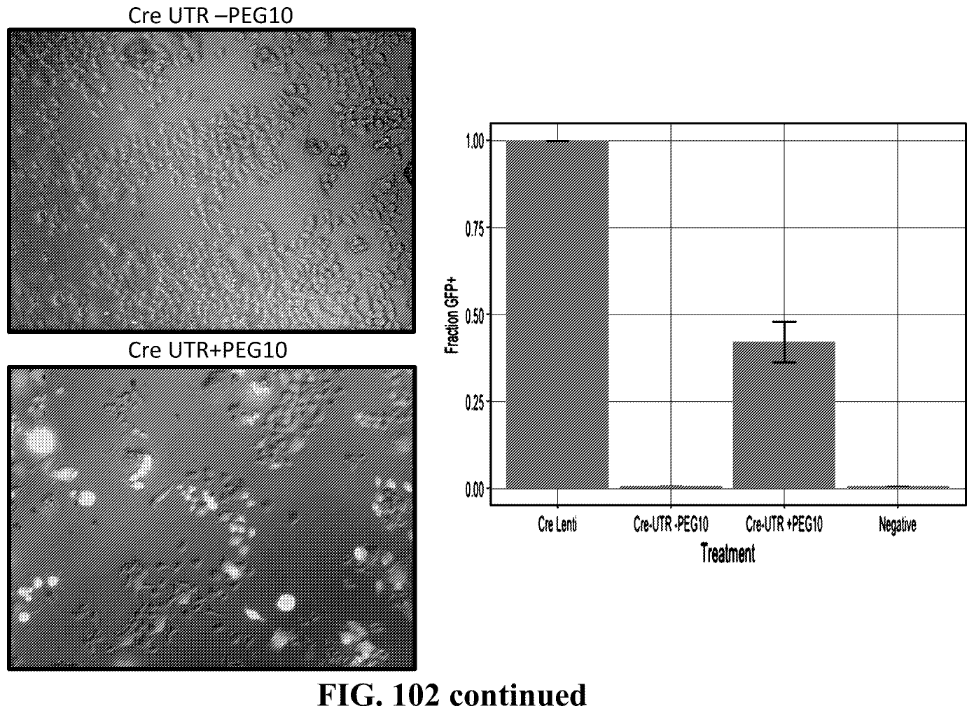

FIG. 102 shows construct maps for evaluating trans packaging with PEG10 UTR flanked cargos and results demonstrating transfer of functional mRNA from the vesicles carrying the packaged cargo. Cre was flanked with PEG10 UTRs and constructs were co-transfected in HEK cells with VSV-G. Vesicles were purified as shown in the purification schematic and added to N2A Cre reporter cells. 3 days later results were obtained using flow cytometry. The results in shown in the graphs and images demonstrate that the UTR flanked Cre VLPs transferred functional mRNA.

### Example 11—Effect of PEG 10 VLPs on Target Cell Transcriptional State

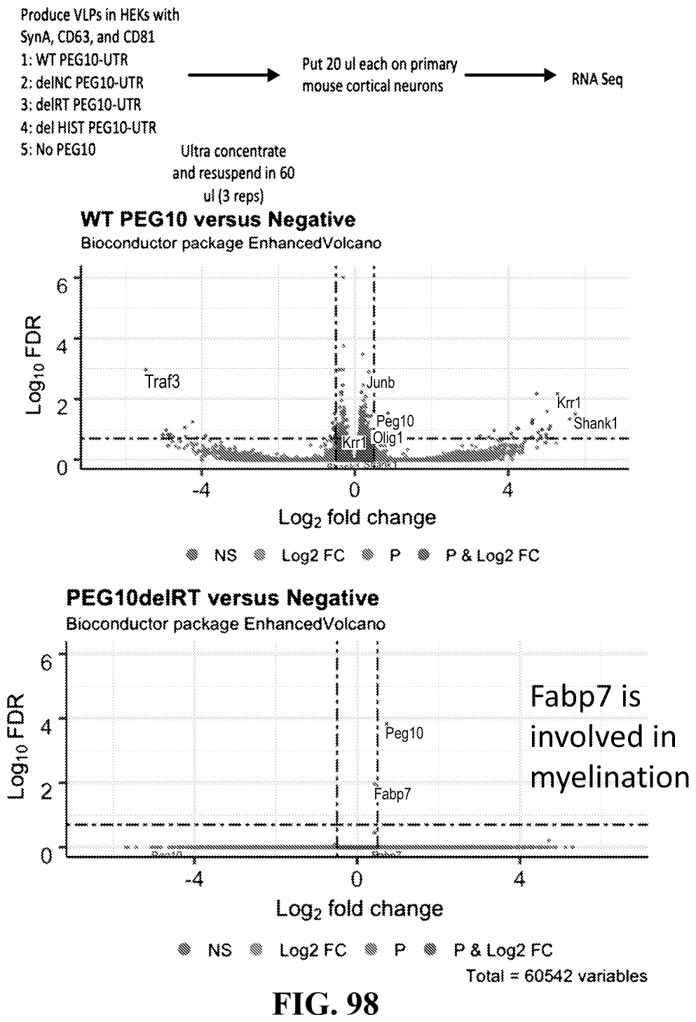

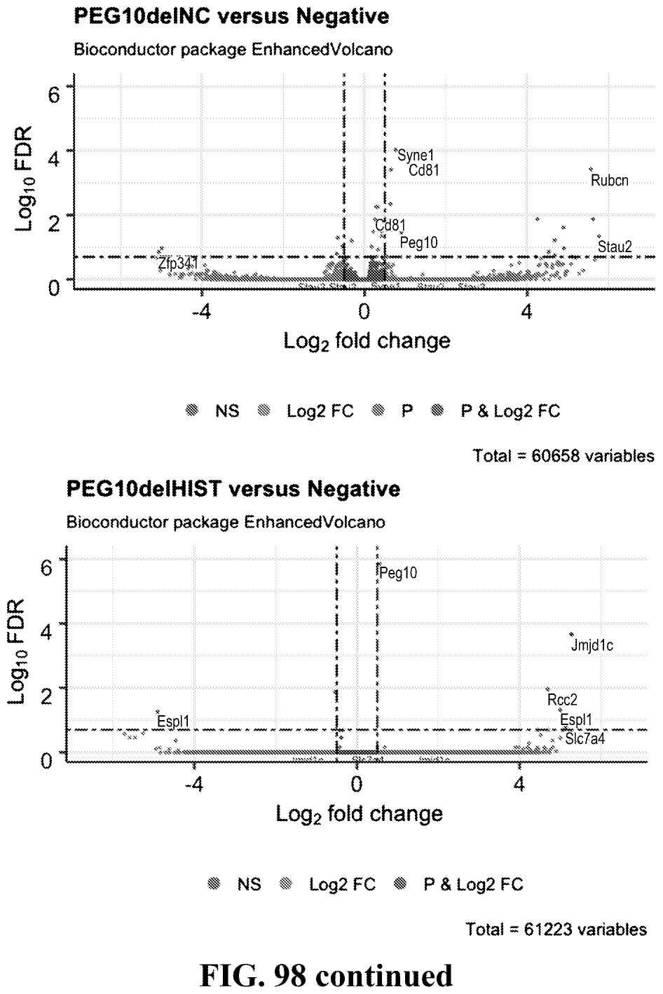

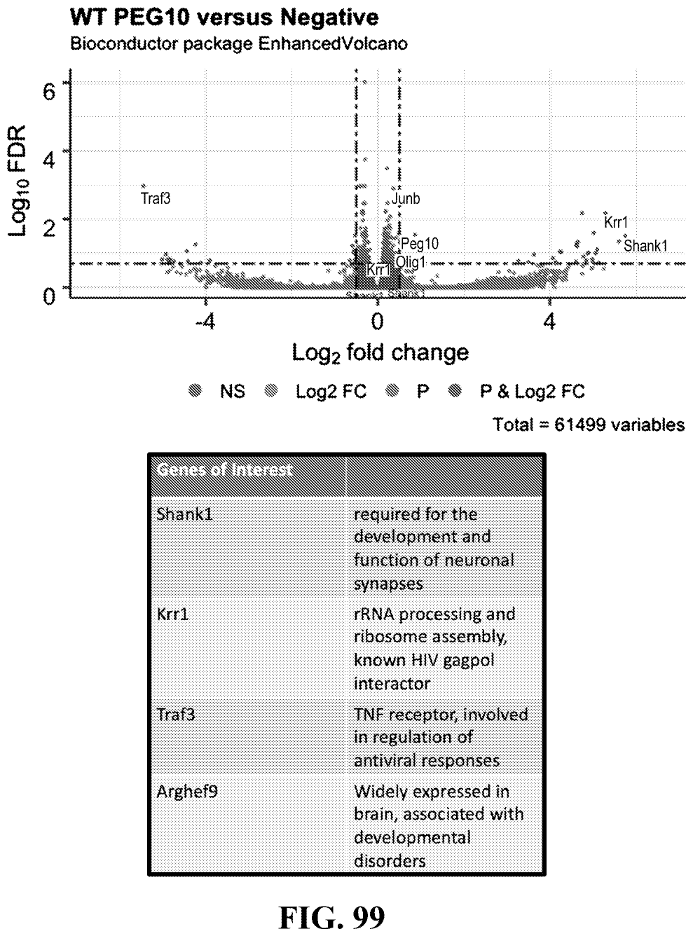

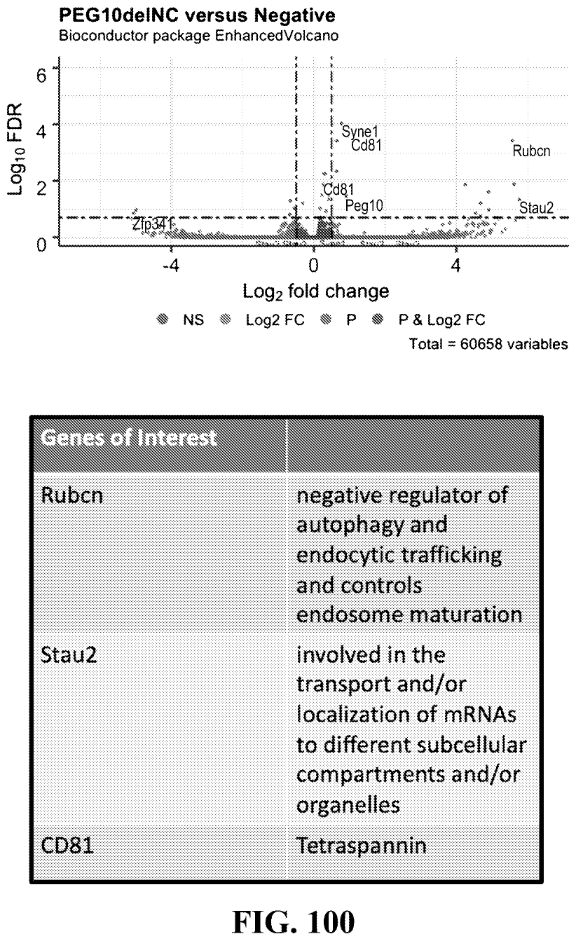

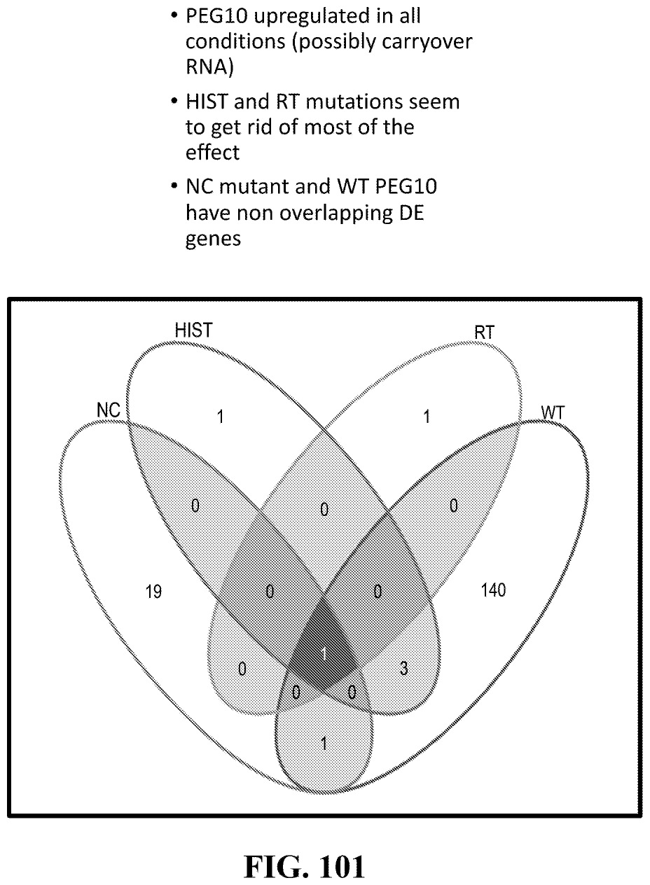

FIGS. 98-100 show an experimental strategy and results that can demonstrate the effect of PEG10 VLPs on the transcriptional state of target cells. FIG. 101 shows a venn diagram demonstrating DE gene overlap in response to the introduction of PEG10 VLPs to cells as shown in relation to FIGS. 98-100. PEG10 was upregulated in all conditions. HIST and RT mutations were observed to reduce or eliminate the this observed effect. The NC mutant and WT PEG10 were observed to have overlapping DE genes.

### Example 12—Mammalian Retrovirus-Like Protein PEG10 Packages its Own mRNA and can be Pseudotyped for Intercellular mRNA Delivery

### Introduction

Eukaryotic genomes contain domesticated genes from integrating viruses and mobile genetic elements. Among these are homologs of the capsid protein (known as Gag) of long terminal repeat (LTR) retrotransposons and retroviruses. Applicant identifies several mammalian Gag homologs that form virus-like particles (VLPs) and one LTR retrotransposon homolog, PEG10, that preferentially binds and facilitates vesicular secretion of its own mRNA. Applicant shows the mRNA cargo of PEG10 can be reprogrammed by flanking genes of interest with Peg10's untranslated regions (UTRs). Taking advantage of this reprogrammability, Applicant developed Selective Endogenous eNcapsidation for cellular Delivery (SEND) by engineering both mouse and human PEG10 to package, secrete, and deliver specific RNAs. Together, these results demonstrate SEND as a modular platform suited for development as an efficient therapeutic delivery modality.

More than 8% of the human genome is composed of sequences derived from LTR retroelements, including retroviruses, that have integrated into mammalian genomes throughout evolution (1-5). Retroviruses and retrotransposons have many common mechanistic features including the core structural gene (known as gag), however, while retrotransposons replicate intracellularly, the acquisition of the envelope (env) gene by retroviruses has enabled intercellular replication (6). Most endogenous retroelements have lost their original functions, but some of their genes have been recruited for diverse roles in normal mammalian physiology. For example, the fusogenic syncytins evolved from retroviral env proteins (7). The gag homolog, Arc, which forms capsids and has been reported to transfer its own mRNA (8-10), is involved in memory consolidation and regulates inflammation in the skin (11, 12). Another gag homolog, the LTR retrotransposon-derived protein PEG10, which has been reported to bind RNA and also forms capids (13), is involved in mammalian placenta formation (14, 15). These examples raise the possibility that additional retroelement-derived proteins encoded in the mammalian genome transfer specific nucleic acids, providing a potentially programmable mechanism for intercellular communication.

To identify genes with the potential to transfer specific nucleic acids, Applicant focused on homologs of gag containing the core capsid (CA) domain, which protects the genome of both retrotransposons and exogenous retroviruses (16, 17). Previous genome analyses identified many endogenous gag homologs in mammalian genomes (18), and experimental efforts have validated the ability of some of these proteins including MmArc and MmPeg10 to form capsid-like particles that are secreted within extracellular vesicles (EVs) (10, 13). To ensure a complete list of candidates, Applicant searched the human and mouse genomes for gag homologs. This search identified 48 gag-derived genes in the human genome and 102gag homologs in the mouse genome; for 19 human genes, an orthologous relationship between human and mouse was readily traced (in several cases, with additional mouse paralogs), whereas the remaining ones appeared to be species-specific (Table 12 and Table 13). For Table 12, Most orthologous groups with proteins from both species include one human sequence and a few mouse sequences, in most cases these mouse sequences in one group correspond to the same gene but differ mostly by truncation or point mutations. In two groups, namely with human proteins NP_776159.1 and NP_001299820.1 the mouse proteins are clearly paralogous but are practically equally close to human group members. Overall, there are 19 orthologous groups with proteins from both species, two of which are with obvious mouse paralogs, and 15 human and 48 mouse singletons.

TABLE 12
Summary of orthologous groups of proteins with domains homologous to Gag
capsid protein in human and mouse based on sequence alignments.

| Candidates (Homo sapiens) | | | Orthologous candidates (Mus musculus) | | |
|---|---|---|---|---|---|
| | Pfam domain | | | Pfam domain | |
| Identified protein | architecture | Accession | Identified protein | architecture | Accession |
| endogenous | Retroviral GAG p10 | XP_011511643.1 | — | | — |
| retrovirus group | protein; gag gene protein | | | | |
| K member 5 Gag | p24 (core nucleocapsid | | | | |
| polyprotein | protein); Zinc knuckle; | | | | |
| | GAG-polyprotein viral | | | | |
| | zinc-finger | | | | |
| endogenous | Retroviral GAG p10 | XP_016863109.1 | — | | — |
| retrovirus group | protein; gag gene protein | | | | |
| K member 7 Gag | p24 (core nucleocapsid | | | | |
| polyprotein | protein); Zinc knuckle; | | | | |
| | GAG-polyprotein viral | | | | |
| | zinc-finger | | | | |
| uncharacterized | Retroviral GAG p10 | XP_016883063.1 | — | | — |
| protein | protein; gag gene protein | | | | |
| LOC107985332 | p24 (core nucleocapsid | | | | |
| | protein); Zinc knuckle | | | | |
| paraneoplastic | PNMA | NP_001096620.1 | — | | — |
| antigen-like | | | | | |
| protein 5 | | | | | |
| retrotransposon- | Domain of unknown | NP_001165908.1 | Retrotransposon- | | sp\|Q7TN75 |
| derived protein | function (DUF4939); | | derived protein | | |
| PEG10 isoform 3 | GAG-polyprotein viral | | PEG10 | | |
| retrotransposon- | zinc-finger; Retroviral | NP_001035242.1 | retrotransposon- | Domain of | NP_001035701.1 |
| derived protein | aspartyl protease | | derived protein | unknown | |
| PEG10 isoform 2 | | | PEG10 isoform 2 | function | |
| | | | | (DUF4939); | |
| | | | | GAG-polyprotein | |
| | | | | viral zinc-finger; | |
| | | | | Retroviral | |
| | | | | aspartyl protease | |
| retrotransposon- | | NP_001165909.1 | retrotransposon- | | NP_570947.2 |
| derived protein | | | derived protein | | |
| PEG10 isoform 4 | | | PEG10 isoform 1 | | |
| retrotransposon- | | NP_001171890.1 | | | |
| derived protein | | | | | |
| PEG10 isoform 5 | | | | | |
| retrotransposon- | | NP_001171891.1 | | | |
| derived protein | | | | | |
| PEG10 isoform 6 | | | | | |
| retrotransposon- | | NP_055883.2 | | | |
| derived protein | | | | | |
| PEG10 isoform 1 | | | | | |
| retrotransposon | Domain of unknown | NP_001071639.1 | — | | — |
| Gag-like protein | function (DUF4939) | | | | |
| 8B | | | | | |
| retrotransposon | Domain of unknown | NP_001071641.1 | — | | — |
| Gag-like protein | function (DUF4939) | | | | |
| 8B | | | | | |
| retrotransposon | Domain of unknown | NP_001071640.1 | — | | — |
| Gag-like protein | function (DUF4939) | | | | |
| 8A isoform 1 | | | | | |
| paraneoplastic | PNMA | NP_001269464.1 | unnamed protein | PNMA; Zinc | BAE24735.1 |
| antigen Ma3 | | | product, partial | knuckle | |
| isoform 2 | | | | | |
| [Homo sapiens] | | | | | |
| paraneoplastic | | NP_037496.4 | paraneoplastic | | NP_694809.1 |
| antigen Ma3 | | | antigen Ma3 | | |
| isoform 1 | | | homolog | | |
| [Homo sapiens] | | | | | |
| paraneoplastic | PNMA | NP_116271.3 | mCG1032934 | PNMA | EDL29902.1 |
| antigen-like | | | | | |
| protein 6A | | | | | |
| paraneoplastic | PNMA | NP_443158.1 | — | | — |
| antigen-like | | | | | |
| protein 5 | | | | | |
| paraneoplastic | PNMA | NP_006020.4 | paraneoplastic | PNMA | NP_081714.2 |
| antigen Ma1 | | | antigen Ma1 | | |
| | | | homolog | | |
| modulator of | PNMA | NP_071434.2 | modulator of | PNMA | NP_071718.1 |
| apoptosis 1 | | | apoptosis 1 | | |
| | | | modulator of | | NP_001136409.1 |
| | | | apoptosis 1 | | |
| paraneoplastic | PNMA | NP_009188.1 | mKIAA0883 | | BAD90245.1 |
| antigen Ma2 | | | protein, partial | | |
| paraneoplastic | | XP_011542667.1 | paraneoplastic | | EDL35986.1 |
| antigen Ma2 | | | antigen MA2, | | |
| isoform X1 | | | isoform CRA_a, | | |
| | | | partial | | |
| | | | paraneoplastic | PNMA | EDL35987.1 |
| | | | antigen MA2, | | |
| | | | isoform CRA_b | | |
| | | | paraneoplastic | | NP_780707.1 |
| | | | antigen Ma2 | | |
| | | | homolog | | |
| | | | paraneoplastic | | XP_006519052.1 |
| | | | antigen Ma2 | | |
| | | | homolog isoform | | |
| | | | X1 | | |
| | | | paraneoplastic | | XP_006519053.1 |
| | | | antigen Ma2 | | |
| | | | homolog isoform | | |
| | | | X1 | | |
| paraneoplastic | PNMA | NP_065760.1 | PNMA-like | PNMA | NP_001093106.1 |
| antigen-like | | | protein 2 | | |
| protein 8B | | | | | |
| protein Bop | Domain of unknown | NP_078903.3 | — | | — |
| | function (DUF4939) | | | | |
| retrotransposon | Domain of unknown | NP_689907.1 | retrotransposon | Domain of | NP_955762.1 |
| Gag-like protein | function (DUF4939); | | Gag-like protein | unknown | |
| 3 | Zinc knuckle | | 3 | function | |
| | | | | (DUF4939); Zinc | |
| | | | | knuckle | |
| retrotransposon | Domain of unknown | NP_001019626.1 | retrotransposon | Domain of | NP_001265463.1 |
| Gag-like protein | function (DUF4939) | | Gag-like protein | unknown | |
| 5 | | | 5 | function | |
| | | | | (DUF4939) | |
| | | | mKIAA2001 | | BAD90267.1 |
| | | | protein, partial | | |
| retrotransposon- | Domain of unknown | NP_001128360.1 | — | | — |
| like protein 1 | function (DUF4939); | | | | |
| | RNase H-like domain | | | | |
| | found in reverse | | | | |
| | transcriptase | | | | |
| retrotransposon | Domain of unknown | NP_115663.2 | retrotransposon | Domain of | NP_808298.2 |
| Gag-like protein | function (DUF4939) | | Gag-like protein | unknown | |
| 6 | | | 6 | function | |
| | | | | (DUF4939) | |
| protein LDOC1 | Domain of unknown | NP_036449.1 | — | | — |
| | function (DUF4939) | | | | |
| zinc finger | PNMA | NP_001299820.1 | zinc finger | | NP_001345405.1 |
| CCHC domain- | | | CCHC domain- | | |
| containing | | | containing | | |
| protein 12 | | | protein 12 | | |
| zinc finger | | NP_776159.1 | zinc finger | | NP_001345406.1 |
| CCHC domain- | | | CCHC domain- | | |
| containing | | | containing | | |
| protein 12 | | | protein 12 | | |
| | | | zinc finger | | NP_001345407.1 |
| | | | CCHC domain- | | |
| | | | containing | | |
| | | | protein 12 | | |
| | | | zinc finger | | NP_001345408.1 |
| | | | CCHC domain- | | |
| | | | containing | | |
| | | | protein 12 | | |
| | | | zinc finger | | NP_001345409.1 |
| | | | CCHC domain- | | |
| | | | containing | | |
| | | | protein 12 | | |
| | | | zinc finger | | XP_006541462.1 |
| | | | CCHC domain- | | |
| | | | containing | | |
| | | | protein 12 | | |
| | | | isoform X1 | | |
| | | | zinc finger | PNMA | XP_011249276.1 |
| | | | CCHC domain- | | |
| | | | containing | | |
| | | | protein 12 | | |
| | | | isoform X1 | | |
| | | | zinc finger | | XP_017174119.1 |
| | | | CCHC domain- | | |
| | | | containing | | |
| | | | protein 12 | | |
| | | | isoform X1 | | |
| | | | zinc finger | | XP_036017996.1 |
| | | | CCHC domain- | | |
| | | | containing | | |
| | | | protein 12 | | |
| | | | isoform X1 | | |
| | | | zinc finger | | XP_036017997.1 |
| | | | CCHC domain- | | |
| | | | containing | | |
| | | | protein 12 | | |
| | | | isoform X1 | | |
| | | | zinc finger | | XP_036017998.1 |
| | | | CCHC domain- | | |
| | | | containing | | |
| | | | protein 12 | | |
| | | | isoform X1 | | |
| | | | zinc finger | | NP_082601.1 |
| | | | CCHC domain- | | |
| | | | containing | | |
| | | | protein 12 | | |
| zinc finger | PNMA; Zf-CCHC | XP_011529314.1 | zinc finger, | | EDL23888.1 |
| CCHC domain- | | | CCHC domain | | |
| containing | | | containing 18, | | |
| protein 18 | | | isoform CRA_a, | | |
| isoform X1 | | | partial | | |
| zinc finger | | NP_001137450.1 | unnamed protein | | BAB23950.1 |
| CCHC domain- | | | product | | |
| containing | | | | | |
| protein 18 | | | | | |
| | | | zinc finger | | NP_001030586.1 |
| | | | CCHC domain- | | |
| | | | containing | | |
| | | | protein 18 | | |
| | | | zinc finger | | NP_001030587.1 |
| | | | CCHC domain- | | |
| | | | containing | | |
| | | | protein 18 | | |
| | | | zinc finger | | NP_001345366.1 |
| | | | CCHC domain- | | |
| | | | containing | | |
| | | | protein 18 | | |
| | | | zinc finger | PNMA; Zf- | NP_001345368.1 |
| | | | CCHC domain- | CCHC | |
| | | | containing | | |
| | | | protein 18 | | |
| | | | zinc finger | | NP_001345369.1 |
| | | | CCHC domain- | | |
| | | | containing | | |
| | | | protein 18 | | |
| | | | zinc finger | | NP_001345370.1 |
| | | | CCHC domain- | | |
| | | | containing | | |
| | | | protein 18 | | |
| | | | zinc finger | | NP_080169.2 |
| | | | CCHC domain- | | |
| | | | containing | | |
| | | | protein 18 | | |
| | | | zinc finger | | XP_030107344.1 |
| | | | CCHC domain- | | |
| | | | containing | | |
| | | | protein 18 | | |
| | | | isoform X1 | | |
| | | | zinc finger | | XP_030107345.1 |
| | | | CCHC domain- | | |
| | | | containing | | |
| | | | protein 18 | | |
| | | | isoform X1 | | |
| retrotransposon | Retrotransposon gag | NP_065820.1 | retrotransposon | | XP_011246109.1 |
| Gag-like protein | protein domain | | Gag-like protein | | |
| 9 | | | 9 isoform X1 | | |
| | | | retrotransposon | Retrotransposon | XP_011246110.1 |
| | | | Gag-like protein | gag protein | |
| | | | 9 isoform X1 | domain | |
| | | | retrotransposon | | NP_001035524.2 |
| | | | Gag-like protein | | |
| | | | 9 | | |
| activity-regulated | Arc C-lobe | NP_056008.1 | activity-regulated | | NP_001263613.1 |
| cytoskeleton- | | | cytoskeleton- | | |
| associated | | | associated protein | | |
| protein | | | activity-regulated | Arc C-lobe | NP_061260.1 |
| | | | cytoskeleton- | | |
| | | | associated protein | | |
| retrotransposon | Domain of unknown | NP_001004308.2 | — | | — |
| Gag-like protein | function (DUF4939); | | | | |
| 4 | Zinc knuckle | | | | |
| endogenous | Retroviral GAG p10 | XP_011526763.1 | — | | — |
| retrovirus group | protein | | | | |
| K member 8 Gag | | | | | |
| polyprotein-like | | | | | |
| paraneoplastic | PNMA | NP_001171853.1 | paraneoplastic | | NP_001093931.1 |
| antigen-like | | | antigen-like | | |
| protein 5 | | | protein 5 | | |
| paraneoplastic | | NP_001096620.1 | | | |
| antigen-like | | | | | |
| protein 5 | | | | | |
| paraneoplastic | | NP_001096621.1 | | PNMA | |
| antigen-like | | | | | |
| protein 5 | | | | | |
| paraneoplastic | | XP_016884741.1 | | | |
| antigen-like | | | | | |
| protein 5 | | | | | |
| paraneoplastic | | XP_016884742.1 | | | |
| antigen-like | | | | | |
| protein 5 | | | | | |
| paraneoplastic | | NP_443158.1 | | | |
| antigen-like | | | | | |
| protein 5 | | | | | |
| retroviral-like | PNMA; gag-polyprotein | NP_690005.2 | Asprv1 protein, | PNMA; gag- | AAH57938.1 |
| aspartic protease | putative aspartyl protease | | partial | polyprotein | |
| 1 | | | | putative aspartyl | |
| | | | | protease | |
| paraneoplastic | PNMA | NP_001341909.1 | mCG1032934 | PNMA | EDL29902.1 |
| antigen Ma6F | | | | | |
| natural | Immunoglobulin V-set | NP_001189368.1 | | | |
| cytotoxicity | domain; Immunoglobulin | | | | |
| triggering | C1-set domain; Matrix | | | | |
| receptor 3 ligand | protein (MA), p15 | | | | |
| 1 precursor | | | | | |
| natural | | XP_011518374.1 | | | |
| cytotoxicity | | | | | |
| triggering | | | | | |
| receptor 3 ligand | | | | | |
| 1 isoform X1 | | | | | |
| natural | | XP_011518375.1 | | | |
| cytotoxicity | | | | | |
| triggering | | | | | |
| receptor 3 ligand | | | | | |
| 1 isoform X1 | | | | | |
| natural | | XP_011518376.1 | | | |
| cytotoxicity | | | | | |
| triggering | | | | | |
| receptor 3 ligand | | | | | |
| 1 isoform X1 | | | | | |
| natural | | XP_011518377.1 | | | |
| cytotoxicity | | | | | |
| triggering | | | | | |
| receptor 3 ligand | | | | | |
| 1 isoform X1 | | | | | |

TABLE 13
Summary of additional proteins with domains homologous to Gag capsid protein
in mouse based on sequence alignments. In addition to those listed in Table 12, 48 additional
proteins with domains homologous to Gag capsid protein are identified in mouse.
Candidates (Mus musculus)

| Identified protein | Domain | Accession |
|---|---|---|
| protein LDOC1 | DUF4939 | NP_001018097.1 |
| PREDICTED: agouti-signaling protein isoform X1 | Gag_p24 | XP_011237991.1 |
| gag protein | Gag_p24 | AAC12789.1 |
| Gag | Gag_p24 | AAC52922.1 |
| BC005685 protein, partial | Gag_p24 | AAH05685.1 |
| unnamed protein product | Gag_p24 | BAC38137.1 |
| gag | Gag_p24 | BAC79170.1 |
| gag | Gag_p24 | BAF81988.1 |
| TPA_exp: gag protein | Gag_p24 | DAA01924.1 |
| TPA_exp: gag protein | Gag_p24 | DAA01925.1 |
| TPA_exp: gag protein | Gag_p24 | DAA01928.1 |
| mCG142377, partial | Gag_p24 | EDL00544.1 |
| PREDICTED: uncharacterized protein LOC108167332 | Gag_p24 | XP_011239845.1 |
| IgE-binding protein | Gag_p24 | sp\|P03975.1\|IGEB_MOUSE |
| mCG1044120, partial | Gag_p24 | EDL07694.1 |
| PREDICTED: endogenous retrovirus group K member 24 | Gag_p24 | XP_011245081.1 |
| PREDICTED: endogenous retrovirus group K member 8 Gag polyprotein-like | Gag_p24 | XP_017167946.1 |
| gag-myb protein, partial | Gag_p30 | AAA39784.1 |
| putative | Gag_p30 | AAA51041.1 |
| Gag-Pol polyprotein | Gag_p30 | AAB06450.1 |
| gag protein | Gag_p30 | AAN46638.1 |
| truncated polyprotein | Gag_p30 | AAY27069.1 |
| gag po238tiliztein pr65 | Gag_p30 | ABD14432.1 |
| gag-pro-pol polyprotein | Gag_p30 | ABD14433.1 |
| gag polyprotein pr65 | Gag_p30 | ABD14435.1 |
| gag-pro-pol polyprotein | Gag_p30 | ABD14436.1 |
| glyco-gag polyprotein | Gag_p30 | AID54952.1 |
| gag polyprotein | Gag_p30 | AID54953.1 |
| gag-pro-pol polyprotein | Gag_p30 | AID54954.1 |
| gag, partial | Gag_p30 | AMK48512.1 |
| putative gag-pro-pol polyprotein | Gag_p30 | ARB03507.1 |
| unnamed protein product | Gag_p30 | BAC41106.1 |
| unnamed protein product | Gag_p30 | BAC41107.1 |
| truncated gag-pro-pol polyprotein | Gag_p30 | CCD57102.1 |
| gag-pro-pol polyprotein | Gag_p30 | CCD57104.1 |
| gag protein | Gag_p30 | CCD57105.1 |
| mCG144922, isoform CRA_b, partial | Gag_p30 | EDL00999.1 |
| LOC72520 protein, partial | Gag_p30 | AAH21868.1 |
| BC040756 protein, partial | Gag_p30 | AAH40756.1 |
| LOC72520 protein, partial | Gag_p30 | AAH44668.2 |
| PREDICTED: uncharacterized protein LOC108167440 isoform X1 | Gag_p30 | XP_017167935.1 |
| PREDICTED: uncharacterized protein LOC108167440 isoform X2 | Gag_p30 | XP_017167936.1 |
| PREDICTED: uncharacterized protein LOC108167440 isoform X3 | Gag_p30 | XP_017167937.1 |
| unnamed protein product, partial | PNMA | BAC37719.1 |
| mCG1050067, isoform CRA_a | PNMA | EDL42061.1 |
| coiled-coil domain-containing protein 8 homolog | PNMA | NP_001095005.1 |
| predicted gene, 42372 | PNMA | NP_001357780.1 |
| PREDICTED: paraneoplastic antigen Ma2 homolog | PNMA | XP_011249051.1 |

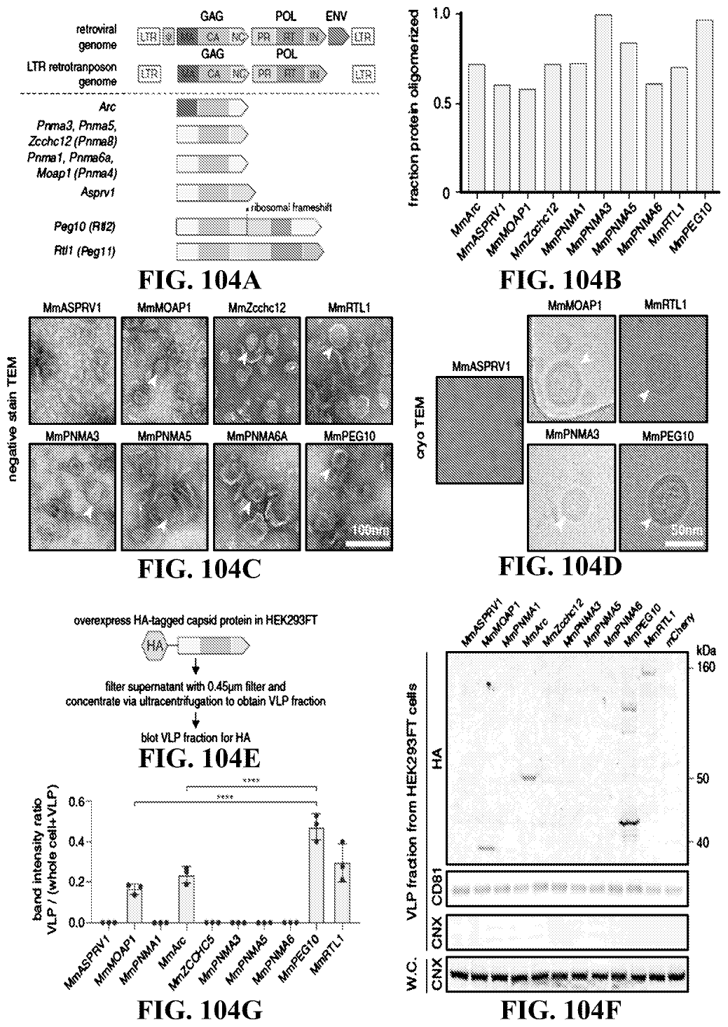

Canonical genomes of both LTR retrotransposons and retroviruses encode a long polyprotein consisting of several conserved domains: The matrix (MA), CA, and nucleocapsid (NC) form the gag subdomain and are responsible for membrane attachment, capsid formation, and genome binding, respectively. The pol subdomain contains the protease (PRO), responsible for cleaving the polyprotein, the reverse transcriptase (RT), which converts retroelement RNA into DNA, and the integrase (IN) domain, which integrates the genome into that of the host. Some families of endogenous Gag proteins, such as the PNMA family, contain only the CA and NC domains, whereas others, such as RTL1 and PEG10 (also known as RTL2 or Mart2), additionally contain the PRO domain and a predicted RT-like domain (FIG. 104A, Table 12). Phylogenetic analysis of Peg10 and its homologs supports the origin of this gene from LTR retrotransposons (FIGS. 122A-122B).

Among these genes, Arc is the most well studied. Drosophila Arc1 (darc1) is a gag homolog which contains the MA, CA, and NC domains. It has been shown to form capsids, bind its own mRNA, and transfer it from motor neurons to muscles at the neuromuscular junction (9). darc1 mRNA binding is dependent on its own 3′ UTR, and fusion of this sequence to heterologous mRNAs can initiate their export and transfer as well. Mus musculus Arc (MmArc), by contrast, contains only the CA domain and has also been shown to form capsids and transfers its own mRNA across synapses (10), but thus far this transfer has not been shown to be specific to Arc mRNA, likely due to the lack of a NC domain.

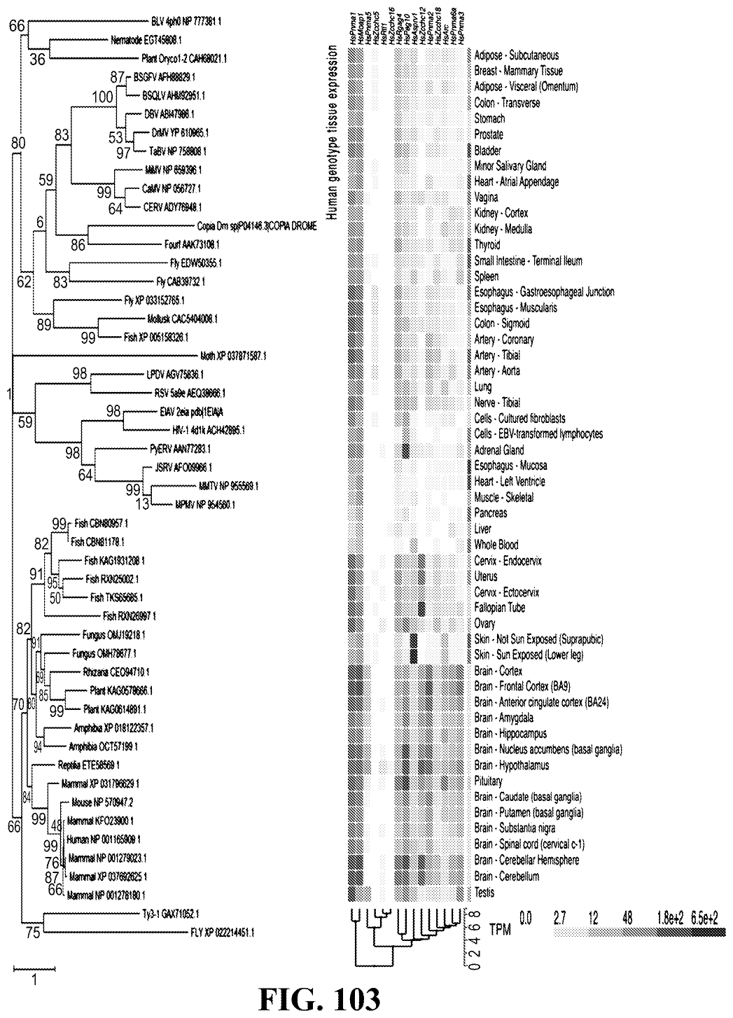

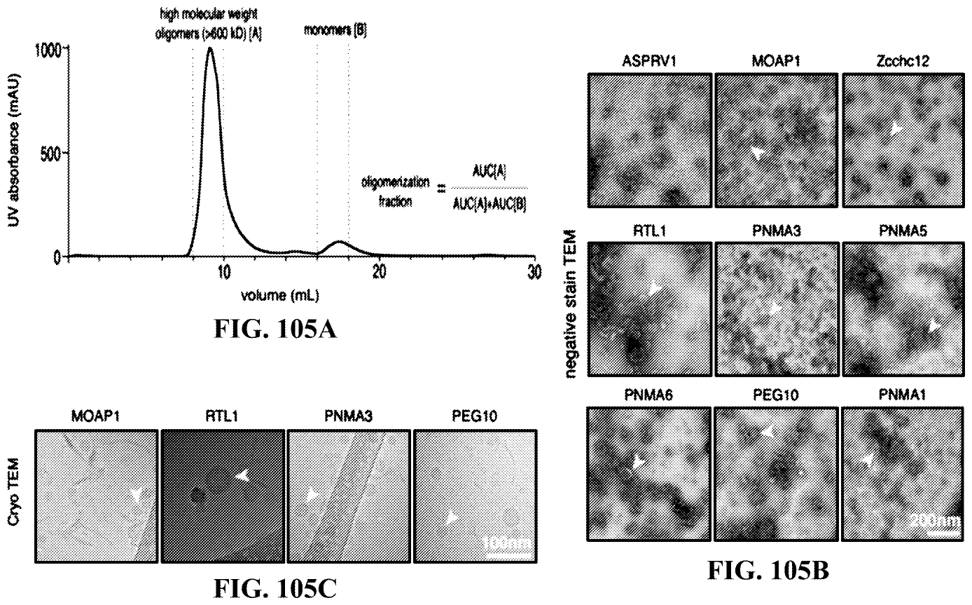

To narrow down the scope of the analysis, Applicant focused on CA-domain containing proteins that are conserved between human and mouse and have detectable levels of mRNA in adult human tissues, reasoning that such proteins were most likely to have been co-opted for important physiological roles in mammals (FIG. 103). Applicant produced mouse versions of the selected CA-containing proteins in E. coli and found that a number of these formed higher molecular weight oligomers identified by size exclusion (FIG. 104B, and FIG. 105A), as previously noted for some of these proteins, such as MmArc (10). Electron microscopy of these aggregated proteins showed that MmMOAP1, MmZCCHC12, MmRTL1, MmPNMA3, MmPNMA5, MmPNMA6a, and MmPEG10 self-assemble into capsid-like particles, many of which appear spherical (FIGS. 104C-104D, and FIGS. 105B-105C).

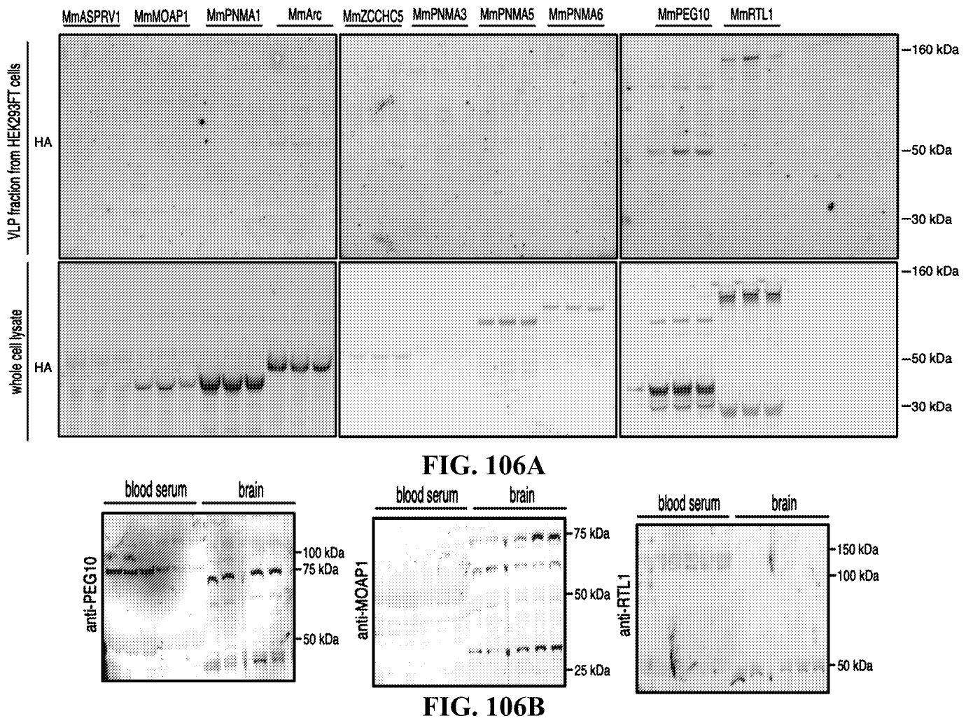

To determine if these proteins are secreted within an EV, Applicant overexpressed an epitope tagged mouse ortholog of each CA-containing gene in HEK293FT cells and harvested both the whole cell lysate and the VLP fraction by clarification and ultracentrifugation of the culture media (FIG. 104E). Applicant found that MmMOAP1, MmARC, MmPEG10, and MmRTL1 were all present in the VLP fraction (FIG. 104F, and FIG. 106A), but MmPEG10 was the most abundant protein in the VLP fraction (FIG. 104G). Additionally, endogenous MmPEG10, but not MmMOAP1 or MmRTL1, was readily detectable in cell-free adult mouse serum (FIG. 106B).

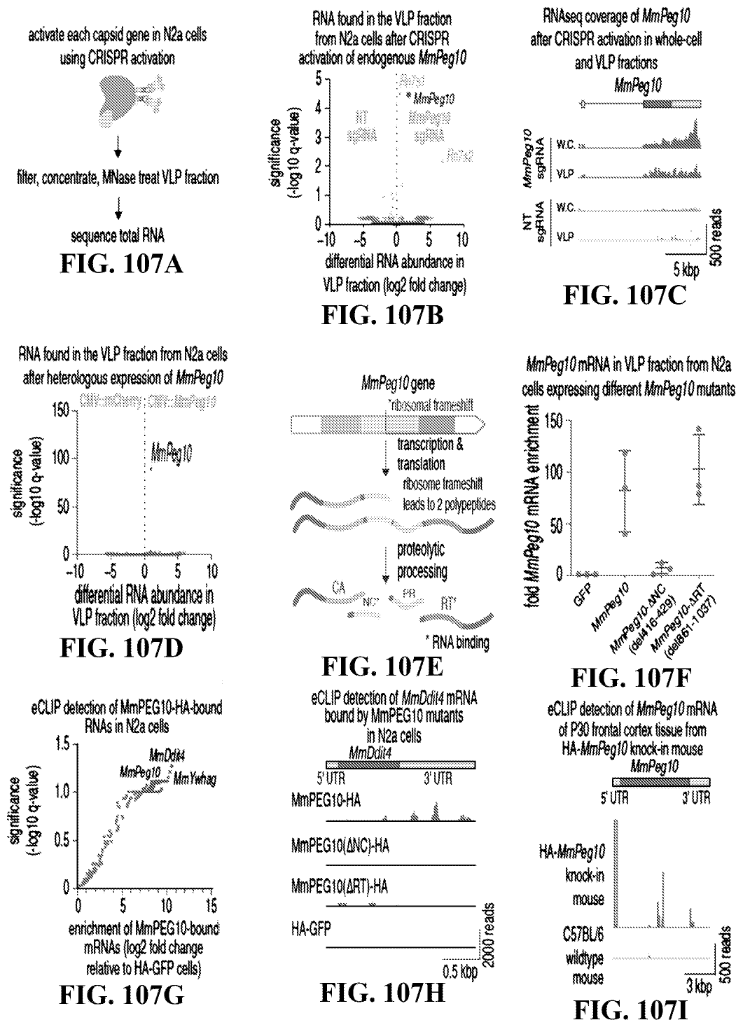

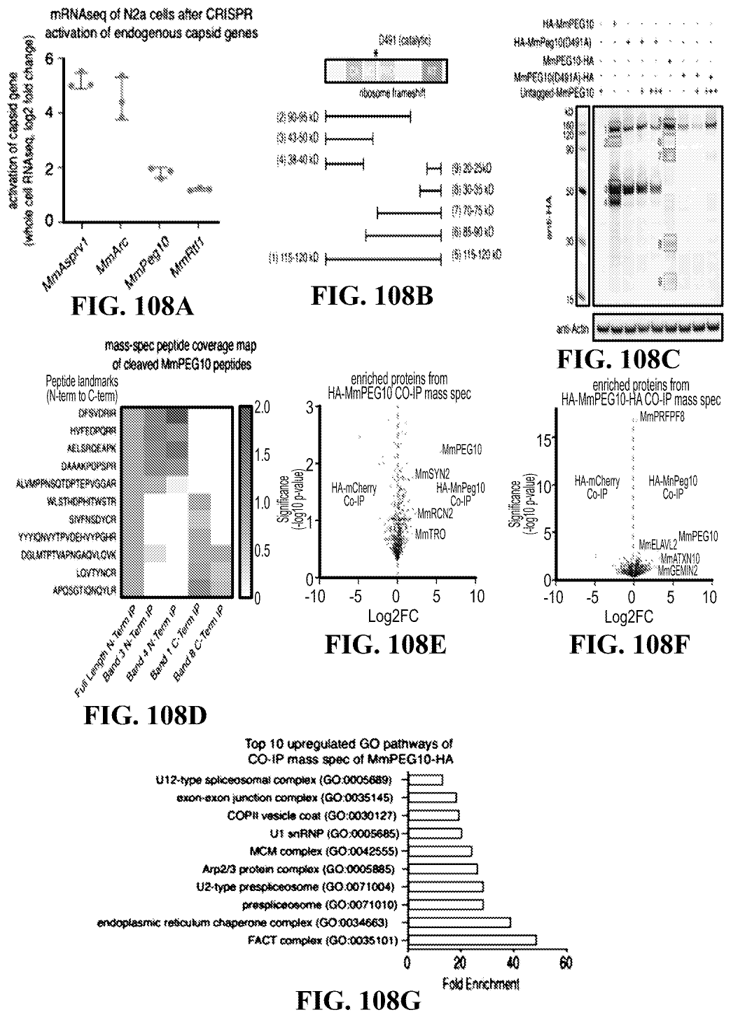

Applicant next tested whether any of the capsid-like particles formed by Gag homologs contained specific mRNAs using RNA sequencing. To avoid the possibility of transfected Gag homolog expression plasmids contributing to high background signal during sequencing, Applicant used CRISPR activation (19) to induce expression of endogenous genes in mouse N2a cells (FIGS. 107A and 108A). Applicant performed mRNA sequencing on whole cell lysate and the VLP fraction (following nuclease treatment to remove any residual, unencapsidated RNA) to identify RNA species in the VLP fraction. Applicant found MmPeg10 transcriptional activation led to accumulation of significant amounts of full length MmPeg10 mRNA transcripts in the VLP fraction (FIGS. 107B-107C). Previous work on MmPEG10 demonstrated that it binds a number of mRNAs inside of trophoblast stem cells including itself (13), however, here Applicant further shows that MmPEG10 binds and secretes its own mRNA into the VLP fraction. An important caveat of this experiment is that some of these proteins, particularly MmArc, are subject to regulation at the level of translation, so lack of enrichment in the VLP fraction could be due to low protein expression (20).

To confirm the observation for MmPeg10, Applicant transiently transfected overexpression plasmids of UTR-flanked MmPeg10 into N2a cells and found only enrichment for MmPeg10 mRNA in the VLP fraction (FIG. 107D) under this overexpression condition. PEG10 contains two putative nucleic acid binding domains, namely the NC and RT, which are released from the polypeptide upon PEG10 self processing (21) (FIG. 107E, Supplemental Note 1, FIGS. 108B-108D). Applicant generated deletions of these domains and found that mRNA export depends on the MmPEG10 NC, as loss of the nucleic acid-binding zinc finger CCHC motif (residues 416-429) from the MmPEG10 NC substantially reduced export of its mRNA (FIG. 107F).

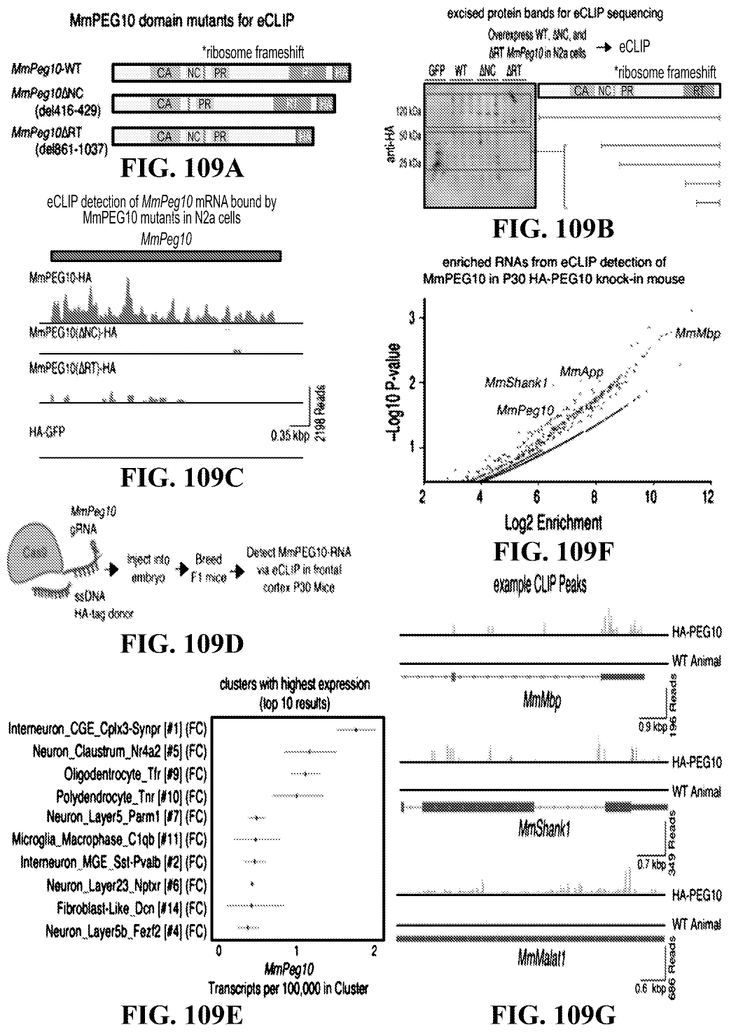

To better understand the roles of the nucleic acid binding domains of MmPEG10 in RNA binding, Applicant performed eCLIP in N2a cells after transient transfection with HA-tagged MmPeg10 as well as the NC and RT mutants (FIGS. 109A-109B). Compared to the control, MmPEG10 strongly bound a number of mRNAs in N2a cells including its own mRNA (FIG. 107G). Importantly, both the NC and the RT domains are required for the binding of these mRNAs by MmPEG10 (FIG. 107H and FIG. 109C). To confirm MmPEG10's cellular role in an in vivo context, Applicant generated knock-in mice carrying an N-terminal HA-tag on the endogenous MmPEG10 protein (FIG. 109D). Expression of MmPeg10 in cortical neurons has been demonstrated previously (FIG. 109E) (22). Endogenous MmPEG10 was also found to bind its own mRNA as well as other transcripts abundant in neurons (FIGS. 109F-109G); in contrast to previous datasets, Applicant detected strong MmPEG10 binding in the 5′ UTR as well as some additional binding near the boundary between the nucleocapsid and protease coding sequences and in the beginning of the 3′ UTR (FIG. 107I) (13).

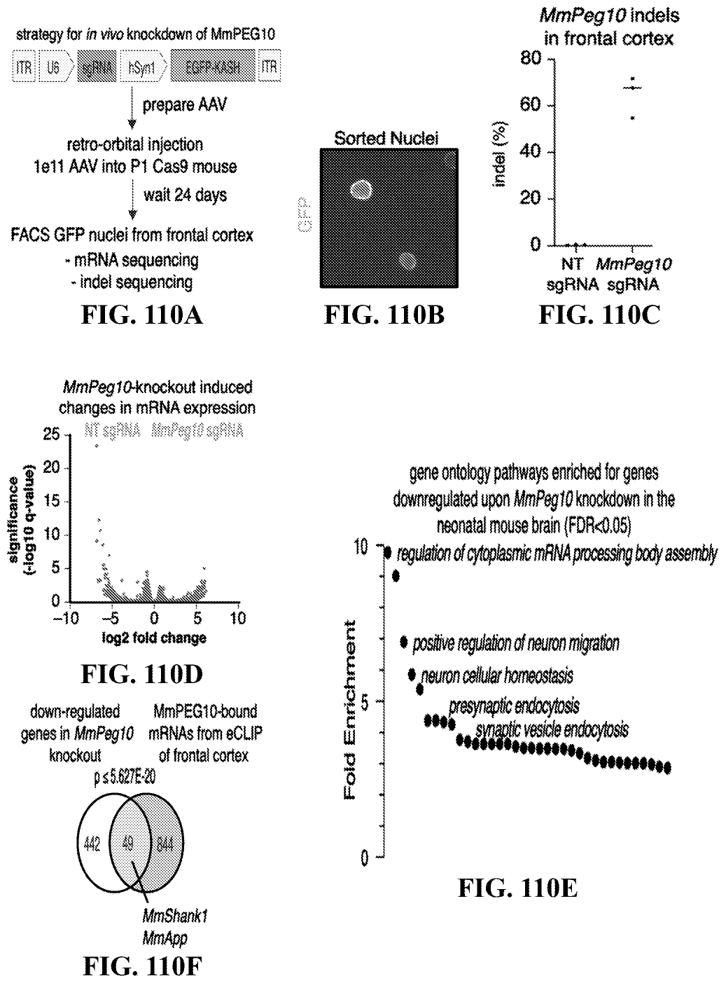

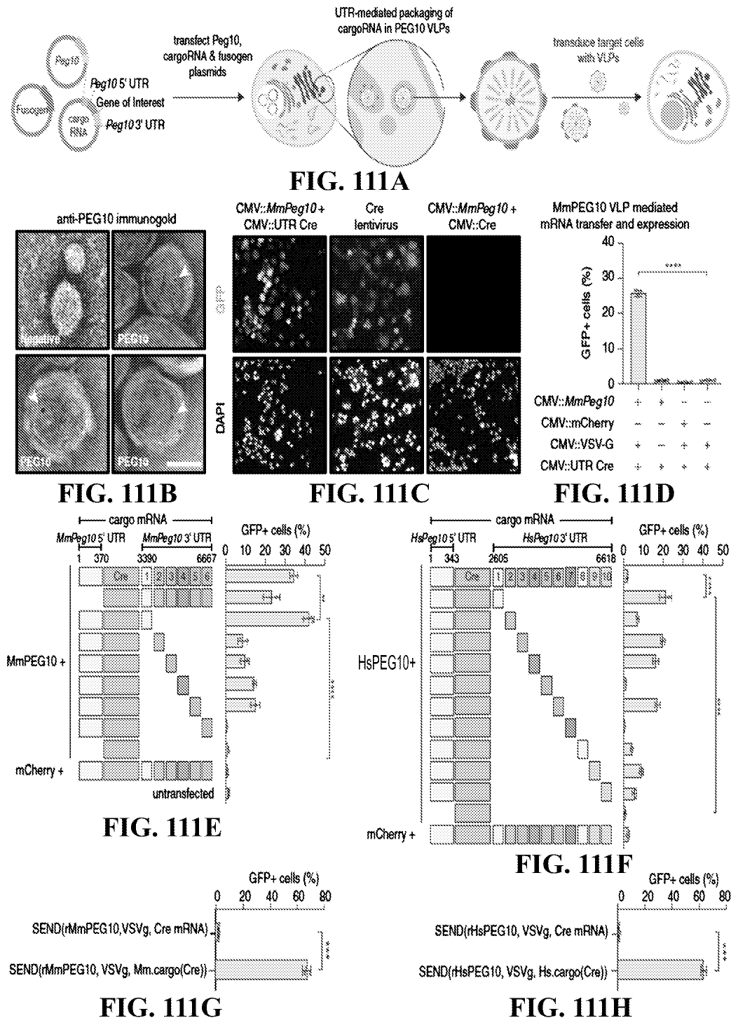

MmPEG10's binding of mRNA has been reported to increase the cellular abundance of target transcripts (13). To confirm this role of MmPEG10 in its native context in vivo, Applicant perturbed MmPeg10 gene expression in the postnatal mouse brain and assessed expression changes of MmPEG10 bound transcripts (Supplemental Note 2). Applicant found that the mRNAs of 49 genes downregulated in the brain upon MmPeg10 knockout are bound to MmPEG10 in the age-matched mouse brain (FIG. 110F), suggesting that one of the functions of MmPEG10 is to bind and stabilize mRNAs with fundamental roles in neurodevelopment.

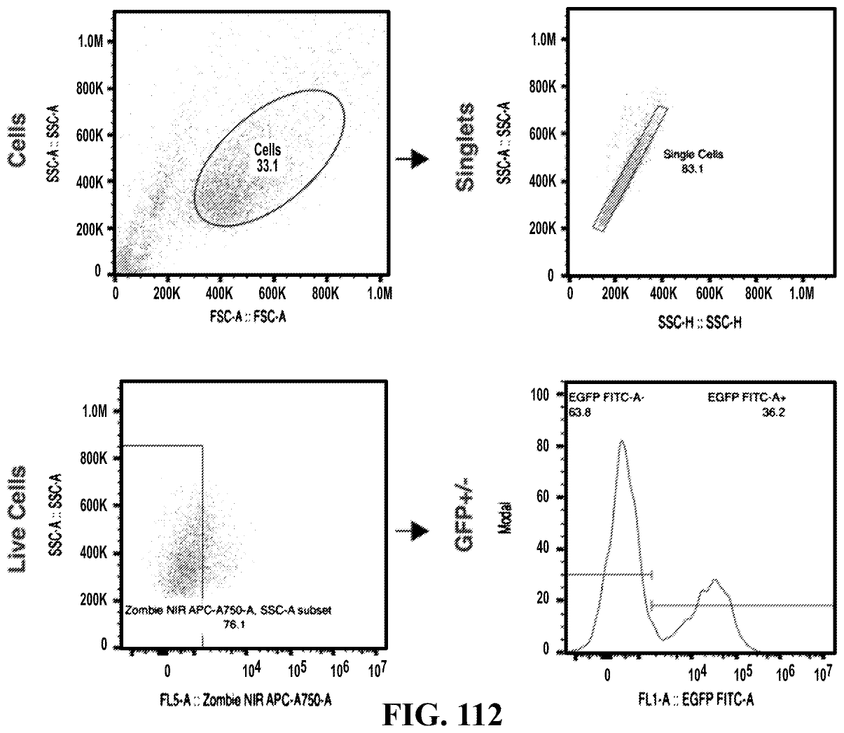

To reprogram MmPEG10 to bind and package heterologous RNA, Applicant tested whether a cargo mRNA consisting of both the 5′ and 3′ UTR of MmPeg10 flanking a gene of interest would be efficiently packaged, exported, delivered, and translated in recipient cells (FIG. 111A). This UTR grafting approach has been demonstrated for the Ty3 retroelement and darc1 (9, 23). Applicant first used a Cre-loxP system, a highly sensitive system for tracking RNA exchange that has been used previously with exosomes in vivo (24). Applicant flanked the Cre recombinase coding sequence with the MmPeg10 UTRs and co-transfected it with MmPeg10 with and without a fusogen, the Vesicular Stomatitis Virus envelope protein (VSVg) (FIG. 111A). Applicant found that MmPEG10 VLPs pseudotyped with VSVg are secreted within EVs that mediate transfer of Cre mRNA, not protein, into target loxP-GFP reporter N2a cells in a VSVg- and MmPeg10 UTR-dependent manner (FIGS. 111B-111D, and FIG. 112, Supplemental Note 3). This result suggests that addition of the Peg10 UTRs enables functional intercellular transfer of an mRNA via VLPs and that these VLPs require a fusogenic protein for cell entry.

Applicant next asked if there is a minimal UTR packaging signal for mediating efficient packaging and functional transfer. The 3′ UTR of MmPeg10 is approximately 4-kB long, but eCLIP indicates only portions of the 3′ UTR are bound by MmPEG10 (FIG. 107I). Applicant created constructs encoding the MmPeg10 5′ UTR, Cre, and 500-bp segments of the MmPeg10 3′ UTR. Applicant found that the proximal 500 bp of the MmPeg10 3′ UTR are sufficient for efficient functional transfer of Cre mRNA into target reporter cells (FIG. 111E). Importantly, no efficient functional mRNA transfer was observed for non-UTR flanked Cre or for Cre without the proximal 500 bp of the 3′ UTR. Moving forward, Applicant refers to RNA cargo flanked by the MmPeg10 5′ UTR and the proximal 500 bp of the 3′ UTR as Mm.cargo(RNA), where “(RNA)” specifies the cargo being flanked (e.g., Mm.cargo(Cre)).

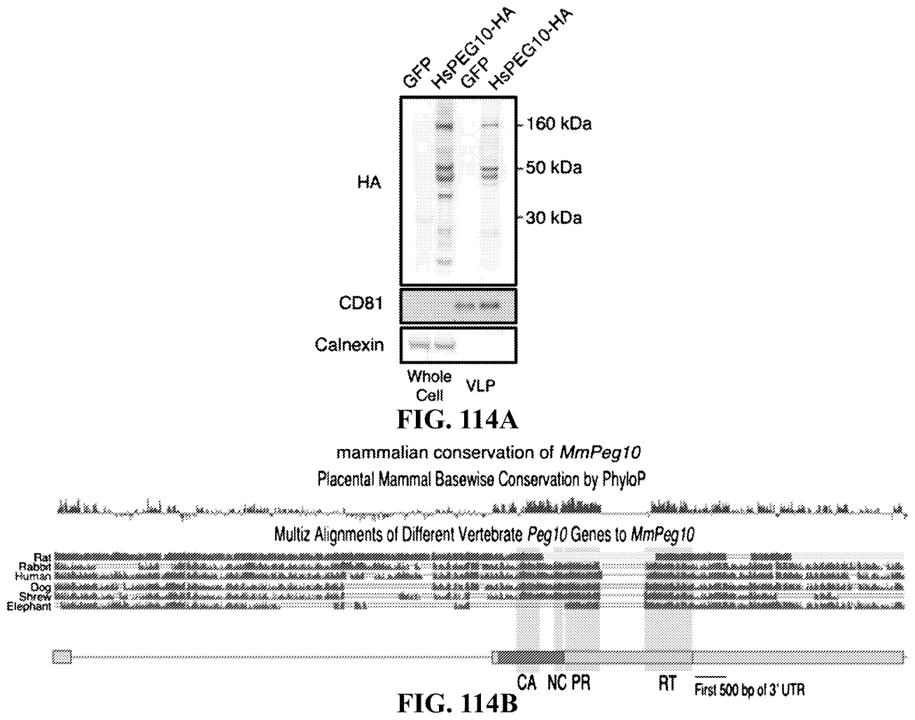

Like the mouse ortholog, HsPEG10 is an abundantly secreted protein in the VLP fraction (FIG. 114A). Using the same approach Applicant employed with MmPeg10, Applicant identified that the 5′ UTR and the first 500 bp of the HsPEG10 3′ UTR are sufficient to mediate functional transfer of Cre mRNA, hereafter denoted as Hs.cargo(RNA) (FIG. 111F). Interestingly, these functional regions of the UTRs are highly conserved across mammals (FIG. 114B). Similar to its mouse ortholog, the human system is specific, requiring HsPEG10 UTR sequences for functional mRNA transfer whereas non-flanked Cre produced only minimal reporter cell activity (FIG. 111F).

To further boost the packaging of a cargo(RNA) by PEG10, Applicant explored the impact of removing any additional PEG10 cis binding elements within the MmPeg10/HsPEG10 coding sequence. For both human and mouse, transfer was increased as a result of recoding the sequence between the NC and the PRO domains, which corresponds to the MmPEG10-bound region in the eCLIP experiments (FIG. 107I, Supplemental Note 4).

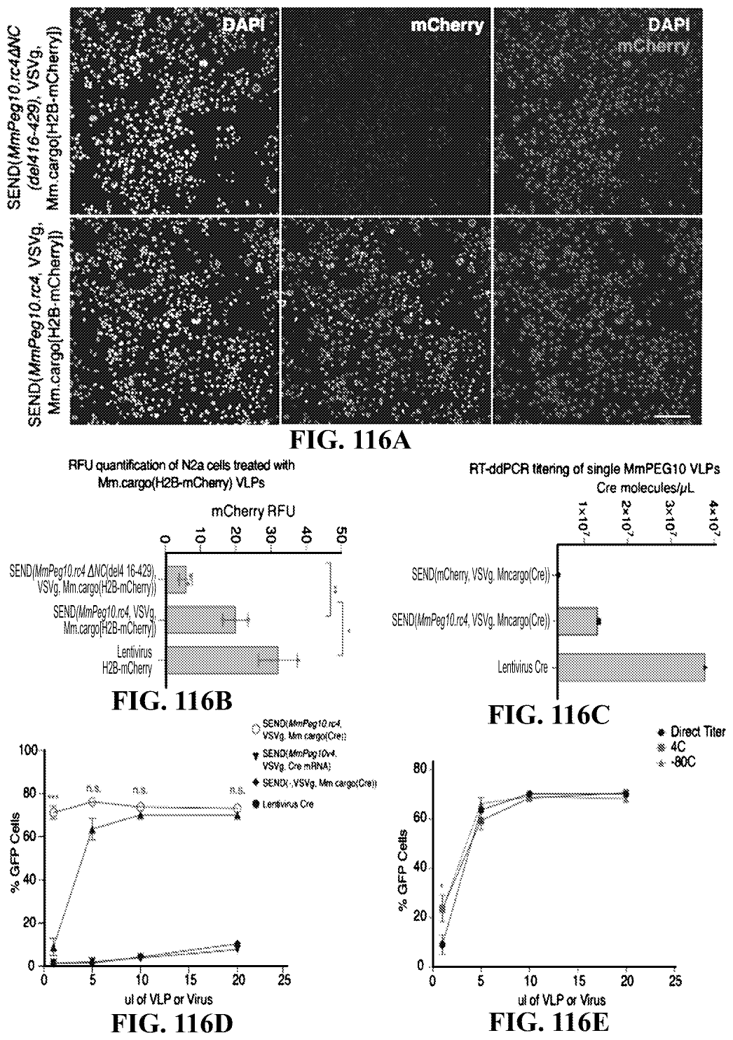

Combining these optimizations, Applicant produced VLPs with the recoded mouse and human PEG10 (rMmPEG10 or rHsPEG10), VSVg, and the optimized cargo(RNA) containing the first 500 bp of the 3′ UTR; Applicant refers to this system as Selective Endogenous eNcapsidation for cellular Delivery (SEND). With SEND Applicant detected a substantial (up to 60%) increase in functional transfer of cargo(Cre) into N2a cells for both human and mouse PEG10 (FIGS. 111G and 111H). Furthermore, Applicant shows that VLPs produced with rMmPEG10 can mediate the functional transfer of H2B-mCherry (FIGS. 116 A and 116B). Comparison of SEND to previously developed delivery vectors showed that SEND is 4-5 fold less potent than an integrating lentiviral vector as assayed by digital droplet PCR and functional titration (FIG. 116B-116E). However, given that SEND delivers mRNA rather than integrating an overexpression cassette, Applicant expects it to perform competitively against other mRNA delivery vehicles.

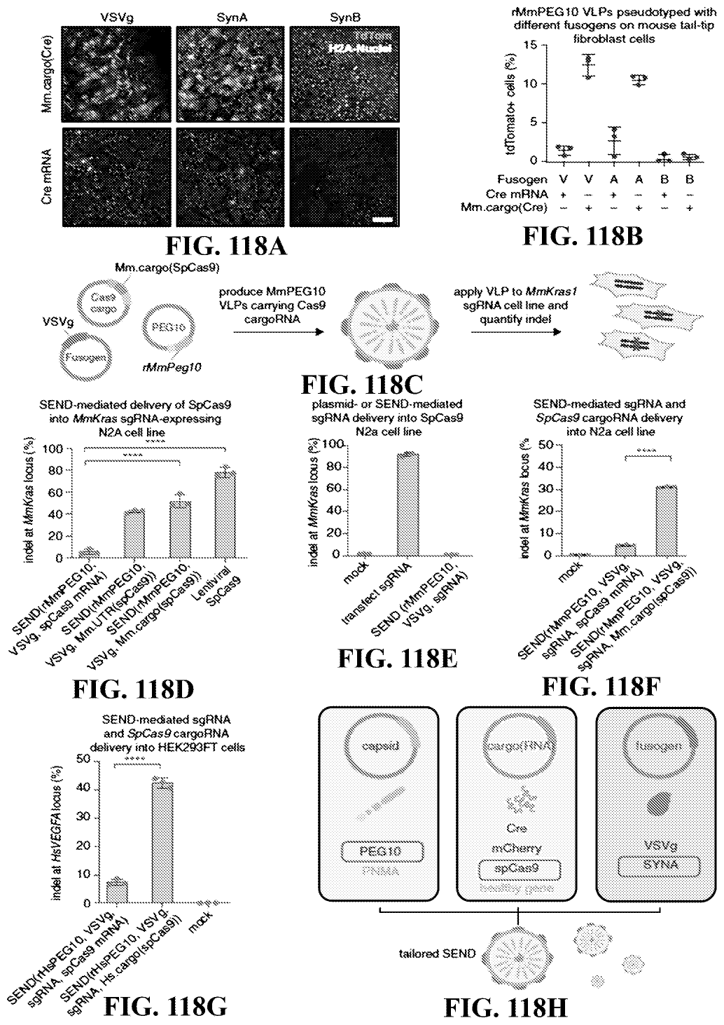

To generate a fully endogenous SEND system, Applicant tested whether VSVg can be replaced with an endogenous fusogenic transmembrane protein. Given the overlapping tissue expression of MmPeg10/HsPEG10 and syncytin genes (Supplemental Note 5), Applicant tested the feasibility of pseudotyping the mouse SEND system with MmSYNA, or MmSYNB compared to pseudotyping with VSVg. Pseudotyped particles were incubated with tail-tip fibroblasts from loxP-tdTomato reporter mice, a cell type which we have found amenable to transduction by these fusogens. Based on previous reports, we added the transduction enhancer vectofusin-1 to the supernatant for MmSYNA and MmSYNB particles to enhance in vitro transduction (25). In these primary cells, both VSVg and MmSYNA enabled SEND-mediated functional transfer of Mm.cargo(Cre) while MmSYNB did not (FIGS. 118A-118B). Again, this packaging was highly specific, as only UTR flanked mRNA (i.e., Mm.cargo(Cre)) was functionally transferred. Together with MmSYNA, SEND can be configured as a fully endogenous system for functional gene transfer.

Supported by the understanding of the minimal requirements for PEG10-mediated mRNA delivery (i.e., UTRs and an endogenous fusogen), Applicant could begin to probe the endogenous role of MmPEG10-mediated MmPeg10 RNA delivery in neurons. Functional transfer of MmSYNA pseudotyped VLPs carrying the native PEG10 transcript into primary mouse cortical neurons led to upregulation of a number of genes involved in neurodevelopment (Supplemental Note 6). This finding reinforces the notion that one role of endogenous MmPeg10 delivery is binding and stabilizing specific mRNA transcripts in recipient cells. RNA sequencing of N2a cells receiving Mm.cargo(Peg10) revealed substantial gene expression changes upon MmPeg10 delivery that were largely abrogated with PEG10 mediated Mm.cargo(Cre) delivery (Supplemental Note 7). This suggests transferring a reprogrammed cargo does not have the same impact on recipient cells as transferring MmPeg10 and indicates that MmPeg10 transcript delivery rather than the delivery of MmPEG10 protein is responsible for the observed gene expression changes. It remains unclear whether MmPEG10 VLPs are natively pseudotyped by the endogenous fusogen MmSYNA to enable cellular uptake of PEG10 VLPs in the central nervous system.

To further characterize the modularity of the components of this system, Applicant tested different cargoRNAs. Using the same pipeline developed for cargo(Cre), Applicant tested whether SEND could mediate the functional transfer of a large ˜5 kb Mm.cargo(SpCas9) into N2a cell lines constitutively expressing an sgRNA against MmKras (FIG. 118C). SEND was able to functionally transfer SpCas9, leading to ˜60% indels in recipient cells (FIG. 118D); similar to the results with Cre, SEND is specific and only able to efficiently functionally transfer SpCas9 flanked by either the full length or optimized Peg10 UTR sequences.

To create an all-in-one vector for delivery of sgRNA and SpCas9, Applicant first tested if an sgRNA can be efficiently delivered by SEND. Applicant independently packaged an sgRNA targeting Kras into rMmPeg10 VLPs by co-expressing rMmPeg10 with VSVg and a U6 driven sgRNA and incubated them with Cas9 expressing N2a cells; Applicant saw very little activity even though direct transfection of the guide showed robust indel formation (FIG. 118E). Applicant found, however, that co-packaging the guide alongside Mm.cargo(SpCas9) by co-expressing Mm.cargo(SpCas9) with a U6 driven sgRNA on a separate plasmid was sufficient to mediate 30% indels (FIG. 118F). To determine the reproducibility of this genome editing approach, Applicant repeated this co-packaging strategy with the human SEND system and were able to generate ˜40% indels in HEK293FT cells at the HsVEGFA locus (FIG. 118G).

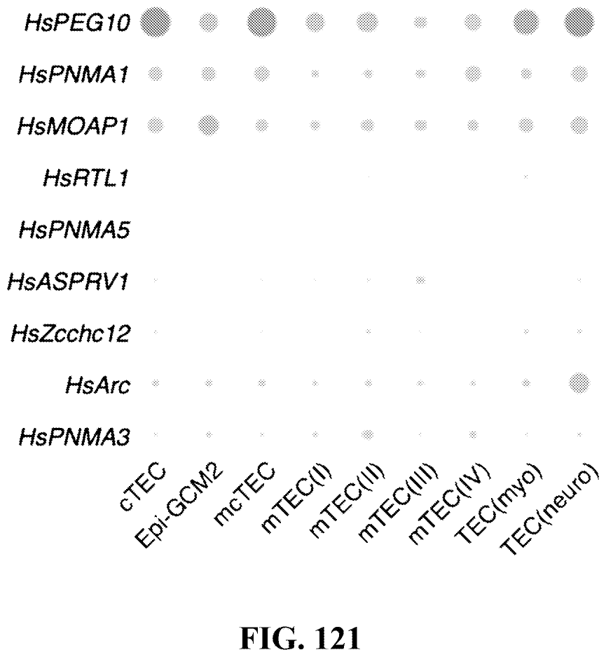

The development of SEND from an endogenous retroelement complements existing delivery approaches using lipid nanoparticles (26), VLPs derived from bona fide retroviruses (27-29), and active mRNA loading approaches in EVs (30, 31). Moreover, SEND may have reduced immunogenicity compared to currently available viral vectors (32) due to its use of endogenous human proteins. Supporting this is gene expression data from the developing human thymus which demonstrates that HsPEG10 is highly expressed compared to other CA-containing genes in the thymic epithelium (33) (FIG. 121), which is responsible for T cell tolerance induction. As a modular, fully endogenous system, SEND has the potential to be extended into a minimally immunogenic delivery platform that can be repeatedly dosed, greatly expanding the applications for nucleic acid therapy.

### References for Example 12

1. J. L. Goodier, H. H. Kazazian Jr, Retrotransposons revisited: the restraint and rehabilitation of parasites. Cell. 135, 23-35 (2008).
2. A. F. Smit, Interspersed repeats a-d other mementos of transposable elements in mammalian genomes. Curr. Opin. Genet. Dev. 9, 657-663 (1999).
3. M. R. Patel, M. Emerman, H. S. Malik, Paleovirology—ghosts and gifts of viruses past. Curr. Opin. Virol. 1, 304-309 (2011).
4. L. Guio, J. Gonzilez, New Insights on the Evolution of Genome Content: Population Dynamics of Transposable Elements in Flies and Humans. Methods Mol. Biol. 1910, 505-530 (2019).
5. C. Feschotte, C. Gilbert, Endogenous viruses: insights into viral evolution and impact on host biology. Nat. Rev. Genet. 13, 283-296 (2012).
6. F. J. Kim, J.-L. Battini, N. Manel, M. Sitbon, Emergence of vertebrate retroviruses and envelope capture. Virology. 318, 183-191 (2004).
7. A. Dupressoir, C. Vemochet, O. Bawa, F. Harper, G. Pierron, P. Opolon, T. Heidmann, Syncytin-A knockout mice demonstrate the critical role in placentation of a fusogenic, endogenous retrovirus-derived, envelope gene. Proc. Natl. Acad. Sci. U.S.A 106, 12127-12132 (2009).
8. C. Myrum, A. Baumann, H. J. Bustad, M. I. Flydal, V. Mariaule, S. Alvira, J. Cudllar, J. Haavik, J. Soulé, J. M. Valpuesta, J. A. Márquez, A. Martinez, C. R. Bramham, Arc is a flexible modular protein capable of reversible self-oligomerization. Biochem. J. 468, 145-158 (2015).
9. J. Ashley, B. Cordy, D. Lucia, L. G. Fradkin, V. Budnik, T. Thomson, Retrovirus-like Gag Protein Arc1 Binds RNA and Traffics across Synaptic Boutons. Cell. 172, 262-274.e11 (2018).
10. E. D. Pastuzyn, C. E. Day, R. B. Keans, M. Kyrke-Smith, A. V. Taibi, J. McCormick, N. Yoder, D. M. Belnap, S. Erlendsson, D. R. Morado, J. A. G. Briggs, C. Feschotte, J. D. Shepherd, The Neuronal Gene Arc Encodes a Repurposed Retrotransposon Gag Protein that Mediates Intercellular RNA Transfer. Cell. 173, 275 (2018).
11. S. F. Erica Korb, Arc in synaptic plasticity: from gene to behavior. Trends Neurosci. 34, 591 (2011).
12. P. Barragan-Iglesias, J. B. De La Pena, T. F. Lou, S. Loerch, Intercellular Arc signaling regulates vasodilation (2020) (available at https://papers.ssm.com/sol3/papers.cfm?abstract_id=3684856).
13. M. Abed, E. Verschueren, H. Budayeva, P. Liu, D. S. Kirkpatrick, R. Reja, S. K. Kummerfeld, J. D. Webster, S. Gierke, M. Reichelt, K. R. Anderson, R. J. Newman, M. Roose-Girma, Z. Modrusan, H. Pektas, E. Maltepe, K. Newton, V. M. Dixit, The Gag protein PEG10 binds to RNA and regulates trophoblast stem cell lineage specification. PLoS One. 14, e0214110 (2019).
14. R. Ono, K. Nakamura, K. Inoue, M. Naruse, T. Usami, N. Wakisaka-Saito, T. Hino, R. Suzuki-Migishima, N. Ogonuki, H. Miki, T. Kohda, A. Ogura, M. Yokoyama, T. Kaneko-Ishino, F. Ishino, Deletion of Peg10, an imprinted gene acquired from a retrotransposon, causes early embryonic lethality. Nat. Genet. 38, 101-106 (2006).
15. C. Henke, P. L. Strissel, M.-T. Schubert, M. M-chell, C. C. Stolt, F. Faschingbauer, M. W. Beckmann, R. Strick, Selective expression of sense and antisense transcripts of the sushi-ichi-related retrotransposon-derived family during mouse placentogenesis. Retrovirology. 12, 9 (2015).
16. M. Krupovic, E. V. Koonin, Multiple origins of viral capsid proteins from cellular ancestors. Proc. Nati. Acad. Sci. U.S.A 114, E2401-E2410 (2017).
17. S. O. Dodonova, S. Prinz, V. Bilanchone, S. Sandmeyer, J. A. G. Briggs, Structure of the Ty3/Gypsy retrotransposon capsid and the evolution of retroviruses. Proc. Natl. Acad. Sci. U.S.A. 116, 10048-10057 (2019).
18. M. Campillos, T. Doerks, P. K. Shah, P. Bork, Computational characterization of multiple Gag-like human proteins. Trends Genet. 22, 585-589 (2006).
19. S. Konermann, M. D. Brigham, A. E. Trevino, J. Joung, O. O. Abudayyeh, C. Barcena, P. D. Hsu, N. Habib, J. S. Gootenberg, H. Nishimasu, O. Nureki, F. Zhang, Genome-scale transcriptional activation by an engineered CRISPR-Cas complex. Nature. 517, 583-588 (2015).
20. C. S. Wallace, G. L. Lyford, P. F. Worley, O. Steward, Differential intracellular sorting of immediate early gene mRNAs depends on signals in the mRNA sequence. J. Neurosci. 18, 26-35 (1998).
21. M. Golda, J. A. Móltyán, M. Mahdi, J. Tözsér, Functional Study of the Retrotransposon-Derived Human PEG10 Protease. Int. J. Mol. Sci. 21 (2020), doi:10.3390/ijms21072424.
22. A. Saunders, E. Z. Macosko, A. Wysoker, M. Goldman, F. M. Krienen, H. de Rivera, E. Bien, M. Baum, L. Bortolin, S. Wang, A. Goeva, J. Nemesh, N. Kamitaki, S. Brumbaugh, D. Kulp, S. A. McCarroll, Molecular Diversity and Specializations among the Cells of the Adult Mouse Brain. Cell. 174 (2018), pp. 1015-1030.e16.
23. K. Clemens, V. Bilanchone, N. Beliakova-Bethell, Sequence requirements for localization and packaging of Ty3 retroelement RNA. Virus Res. (2013) (available at https://www.sciencedirect.com/science/article/pii/S0168170212003784).
24. K. Ridder, A. Sevko, J. Heide, M. Dams, A.-K. I J. Macas, J. Starmann, M. Tjwa, K. H. Plate, H. Sultmann, P. Altevogt, V. Umansky, S. Momma, Extracellular vesicle-mediated transfer of functional RNA in the tumor microenvironment. Oncoimmunology. 4, e1008371 (2015).
25. Y. Coquin, M. Ferrand, A. Seye, L. Menu, A. Galy, Syncytins enable novel possibilities to transduce human or mouse primary B cells and to achieve well-tolerated in vivo gene transfer. Cold Spring Harbor Laboratory (2019), p. 816223.
26. P. S. Kowalski, A. Rudra, L. Miao, D. G. Anderson, Delivering the Messenger: Advances in Technologies for Therapeutic mRNA Delivery. Mol. Ther. 27, 710-728 (2019).
27. U. Mock, K. Riecken, B. Berdien, W. Qasim, E. Chan, T. Cathomen, B. Fehse, Novel lentiviral vectors with mutated reverse transcriptase for mRNA delivery of TALE nucleases. Sci. Rep. 4, 6409 (2014).
28. S. J. Kaczmarczyk, K. Sitaraman, H. A. Young, S. H. Hughes, D. K. Chatterjee, Protein delivery using engineered virus-like particles. Proc. Natl. Acad. Sci. U.S.A 108, 16998-17003 (2011).
29. P. E. Mangeot, V. Risson, F. Fusil, A. Mamef, E. Laurent, J. Blin, V. Moumetas, E. Massourides, T. J. M. Sohier, A. Corbin, F. Aubé, M. Teixeira, C. Pinset, L. Schaeffer, G. Legube, F.-L. Cosset, E. Verhoeyen, T. Ohlmann, E. P. Ricci, Genome editing in primary cells and in vivo using viral-derived Nanoblades loaded with Cas9-sgRNA ribonucleoproteins. Nat. Commun. 10, 45 (2019).
30. R. Kojima, D. Bojar, G. Rizzi, G. C.-E. Hamri, M. D. El-Baba, P. Saxena, S. Ausländer, K. R. Tan, M. Fussenegger, Designer exosomes produced by implanted cells intracerebrally deliver therapeutic cargo for Parkinson's disease treatment. Nat. Commun. 9, 1305 (2018).
31. M. E. Hung, J. N. Leonard, A platform for actively loading cargo RNA to elucidate limiting steps in EV-mediated delivery. J Extracell Vesicles. 5, 31027 (2016).
32. J. L. Shirley, Y. P. de Jong, C. Terhorst, R. W. Herzog, Immune Responses to Viral Gene Therapy Vectors. Mol. Ther. 28, 709-722 (2020).
33. J.-E. Park, R. A. Botting, C. Dominguez Conde, D.-M. Popescu, M. Lavaert, D. J. Kunz, I. Goh, E. Stephenson, R. Ragazzini, E. Tuck, A. Wilbrey-Clark, K. Roberts, V. R. Kedlian, J. R. Ferdinand, X. He, S. Webb, D. Maunder, N. Vandamme, K. T. Mahbubani, K. Polanski, L. Mamanova, L. Bolt, D. Crossland, F. de Rita, A. Fuller, A. Filby, G. Reynolds, D. Dixon, K. Saeb-Parsy, S. Lisgo, D. Henderson, R. Vento-Tormo, O. A. Bayraktar, R. A. Barker, K. B. Meyer, Y. Saeys, P. Bonfanti, S. Behjati, M. R. Clatworthy, T. Taghon, M. Haniffa, S. A. Teichmann, A cell atlas of human thymic development defines T cell repertoire formation. Science. 367 (2020), doi:10.1126/science.aay3224.

### Example 13—Supplemental Notes and Materials and Methods for Example 12

### Supplemental Note 1

The full length MmPEG10 polyprotein encodes four conserved retroelement domains: CA, NC, PRO, and a catalytically inactive RT (34, 35). Like some retroelements, the transcript is translated in two protein isoforms, namely, reading frame 1 (RF1, gag), which encodes only CA and NC, and reading frame 2 (RF2, pol), which is generated via a −1 ribosomal frameshift (36) and encodes all four domains (FIG. 104A). The MmPEG10 polyprotein is then processed in cis by the protease into individual proteins (FIG. 107E) as shown previously (21) and confirmed by Western blotting. The band sizes on the Western blot correspond to the sizes of the predicted functional domains of MmPEG10, as confirmed by examination of the peptide signatures from these bands by mass spectroscopy (FIGS. 108B-108D).

To characterize protein interactions that might be relevant for the functions of MmPEG10, Applicant performed Co-IP mass spectroscopy on N and C-terminally HA-tagged MmPeg10 that was transiently transfected into N2a cells (FIGS. 108E and 108F) and found that MmPEG10 is associated with RNA processing proteins, such as MOV10 and GEMIN2 (FIG. 108G).

### Supplemental Note 2

Applicant injected AAV-PHP.eB encoding a neuron specific expression of nuclear membrane GFP protein (GFP-KASH) and sgRNAs against MmPeg10 into P1 spCas9 mice (FIG. 110A). After 24 days, Applicant harvested and performed FACS for GFP+ nuclei (FIG. 110B) from the frontal cortex of the MmPeg10 knockout mice and non-targeting control mice. Next generation sequencing for indels in theMmPeg10 locus confirmed robust gene knockout with 55-70% indels (FIG. 110C). In cortical neurons, many genes were significantly downregulated and a few genes were upregulated in MmPeg10 knockout nuclei compared to the control sorted nuclei (FIG. 110D). Pathway analysis of these genes shows significant enrichment of downregulated genes responsible for cytoplasmic RNA processing, as well as pathways involved in neuron migration, homeostasis, and synaptic endocytosis (FIG. 110E).

### Supplemental Note 3

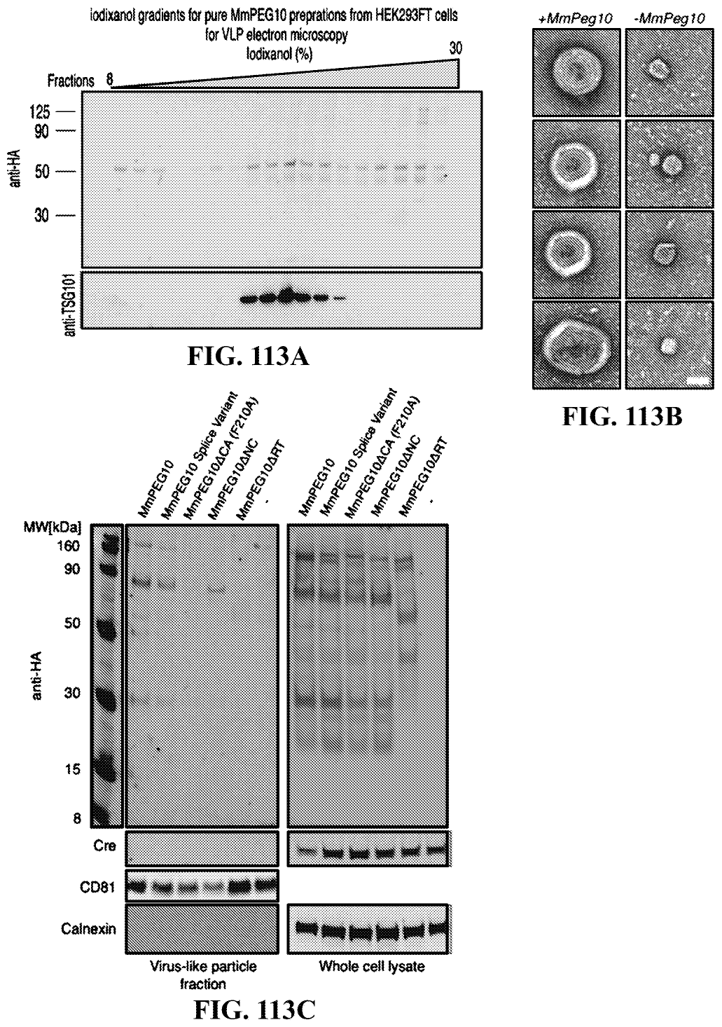

Immunogold TEM analysis of VLPs derived from HEK293FT cells demonstrates MmPEG10 VLPs are membrane bound and confirms their size to be around 100 nm (FIG. 111B). MmPEG10 particles fractionate alongside the exosomal marker TSG101 and contain visible capsid-like structures (FIGS. 113A-113B). To ensure that MmPEG10 VLPs transferred mRNA rather than protein, Applicant blotted the VLPs for HA as well as Cre recombinase, calnexin, and CD81 (FIG. 113C). The VLP fraction contained HA tagged MmPEG10 and CD81, but not Cre recombinase or calnexin, a marker of cell contamination. By contrast, Calnexin, Cre, and MmPEG10 were readily detectable in the whole cell lysate.

### Supplemental Note 4

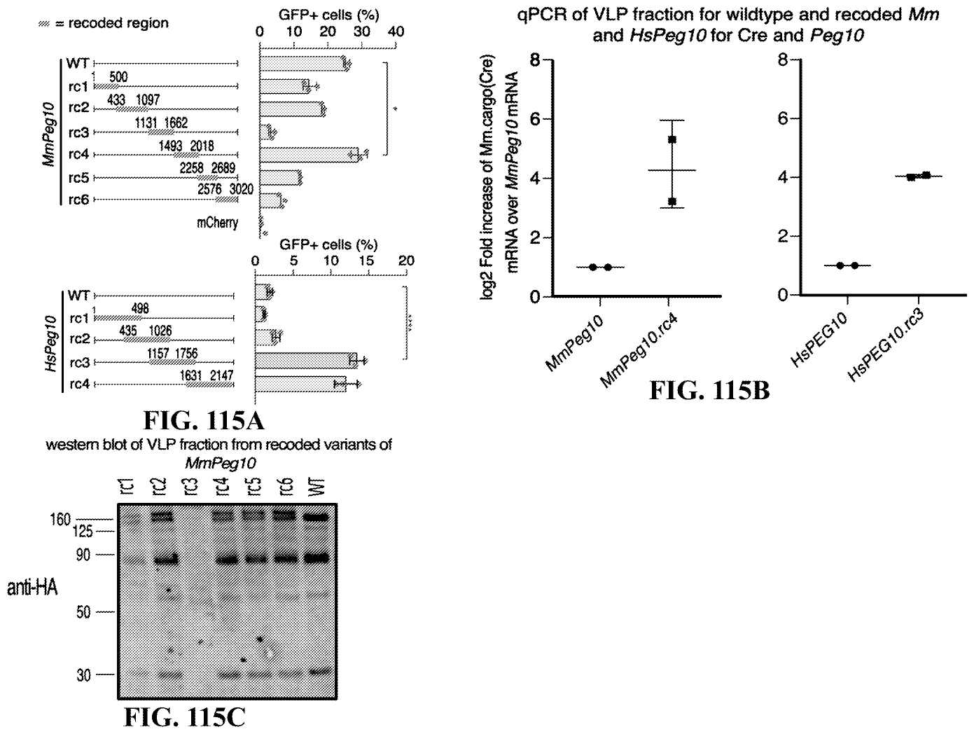

While both UTRs of the MmPeg10 mRNA are bound by the MmPEG10 protein, eCLIP data indicate that there is also binding inside theMmPeg10 coding sequence (FIG. 107I). Without being bound by theory, Applicant hypothesized that this affinity led to MmPeg10 mRNA packaging, thereby reducing the efficiency of cargo(RNA) transfer. Applicant generated six mouse and five human Peg10/PEG10 overexpression constructs, each with an overlapping 500-700 bp window of recoded codons to eliminate self mRNA binding (FIG. 115A). For mouse, the most effective was rc4, referred to hereafter as rMmPeg10, and for human rc3, referred to hereafter as rHsPeg10. For VLPs produced with both rHs and rMmPeg10 there was an increase in the ratio of secreted cargo(Cre) relative to MmPeg10/HsPEG10 ‘self’ RNA by 10-20 fold in the MNaAse treated VLP fraction without a corresponding change in vesicle secretion (FIGS. 115B-115C).

### Supplemental Note 5

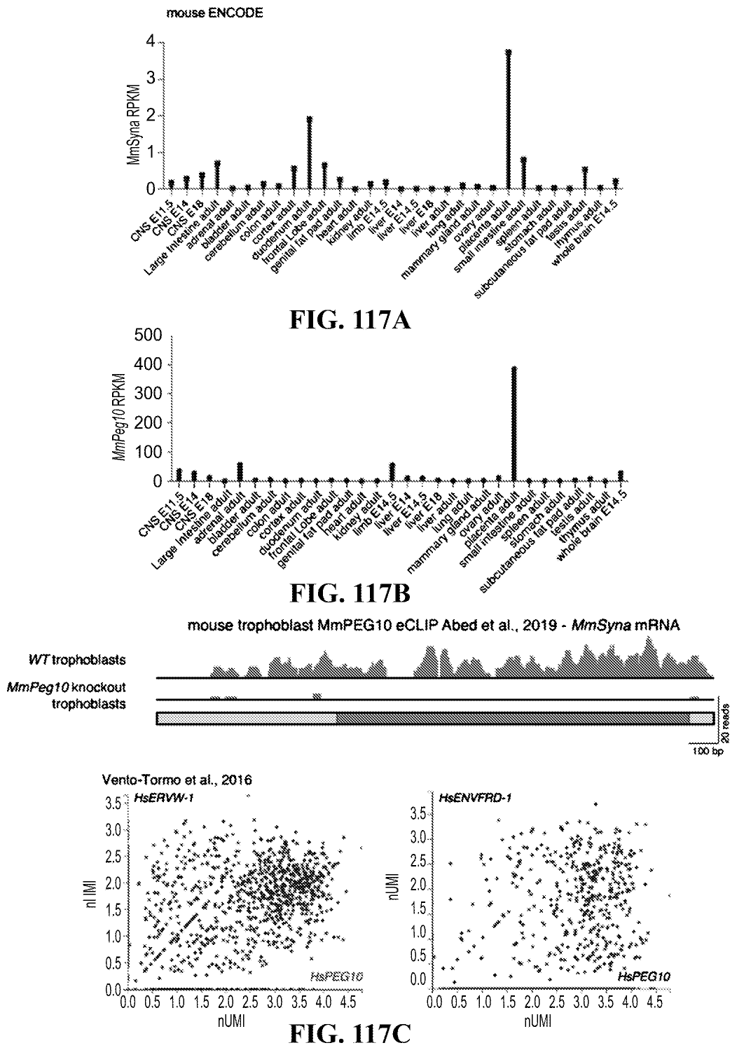

Publicly available tissue-wide sequencing databases show that MmPeg10 is highly expressed in the placenta (FIG. 117A) (13), which also expresses the fusogenic syncytin proteins MmSYNA and MmSYNB; these can be used to pseudotype lentivirus for efficient transgene delivery (25). Re-analyzing previous MmPEG10 eCLIP data in mouse trophoblast stem cells shows a direct interaction between MmPEG10 and the mouse syncytin transcript MmSyna (FIG. 117B). Previously published single-cell sequencing databases of human syncytiotrophoblasts confirms that analogous endogenous fusogens (ERVW-1 and ERVFRD-1) are expressed in the same cells as HsPEG10 (FIG. 117C) (37).

### Supplemental Note 6

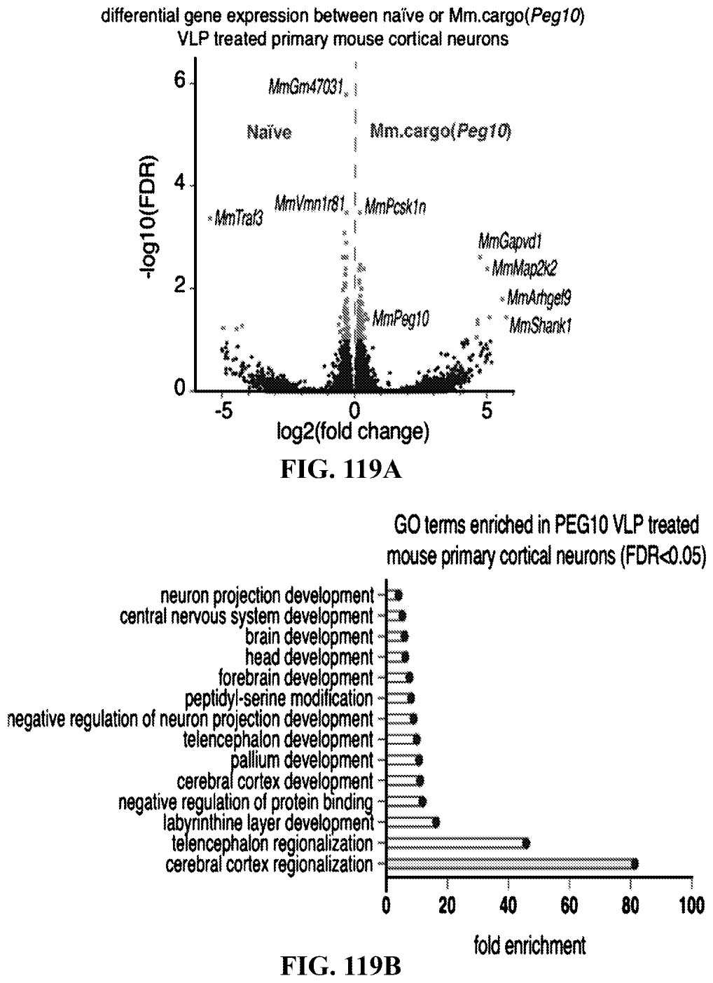

To understand the effects of MmPEG10 VLPs on target cells, which may indicate a potential biological function for MmPeg10-mediated intercellular mRNA transfer, Applicant treated primary mouse cortical neurons with MmSYNA pseudotyped MmPEG10 VLPs containing Mm.cargo(Peg10). After 72 hours Applicant performed mRNA sequencing on the recipient neurons and identified a large number of key developmental genes that are differentially expressed (FIG. 119A). For example, Applicant detected upregulation of MmShank1, which Applicant showed to be bound by PEG10 protein in our eCLIP data (FIGS. 109F-109G) and downregulated upon knockout of MmPeg10 in the developing mouse brain (FIGS. 110D and 110F). Pathway analysis suggests these upregulated genes are involved in neurodevelopment more broadly (FIG. 119B).

### Supplemental Note 7

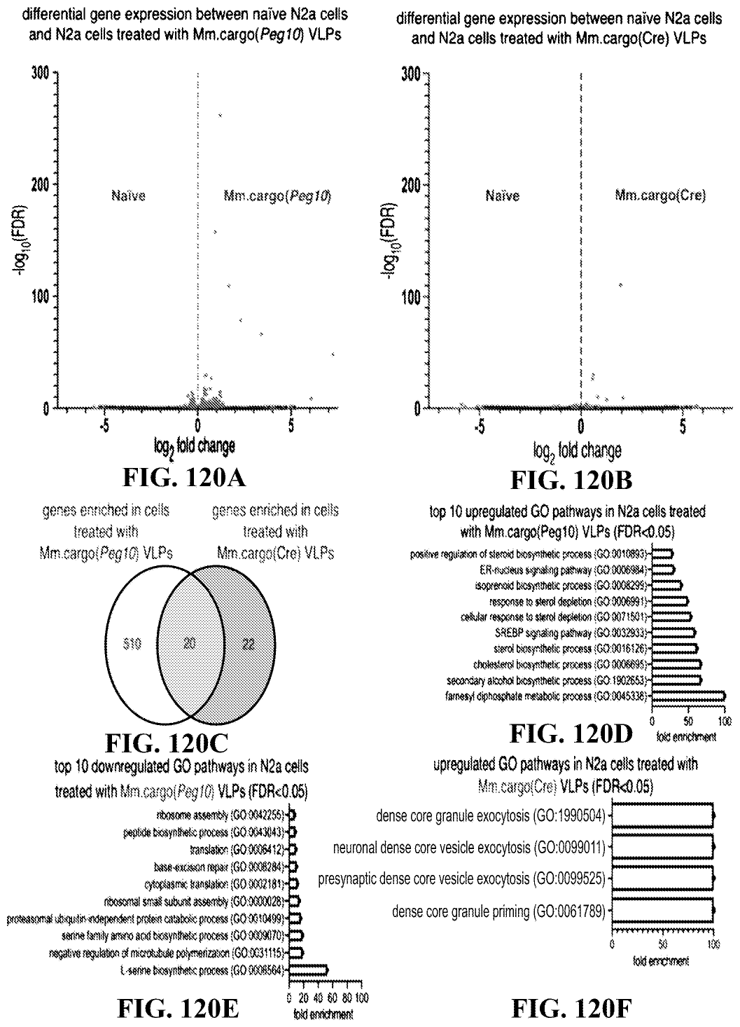

Applicant also investigated whether our SEND VLPs delivering alternative cargos like Cre would have substantial effects on target cells compared to delivering the native MmPeg10 mRNA. This is particularly important since PEG10 has been implicated in cancer and can regulate cell growth and apoptosis (38-41). To test this, Applicant produced VSVg pseudotyped VLPs containing either Mm.cargo(Peg10) or Mm.cargo(Cre) and treated loxP-GFP-N2a cells with equal amounts of the VLP (as assayed by copy number of cargoRNA). 72 hours later Applicant performed mRNA sequencing on these cells and naïve cells and found that cells treated with Mm.cargo(Peg10) VLPs had over 500 differentially expressed genes compared to naïve cells (FIG. 120A), while those treated with Mm.cargo(Cre) had 42 (FIGS. 120B and 120C). Cells treated with Mm.cargo(Peg10) VLPs upregulated genes involved in metabolic pathways (FIG. 120D) and downregulated genes involved in translation, protein catabolism, and some biosynthetic pathways (FIG. 120E). Cells treated with Mm.cargo(Cre) differentially expressed genes involved in vesicle exocytosis (FIG. 120F). This suggests transferring a reprogrammed cargo does not have the same impact on recipient cells as transferring Peg10, although further studies are needed to explore the effect on recipient cells in vivo.

### Materials and Methods

### Cell Culture

Human embryonic kidney cells (HEK-293FT, ThermoFisher R70007) were maintained at 37° C. with 5% CO₂ in DMEM+GlutaMAX (Gibco) supplemented with 10% fetal bovine serum (VWR Seradigm 1500-500) and 1% penicillin/streptomycin (Gibco). Neuro-2A (N2a, ATCC #CRL-131) cells were maintained similarly to HEK293FT cells except in the absence of penicillin/streptomycin. HEK and N2a cells at 80-90% confluence were transfected with Lipofectamine 3000 (ThermoFisher, L3000001) according to manufacturer's guidelines. Mouse primary cortical neurons (ThermoFisher, A15585) were cultured according to manufacturer's guidelines.

### Plasmids

A list of relevant plasmids can be found in Table 15. All coding sequences of mouse orthologs were amplified from a mouse embryonic stem cell cDNA library with PCR using gene-specific primers designed from the mm10 annotation. The cDNA library was prepared from a P30 mouse brain using the Protoscript II First Strand cDNA synthesis kit (NEB, E6300S). RNA from mouse brains were purified using the Directzol RNA Miniprep kit (Zymo, R2061). Plasmids were cloned using PCR amplification with Phusion Flash 2× Master Mix (Thermo Fisher Scientific F548S) and assembled with Gibson Assembly 2× Master Mix (NEB, E2611L), or blunt-end ligated with KLD Enzyme Mix (NEB, M0554S).

TABLE 15
Table of plasmid sequences provided as genbank files.

| ID | plasmid name | description |
|---|---|---|
| 1 | cmv_Hs.annotated.recoded.tiles.gb | Annotated map of recoded regions for human Peg10 |
| 2 | cmv_Hs.cargorna_cas9.gb | Plasmid encoding optimized human cargoRNA for SpCas9 |
| | | (Hs.cargo[Cas9]) |
| 3 | cmv_Hs.cargorna_cre.gb | Plasmid encoding optimized human cargoRNA for Cre |
| | | recombinase (Hs.cargo[Cre]) |
| 4 | cmv_Hs.cre.utr.tiling.annotated.gb | Annotated map of UTR tiles tested for human Peg10 |
| 5 | cmv_Mm.annotated.recoded.tiles.gb | Annotated map of recoded regions for human Peg10 |
| 6 | cmv_Mm.cargorna_cas9.gb | Plasmid encoding optimized mouse cargoRNA for SpCas9 |
| | | (Mm.cargo[Cas9]) |
| 7 | cmv_Mm.cargorna_cre.gb | Plasmid encoding optimized mouse cargoRNA for Cre |
| | | recombinase (Mm.cargo[Cre]) |
| 8 | cmv_Mm.cargorna_h2b_mcherry.gb | Plasmid encoding optimized mouse cargoRNA for H2B- |
| | | mCherry (Mm.cargo[H2B-mCherry]) |
| 9 | cmv_Mm.cre.utr.tiling.annotated.gb | Annotated mouse cargo(Cre) with the various tiles annotated |
| | | of the 3′UTR |
| 10 | cmv_Mm.Peg10_ctermHA_delnc_mut.gb | Mutant mouse Peg10 with deletion in the nucleocapsid domain |
| 11 | cmv_Mm.Peg10_ctermHA_delrt_mut.gb | Mutant mouse Peg10 with deletion in the reverse transcriptase- |
| | | like domain |
| 12 | HsPeg10.rc3.gb | Optimized and recoded human Peg10 overexpression plasmid |
| 13 | MmPeg10.rc4.gb | Optimized and recoded mouse Peg10 overexpression plasmid |
| 14 | plv-cmv-cre.gb | Lentiviral overexpression vector used to deliver Cre |
| 15 | pmd2-g-syna.gb | Mouse endogenous fusogen used for functional transfer |
| 16 | pmd2-g-synb.gb | Mouse endogenous fusogen used for functional transfer |

### Western Blot

Whole cell lysate of cultured HEK293FT and N2a cells were harvested using M-PER Mammalian Protein Extraction Reagent (ThermoFisher, 78501) with Halt Protease Inhibitor Cocktail (ThermoFisher, 78429). Harvested mouse brain tissue was homogenized using a glass dounce homogenizer in M-PER containing Protease Inhibitor Cocktail. Harvested mouse plasma was prepared from blood collected by cardiac puncture and spun with 5 μL of 0.5M EDTA (ThermoFisher, AM9260G). Plasma particles were precipitated using the Total Exosome Isolation Kit, according to the manufacturer's guidelines (ThermoFisher, 4484450). Protein concentrations were analyzed with the Pierce 660 nm Protein Assay Reagent (ThermoFisher, 22660) by Nanodrop. Protein samples were loaded onto 4-12% Bis-Tris gels (ThermoFisher, NW04125) and electrophoresis was performed according to manufacturer's guidelines. Proteins were transferred to PVDF membranes using the iBlot Transfer Stack (ThermoFisher, 1B401031). Blots were blocked for 1 hour in Intercept (TBS) Blocking Buffer (LI-COR, 92760001), incubated with primary antibodies in Intercept (TBS) Blocking Buffer overnight at 4° C., washed 3× in 1×TBST, incubated with secondary antibodies in Intercept (TBS) Blocking Buffer for 1 h at room temperature, washed 3× in 1×TBST, then imaged with BioRad ChemiDoc XRS+. Quantification was performed using ImageJ Western blot analysis toolkit.

### RT-qPCR

For all RT-qPCR experiments, total RNA was extracted with TRI Reagent (Zymo Research, R2050-1-200) and purified using the Direct-zol RNA Microprep Kit (Zymo Research, R2061). Total RNA was reverse transcribed using the Agilent cDNA qPCR Synthesis Kit (Agilent, 600559) according to manufacturer's guidelines. Gene-specific primers used for RT-qPCR are shown in Table 14. PCR was performed using Fast SYBR Green Master Mix (ThermoFisher, 4385610) on a Roche Lightcycler 480 according to manufacturer's guidelines.

TABLE 14
List of reagents used in Examples 12-13
Antibodies

| Items | Vendor | Dilution (for WB) |
|---|---|---|
| anti HA (mouse) | Cell Signalling, #2367 | 1:1000 |
| anti HA (rabbit) | Abcam, ab910 | 1:1000 |
| anti CD81 | Santa Cruz, sc-166029 | 1:500 |
| anti beta-Actin | Abcam, ab8227 | 1:5000 |
| anti Moap1 | Sigma, SAB1411249 | 1:1000 |
| anti Peg10 | Proteintech, A4412 | 1:1000, 1:25 for |
| | | immunogold |
| anti Rtl1 | ThermoFisher, PA566887 | 1:1000 |
| IRDye 680RD Donkey | LI-COR 926-68072 | 1:10,000 |
| anti-Mouse IgG (H + L) | | |
| IRDye 680RD Donkey | LI-COR 926-68073 | 1:10,000 |
| anti-Rabbit IgG (H + L) | | |
| IRDye® 800CW Goat | LI-COR 926-32211 | 1:10,000 |
| anti-Rabbit IgG | | |
| Secondary Antibody | | |
| Goat anti-rabbit 6 nm | Electron Microscopy Sciences 25103 | 1:40 |
| gold conjugate | | |

Plasmids

| Items | Source | Application |
|---|---|---|
| PB-UniSAM | Addgene, 99866 | CRISPR A |
| pUCmini-iCAP-PHP.eB | Addgene, 103005 | CRISPR KO |
| CAG-GFP-IRES-CRE | Addgene, 48201 | Cre cargoRNA cloning |
| pHelper | pHelper was a gift from CRUK | AAV production |
| PX552 | Addgene, 60958 | AAV production |
| pMD2.G | Addgene, 12259 | Lentivirus |
| psPAX | Addgene, 12260 | Lentivirus |
| RV-Cag-Dio-GFP | Addgene, 87662 | Cre reporter N2a cell line |
| pBS-Cas9-Bsd | Custom | Cas9 cell line |
| pGuide-H2B-mCherry | Custom | sgRNA cell lines |

Oligos

| Target | Sequence | Application |
|---|---|---|
| Asprv1 | F: AGGAACCCTGGGGGCCCA (SEQ ID NO: 64) | Gibson assembly of the |
| | R: GTGGGAGCCCTCCGGTGC (SEQ ID NO: 65) | coding sequence from |
| Moap1 | F: ACACTGAGACTTCTAGAAGACTGG (SEQ ID NO: 66) | species-specific cDNA |
| | R: AGTGCAATAGCCTTCTAATTCG (SEQ ID NO: 67) | library into mammalian |
| Pnma1 | F: GCTATGACACTATTGGAAGACTGGTGC (SEQ ID NO: 68) | overexpression vectors |
| | R: GAAGTGCCCCTCCAGGCC (SEQ ID NO: 69) | |
| Arc | F: GAGCTGGACCATATGACCACC (SEQ ID NO: 70) | |
| | R: TTCAGGCTGGGTCCTGTCACT (SEQ ID NO: 71) | |
| Zcchc12 | F: GCTAGCATCCTTTCACGTTTGG (SEQ ID NO: 72) | |
| | R: CTGTGGTTCAGATAGGCCAATG (SEQ ID NO: 73) | |
| Pnma3 | F: ATGAAACAGCGAAGGAAGCCTC (SEQ ID NO: 74) | |
| | R: ATGTGCTGGATGCAGTGGCT (SEQ ID NO: 75) | |
| Pnma5 | F: GCCGTGGCTCTATTAGATGA (SEQ ID NO: 76) | |
| | R: CTCACGAAAGGACTCAAGGG (SEQ ID NO: 77) | |
| Pnma6 | F: GTTATCACATTCCTCCAGGACG (SEQ ID NO: 78) | |
| | R: ATGGCGGTGACCATGCTG (SEQ ID NO: 79) | |
| Peg10 | F: GCTGCTGCGGGTGGTTCC (SEQ ID NO: 80) | |
| | R: CGCAGCACTGCAGGATGA (SEQ ID NO: 81) | |
| Rtl1 | F: GATAGAACCCTCTGAAGACT (SEQ ID NO: 82) | |
| | R: GTCAAGTTCATCATCTGAGT (SEQ ID NO: 83) | |
| PEG10 | F: GCTGCTGCGGGTGGTTCC (SEQ ID NO: 84) | |
| | R: CGCAGCACTGCAGGATGA (SEQ ID NO: 85) | |
| Peg10 | F: GCAGCCCCTATCCCAAACTT (SEQ ID NO: 86) | RT-qPCR |
| | R: CGATCAGCATGCTTGTCACG (SEQ ID NO: 87) | |
| Peg10 CDS A1468C | F: GCTTCTGGTGCATCTGGCAAC (SEQ ID NO: 88) | Mutagenesis and cloning |
| (for PEG10 D491A) | R: AATCATAGCTCGGACAAACAGGGT (SEQ ID NO: 89) | via KLD enzyme mix |
| Peg10 ΔNC | F: CCAGCGAAAGCCTCCAAG (SEQ ID NO: 90) | |
| | R: CAAATTCATTTTGCGGCGTCTC (SEQ ID NO: 91) | |
| Peg10 ΔRT | F: TGTGCCTGTTGTAATCACCTGGTCT (SEQ ID NO: 92) | |
| | R: CATGTGGTAGAAGAATGGTGGCTG (SEQ ID NO: 93) | |
| Peg10 | F: TGTTTACAGTGCCACAACCGAATT (SEQ ID NO: 94) | Indel sequencing |
| | R: AGATGCTCATGCTGATCTGGAG (SEQ ID NO: 95) | |
| Mm Kras | F: TCTTTTTCAAAGCGGCTGGC (SEQ ID NO: 110) | |
| | R: ACTTGTGGTGGTTGGAGCT (SEQ ID NO: 111) | |
| Hs Vegfa | | |

CRISPR-mediated perturbations

| | Sequence | |
|---|---|---|
| Target | (of spacers unless noted otherwise) | Application |
| Asprv1 | AGGTGTCCCGTAGGTACTGA (SEQ ID NO: 96) | CRISPRa |
| Asprv1 | GGGTGGAGCTTCTAGAACAA (SEQ ID NO: 97) | |
| Arc | GCGAGTAGGCGCGGAAGGCG (SEQ ID NO: 98) | |
| Arc | GGCCCGTGGGCGGCAGCTCG (SEQ ID NO: 99) | |
| Peg10 | AGCGTGCTTCGCGAGCAGCG (SEQ ID NO: 100) | |
| Peg10 | CGCTGCTCGCGAAGCACGCT (SEQ ID NO: 101) | |
| Rtl1 | GGGCGCGGCATGCACTGCTT (SEQ ID NO: 102) | |
| Rtl1 | AGCAATTTAGGTTCTCAAGA (SEQ ID NO: 103) | |
| Peg10 | TGCAGATGCTGATGCATATG (SEQ ID NO: 104) | CRISPR KO |
| Peg10 | TCTGTATCCGGTTATGCACC (SEQ ID NO: 105) | |
| Hs Vegfa | GGTGAGTGAGTGTGTGCGTG (SEQ ID NO: 112) | |
| Ms Kras | GCAGCGTTACCTCTATCGTA (SEQ ID NO: 113) | |
| Non-targeting | GCTTTCACGGAGGTTCGACG (SEQ ID NO: 106) | CRISPRa and CRISPR KO |
| Non-targeting | ATGTTGCAGTTCGGCTCGAT (SEQ ID NO: 107) | |
| Peg10 | AGAGGGGCTTCACTCCCCTG (SEQ ID NO: 108) | CRISPR Knock-in |
| ssDNA donor for | GCTAATAGCGACTGCTCTGAATGAATATGTTGAAT | |
| knock-in | GTATGCTTCTGTTGTCATTTACAGGAACAGGCGGG | |
| | TTTTAAGAACCAAAAGACGCCAACCACGAGGGTC | |
| | CCAGGATCCAGGGCTCCCTCCCCAGGCCACCATG | |
| | TATCCCTATGACGTGCCCGATTATGCCGCTGCTGC | |
| | GGGTGGTTCCTCCAACTGCCCGCCCCCTCCCCCTC | |
| | CCCCTCCTCCCAACAACAACAACAACAACAACAC | |
| | CCCAAAGAGCCCAGGCGTGCCTGACGCCGAAGAT | |
| | GATGATGAACGCAGACACG (SEQ ID NO: 109) | |

Purification of VLPs from Cell Culture Media

Unless otherwise noted, cell culture media containing extracellular VLPs were harvested, clarified by low centrifugation at 2000 g for 10 minutes, filtered through a 0.45 μm Durapore PVDF filter (EMD Millipore, #SE1M003M00), and ultracentrifuged at 120,000×g g for 2 h at 4° C. in a Beckman Coulter Optima XPN-80 ultracentrifuige. The supernatant was decanted, and the remaining pellet was resuspended up to 100 uL 1×PBS. To remove DNA and RNA not protected by a vesicle, purified VLPs were treated with 2.5 μL Micrococcal Nuclease (NEB, M0247S) in 1× Micrococcal Nuclease buffer and 100 ug/mL BSA for 2 h at 37° C. Micrococcal nuclease was then inactivated by the addition of EDTA to a final concentration of 10 mM. RNA was extracted with Tri reagent using the Direct-zol RNA Microprep Kit.

### Mice

Wildtype C57BL/6J-Elite mice were obtained from Charles River Laboratories. spCas9 mice were obtained from a breeding colony maintained by our group (42). All housing, breeding, and procedures were performed according to protocols approved by the Institutional Animal Care and Use Committees (IACUC) of the Broad Institute of MIT and Harvard.

All animal line generation was performed at Harvard Genome Modification Facility (GMF). Briefly, spCas9 RNPs and ssDNA donors with the HA tag and 130 bp of homology were injected into wild type C57BL6J embryos. Neonates were genotyped for correct insertions using NGS on DNA isolated from ear clippings (Lucigen; QE09050)

### Homology-Based Mining of Retroviral Proteins

In order to obtain a comprehensive census of Gag homologs encoded in human and mouse genomes, Applicant ran HHpred search with a multiple alignment of mammalian orthologs of the human Arc protein employed as the query (https://toolkit.tuebingen.mpg.de/tools/hhpred (PMID: 33315308). In addition to the highly significant hits in the human proteome, this search retrieved the homologous domains from the Pfam database: Gag-p24, Gag-p30, gag-gag2, SCAN, PNMA, DUF1759, and DUF4219. Although all these domains are homologous, they belong to two different Pfam clans, namely, clan GAG-polyprotein (CL0523) and clan Viral_Gag (CL0148). HHpred searches were repeated using multiple alignments of each of these Pfam domains as queries. These seed alignments were also used to build HMM profiles (PMID: 22039361). These profiles and the profile for mammalian Arc orthologs were used to search the human proteome (Annotation Release 109.20210226) and the mouse proteome (Annotation Release 109), from NCBI ftp site (https://ftp.ncbi.nlm.nih.gov/genomes/all/GCF/000/001/405/GCF_000001405.39_GRCh38.p13/)(https://ftp.ncbi.nlm.nih.gov/genomes/all/annotation_releases/10090/109/GCF_00000163 5.27_GRCm39/.) In order to confirm the significant hits produced by these search as Gag homologs, they were used as queries for reciprocal searches of the Pfam (PMID: 30357350), SMART (PMID: 33104802), and CDD (PMID: 31777944) databases. The confirmed Gag homologs encoded in the human and mouse genomes with apparent orthologous relationships among them are listed in Table 12. Additional confirmed Gag homologs encoded in the mouse genomes are listed in Table 13. Importantly, retroviruses integrated into mammalian genomes in multiple waves because of which one to one orthologous relationships can be demonstrated only for a subset of retrovirus-derived genes.

### Protein Production, Purification, and Size-Exclusion Chromatography

Gag proteins cloned from mouse cDNA were subcloned into a TwinStrep-SUMO expression backbone and transformed into Rosetta™ 2(DE3) (Millipore Sigma, 71400). Overnight cultures were inoculated with single colonies and grown. The following day 1 mL of overnight culture was used to inoculate 50 mL of Terrific Broth (TB) medium. Cultures were grown at 37° C. until O.D. 0.6-0.7 and induced with 0.3 mM IPTG at 25 C for 16-18 hours. Cultures were spun down and resuspended in 50 mM Tris-HCl, pH 8.0 (Invitrogen), 500 mM NaCl (Millipore Sigma), 1 mM DTT (Millipore Sigma) with the addition of Complete™ Protease Inhibitor Cocktail (Roche). Bacteria were lysed with 25 kpsi of pressure on an LM-20 Microfluidizer (Microfluidics International Corporation). Insoluble components were spun at 20,000×g for 30 mins at 4 C and the supernatant was decanted and 0.45 um filtered. Protein was isolated by flowing over Strep Tactin Superflow Plus resin (Qiagen, 30004) followed by 5 C.V. washing with 50 mM Tris-HCl, pH 8.0, 500 mM NaCl. Protein was eluted with 50 mM Tris-HCl, 500 mM NaCl, 2.5 mM desthiobiotin. The resulting proteins were cleaved overnight with homemade SUMO protease to remove tags and size excluded into 50 mM Tris-HCl, 150 mM NaCl on an ÅKTA Pure system using a Superdex 200 Increase 10/300 GL column (Cytiva).

### Negative Stain TEM and Cryo TEM

Negative stain TEM and cryo TEM microscopy experiments were performed at MIT's Koch Nanotechnology Materials Core Facility. In sample preparation for cryo-electron microscopy, 3 uL of sample and buffer containing solution was dropped on a lacey copper grid coated with a continuous carbon film and blotted to remove excess sample without damaging the carbon layer by Gatan Cryo Plunge III. Grid was mounted on a Gatan 626 single tilt cryo-holder equipped in the TEM column. The specimen and holder tip were cooled down by liquid-nitrogen, which keeps maintaining during transfer into the microscope and subsequent imaging. Imaging on a JEOL 2100 FEG microscope was done using a minimum dose method that was essential to avoid sample damage under the electron beam. The microscope was operated at 200 kV and with a magnification in the range of 10,000-60,000 for assessing particle size and distribution. All images were recorded on a Gatan 2k×2k UltraScan CCD camera.

In sample preparation for negative stain-electron microscopy, 7 uL of sample and buffer containing solution was dropped on a 200 meshes copper grid coated with a continuous carbon film and waited for 60 seconds and removed excess solution by touching the grid with a kimwipes and then 10 uL of negative staining solution, phosphotungstic acid, 1% aqueous solution was dropped on the TEM grid and immediately removed it by kimwipes and 10 uL of the stain is then applied to the grid and after 40 seconds, the excess stain is removed by touching the edge with kimwipes. Finally, dried the grid at room temperature. After that, the grid was mounted on a JEOL single tilt holder equipped in the TEM column. The specimens were cooled down by liquid-nitrogen and imaging on an JEOL 2100 FEG microscope was done using minimum dose methods that were essential to avoid sample damage under the electron beam. The microscope was operated at 200 kV and with a magnification in the range of 10,000˜60,000 for assessing particle size and distribution. All images were recorded on a

Gatan 2k×2k UltraScan CCD camera.

### CRISPRa of CA-Containing-Like Genes in N2a Cells

Guides were selected from the mouse SAM V1 guide library (19) for each of the screened CA-containing genes (Table 14). Guides were annealed and cloned via golden gate into the UniSAM addgene plasmid (Addgene, 99866) as covered in previous publications from our lab (19).

Plasmids were transfected into N2a cells at 80% confluency in 1× T225 flasks per replicate per condition. Cell culture media was replaced with fresh media 5 h post-transfection. VLPs were harvested by filtration and ultracentrifugation 48 hours after transfection as indicated above, treated with micrococcal nuclease, and lysed in trizol, as covered in the previous section.

### Co-IP Mass Spectrometry

N2a cells cultured in 1× T225 flasks per replicate were transiently with lipofectamine 3000 with N-terminal and C-terminally HA tagged-Peg10 overexpression plasmids. Cells were lysed and PEG10 protein was immunoprecipitated using the Pierce™ HA-Tag Magnetic IP/Co-IP Kit (Thermofisher; 88838). Briefly, cells were lysed in the Pierce kit IP lysis buffer and rotated at room temperature for 30 minutes with 25 μL of Pierce HA magnetic beads. Protein was eluted from the beads using the kit's acidic elution buffer and neutralized.

### In-Gel Protein Digestion

Gel bands were cut out, reduced with 5 mM DTT and alkylated with 10 mM iodoacetamide and digested with either trypsin at 37° C. essentially as described previously (43).

### Immunoprecipitation Protein Digestion

Beads were digested according to the S-Trap Micro Spin Column Digestion Protocol (www.protifi.com).

### LC-MS/MS Analysis for Gel Bands

The dried peptide mix was reconstituted in a solution of 2% formic acid (FA) for MS analysis. Peptides were loaded with the autosampler directly onto a 50 cm EASY-Spray C18 column (ES803a, Thermo Scientific). Peptides were eluted from the column using a Dionex Ultimate 3000 Nano LC system with a 14.8 min gradient from 1% buffer B to 23% buffer B (100% acetonitrile, 0.1% formic acid), followed by a 0.2 min gradient to 80% B, and held constant for 0.5 min. Finally, the gradient was changed from 80% buffer B to 99% buffer A (100% water, 0.1% formic acid) over 0.5 min, and then held constant at 99% buffer A for 14 more minutes. The application of a 2.2 kV distal voltage electrosprayed the eluting peptides directly into the Thermo Exploris480 mass spectrometer equipped with an EASY-Spray source (Thermo Scientific). Mass spectrometer-scanning functions and HPLC gradients were controlled by the Xcalibur data system (Thermo Scientific). MS1 scans parameters were 60,000 resolution, AGC at 300%, IT at 25 ms. MS2 scan parameters were 30,000 resolution, isolation width at 1.2, HCD collision energy at 28%, AGC target at 100% and max IT at 55 ms. 15MS/MS scans were taken for each MS1 scan. Expected peptides for Retrotransposon-derived protein PEG10 GN=Peg10 were put into an inclusion list.

### LC-MS/MS Analysis for IPs

The dried peptide mix was reconstituted in a solution of 2% formic acid (FA) for MS analysis. Peptides were loaded with the autosampler directly onto a 50 cm EASY-Spray C18 column (ES803a, Thermo Scientific). Peptides were eluted from the column using a Dionex Ultimate 3000 Nano LC system with a 3 min gradient from 1% buffer B to 5% buffer B (100% acetonitrile, 0.1% formic acid), followed by a 36.8 min gradient to 25%, and a 10.2 min gradient to 35% B, followed by a 0.5 min gradient to 80% B, and held constant for 4.5 min. Finally, the gradient was changed from 80% buffer B to 99% buffer A (100% water, 0.1% formic acid) over 0.1 min, and then held constant at 99% buffer A for 19.9 more minutes. The application of a 2.2 kV distal voltage electrosprayed the eluting peptides directly into the Thermo Exploris480 mass spectrometer equipped with an EASY-Spray source (Thermo Scientific). Mass spectrometer-scanning functions and HPLC gradients were controlled by the Xcalibur data system (Thermo Scientific). MS1 scans parameters were 120,000 resolution, AGC at 300%, IT at 50 ms. MS2 scan parameters were 30,000 resolution, isolation width at 1.2, HCD collision energy at 28%, AGC target at 300% and IT set to Auto. Cycle time for MS2 was 3 sec for each MS1 scan.

### Database Search

Tandem mass 260tilize were searched with Sequest (Thermo Fisher Scientific, San Jose, CA, USA; version IseNode in Proteome Discoverer 2.5.0.400). Sequest was set up to search a mouse uniprot database (database version Mar. 21, 2020; 55699 entries containing common contaminants) assuming the digestion enzyme trypsin. Sequest was searched with a fragment ion mass tolerance of 0.02 Da and a parent ion tolerance of 10.0 PPM. Carbamidomethyl of cysteine was specified in Sequest as a fixed modification. Oxidation of methionine was specified in Sequest as a variable modification. Scaffold (version Scaffold_4.11.1, Proteome Software Inc., Portland, OR) was used to validate MS/MS based peptide and protein identifications. Peptide identifications were accepted if they could be established at greater than 95.0% probability by the Percolator posterior error probability calculation (44). Protein identifications were accepted if they could be established at greater than 99.0% probability and contained at least 2 identified peptides. Protein probabilities were assigned by the Protein Prophet algorithm (45). Proteins that contained similar peptides and could not be differentiated based on MS/MS analysis alone were grouped to satisfy the principles of parsimony. Proteins sharing significant peptide evidence were grouped into clusters.

### Cloning of PEG10 Variants

Structural domains within PEG10 were identified via homology detection of the full Peg10 peptide sequence against known domains via HHPred with the HHsuite v3.0 release at default settings (database: PDB_mmCIF70) (46). The protease domain in PEG10 was made inactive by mutating the catalytic aspartic acid to alanine (D491A) using oligos listed in Supp. Table 14. In-frame deletions of the NC and the RT domains were made using oligos listed in Supp. Table 14. Peg10 codon optimization was performed using the IDT codon optimization tool for human and gene blocks were ordered from IDT.

### AAV PHP.eB Production and Delivery

AAV CRISPR guides were designed using Benchling online CRISPR gRNA design tool and validated for efficiency in mouse embryos and cloned with golden gate method into PX552 (Addgene, 60958). PHP.eB AAV vectors were generated as previously described (47). In brief, HEK293 cells were grown in T225 flasks plastic dishes and transfected with the pHelper plasmid, the PHP.eB capsid plasmid, and the Px552 transgene plasmid. Five days later, cells were lysed, and virus was isolated using Optiprep density-gradient medium (Sigma; D1556) and ultra-centrifuged at 350,000 g. The viral layer was isolated and concentrated using Amicon Ultra-15 centrifugal filter units (Sigma; Z648043-24EA). AAVs titre was determined using SYBR Green qPCR. Two vectors with two separate Peg10 targeting gRNAs were produced in parallel and pooled in equal titer. For in vivo administration of the virus, P1 mice were anesthetized and 5el 1 viral genomes for each virus were retro-orbitally injected into in-house generated spCas9 mice. Mice are publicly available (Jackson labs, 026179).

### FACS of Neuronal Nuclei

Prefrontal cortex of P25 mice was dissected and flash frozen in liquid nitrogen. Nuclei were prepared from fresh frozen brains using the Nuclei EZ Prep kit (Sigma, NUC101), as previously described (48). Briefly, nuclei were dounced exactly 20 times in 1 mL of Nuclei lysis buffer on ice immediately after removal from dry ice and washed twice in Nuclei lysis buffer. Following prep, nuclei were counterstained with DAPI and resuspended in PBS with 0.5% BSA (ThermoFisher, 15260037). For each condition, 20,000 neuronal nuclei were sorted based on DAPI and GFP on a Sony MA900 Cell Sorter directly into Tri reagent. RNA was extracted using the Direct-zol RNA Microprep Kit and mRNA sequencing library was prepared as listed below.

mRNA-Sequencing of Nuclei, Whole Cell RNA and EVs

N2a cells, purified EVs, sorted neuronal nuclei, and primary mouse cortical neurons were lysed with TRI Reagent (Zymo Research, R2050-1-200), and total RNA was extracted using Direct-zol RNA Microprep Kit (Zymo Research, R2061) and treated with DNase I (Zymo Research, E1010). Whole cell mRNA was enriched using the NEBNext Poly(A) mRNA Magnetic Isolation Module (NEB, E7490), and the multiplexed RNA sequencing library was prepared using NEBNext Ultra II Directional RNA Library Prep Kit for Illumina (NEB, E7775). Libraries were sequenced on the Illumina NextSeq 550 with 50 cycles for read 1 (forward), 25 cycles for read 2 (reverse) for a read coverage of approximately 15-20 million reads per sample.

Raw reads were trimmed using Trimmomatic (49) and quality control was performed using fastqc (50). Pseudo alignments and differential gene expression analysis was performed using Kallisto and Sleuth (51, 52). Full read alignments were generated using STAR (53) and converted to indexed BAM files with SAM tools to generate read tracks.

### Indel Sequencing of In Vitro Edited Cells and Sorted Neuronal Nuclei and N2a Cells

In vitro 96 well plates of tissue culture cells were lysed with 20 μL of QuickExtract (Lucigen, QE09050). The target region was amplified from genomic DNA by PCR with primers described in Table 14 and barcoded with indexed Illumina P5 and P7 primers for NGS. Libraries were purified with Ampure XP (Beckman Coulter), quantified with Qubit hsDNA reagent, and sequenced on an Illumina MiSeq system (300 bp read 1). Indels were quantified from the resulting library using CRIS.py, a python based tool used to detect indels from NGS libraries (54).

In Vivo and In Vitro eCLIP

Female prefrontal cortex of P30-P32 HA-tagged mice generated and bred in house were dissected and flash frozen in liquid nitrogen. To prepare the CLIP library, 300 mg of brain was pulverized with a pestle on ice and immediately UV irradiated twice at 400 mJ/cm² (55). The tissue was then processed using an adapted eCLIP protocol (56). Briefly, tissue was resuspended in the recommended CLIP lysis buffer and sonicated for 5 minutes at low intensity on a Bioruptor Pico sonicator (Diagenode, B01080010). For in vitro eCLIP experiments, ˜2 million N2A cells were transiently transfected in a 6-well plate in the same manner as listed above. Three days following transfection, media was changed to PBS (ThermoFisher, 10010023) and UV irradiated once at 400 mJ/cm², resuspended in the CLIP lysis buffer and scraped from the plate.

Unless otherwise stated, all methods and components for the CLIP preparation were exactly as listed previously (56). Briefly, lysed samples were nuclease treated as recommended and immunoprecipitated using Pierce Anti-HA magnetic beads (ThermoFisher, 88836). As per the protocol, immunoprecipitated samples were washed and overnight ligated with the recommended CLIP 3′ RNA adaptor. Simultaneously, a subsample was blotted for HA to determine the efficacy of the precipitation and expected band size. Following overnight ligation, samples were loaded onto a NuPAGE gel (ThermoFis263tilizes26321PK2) and transferred onto a nitrocellulose membrane (ThermoFisher, IB23001). Bands were excised with a scalpel on a sterile bioassay plate, transferred to an eppendorf tube, and sequentially digested in Proteinase K with urea, and then Acid-Phenol:Chloroform (ThermoFisher, AM9722) on an orbital thermoshaker at 37° C. RNA was extracted and prepared for sequencing as recommended from the CLIP protocol. The samples were run on the NextSeq 550 with 50 cycles for read 1 (forward), 25 cycles for read 2 (reverse). Sequencing reads were aligned with STAR, quantified using htseq-count, and differential expression analysis was performed with DESeq2.

### Cre-Reporter Cell Line Generation

A lentiviral loxP-GFP Cre reporter cassette was generated by subcloning the loxP-GFP cassette from RV-Cag-Dio-GFP (Addgene #87662) into a lentiviral transfer plasmid encoding a blasticidin resistance gene. To produce virus HEK293FT cells were seeded at 1e7 cells per 15 cm dish. 16 hours later cells were co-transfected with 5 ug psPAX2 (Addgene #12260), 4.7 ug pMD2.G (Addgene #12259), and 7.7 ug of the Cre reporter plasmid using PEI HCl MAX (Polysciences 24765-1), media was changed 4 hours post transfection. 48 hours later viral supernatant was harvested, spun at 2000 g for 10 mins to remove cell debris, 0.45 um filtered, aliquoted, and frozen at −80° C. for later use.

N2a reporter cell lines were created by seeding cells at 50% confluency in 6 well plates at day 0. 1 mL of clarified virus supernatant was added to the cells along with 8 ug/mL polybrene (TR1003G). Media was changed one day later, and cells were selected for two weeks starting on day 3 with 10 ug/mL Blasticidin-HCl (Thermo Fisher Scientific A1113903). Successful reporter line generation was confirmed by transfection of a Cre encoding plasmid.

### Lentivirus and VLP Production Transfer.

Both lentivirus and Peg10 VLPs packaging Cre-encoding mRNA were produced and purified in an identical manner. HEK293FT cells were seeded at a density of 4e6 cells per 10 cm dish. The next day, cells were transfected with 2 ug each of i) [vector RNA], ii) a Peg10 overexpression plasmid or psPAX2 (Addgene #12260), and iii) pMD2.G (Addgene #12259). Cells were washed with PbS and fresh medium 4 h post-transfection. 48 hours later, the culture supernatant was harvested, and particles were purified in the same manner as the purification of EVs from the CRISPRa experiment described above.

For all N2a transfer experiments, Cre-reporter N2a cells were plated onto 96-well plates at 5e4 cells per well one hour before transfer. 20 ul of purified VLPs or lentivirus derived from a 10 cm dish were applied to N2a cells in triplicate. Cells were incubated for 72 hours before flow cytometry analysis.

For tail tip fibroblast transfer experiments, cells were plated onto gelatin coated 96-well plates at 3e4 cells per well 24 hours before transfer. 20 ul of purified VLPs or lentivirus derived from a 10 cm dish were applied to cells in triplicate the next day. For endogenous fusogen pseudotypes, the transduction enhancer vectofusin-1 (Miltenyi Biotec) was added per manufacturer's instructions. Cells were incubated for 72 hours before analysis.

### Flow Cytometry

N2a cells in 96-well plates were washed once with 1×PBS, and dissociated with TrypLE (ThermoFisher, 12604013). Cells were resuspended with 2% FBS in PBS, centrifuged at 500 g for 5 min, and washed once in PBS. Cells were viability stained for 30 mins with Zombie NIR Fixable Viability Dye (Biolegend, 423105) in PBS at 4 C. Cells were washed twice in 2% FBS in PBS and resuspended in 2% FBS in PBS+2 mM EDTA for analysis on a Beckman Coulter Cytoflex S flow cytometer. Analysis was performed using FlowJo v10.7 (BD Biosciences). Representative gating schemes are shown in FIG. 112.

### Cell Imaging

N2a, Ai9 tail tip fibroblasts, or HEK293FT cells were plated on glass bottom plates (Cellvis, P96-1.5H-N). Following VLP transfer experiments, cells were fixed with 4% PFA in 1×PBS for 15 min at room temperature, washed 3× with 1×PBS for 5 min each, permeabilized with 0.1% Tween in PBS for 5 min, and counterstained with DAPI (ThermoFisher, R37606). Confocal images were obtained using a Leica Stellaris 5 confocal microscope and an Opera Phenix high content imaging system. TTFs were stained with anti-H2A (Abcam, ab18255) and detected with anti-rabbit Alexa fluor 488 (ThermoFisher, A-21206). For H2B-mCherry transfer experiments, cells were stained with anti-mCherry (Abcam, ab167453) and detected with anti-rabbit Alexa fluor 488.

Immunogold labeling and EM

To reduce noise in immunogold labeling, VLPs were purified more extensively than in previous experiments. Supernatant from transfected cells was harvested, spun at 2000×g for 10 minutes to remove cell debris, 0.45 um filtered, and spun at 26,000 rpm for 2 hours at 4° C. through a 20% sucrose cushion. The pellet was resuspended in PBS and further purified over an 8-30% iodixanol gradient (Opti-prep, Sigma-Aldrich) with 2% steps spun at 250,000×g for 1.5 hours at 4° C. The gradient was fractionated in 0.5 mL fractions, desalted with centrifugal filters, and western blotted against HA to determine which fractions contained PEG10 (FIG. S9A). Immunogold labeling protocol was derived from previous work (10), first, PEG10 positive fractions were fixed overnight in 2% PFA at 4° C. Samples were adhered to carbon coated nickel TEM grids (300 mesh, Ted Pella) for 20 minutes at 4° C., washed in PBS, quenched with 50 mM glycine, permeabilized with 0.1% saponin for 20 mins, and blocked for 30 mins in 5% BSA-c (EMS-Aurion). Samples were stained in blocking buffer with a rabbit anti-PEG10 (Proteintech) for 1 hour at room temperature, washed, and stained with 6 nm goat anti-rabbit gold antibody conjugates (EMS-Aurion). Samples were washed in PBS, stabilized in 1% glutaraldehyde, washed again in water, and negative stained with 2% uranyl acetate (EMS). Grids were visualized on a FEI Tecnai G2 Twin Spirit TEM equipped with a Gatan CCD camera at 120 kV at a magnification between 40,000-60,000×.

Cas9 and sgRNA Guide Cell Line Generation

A guide against mouse Kras was cloned using golden gate assembly under control of a U6 promoter into a custom lentiviral vector. Virus was produced using this genome as previously described and supernatant was added to N2a cells at 70% confluency. After 2 days cells were split and selected for 7 days in Zeocin (Thermo Fisher). Cells expressed H2B-mCherry upon transduction, so this was verified visually. Cells were used for transfer experiments as described previously. For spCas9 cell lines, N2a cells were transduced similarly, but with a genome encoding an EF1a driven SpCas9 and blasticidin resistance. Cells were periodically maintained under selection and frozen at low passage.

### Tail-Tip Fibroblast Isolation

Tails were removed from culled Ai9 mice with scissors and doused in 70% ethanol. The outer skin was removed with tweezers and scissors and the tail was minced in warm DMEM. Liberase TH (Roche) was added to a concentration of 200 ug/mL and the samples were incubated for 30 min at 4° C. After washing, the tissue was placed on a 2% gelatin coated tissue culture plate in warm DMEM and fibroblasts were allowed to migrate out for 5 days before a media change and tissue removal. Media was changed every 2 days thereafter until cells reached confluence. Cells were split, frozen, and used for experiments at low passage to prevent senescence.

### References for Example 13

1. J. L. Goodier, H. H. Kazazian Jr, Retrotransposons revisited: the restraint and rehabilitation of parasites. Cell. 135, 23-35 (2008).
2. A. F. Smit, Interspersed repe-ts and other mementos of transposable elements in mammalian genomes. Curr. Opin. Genet. Dev. 9, 657-663 (1999).
3. M. R. Patel, M. Emerman, H. S. Malik, Paleovirology—ghosts and gifts of viruses past. Curr. Opin. Virol. 1, 304-309 (2011).
4. L. Guio, J. Gonzilez, New Insights on the Evolution of Genome Content: Population Dynamics of Transposable Elements in Flies and Humans. Methods Mol. Biol. 1910, 505-530 (2019).
5. C. Feschotte, C. Gilbert, Endogenous viruses: insights into viral evolution and impact on host biology. Nat. Rev. Genet. 13, 283-296 (2012).
6. F. J. Kim, J.-L. Battini, N. Manel, M. Sitbon, Emergence of vertebrate retroviruses and envelope capture. Virology. 318, 183-191 (2004).
7. A. Dupressoir, C. Vemochet, O. Bawa, F. Harper, G. Pierron, P. Opolon, T. Heidmann, Syncytin-A knockout mice demonstrate the critical role in placentation of a fusogenic, endogenous retrovirus-derived, envelope gene. Proc. Natl. Acad. Sci. U.S.A 106, 12127-12132 (2009).
8. C. Myrum, A. Baumann, H. J. Bustad, M. I. Flydal, V. Mariaule, S. Alvira, J. Cuéllar, J. Haavik, J. Soulé, J. M. Valpuesta, J. A. Márquez, A. Martinez, C. R. Bramham, Arc is a flexible modular protein capable of reversible self-oligomerization. Biochem. J. 468, 145-158 (2015).
9. J. Ashley, B. Cordy, D. Lucia, L. G. Fradkin, V. Budnik, T. Thomson, Retrovirus-like Gag Protein Arc1 Binds RNA and Traffics across Synaptic Boutons. Cell. 172, 262-274.e11 (2018).
10. E. D. Pastuzyn, C. E. Day, R. B. Keans, M. Kyrke-Smith, A. V. Taibi, J. McCormick, N. Yoder, D. M. Belnap, S. Erlendsson, D. R. Morado, J. A. G. Briggs, C. Feschotte, J. D. Shepherd, The Neuronal Gene Arc Encodes a Repurposed Retrotransposon Gag Protein that Mediates Intercellular RNA Transfer. Cell. 173, 275 (2018).
11. S. F. Erica Korb, Arc in synaptic plasticity: from gene to behavior. Trends Neurosci. 34, 591 (2011).
12. P. Barragan-Iglesias, J. B. De La Pena, T. F. Lou, S. Loerch, Intercellular Arc signaling regulates vasodilation (2020) (available at https://papers.ssm.com/sol3/papers.cfm?abstract_id=3684856).
13. M. Abed, E. Verschueren, H. Budayeva, P. Liu, D. S. Kirkpatrick, R. Reja, S. K. Kummerfeld, J. D. Webster, S. Gierke, M. Reichelt, K. R. Anderson, R. J. Newman, M. Roose-Girma, Z. Modrusan, H. Pektas, E. Maltepe, K. Newton, V. M. Dixit, The Gag protein PEG10 binds to RNA and regulates trophoblast stem cell lineage specification. PLoS One. 14, e0214110 (2019).
14. R. Ono, K. Nakamura, K. Inoue, M. Naruse, T. Usami, N. Wakisaka-Saito, T. Hino, R. Suzuki-Migishima, N. Ogonuki, H. Miki, T. Kohda, A. Ogura, M. Yokoyama, T. Kaneko-Ishino, F. Ishino, Deletion of Peg10, an imprinted gene acquired from a retrotransposon, causes early embryonic lethality. Nat. Genet. 38, 101-106 (2006).
15. C. Henke, P. L. Strissel, M.-T. Schubert, Mitchell, C. C. Stolt, F. Faschingbauer, M. W. Beckmann, R. Strick, Selective expression of sense and antisense transcripts of the sushi-ichi-related retrotransposon-derived family during mouse placentogenesis. wRetrovirology. 12, 9 (2015).
16. M. Krupovic, E. V. Koonin, Multiple origins of viral capsid proteins from cellular ancestors. Proc. Nati. Acad. Sci. U.S.A 114, E2401-E2410 (2017).
17. S. O. Dodonova, S. Prinz, V. Bilanchone, S. Sandmeyer, J. A. G. Briggs, Structure of the Ty3/Gypsy retrotransposon capsid and the evolution of retroviruses. Proc. Natl. Acad. Sci. U.S.A. 116, 10048-10057 (2019).
18. M. Campillos, T. Doerks, P. K. Shah, P. Bork, Computational characterization of multiple Gag-like human proteins. Trends Genet. 22, 585-589 (2006).
19. S. Konermann, M. D. Brigham, A. E. Trevino, J. Joung, O. O. Abudayyeh, C. Barcena, P. D. Hsu, N. Habib, J. S. Gootenberg, H. Nishimasu, O. Nureki, F. Zhang, Genome-scale transcriptional activation by an engineered CRISPR-Cas9 complex. Nature. 517, 583-588 (2015).
20. C. S. Wallace, G. L. Lyford, P. F. Worley, O. Steward, Differential intracellular sorting of immediate early gene mRNAs depends on signals in the mRNA sequence. J. Neurosci. 18, 26-35 (1998).
21. M. Golda, J. A. Mótyán, M. Mahdi, J. Tözsér, Functional Study of the Retrotransposon-Derived Human PEG10 Protease. Int. J. Mol. Sci. 21 (2020), doi:10.3390/ijms21072424.
22. A. Saunders, E. Z. Macosko, A. Wysoker, M. Goldman, F. M. Krienen, H. de Rivera, E. Bien, M. Baum, L. Bortolin, S. Wang, A. Goeva, J. Nemesh, N. Kamitaki, S. Brumbaugh, D. Kulp, S. A. McCarroll, Molecular Diversity and Specializations among the Cells of the Adult Mouse Brain. Cell. 174 (2018), pp. 1015-1030.e16.
23. K. Clemens, V. Bilanchone, N. Beliakova-Bethell, Sequence requirements for localization and packaging of Ty3 retroelement RNA. Virus Res. (2013) (available at https://www.sciencedirect.com/science/article/pii/S0168170212003784).
24. K. Ridder, A. Sevko, J. Heide, M. Dams, AIRupp, J. Macas, J. Starmann, M. Tjwa, K. H. Plate, H. Sultmann, P. Altevogt, V. Umansky, S. Momma, Extracellular vesicle-mediated transfer of functional RNA in the tumor microenvironment. Oncoimmunology. 4, e1008371 (2015).
25. Y. Coquin, M. Ferrand, A. Seye, L. Menu, A. Galy, Syncytins enable novel possibilities to transduce human or mouse primary B cells and to achieve well-tolerated in vivo gene transfer. Cold Spring Harbor Laboratory (2019), p. 816223.
26. P. S. Kowalski, A. Rudra, L. Miao, D. G. Anderson, Delivering the Messenger: Advances in Technologies for Therapeutic mRNA Delivery. Mol. Ther. 27, 710-728 (2019).
27. U. Mock, K. Riecken, B. Berdien, W. Qasim, E. Chan, T. Cathomen, B. Fehse, Novel lentiviral vectors with mutated reverse transcriptase for mRNA delivery of TALE nucleases. Sci. Rep. 4, 6409 (2014).
28. S. J. Kaczmarczyk, K. Sitaraman, H. A. Young, S. H. Hughes, D. K. Chatterjee, Protein delivery using engineered virus-like particles. Proc. Natl. Acad. Sci. U.S.A 108, 16998-17003 (2011).
29. P. E. Mangeot, V. Risson, F. Fusil, A. Mamef, E. Laurent, J. Blin, V. Moumetas, E. Massouridès, T. J. M. Sohier, A. Corbin, F. Aubé, M. Teixeira, C. Pinset, L. Schaeffer, G. Legube, F.-L. Cosset, E. Verhoeyen, T. Ohlmann, E. P. Ricci, Genome editing in primary cells and in vivo using viral-derived Nanoblades loaded with Cas9-sgRNA ribonucleoproteins. Nat. Commun. 10, 45 (2019).
30. R. Kojima, D. Bojar, G. Rizzi, G. C.-E. Hamri, M. D. El-Baba, P. Saxena, S. Auslinder, K. R. Tan, M. Fussenegger, Designer exosomes produced by implanted cells intracerebrally deliver therapeutic cargo for Parkinson's disease treatment. Nat. Commun. 9, 1305 (2018).
31. M. E. Hung, J. N. Leonard, A platform for actively loading cargo RNA to elucidate limiting steps in EV-mediated delivery. J Extracell Vesicles. 5, 31027 (2016).
32. J. L. Shirley, Y. P. de Jong, C. Terhorst, R. W. Herzog, Immune Responses to Viral Gene Therapy Vectors. Mol. Ther. 28, 709-722 (2020).
33. J.-E. Park, R. A. Botting, C. Dominguez Conde, D.-M. Popescu, M. Lavaert, D. J. Kunz, I. Goh, E. Stephenson, R. Ragazzini, E. Tuck, A. Wilbrey-Clark, K. Roberts, V. R. Kedlian, J. R. Ferdinand, X. He, S. Webb, D. Maunder, N. Vandamme, K. T. Mahbubani, K. Polanski, L. Mamanova, L. Bolt, D. Crossland, F. de Rita, A. Fuller, A. Filby, G. Reynolds, D. Dixon, K. Saeb-Parsy, S. Lisgo, D. Henderson, R. Vento-Tormo, O. A. Bayraktar, R. A. Barker, K. B. Meyer, Y. Saeys, P. Bonfanti, S. Behjati, M. R. Clatworthy, T. Taghon, M. Haniffa, S. A. Teichmann, A cell atlas of human thymic development defines T cell repertoire formation. Science. 367 (2020), doi:10.1126/science.aay3224.
34. R. Ono, S. Kobayashi, H. Wagatsuma, K. Aisaka, T. Kohda, T. Kaneko-Ishino, F. Ishino, A retrotransposon-derived gene, PEG10, is a novel imprinted gene located on human chromosome 7q21. Genomics. 73, 232-237 (2001).
35. J. Volff, C. Körting, M. Schartl, Ty3/Gypsy retrotransposon fossils in mammalian genomes: did they evolve in268tilizes268izationfunctions? Mol. Biol. Evol. 18, 266-270 (2001).
36. 268tilizesemoto, J. Brennan, E. Walls, C. J. Watson, D. Stott, P. W. Rigby, A. D. Reith, Identification and characterisation of a developmentally regulated mammalian gene that utilises −1 programmed ribosomal frameshifting. Nucleic Acids Res. 29, 4079-4088 (2001).
37. R. Vento-Tormo, M. Efremova, R. A. Botting, M. Y. Turco, M. Vento-Tormo, K. B. Meyer, J.-E. Park, E. Stephenson, K. Polanski, A. Goncalves, L. Gardner, S. Holmqvist, J. Henriksson, A. Zou, A. M. Sharkey, B. Millar, B. Innes, L. Wood, A. Wilbrey-Clark, R. P. Payne, M. A. Ivarsson, S. Lisgo, A. Filby, D. H. Rowitch, J. N. Bulmer, G. J. Wright, M. J. T. Stubbington, M. Haniffa, A. Moffett, S. A. Teichmann, Single-cell reconstruction of the early maternal-fetal interface in humans. Nature. 563, 347-353 (2018).
38. H. Okabe, S. Satoh, Y. Furukawa, T. Kato, S. Hasegawa, Y. Nakajima, Y. Yamaoka, Y. Nakamura, Involvement of PEG10 in human hepatocellular carcinogenesis through interaction with SIAH1. Cancer Res. 63, 3043-3048 (2003).
39. J. Liang, N. Liu, H. Xin, Knockdown long non-coding RNA PEG10 inhibits proliferation, migration and invasion of glioma cell line U251 by regulating miR-506. Gen. Physiol. Biophys. 38, 295-304 (2019).
40. Y. Kawai, K. Imada, S. Akamatsu, F. Zhang, R. Seiler, T. Hayashi, J. Leong, E. Beraldi, N. Saxena, A. Kretschmer, H. Z. Oo, A. Contreras-Sanz, H. Matsuyama, D. Lin, L. Fazli, C. C. Collins, A. W. Wyatt, P. C. Black, M. E. Gleave, Paternally Expressed Gene 10 (PEG10) Promotes Growth, Invasion, and Survival of Bladder Cancer. Mol. Cancer Ther. 19, 2210-2220 (2020).
41. S. Kim, D. Thaper, S. Bidnur, P. Toren, S. Akamatsu, J. L. Bishop, C. Colins, S. Vahid, A. Zoubeidi, PEG10 is associated with treatment-induced neuroendocrine prostate cancer. J. Mol. Endocrinol. 63, 39-49 (2019).
42. R. J. Platt, S. Chen, Y. Zhou, M. J. Yim, L. Swiech, H. R. Kempton, J. E. Dahlman, O. Pamas, T. M. Eisenhaure, M. Jovanovic, D. B. Graham, S. Jhunjhunwala, M. Heidenreich, R. J. Xavier, R. Langer, D. G. Anderson, N. Hacohen, A. Regev, G. Feng, P. A. Sharp, F. Zhang, CRISPR-Cas9 knockin mice for genome editing and cancer modeling. Cell. 159, 440-455 (2014).
43. A. Shevchenko, O. N. Jensen, A. V. Podtelejnikov, F. Sagliocco, M. Wilm, O. Vorm, P. Mortensen, A. Shevchenko, H. Boucherie, M. Mann, Linking genome and proteome by mass spectrometry: large-scale identification of yeast proteins from two dimensional gels. Proc. Natl. Acad. Sci. U.S.A 93, 14440-14445 (1996).
44. L. Käll, J. D. Storey, W. S. Noble, Non-parametric estimation of posterior error probabilities associated with peptides identified by tandem mass spectrometry. Bioinformatics. 24, i42-8 (2008).
45. A. I. Nesvizhskii, A. Keller, E. Kolker, R. Aebersold, A statistical model for identifying proteins by tandem mass spectrometry. Anal. Chem. 75, 4646-4658 (2003).
46. L. Zimmermann, A. Stephens, S.-Z. Nam, D. Rau, J. Kubler, M. Lozajic, F. Gabler, J. Söding, A. N. Lupas, V. Alva, A Completely Reimplemented MPI Bioinformatics Toolkit with a New HHpred Server at its Core. J. Mol. Biol. 430, 2237-2243 (2018).
47. R. C. Challis, S. R. Kumar, K. Y. Chan, C. Challis, M. J. Jang, P. S. Rajendran, J. D. Tompkins, K. Shivkumar, B. E. Deverman, V. Gradinani, Widespread and targeted gene expression by systemic AAV vectors: Production, purification, and administration. Cold Spring Harbor Laboratory (2018), p. 246405.
48. N. Habib, I. Avraham-Davidi, A. Basu, T. Burks, K. Shekhar, M. Hofree, S. R. Choudhury, F. Aguet, E. Gelfand, K. Ardlie, D. A. Weitz, O. Rozenblatt-Rosen, F. Zhang, A. Regev, Massively parallel single-nucleus RNA-seq with DroNc-seq. Nature Methods. 14 (2017), pp. 955-958.
49. A. M. Bolger, M. Lohse, B. Usadel, Trimmomatic: a flexible trimmer for Illumina sequence data. Bioinformatics. 30, 2114-2120 (2014).
50. S. Andrews, FastQC: A quality control tool for high throughput sequence data. Online publication (2010).
51. H. Pimentel, N. L. Bray, S. Puente, P. Melsted, L. Pachter, Differential analysis of RNA-seq incorporating quantification uncertainty. Nat. Methods. 14, 687-690 (2017).
52. N. L. Bray, H. Pimentel, P. Melsted, L. Pachter, Near-optimal probabilistic RNA-seq quantification. Nat. Biotechnol. 34, 525-527 (2016).
53. A. Dobin, C. A. Davis, F. Schlesinger, J. Drenkow, C. Zaleski, S. Jha, P. Batut, M. Chaisson, T. R. Gingeras, STAR: ultrafast universal RNA-seq aligner. Bioinformatics. 29, 15-21 (2013).
54. J. P. Connelly, S. M. Pruett-Miller, CRIS.py: A Versatile and High-throughput Analysis Program for CRISPR-based Genome Editing. Scientific Reports. 9 (2019), doi:10.1038/s41598-019-40896-w.
55. M. J. Moore, C. Zhang, E. C. Gantman, A. Mele, J. C. Danell, R. B. Danell, Mapping Argonaute and conventional RNA-binding protein interactions with RNA at single-nucleotide resolution using HITS-CLIP and CIMS analysis. Nature Protocols. 9 (2014), pp. 263-293.
56. E. L. Van Nostrand, T. B. Nguyen, C. Gelboin-Burkhart, R. Wang, S. M. Blue, G. A. Pratt, A. L. Louie, G. W. Yeo, Robust, Cost-Effective Profiling of RNA Binding Protein Targets with Single-end Enhanced Crosslinking and Immunoprecipitation (seCLIP). Methods Mol. Biol. 1648, 177-200 (2017).

### Example 14—Sushi Family Proteins for Delivery

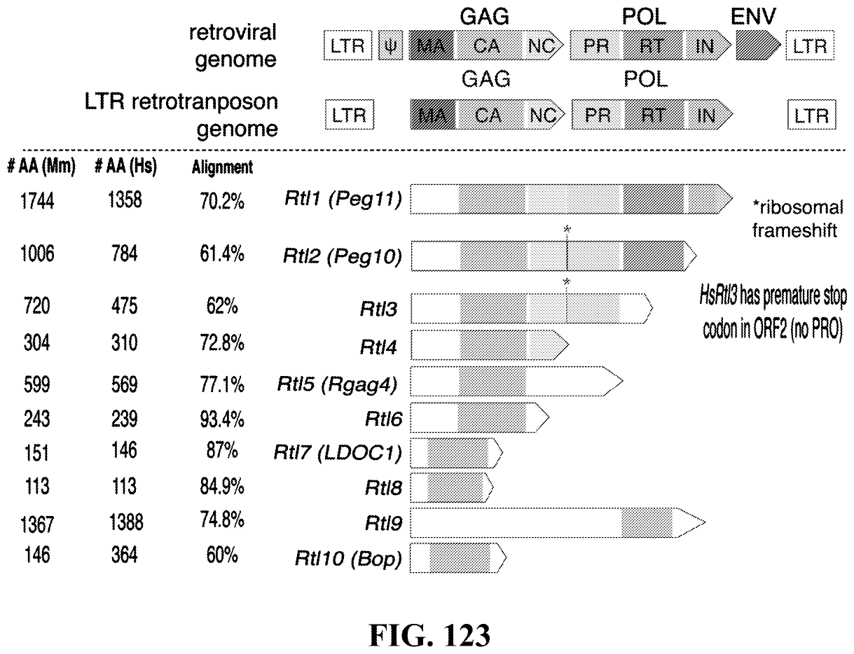

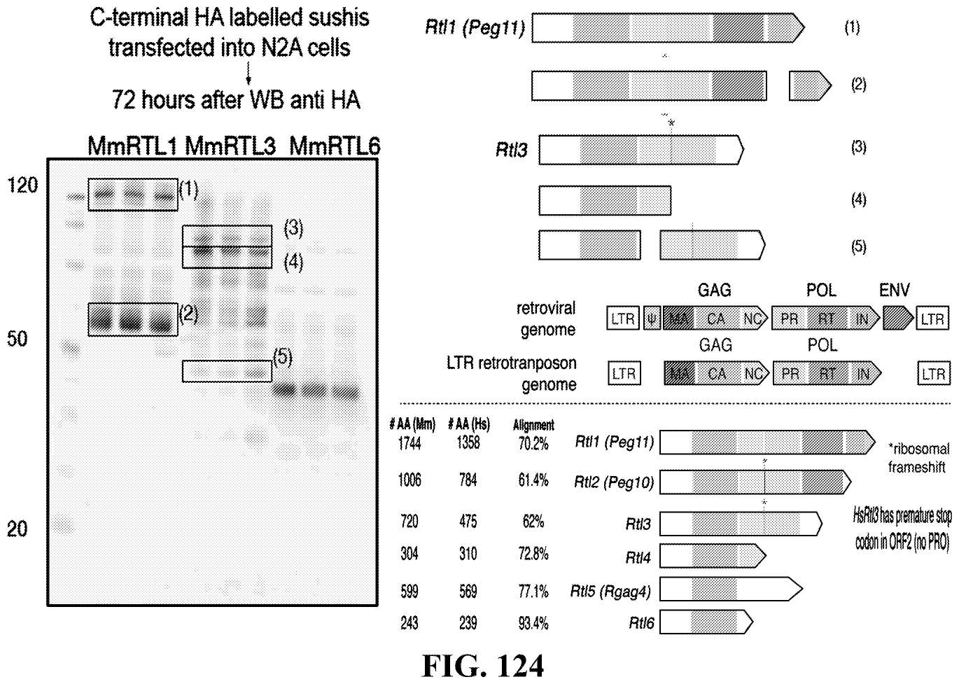

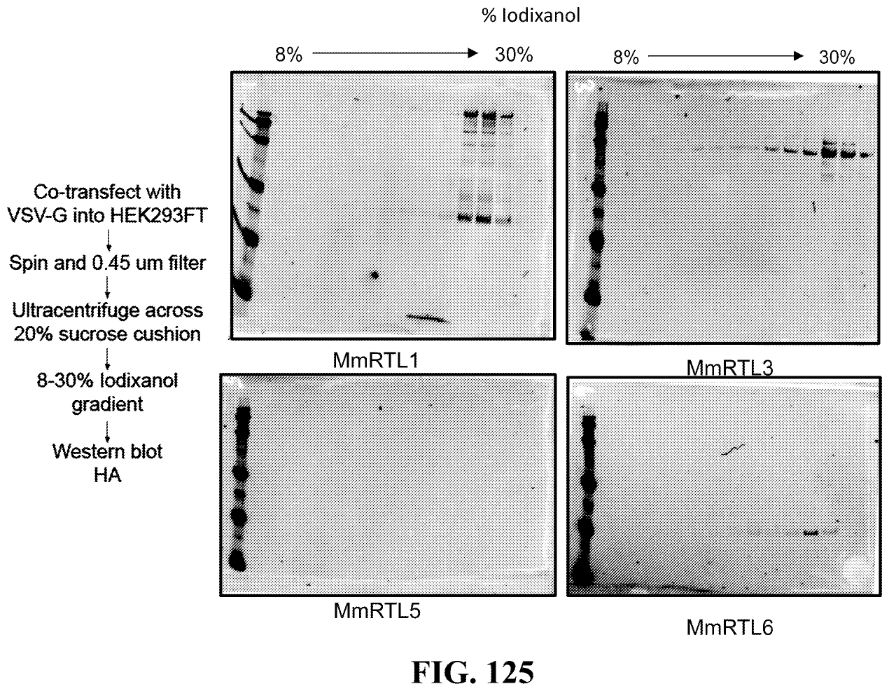

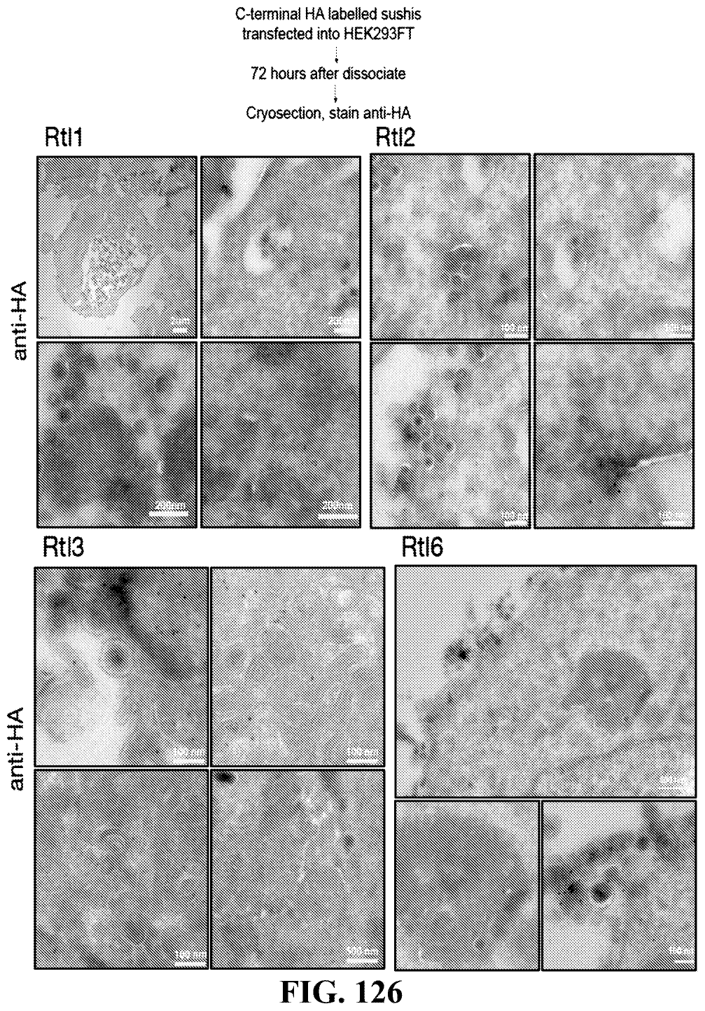

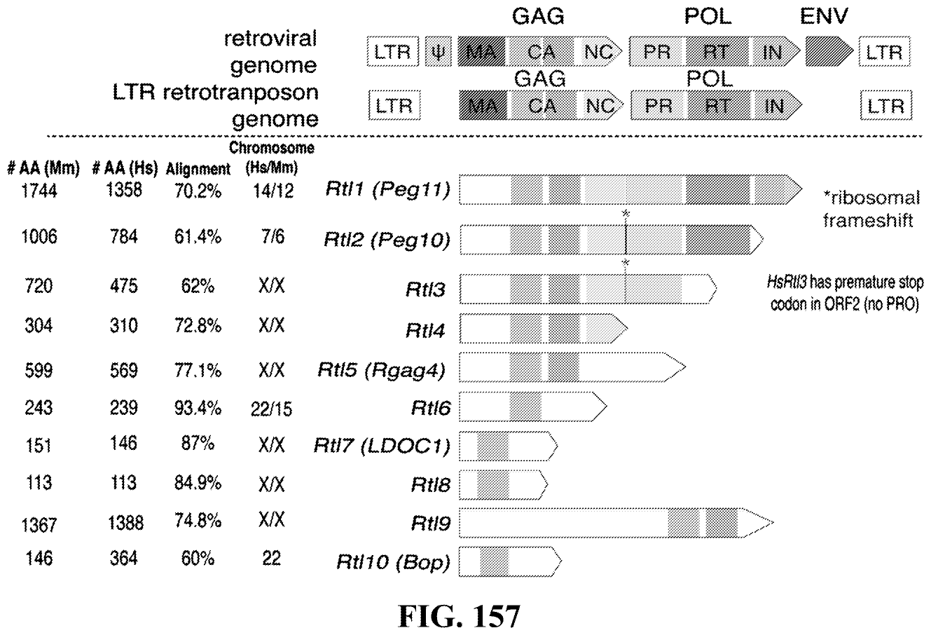

As previously discussed elsewhere herein, the Gag homolog can be a Sushi Class protein. This example explores the diversity of the Sushi family, characterizes their function, and demonstrates exploitation of their function for delivery of a cargo in vesicles (see e.g., FIG. 123, FIG. 157). Applicant demonstrated that Sushi family members containing a protease domain were processed. Briefly, Sushis were C-terminal HA tagged and transfected into N2A cells. 72 hours post transfection, a western blot against the HA tagged was performed (FIG. 124). Applicant next tested which Sushi family members are secreted into the VLP fraction. Briefly as outlined in FIG. 125, constructs expressing the Sushi family members were individually co-transfected with a VSV-G construct into HEK293FT cells. After sufficient time for packing and secretion, media was collected, spun and filtered, and peleted through a sucrose cushion and then further purified using an iodixanol gradient by ultracentrifugation to obtain the VLP fraction. A western blot of the VLP fraction was performed against the HA tag. Results are shown in FIG. 125. As shown in FIG. 125, some sushi family members are secreted into the VLP fraction. Next, Applicant determined that Sushi family members are likely secreted in MVBs as capsids (see e.g., FIG. 126). Briefly, the HA tagged Sushi family members were transfected into HEK293FT cells. After 72 hours they were dissociated, cryosectioned, and stained against the HA tag.

### Example 15—Further Investigation of Sushi Family Proteins for Delivery of Cargos

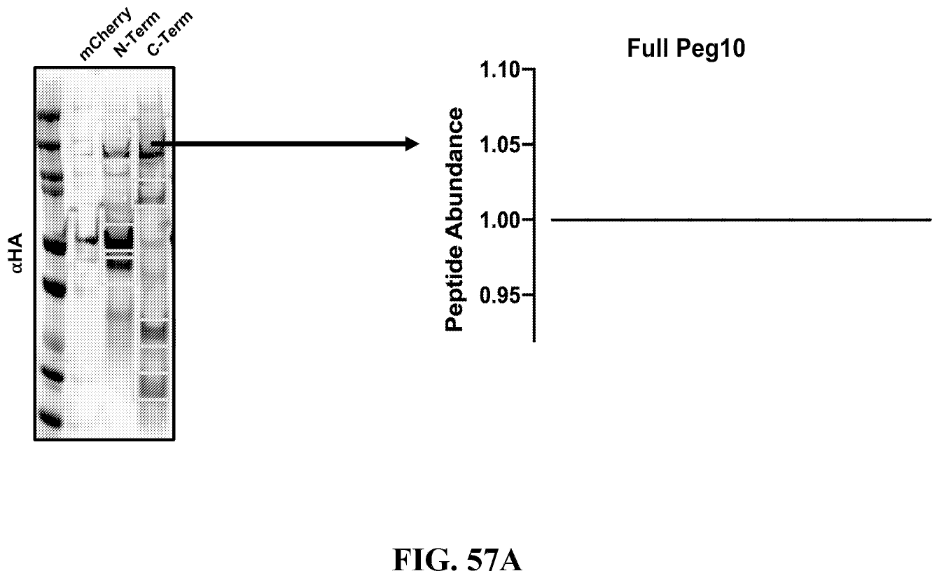

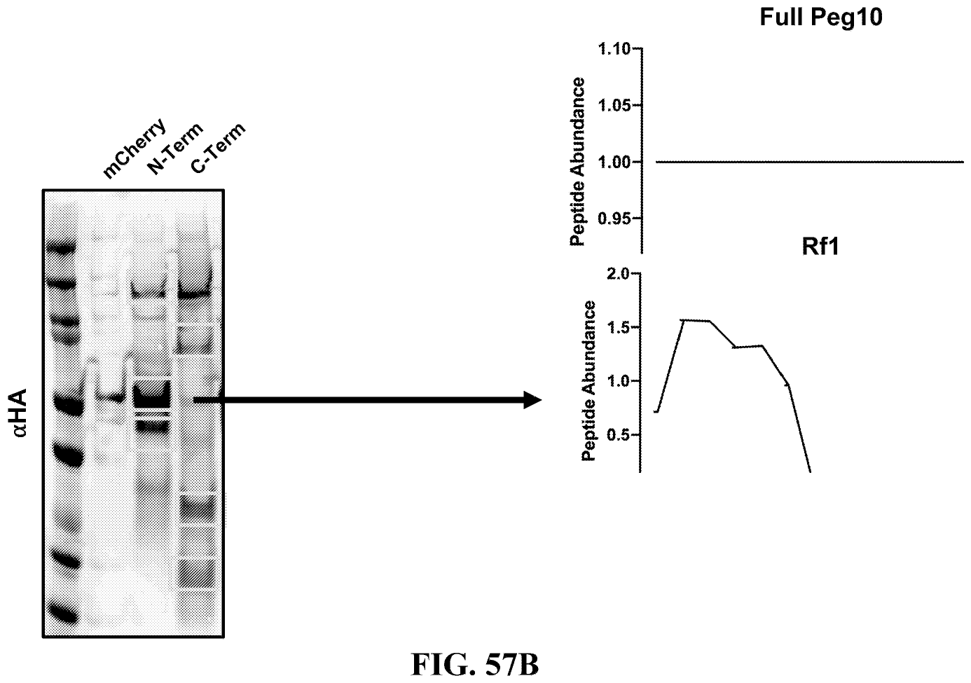

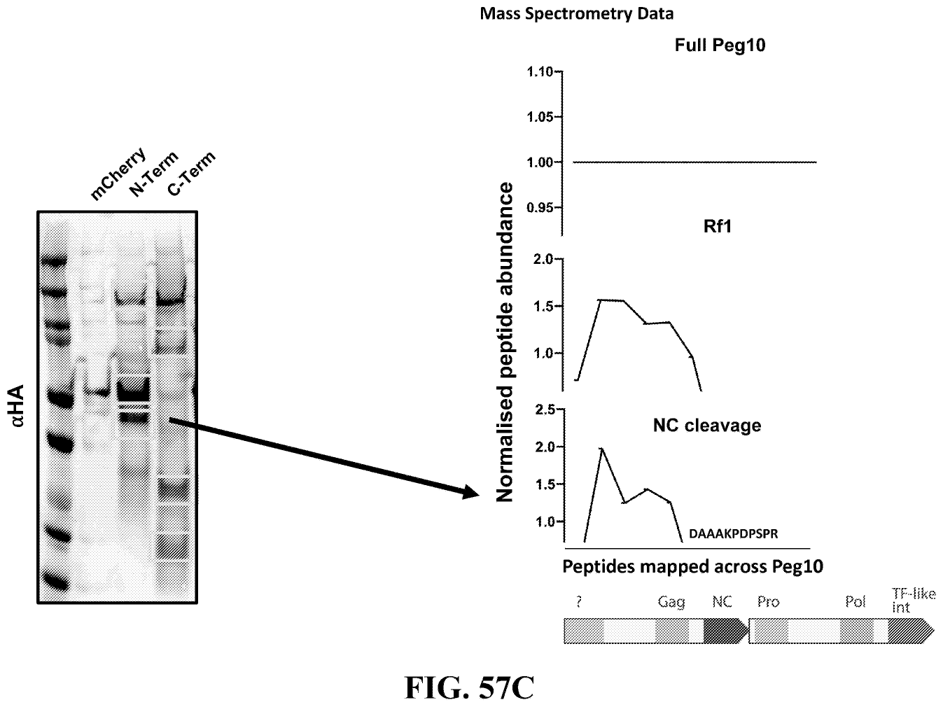

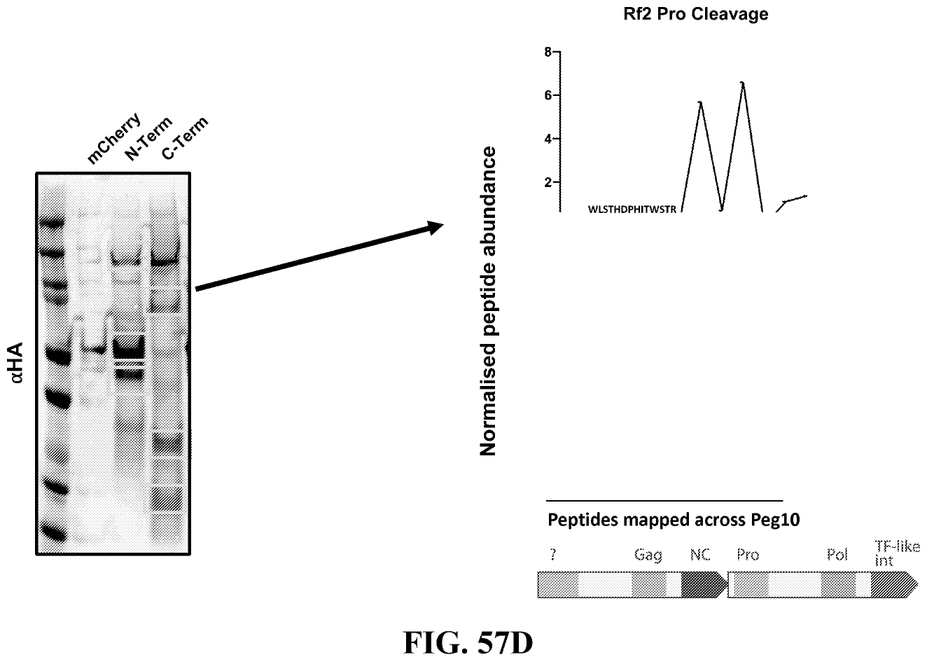

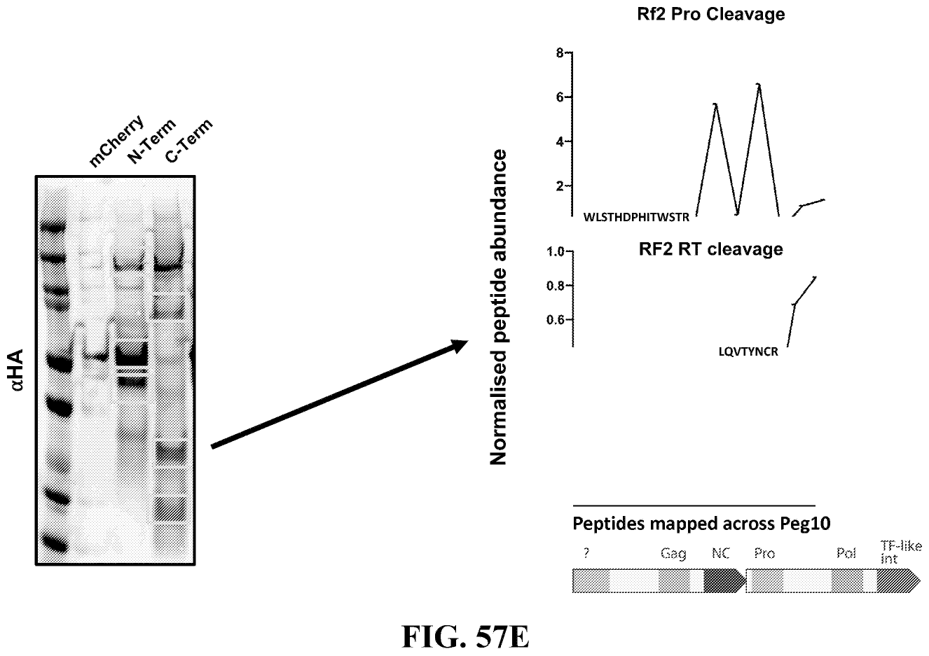

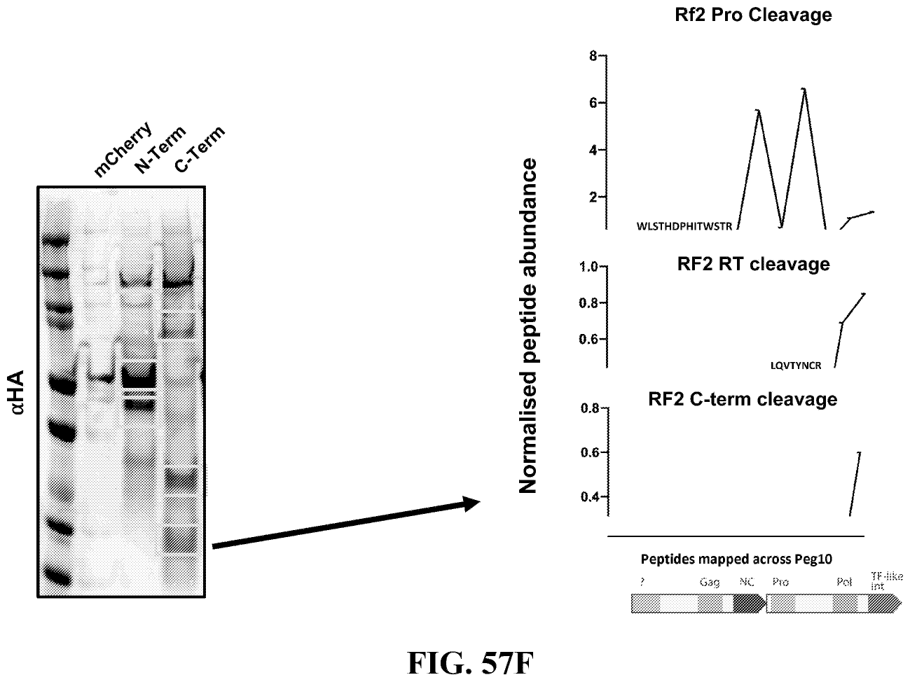

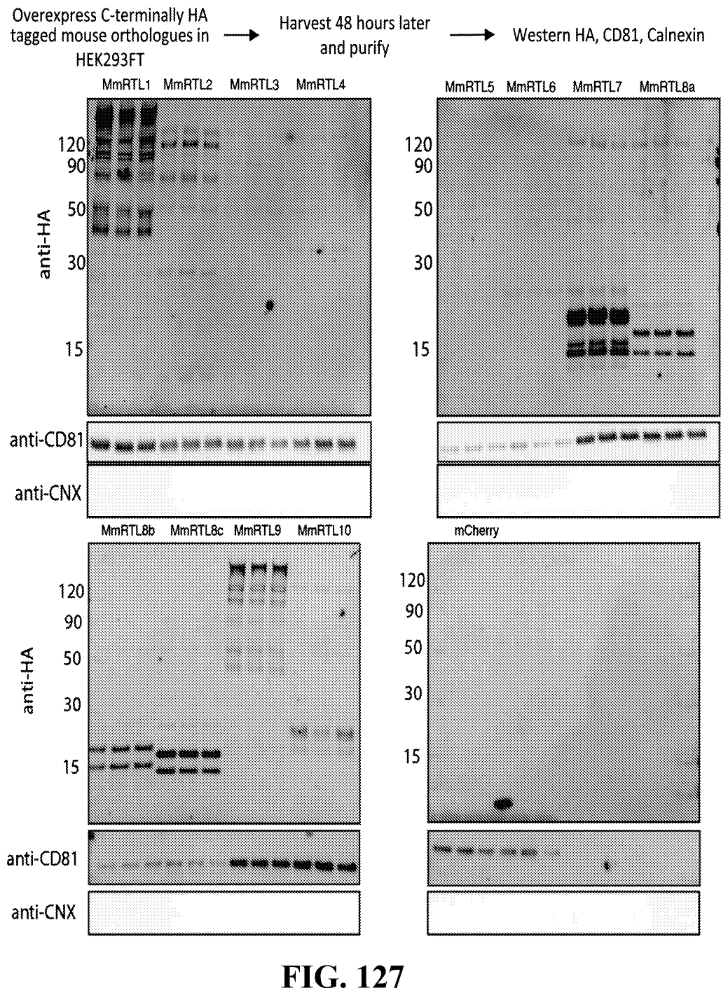

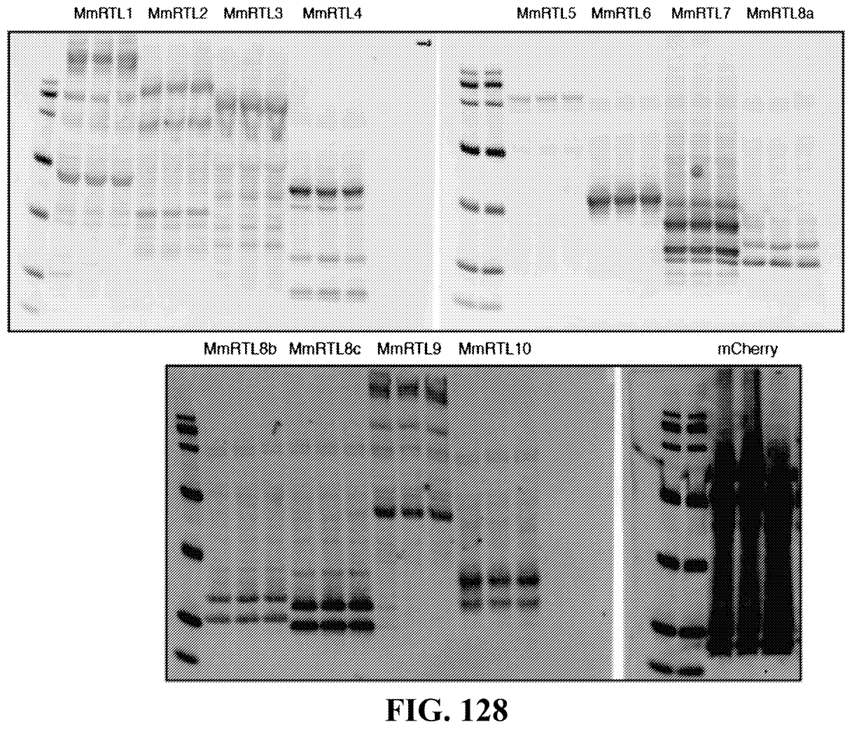

Applicants further explored the sushi-ichi family of proteins identifying expression (FIG. 128) and secretion (FIG. 127) and their binding motifs. Applicants overexpressed C-terminally HA tagged mouse orthologues in HEK0-293FT cells, harvested the cells 48 hours later and purified, and performed Western against HA tag, CD81 and Calnexin (FIG. 57) showing many sushi family members are secreted. Mouse sushi family members tested, MmRTL1, MmRTL2, MmRTL3, MmRTL4, MmRTL5, MmRTL6, MmRTL7, MmRTL8a, MmRTL8b, MmRTL8c, MmRTL9 and MmRTL10, are all expressed. (FIG. 128).

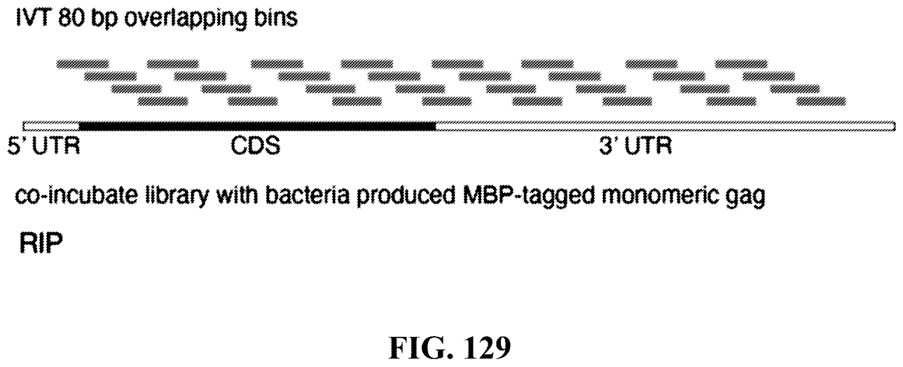

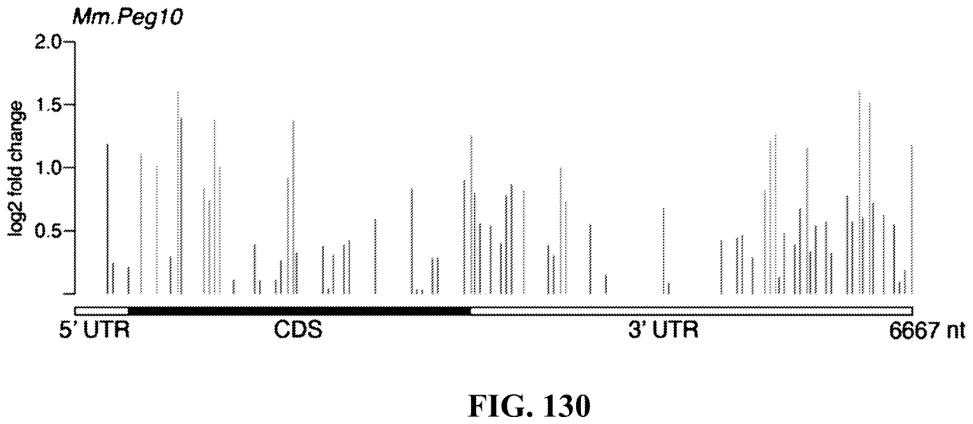

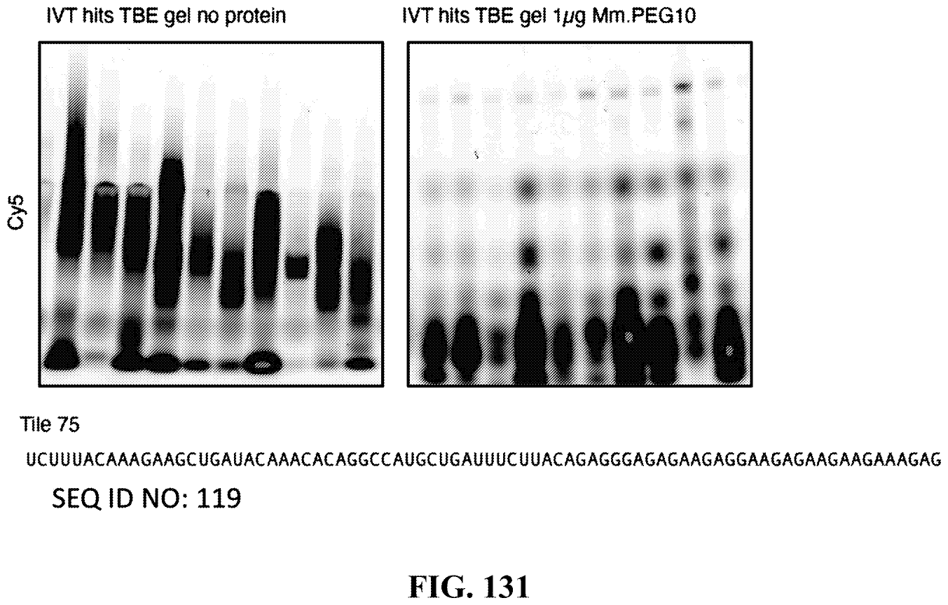

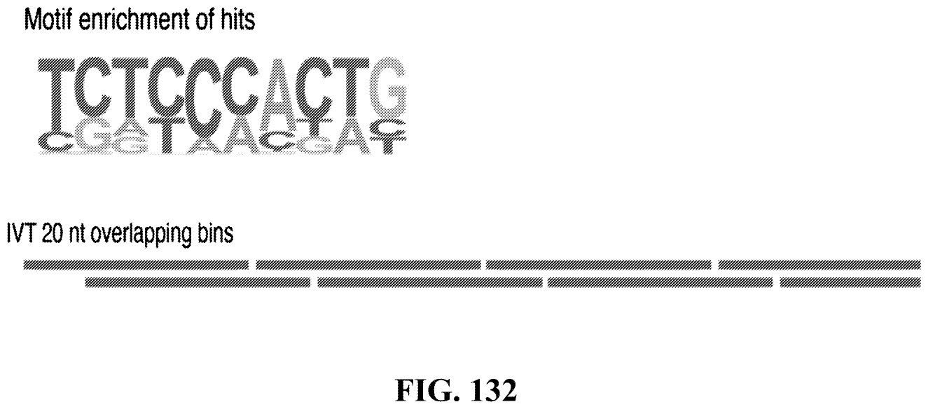

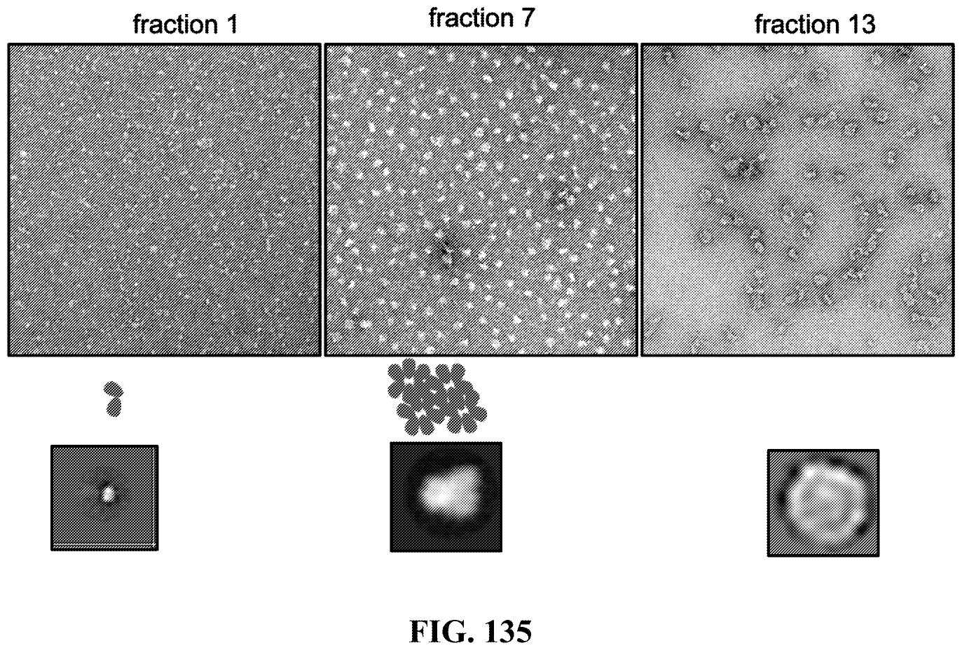

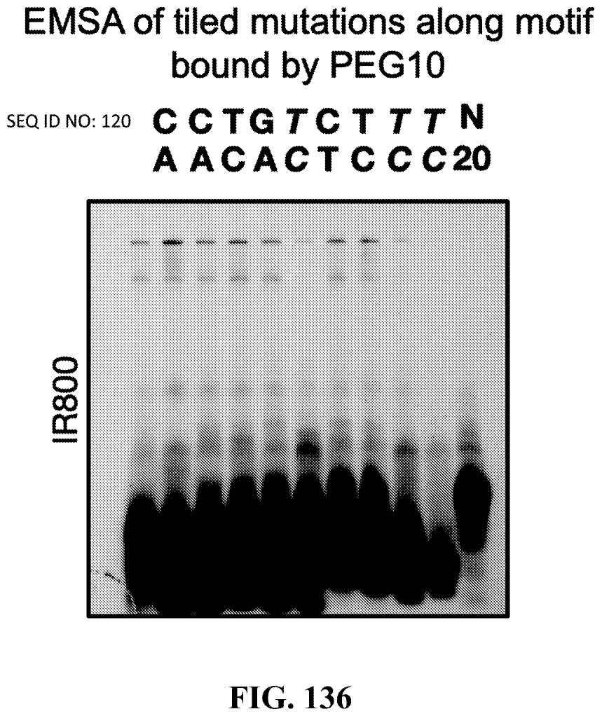

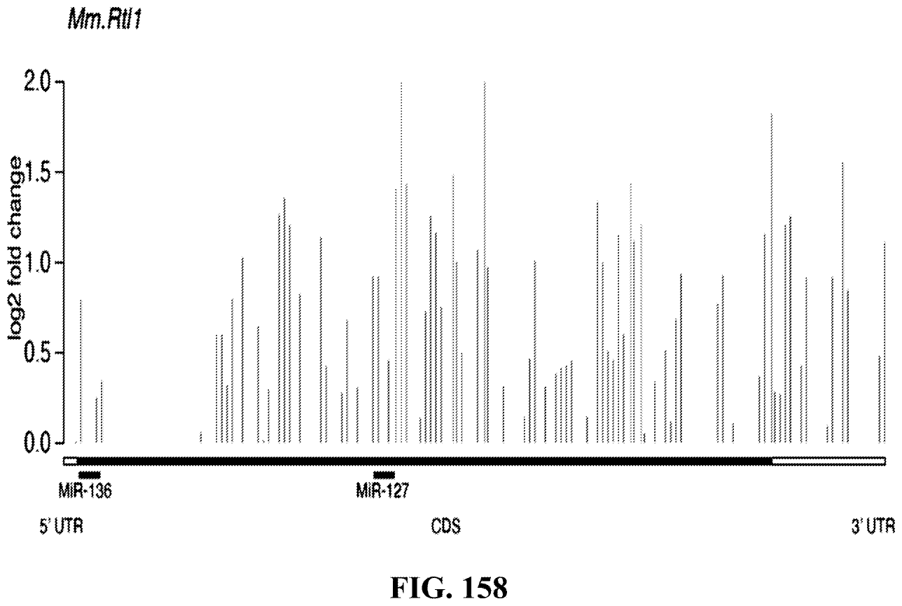

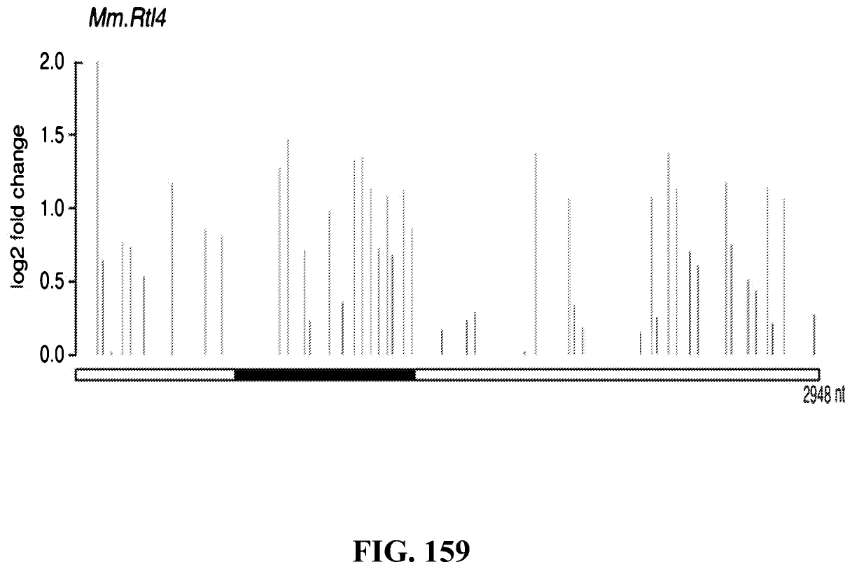

Applicants further investigated packaging elements of the Sushi family of polypeptides. FIG. 129 provides an exemplary approach to identifying protein-RNA binding motifs, e.g., packaging signal (also referred to as a packaging element). MmPeg10 packaging elements were explored (FIGS. 130-132) with a graph of packaging signal(s) across Mm.Peg10 sequence, TBE gel of in vitro transcribed hits, and weblogo motif enrichment of in vitro transcribed hits for identification of protein-RNA binding motifs (e.g., packaging elements or signals) electrophoretic mobility shift assay (EMSA) of tiled mutations along motif bound by PEG10; italic indicates inversion of base. (FIG. 136). FIG. 136 shows PEG10 binds a U rich motif found in its 3′ UTR, which may comprise a UNNUU binding motif. Applicants envision use of this motif to, inter alia, improve trans packaging and/or eliminate cis packaging. Generation of exemplary protein capsids in vitro were imaged in FIG. 135. Applicants also investigated the Mm.Rtl1 and Mm.Rtl4 sequences for packaging elements (FIGS. 158 and 159).

### Example 16 Generating In Vitro Capsids for mRNA Delivery

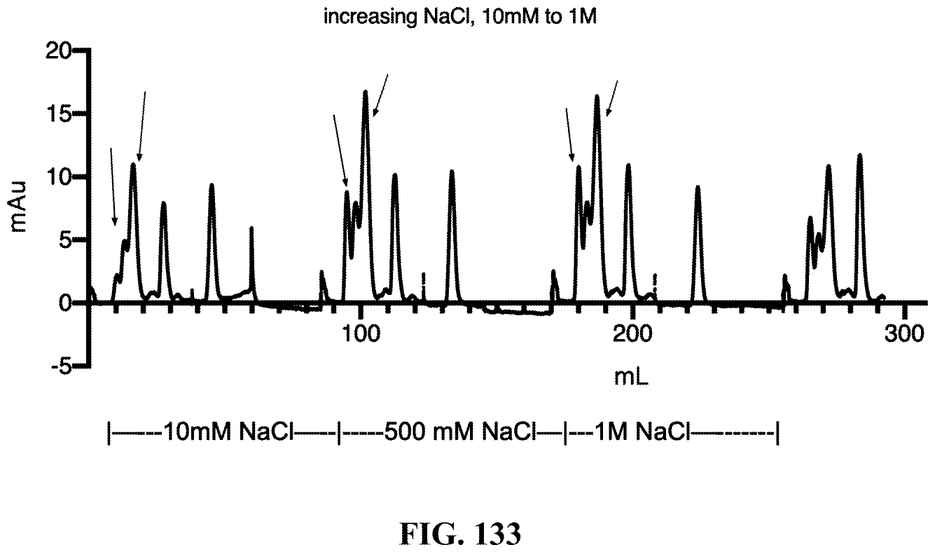

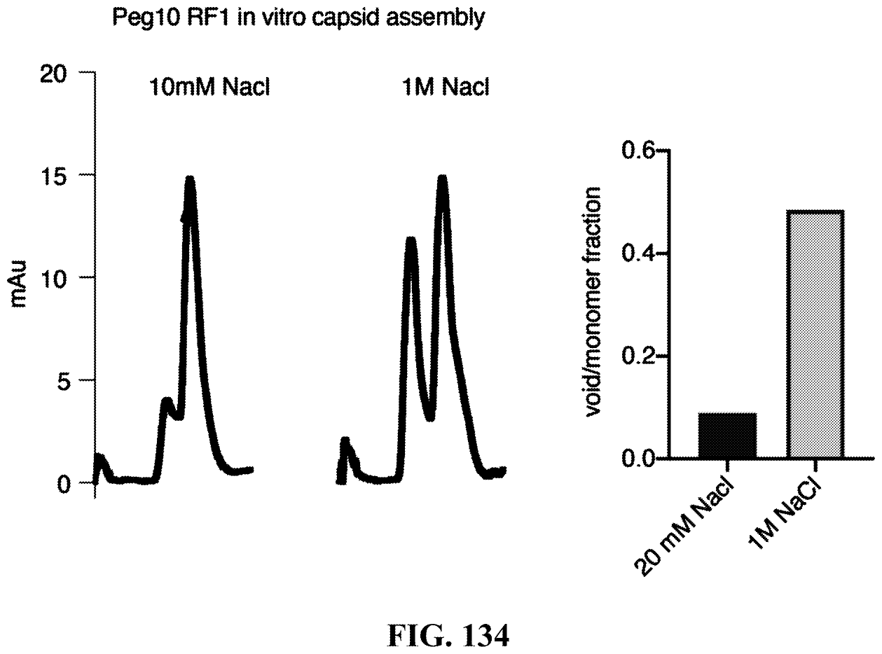

Applicants explored buffer conditions that promote RNA binding and encapsidation. Varying NaCl concentrations were measured. (FIGS. 133-134) Buffer is added to 15 ug of Purified MBP-bdSUMO-MmPEG10 RF1+1 ug Mm.cargo (Cre). NaCl concentrations of 0,100 mM, 500 mM, 1M and concentrations of ZnSO₄: 0, 50 uM, 500 uM, 1 mM are provided in buffer, SENP added, followed by Benzonase treatment, RNA extraction, and RT-qPCR. Results are shown in FIG. 137.

### Example 17 In Vivo Delivery of mRNA with Sushi Family Polypeptide Capsids

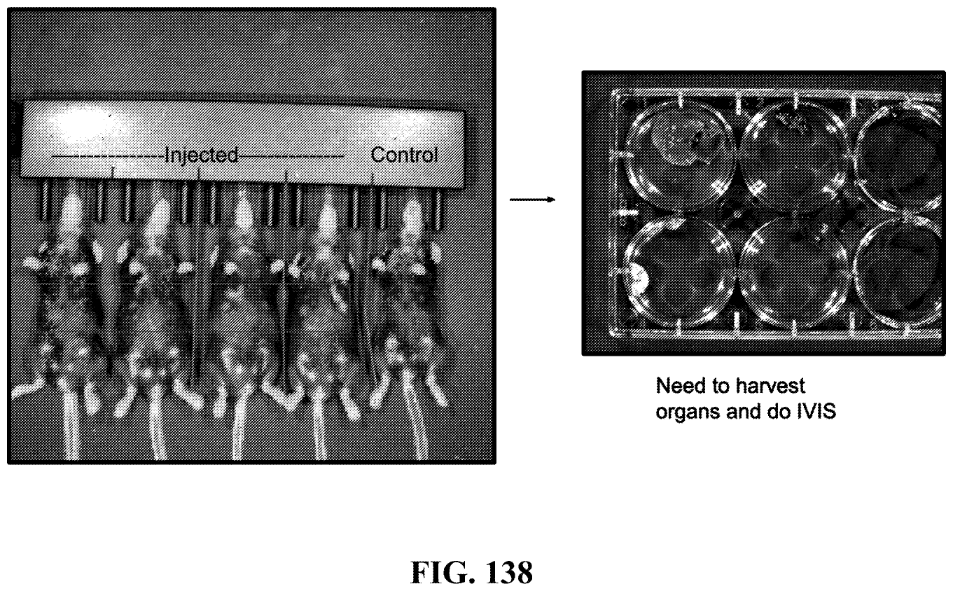

Scaling PEG10 production for in vivo use will include production of a cell suspension at scale. A variety of injection routes will be tested, including via intravenous system administration, intraocular delivery, and intracerebral/intraventricular delivery. For systemic-IV administration, both Cre and Cas9 cargo will be evaluated. For Cas9, guides will be first verified in vitro, then VLPs will be injected against PCSK9. Results of systemic-IV administration of PEG10 VLP with Cre cargo was administered in in vivo experiments (FIG. 138). Harvesting of organs and further imaging will be performed for the in vivo experiments.

### Example 18—PEG 10 Particles can be Reprogrammed for Cargo Delivery and Resemble Viral Particles

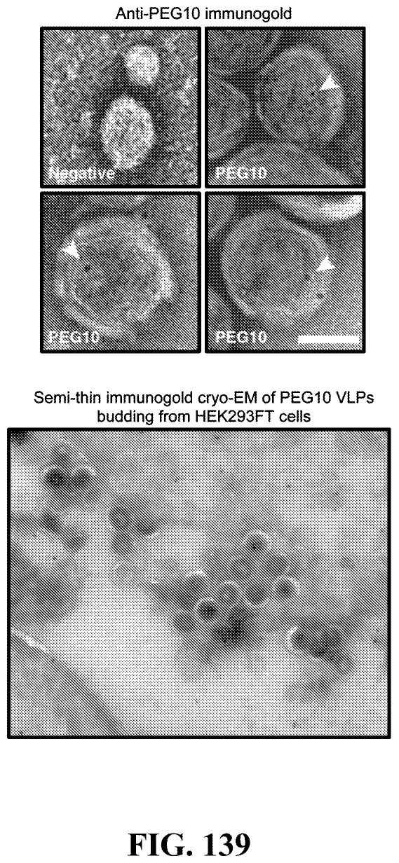

Applicants grafted the 5′ and 3′ UTRs of PEG10 onto a Cre recombinase mRNA since these regions appeared to be bound. Applicant co-transfected this plasmid with VSV-G and a plasmid encoding mouse PEG10 into HEK 239FT cells and 2 days later Applicant harvested the media containing the engineered delivery vesicles via ultracentrifugation. Applicant put these VLPs onto N2a Cre reporter cells containing a loxP-STOP-loxP GFP cassette which will express GFP upon cre recombination and used flow cytometry to quantify transfer 3 days after addition of the VLPs. Applicant observed that PEG10 VLPs are capable of transferring a UTR flanked Cre cassette into target cells with up to 25% efficiency without optimization. Without being bound by theory, transfer appeared dependent on both the fusogen VSV-G as well as PEG10 itself, and the presence of the UTRs as indicated in the bottom image of FIG. 139. As is at least demonstrated in FIG. 139, endogenous capsid-containing proteins can be reprogrammed to mediate functional transfer of mRNA.

### Example 19—Scaling SEND Production

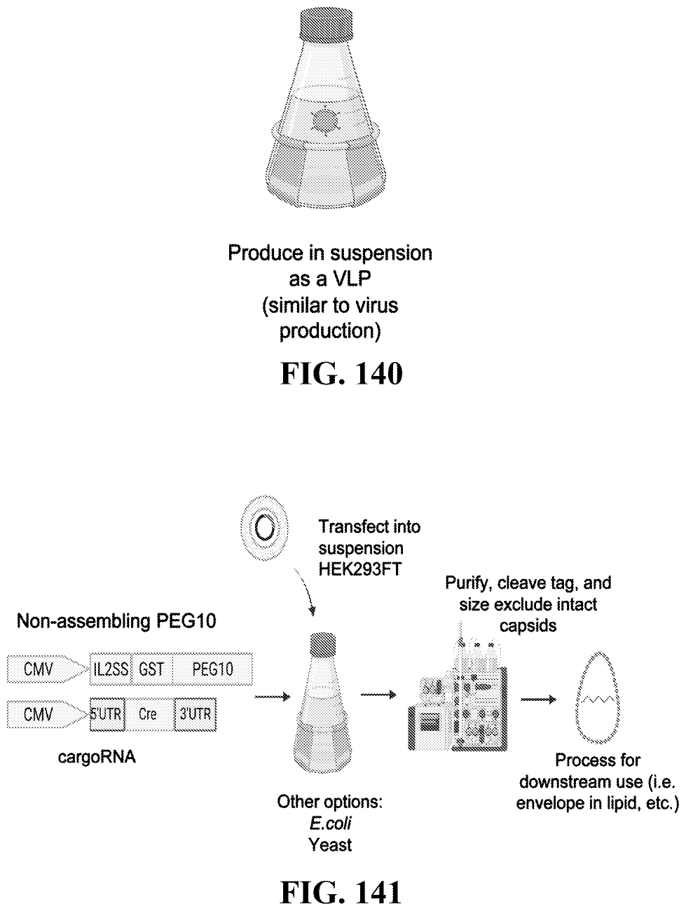

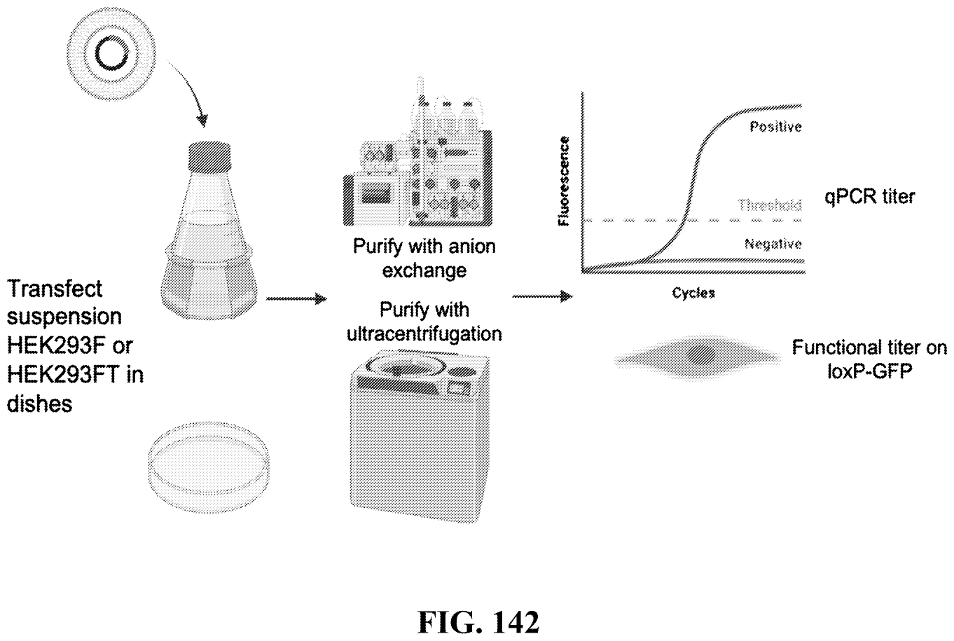

Small scale production of SEND yields about 1×10⁸-1×10⁹ particles per mL. Without being bound by theory administration for delivery of a cargo using the engineered delivery vesicle particles may use 1×10⁷-1×10⁸ particles per administration (based on mouse data). This is on par or less than the amount required by other delivery systems such as viral particles (typically about 1×10⁷-1×10⁹ particles per administration in mice) and LNPs (typically about 1×10¹¹-1×10¹³ particles per administration in mice). Applicant scaled production of the PEG10 engineered delivery vesicles. FIGS. 140-142 show a production strategy for scaling engineered delivery vesicle production from an engineered delivery vesicle generation system. Briefly, engineered delivery vesicle generation system(s) or components thereof can be delivered (e.g., transfections) to producer cells grown in suspension.

Exemplary producer cells include, without limitation, HEK293F, HEK293FT, E. coli, yeast cells, insect cells, plant cells, and/or the like. Non-eukaryotic cells can be cells appropriate for protein production. For example, such cells can be cells that have been engineered or otherwise selected for their ability to fold proteins, improve protein solubility, improved translation, add post-translation modifications (such as phosphorylations and glycoslyations), produce in the presence of toxic proteins, and/or the like. Exemplary bacterial lines include, BL21(DE3) (Novagen), BL21(DE3)-pLysS (Novagen), BL21 Star-pLysS (Novagen), BL21-SI (Invitrogen), BL21-AI (Invitrogen), Tuner (Novagen), Tuner pLysS (Novagen), Origami cells (Novagen), Origami B cells (Novagen), Origami B pLysS (Novagen), Rosetta (Novagen), Rosetta pLysS (Novagen), Rosetta-gami-pLysS (Novagen), BL21 CodonPlus (Novagen), AD494, BL21trxB (Novagen), HMS174 (Novagen), NovaBlue(DE3) (Novagen), BLR (Novagen), C41(DE3) (Lucigen), C43(DE3) (Lucigen), Lemo21(DE3) (New England Biolabs), SHuffle T7 (New England Biolabs), ArcticExpress (Agilent Technologies), and ArcticExpress (DE3)(Agilent Technologies), S. cerevisiae strains, P. pastoris, K. lactis strains, Y. lipolytica strains, and K phaffi strains (See e.g., Gomes et al., 2018. Microorganisms. 6, 28. 1-23). Other suitable producer cells are described in greater detail elsewhere herein.

The producers cells secret the produced particles (SEND or lentiviral) into the suspension media. Unsecreted particles produced can also be obtained from cell lysate. Produced particles were isolated from the media, purified, and processed for use. Purification included ultracentrifugation across a sucrose gradient or anion exchange. In some embodiments, the produced VLP can be enveloped in a lipid when necessary. The resulting product can be tittered using any suitable method(s), such as qPCR and functional expression of e.g., a reporter or other functional cargo.

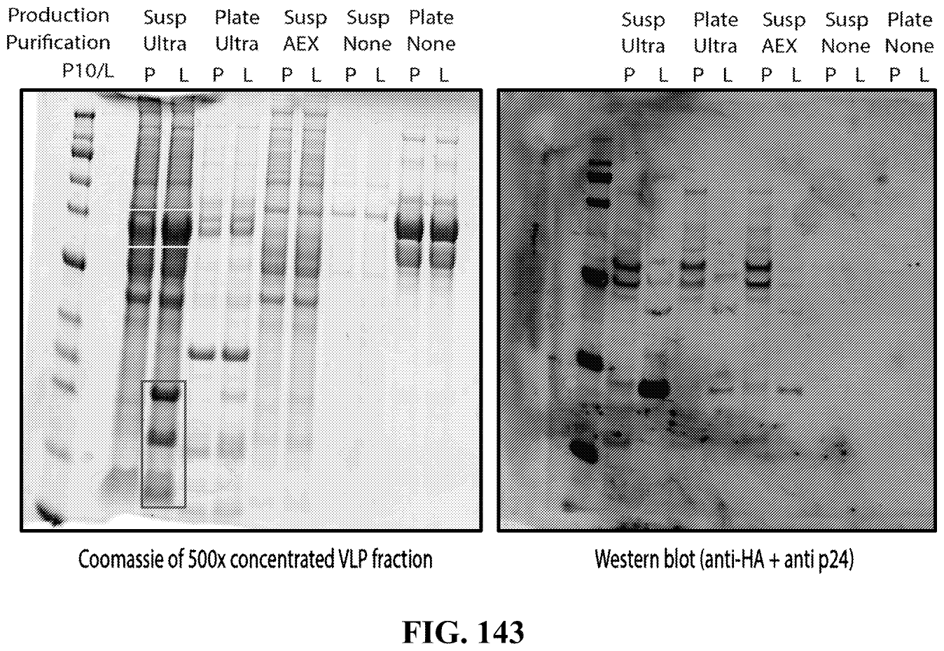

FIG. 143 shows results of optimization of purification of PEG10 engineered delivery vesicles) (“P”) produced in cells grown in suspension and a comparison of production from producer cells grown in suspension (HEK293F cells) and plated (HEK293FT cells). Purification was done by ultracentrifugation (Ultra) or anion exchange (AEX). In this Example, ultracentrufgation was performed at 120,000×g for about 2 hourse through a sucrose gradient. The anion exchange column used was a Mustang QXT5 anion exchange column. As a comparison, the same cargo was packaged in conventional lentiviral particles. (“L”)

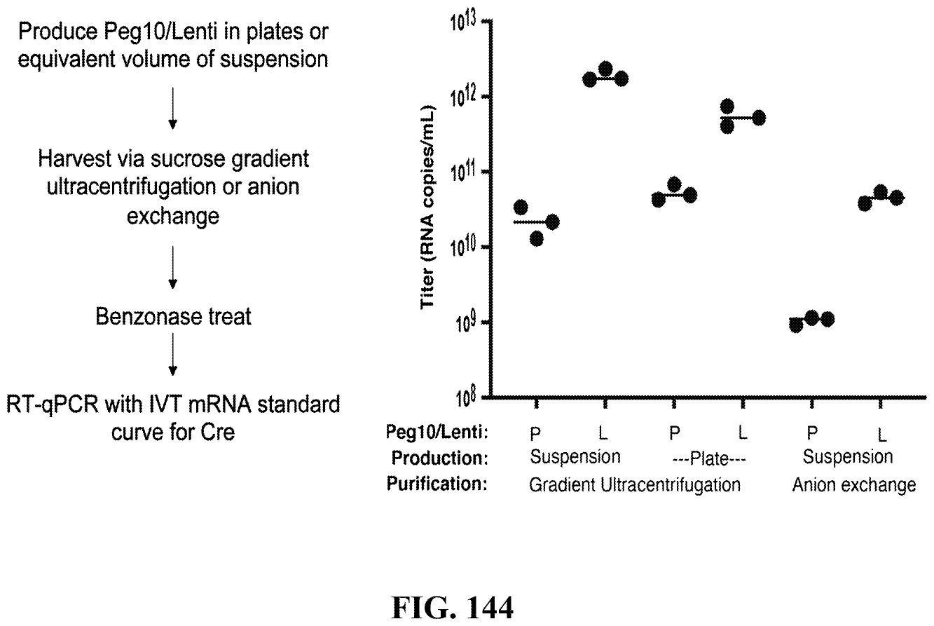

Applicant evaluated the effect of nucleic acid removal on engineered delivery vesicle production. Briefly producer cells were transfected with a PEG10 engineered delivery vesicle generation system or lentiviral system using lipofectamine 3000 (HEK293FT cells) or an LVMAX transfection kit (LVMAX cells). Cells were grown in suspension or plated for about 48 hours after which the PEG10 engineered delivery vesicles or lentiviral particles (both carrying a Cre cargo) were harvested from the media. As shown in FIG. 144, cells in suspension (HEK293F cells, specifically LVMAX cells (Thermo)) or plated (HEK293FT cells). were transfected with PEG engineered delivery systems or a control lentiviral system along with cargo to be packaged. PEG10 engineered delivery vesicles and control lentiviral particles were harvested via sucrose gradient ultracentrifugation or anion exchange. Nucleic acids were removed via a benzoase treatment. Titers were obtained using RT-qPCR with an in-vitro transcribed mRNA standard curve for Cre (the cargo) and shown in FIG. 144.

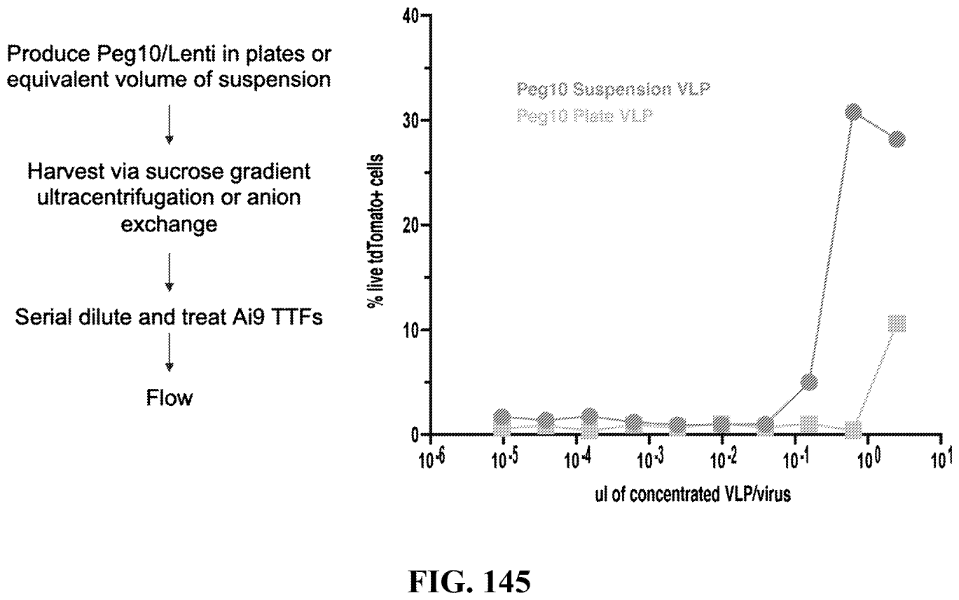

The PEG10 engineered delivery particles grown on plates and suspension were also tested in tail-tip fibroblasts obtained from Ai9 Cre reporter mice (Ai9 TTFs), which contains a lox-P-dTomato construct that activates dTomato upon Cre-recombinase 274ctivity (FIG. 145). Briefly, engineered PEG10 delivery vesicles were produced from producer cells in suspension and on plates as previously described. The particles were purified, serial diluted, and used to treat Ai9 TTFs. The cargo was a Cre recombinase, which activates dTomato fluorescence in Ai9 cells after delivery to a cell. Successful delivery of an active cargo was tested by sorting based on dTomato expression in live cells by flow cytometry using dTomato fluorescence.

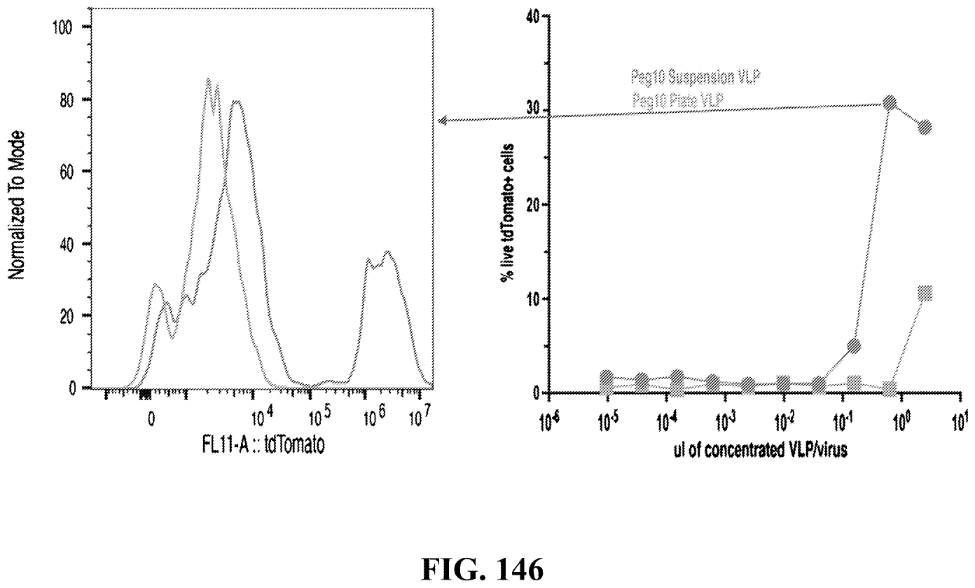

FIG. 146 shows graphs demonstrating that engineered PEG10 delivery vesicles produced in producer cells grown in suspension are more effective at cargo delivery than PEG10 delivery vesicles produced in producer cells grown on plates as previously described. In this experiment, delivery of the PEG10 delivery vesicles were delivered to Ai9cells as previously described and successful delivery and cargo functionality was evaluated as before by measuring dTomato expression/fluorescence.

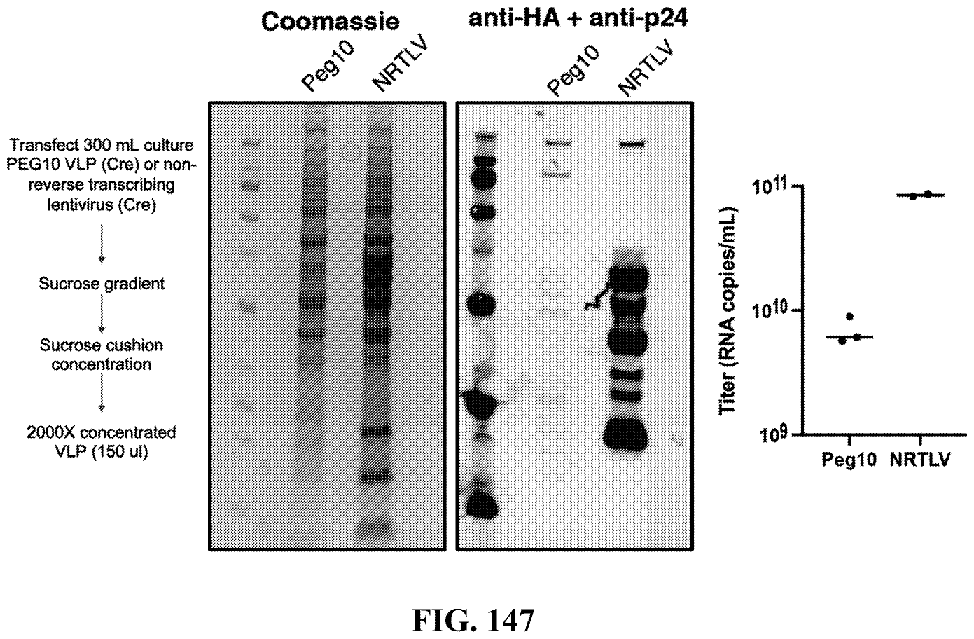

Applicant evaluated production of PEG10 engineered delivery vesicles produced suspension for in vivo delivery (FIG. 147). Briefly, a 300 mL suspension culture of producer cells (HEK293F) cells were transfected with PEG10 engineered delivery particle generation system with a Cre recombinase cargo (Peg10) or a non-reverse transcribing lentivirus generation system with a Cre recombinase cargo (NRTLV). Particles produced were harvested and purified via a sucrose gradient and concentrated against sucrose cushion gradient using ultracentrifugation. This yielded about 150 μL of a 2000× concentrated particle compositions. Concentration was performed as described in Brown et al., STAR Protocols 1, 100152, Dec. 18, 2020. https://doi.org/10.1016/j.xpro.2020.100152, particularly at pages 8-9 “Concentration of vectors for in vivo use”. The graph in FIG. 147 demonstrates titer results. Example 20—Evaluation of the Innate immune response to engineered PEG10 delivery particles

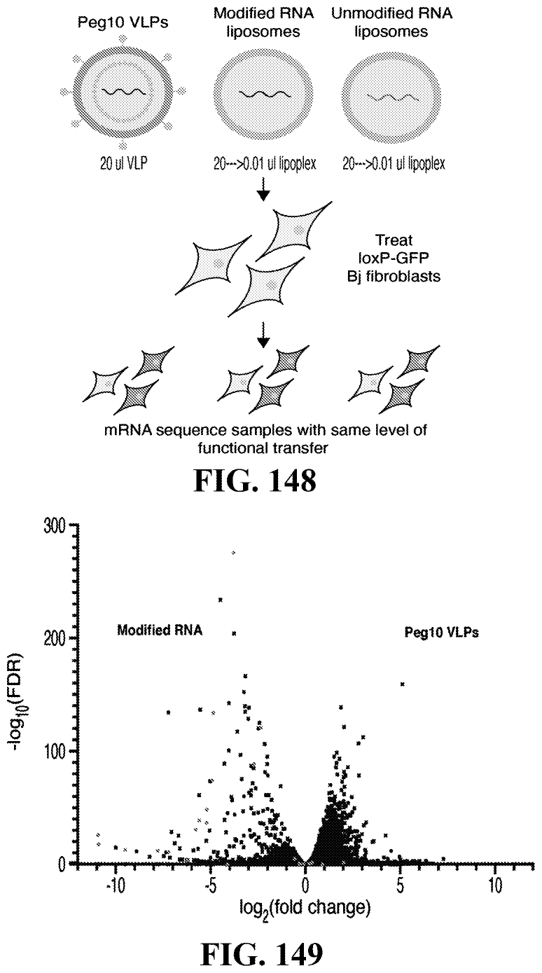

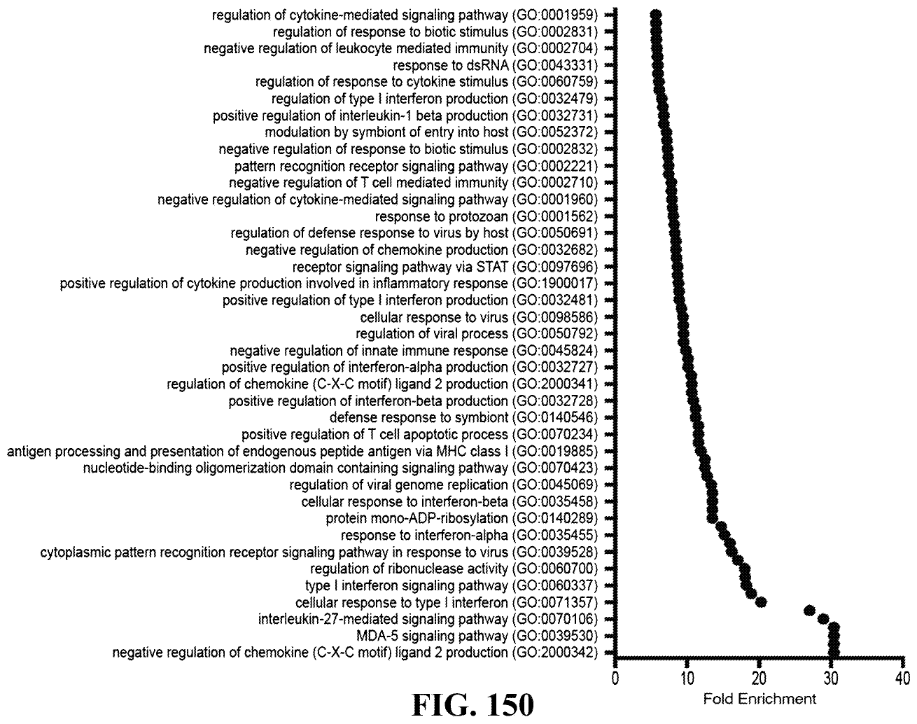

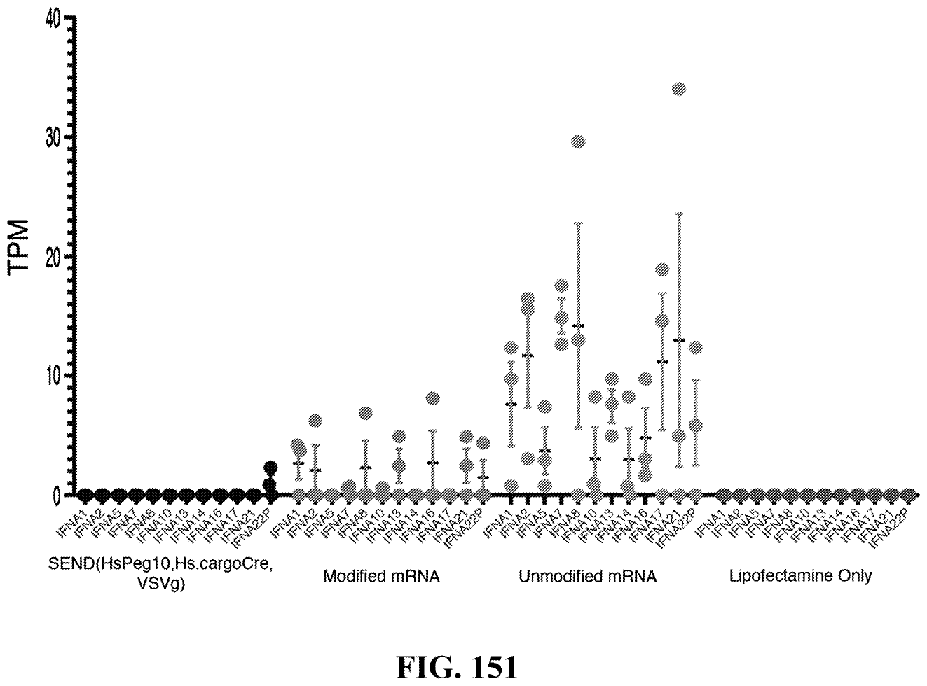

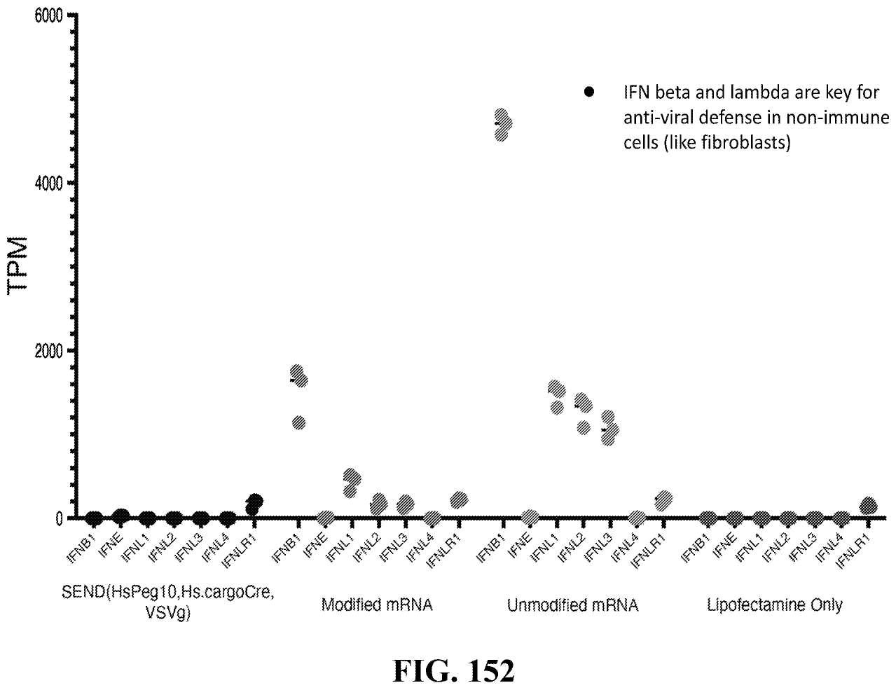

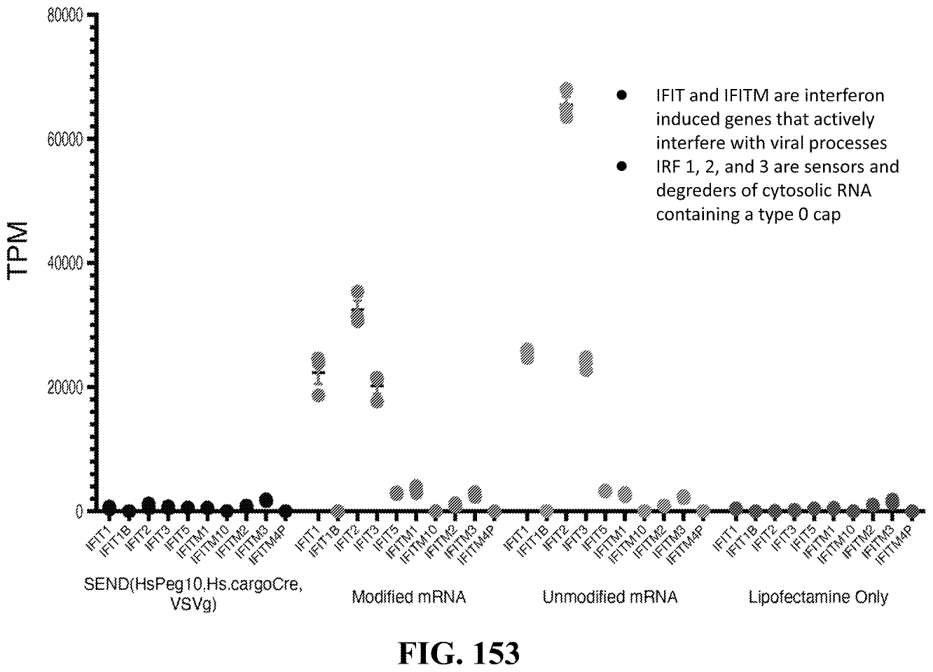

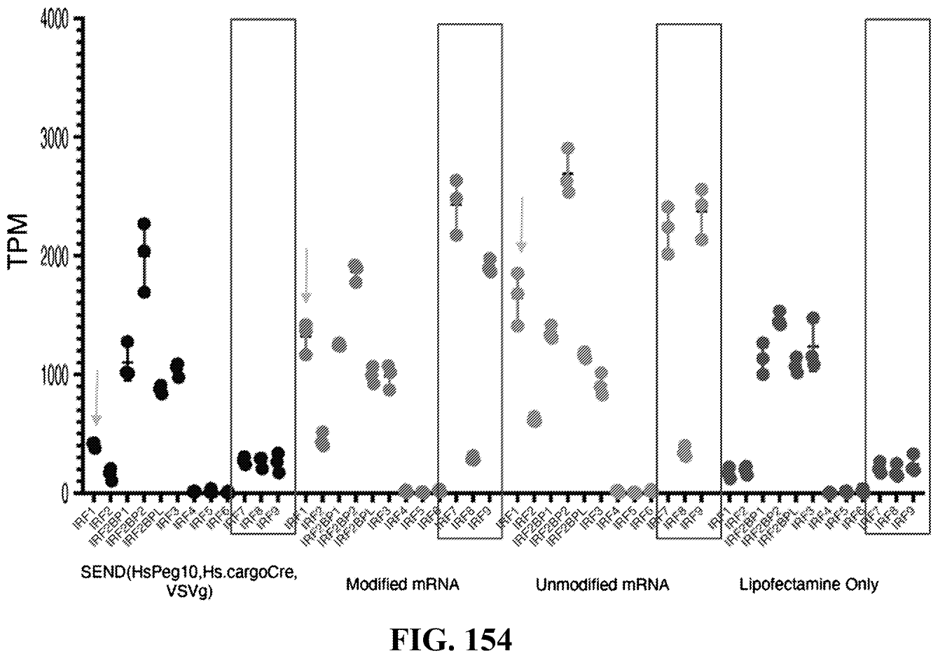

Applicants evaluated if the engineered PEG10 delivery particles initiate the innate immune response when delivering cargo RNA. FIG. 148 shows the strategy to evaluate stimulation of the innate immune response by various delivery particles (including the engineered PEG10 delivery particles (“Peg10 VLPs”). FIGS. 149-156 show results from this evaluation. FIG. 149 shows the effect of delivery via engineered PEG10 delivery particles on genes involved in the innate immune response. It was observed that genes involved in the innate immune response are upregulated upon mRNA lipoplex delivery compared to engineered PEG10 delivery vesicle mediated delivery. FIG. 150 shows genes downregulated in engineered PEG10 delivery vesicle mediated delivery vs mRNA lipoplex delivery. The genes down regulated were involved in the anti-viral response. FIG. 151 shows that the IFNa response is marginally upregulated in response to mRNA liposome delivery as compared to engineered PEG10 delivery vesicle mediated delivery (SEND). FIG. 152 shows that the IFNβ/λ response is upregulated when delivering mRNA in liposomes compared to delivery with engineered PEG10 delivery vesicles (SEND). FIG. 153 shows that delivery via engineered PEG10 delivery vesicles (SEND) circumvents activation of IFITs. FIG. 154 shows that IRF1, 7, and 9 are upregulated by liposomal delivery but not by engineered PEG10 delivery vesicle (SEND) delivery. Interferon response factors (IRFs) are transcription factors that regulate production of interferon. IRF 1, 7, and 9 were upregulated by liposomal delivery. These primarily activate expression of type 1 IFN (alpha and beta). They also induce expression of inflammatory cytokines.

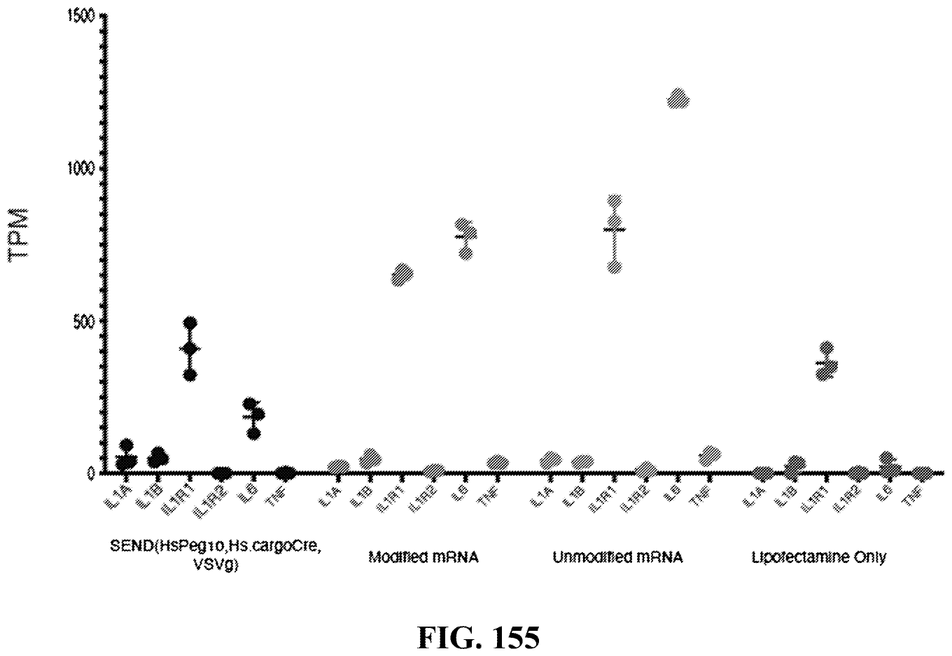

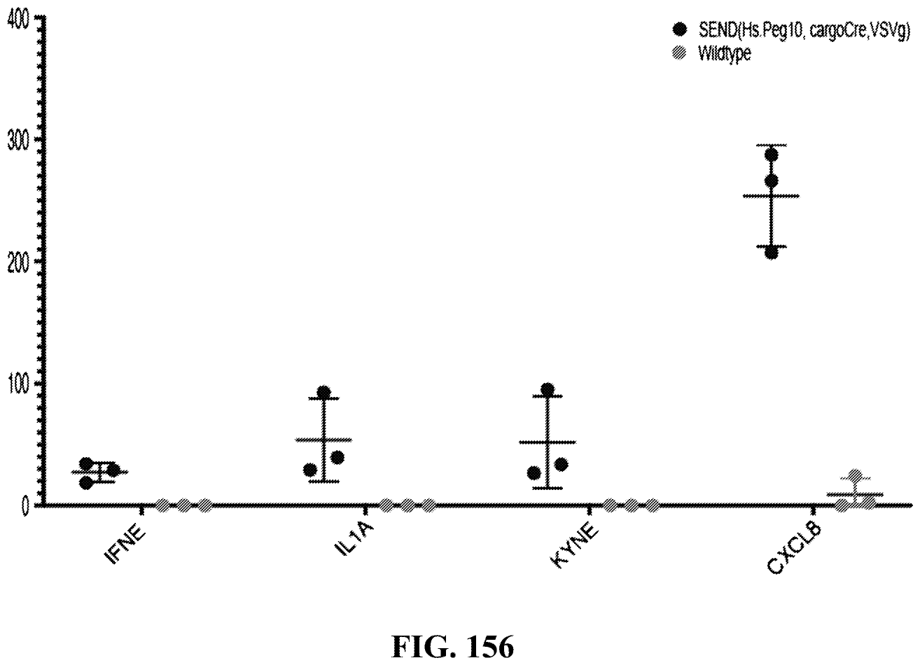

FIG. 155 shows inflammatory cytokines that are upregulated by mRNA liposome delivery as compared to engineered PEG10 delivery vesicle mediated delivery. FIG. 156 shows the effect of mRNA liposome delivery as compared to engineered PEG10 delivery vesicle mediated delivery on genes associated with the anti-viral response. IFNE is the type III IFN responsible for antiviral signaling at the epithelial barrier. IL1A is a potent inflammatory cytokine. KYNE is a metabolizer of kyneurinine and T cell activator. CXCL8 is a potent granulocyte chemotactic factor that is involved in early immune response.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. An engineered delivery vesicle generation system comprising:
a. a polynucleotide encoding an endogenous long-terminal repeat (LTR) retroelement polypeptide comprising a capsid domain, a nucleocapsid domain, a protease domain, and a reverse transcriptase domain;
b. one or more heterologous cargo polynucleotides; and
c. one or more packaging elements operatively coupled to the one or more heterologous cargo polynucleotides.

2. The system of claim 1, further comprising
d. a polynucleotide encoding a fusogenic polypeptide.

3. The system of any one of claims 1-2, wherein the endogenous LTR retroelement polypeptide is an endogenous Gag polypeptide, optionally a Sushi family polypeptide or orthologue thereof.

4. The system of claim 3, wherein the Gag polypeptide is a PEG10 polypeptide or orthologue thereof, an RTL1 polypeptide or orthologue thereof, an RTL3 polypeptide or orthologue thereof, an RTL5 polypeptide or orthologue thereof, an RTL6 polypeptide or orthologue thereof, or any combination thereof.

5. The system of claim 1, wherein the polynucleotide encoding the endogenous LTR retroelement polypeptide comprises one or more modifications that enhance binding specificity and/or packaging of the cargo polynucleotide and/or reduce endogenous LTR retroelement polypeptide binding to an endogenous LTR retroelement polypeptide mRNA.

6. The system of claim 5, wherein the one or more modifications are in the polynucleotide encoding the endogenous LTR retroelement polypeptide at the boundary between the nucleocapsid domain encoding region and protease domain encoding region.

7. The system of claim 1, wherein the one or more packaging elements are capable of complexing with one or more domains of the endogenous LTR retroelement polypeptide.

8. The system of claim 7, wherein the one or more packaging elements comprise one or more 5′ untranslated regions (UTRs) or portion thereof, one or more 3′ UTRs or portion thereof, or both, and wherein one or more the 5′ UTR or portion thereof, the 3′ UTRs or portion thereof or both are capable of complexing with one or more domains of the endogenous LTR retroelement polypeptide, and optionally wherein at least one of the one or more 3′ UTRs or portion thereof comprises about 500 bp of a proximal end of a 3′UTR of an mRNA encoding an endogenous LTR retroelement polypeptide.

9. The system of claim 8, wherein one or more of the one or more 5′ UTRs or portion thereof are derived from a UTR of an mRNA encoding an endogenous LTR polypeptide, wherein one or more of the one or more 3′ UTRs are derived from a UTR of an mRNA encoding an endogenous LTR polypeptide, or both.

10. The system of claim 7, wherein one or more of the one or more packaging elements comprises at least a 3′ UTR or portion thereof derived from a UTR of an mRNA encoding an endogenous LTR retroelement polypeptide.

11. The system of claim 10, wherein the 3′UTR or portion thereof comprises about 500 bp of a proximal end of a 3′UTR of an mRNA encoding an endogenous LTR retroelement polypeptide.

12. The system of claim 8, wherein the mRNA encoding an endogenous LTR retroelement polypeptide is an mRNA encoding an endogenous Gag polypeptide, optionally a Sushi family protein.

13. The system of claim 12, wherein the mRNA encoding an endogenous LTR retroelement polypeptide is an mRNA encoding a PEG10 polypeptide or orthologue thereof, an RTL1 polypeptide or orthologue thereof, an RTL3 polypeptide or orthologue thereof, an RTL5 polypeptide or orthologue thereof, an RTL6 polypeptide or orthologue thereof.

14. The system of any one of claims 8-13, wherein the packaging element is a polynucleotide comprising a polynucleotide motif having a sequence of UNNUU, wherein each N is independently selected from A, T, C, G, or U.

15. The system of claim 1, wherein the fusogenic polypeptide is specific for a target cell type to which the cargo polynucleotide is targeted for delivery.

16. The system of claim 1, wherein the fusogenic polypeptide is a tetraspanin (TSPAN), a G envelope protein, an epsilon-sarcoglycan (SGCE), a syncitin, or a combination thereof.

17. The system of claim 16, wherein the TSPAN is CD81, CD9, CD63 or a combination thereof.

18. The system of claim 16, wherein the G envelope protein is a vesicular stomatitis virus G envelope protein (VSV-G).

19. The system of any one of claims 1-2, wherein (a), (b), (c), and (d) are encoded on one or more vectors comprising one or more regulatory elements, and wherein (a), (b), (c) and/or (d) are optionally operatively coupled to the one or more regulatory elements.

20. The system of any one of claims 1-2, wherein (a), (b), and (c) are encoded on the same vector.

21. The system of claim 1, wherein the at least one or more heterologous cargo polynucleotides are RNA, DNA, or hybrid RNA/DNA.

22. The system of claim 21, wherein the at least one or more heterologous cargo polynucleotides comprise one or more modifications capable of modifying the functionality, packaging ability, stability, localization, or any combination thereof, of the at least one or more heterologous cargo polynucleotides.

23. The system of claim 1, wherein at least one of the one or more heterologous cargo polynucleotides encodes an RNA guided nuclease system or component thereof, optionally an RNA guided nuclease.

24. The system of claim 23, wherein the RNA guided nuclease system is a Cas-based system or an IscB system and wherein the optional RNA guided nuclease is a Cas polypeptide or an IscB polypeptide.

25. The system of any one of claims 23-24, wherein at least one of the one or more heterologous cargo polynucleotides comprises a guide polynucleotide and/or a polynucleotide encoding a guide polynucleotide, optionally wherein the guide polynucleotide and/or the polynucleotide encoding a guide polynucleotide are operatively coupled to one or more of the one or more packaging elements.

26. The system of claim 25, wherein the at least one of the one or more heterologous cargo polynucleotides that encodes an RNA guided nuclease further comprises a guide polynucleotide or a polynucleotide encoding a guide polynucleotide.

27. The system of claim 26, wherein the guide polynucleotide or the polynucleotide encoding a guide polynucleotide is operatively coupled to the same packaging elements as one or more at least one heterologous cargo polynucleotides that encodes an RNA guided nuclease.

28. The system of claim 1, wherein the polynucleotide encoding an endogenous long-terminal repeat (LTR) retroelement polypeptide, optionally the capsid domain, comprises a targeting moiety and wherein the polynucleotide is configured such that the targeting moiety is present on an external capsid surface when expressed and formed into a capsid.

29. An engineered delivery vesicle comprising:
a. a polynucleotide encoding an endogenous LTR retroelement polypeptide comprising a capsid domain, a nucleocapsid domain, a protease domain, and a reverse transcriptase domain;
b. one or more heterologous cargo polynucleotides
c. one or more packaging elements, wherein the one or more packaging elements are operatively coupled to at least one of the one or more heterologous cargo polynucleotides; and
d. a fusogenic polypeptide.

30. The delivery vesicle of claim 29, wherein the endogenous LTR retroelement polypeptide is an endogenous Gag polypeptide, optionally a Sushi family polypeptide or orthologue thereof.

31. The delivery vesicle of claim 30, wherein the Sushi family polypeptide is a PEG10 polypeptide or orthologue thereof, an RTL1 polypeptide or orthologue thereof, an RTL3 polypeptide or orthologue thereof, an RTL5 polypeptide or orthologue thereof, an RTL6 polypeptide or orthologue thereof, or any combination thereof.

32. The delivery vesicle of claim 29-31, wherein the endogenous LTR retroelement polypeptide comprises one or more modifications that enhance the binding specificity and/or packaging of a heterologous cargo polynucleotide and/or reduce the endogenous LTR retroelement polypeptide binding to an endogenous LTR retroelement polypeptide mRNA.

33. The delivery vesicle of claim 32, wherein the one or more modifications are at or near the boundary of the nucleocapsid domain and the protease domain of the endogenous LTR retroelement polypeptide.

34. The delivery vesicle of claim 29, wherein the one or more packaging elements are capable of complexing with one or more domains of the endogenous LTR retroelement polypeptide.

35. The delivery vesicle of claim 34, wherein the one or more packaging elements comprise one or more 5′ untranslated regions (UTRs) or portion thereof, one or more 3′ UTRs or portion thereof, or both, and wherein the one or more 5′ UTRs or portion thereof, the one or more 3′ UTRs or portion thereof, or both are capable of complexing with one or more domains of the endogenous LTR retroelement polypeptide, and optionally wherein at least one of the one or more 3′ UTRs or portion thereof comprises about 500 bp of a proximal end of a 3′UTR of an mRNA encoding an endogenous LTR retroelement polypeptide.

36. The delivery vesicle of claim 35, wherein one or more of the one or more 5′ UTRs or portion thereof are derived from a UTR of an mRNA encoding an endogenous LTR polypeptide, wherein one or more of the one or more 3′ UTRs are derived from a UTR of an mRNA encoding an endogenous LTR polypeptide, or both.

37. The delivery vesicle of claim 34, wherein one or more of the one or more packaging elements comprises at least a 3′ UTR or portion thereof derived from a UTR of an mRNA encoding an endogenous LTR retroelement polypeptide.

38. The delivery vesicle of claim 37, wherein the 3′UTR or portion thereof comprises about 500 bp of a proximal end of a 3′UTR of an mRNA encoding an endogenous LTR retroelement polypeptide.

39. The delivery vesicle of claim 34, wherein the mRNA encoding an endogenous LTR retroelement polypeptide is an mRNA encoding an endogenous Gag polypeptide, optionally a Sushi family protein.

40. The delivery vesicle of claim 39, the mRNA encoding an endogenous LTR retroelement polypeptide is an mRNA encoding a PEG10 polypeptide or orthologue thereof, an RTL1 polypeptide or orthologue thereof, an RTL3 polypeptide or orthologue thereof, an RTL5 polypeptide or orthologue thereof, an RTL6 polypeptide or orthologue thereof.

41. The delivery vesicle of any one of claims 34-40, wherein the packaging element is a polynucleotide comprising a polynucleotide motif having a sequence of UNNUU, wherein each N is independently selected from A, T, C, G, or U.

42. The delivery vesicle of claim 29, wherein the fusogenic polypeptide is specific for a target cell type to which the cargo polynucleotide is targeted for delivery.

43. The delivery vesicle of claim 42, wherein the fusogenic polypeptide is a tetraspanin (TSPAN), a G envelope protein, an epsilon-sarcoglycan (SGCE), a syncitin, or a combination thereof.

44. The delivery vesicle of claim 43, wherein the TSPAN is CD81, CD9, CD63 or a combination thereof.

45. The delivery vesicle claim 43, wherein the G envelope protein is a vesicular stomatitis virus G envelope protein (VSV-G).

46. The delivery vesicle of claim 29, wherein the at least one or more heterologous cargo polynucleotides are RNA, DNA, or hybrid RNA/DNA.

47. The delivery vesicle of claim 46, wherein the at least one or more heterologous cargo polynucleotides comprise one or more modifications capable of modifying the functionality, packaging ability, stability, localization, or any combination thereof, of the at least one or more heterologous cargo polynucleotides.

48. The delivery vesicle of claim 29, wherein at least one of the one or more heterologous cargo polynucleotides encodes an RNA guided nuclease system or component thereof, optionally an RNA guided nuclease.

49. The delivery vesicle of claim 48, wherein the RNA guided nuclease system is a Cas-based system or an IscB system and wherein the optional RNA guided nuclease is a Cas polypeptide or an IscB polypeptide.

50. The delivery vesicle of any one of claims 48-49, wherein at least one of the one or more heterologous cargo polynucleotides comprises a guide polynucleotide and/or a polynucleotide encoding a guide polynucleotide, optionally wherein the guide polynucleotide and/or the polynucleotide encoding a guide polynucleotide are operatively coupled to one or more of the one or more packaging elements.

51. The delivery vesicle of claim 50, wherein the at least one of the one or more heterologous cargo polynucleotides that encodes an RNA guided nuclease further comprises a guide polynucleotide or a polynucleotide encoding a guide polynucleotide.

52. The delivery vesicle of claim 51, wherein the guide polynucleotide or the polynucleotide encoding a guide polynucleotide is operatively coupled to the same packaging elements as one or more at least one heterologous cargo polynucleotides that encodes an RNA guided nuclease.

53. A delivery vesicle, optionally a delivery vesicle of any one of claims 29-52, wherein the delivery vesicle is generated by a system of any one of claims 1-28.

54. A method of generating engineered delivery vesicles loaded with one or more cargo polynucleotides, comprising:
a. delivering to and/or incubating a delivery vesicle generation system of any one of claims 1-28 in one or more bioreactors; and
b. isolating generated engineered delivery vesicles from the one or more bioreactors.

55. The method of claim 54, wherein the one or more bioreactors are one or more cells, optionally one or more eukaryotic cells or prokaryotic cells.

56. The method of claim 55, wherein the cells are cultured in suspension during incubation.

57. The method of any one of claims 54-56, further comprising purifying isolated engineered delivery vesicles.

58. The method of any one of claims 54-57, further comprising concentrating the isolated and/or purified engineered delivery vesicles, optionally 1-5000×.

59. A delivery vesicle generated according to the method of any one of claims 54-58.

60. A cell comprising: an engineered delivery vesicle generation system of any one of claims 1-28 and/or a delivery vesicle of any one of claims 29-53 or 59.

61. A co-culture system comprising two or more cell types, wherein at least one cell type of the two or more cell types, all cell types of the two or more cell types, or a sub-combination of cell-types of the two or more cell types comprise an engineered delivery system of any one of claims 1-28.

62. A method of cellular delivery of a cargo comprising:
delivering, to a donor cell type, an engineered delivery vesicle generation system of any one of claims 1-28, wherein expression of the engineered delivery vesicle generation system in the donor cell types results in generation of the engineered delivery vesicles and thereby delivery of the engineered delivery vesicles to one or more recipient cell types.

63. The method of cellular delivery of claim 62, wherein expression of the engineered delivery vesicle generation system and generation of the engineered delivery vesicles, delivery of the engineered delivery vesicles to the one or more recipient cell types, or any combination thereof of, each independently occurs in vitro, ex vivo, or in vivo.

64. A method of cellular delivery of a cargo comprising:
delivering an engineered delivery vesicle of any one of claims 28-53 or 57 to a cell.

65. A formulation, optionally a pharmaceutical formulation, comprising:
a. an engineered delivery vesicle generation system of any one of claims 1-28;
b. an engineered delivery vesicle of any one of claims 29-53 or 57;
c. a cell as in claim 60 or a population thereof,
d. a co-culture system of claim 61; or
e. any combination thereof.

66. A method comprising:
delivering, to a subject,
a. an engineered delivery vesicle generation system of any one of claims 1-28;
b. an engineered delivery vesicle of any one of claims 29-53 or 57;
c. a cell as in claim 60 or a population thereof,
d. a co-culture system of claim 61;
e. a formulation of claim 65; or
f. any combination thereof.

67. A formulation comprising:
an engineered delivery vesicle generation system of any one of claims 1-28; and
a buffer optimized for RNA binding and/or encapsidation.

68. The formulation of claim 67, wherein the buffer comprises an optimized concentration of a salt, optionally NaCl, and an optimized concentration of ZnSO₄.

69. The formulation of claim 68, wherein the optimized concentration of NaCl ranges from 0 mM to 1 M.

70. The formulation of any one of claims 68-69, wherein the optimized concentration of ZnSO₄ ranges from 0 μM to 1 mM.

71. The formulation of any one of claims 68-70, wherein the optimized concentration of NaCl is about 1 M and the optimized concentration of ZnSO₄ is about 0.5 mM.

72. The formulation of any one of claims 68-71, wherein the optimized concentration of NaCl is about 0 M and the optimized concentration of ZnSO₄ ranges from about 0.05 mM to about 0.5 mM.

73. The formulation of claim 72, wherein the optimized concentration of ZnSO₄ is about 0.05 mM or about 0.5 mM.

74. The formulation of any one of claims 67-73, further comprising a pharmaceutically acceptable carrier.
